SEQUENCE LISTING (287 sequences)

SEQ ID NO: 1 - DNA, 1000 bp, Homo sapiens
gctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctattctcgcaactgacaatg

SEQ ID NO: 2 - DNA, 1000 bp, Homo sapiens
tgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccgggctcccgctccggctcccgctcgggatctcgctccggatctcgctccgggtcccgcagtgggtcccggagaggaagctttgacgccacagggaattcttcctactcttattcatactcatttagcagtagtagcattgggcac

SEQ ID NO: 3 - DNA, 1000 bp, Homo sapiens
tttatttttatttttatttttttgagatggagtcttactctgtcaccaggttggagtgcagtggtacgatctcggctcactgcaacctccagttcctgggttcaagcgattctcctgcgtcagcttcccgagtaggtgggactacaggtgtgcgccaccacacccgactaatttttgtatttttagtagagatagggtttcaccgtgttggccaggatggtctcaatctcctgatttcgtgattgagccacctcggcctcccaaagtgctgggattacaggcgtgagccaccacgcccagccttagactgggtaatttataatgaatggaaatttatttggctcccagttccaaaggctggaaagtccaagattggaggtctgaatctggcgagggccttcttgctgtcatccattggcagaagggtgagagcaagatagaaagggggcataatcatccttttaatcagcaacccactcttgtgataatagcattactctattcaggaaggcagaggcctcatgacctgaatcatctctcgaaggtcccacctctcaactcttgcatttaagggttacgtttccaacacatgaactttgggggacacactagaaccatagcactgagttttacttgaattaataatgaaaacatctggtttaaagagcacacaagagaaaaacagcccaaagccctgttgtagacattagtcctttctcctctttaggccaactgcattgactccacagcctcagccgaggccgtgtttgcctccgaagtgaaaaagatgcaacaggagaacatgaagccgcaggagcagttgacccttgagccatatgaaagagaccatgccgtggtcgtgggagtgtacaggtgagcaggggcccagcaatacaccaagacagacatctctgtcccttgcaccccgagtgccatgatcctggggaccctccttcatcacctatcttcctctcacaggccacctcctaaagtgaagaac

SEQ ID NO: 4 - DNA, 999 bp, Homo sapiens
taaaggctggtacttggaacctgcaagccgtgcatttggaacctcggactcaagtgcctattacgtaattccacagcgtcccggcctccaggccgtttcccgagccctccagcggagcgggggataaggttaccacgcccgcggtggccggggacactctgagtttcgcgtgtggcttttagggacgtttatatttgaatttccctgaaccgccgagtgtgggcggtggcgcagatccgtcccggaaacctccgggctccttcccgcctttctcaggcccggcccctccaaggggtccccgcggggcggcgggagggccctgggcccagagccgcgcgggtgggcagtcccaggcgtccttccttacagccctgagcctggtccgggaaccgcccagccgggagggccgagctgacggttgcccaagggccagattttaaatttacaggcccggcccccgaaccgccgaagcgcgctgcctgctccccattggcccatggtagtcacgtggaggcgccggggcgtgccggccatgttggggagtgcggcgccgcggcccgcgccacctccgccccccgcggcttgcctccagcccgcccctcccggccctcctccccccgcccgccgctccgtgcagcctgagaggaaacaaagtgctgcgagcaggagacggcggcggcgcgaaccctgctgggcctccagtcaccctcgtcttgcattttcccgcgtgcgtgtgtgagtgggtgtgtgtgttttcttacaaagggtatttcgcgatcgatcgattgattcgtagttcccccccgcgcgcctttgccctttgtgctgtaatcgagctcccgccatcccaggtgcttctccgttcctctaaacgccagcgtctggacgtgagcgcaggtcgccggtttgtgccttcggtccccgcttcgccccctgccgtcccctccttatcacggtcccgctcgcggcctcgccgccccgctgtctccgccgcccgcc

SEQ ID NO: 5 - DNA, 1000 bp, Homo sapiens
aagtgatggagcttcctctgctagccctttgtgagccaatggtaaatgggtgctaaataaaacaactaggtcttgagatacattaattgtaaatgtcacagaaccagtactttcctcaatgtggctaagatagttgatggttcctttttcttctgcactggtcagaccatatctgggctatgatgtttgcttctgggccacacactttagagggaagacaagcagcatgttggagtctgtttaggcgagagatccggcaggggaaggagtcttggtgaatgaggggtggaggagctgcaggatgggaatagaggcctgaactgctaccatgacatattcaaaaggctgccgtgtgaagccaagttatgcttgtcttttgtggtcccagttgatcacattaagacctcatggggccatagaaaagctagagggagactgatttgggttattcataagaagaactttaagtctgttatctgagggtagaatgagaggcatgttttagattctttagattctttactcttctgacaatcatgtgtttttgtagctgtttccttgtggtcatattaattctggtaccacttcatgaaccttttattacccatctttgttttctttttttttttcccttcttaactccctgtttaatttgtggtgagggtgaaagaggagataaagaaaaaaaagggtcaacttgtaactttgccttttcttttcttttcttttcttttttttgccctcagtaactgagggcaaacccatcagacaaccagagccataatttgtggtcaccgctgaaatttaccttggaaactcggttagtatggctgtgaagaggtataccccagctccttaacacagagttaatgcttaatctaaggttttaagtttcttagaaaagaaaaacgtgtacattcttttgtttcttaaacatctaattttgccctcctcctcttctcttagaggcaattgcttttggatcgttccatttacaatg

SEQ ID NO: 6 - DNA, 1000 bp, Homo sapiens
cctctgcctgctgagttccagtgattctcccgcctcagcctcccaagtagctgagattacaggcacacgcccccatgcctggctaattttttgtattttcagtagagacggggtttcaccatgttggccaagctggtcttgaactcctgacctcaggtgatccgcctcccttggcctcccaaagtgctgggattacaagtgtgagccacctcacccggcccctctcagagccttttctacctatatgtgatgtgaatctccaatgagaatctaggaggcagagtttgactacagaccagtgtcacacctgtgtttctgggaacactgttacagccacctggctaagtgctcaggagtcagacctgtgtatgaatccaggctgtgacctcagtagctgcatgaccctgggcaagttacttcacctgtgtgcttcagttgcctcccctgttgggagaactaaataatcccagccctgtgggaggccgaggtgggaggattgcaggaggccacatttgaccagcatgggcaatatagtgagacccccatctctacaaaaaaaatttatttaataaaataaaaatgaaaaatgagcgtttaggacaacacggcacatgggaaacgcctagcaagtaggagtcactccgagcgtgccgactatgcccacctcggccccatcacacagggtgcagctctagcccgaggggcagctccctgagcccctctctccgcctggcaggaatgcttcacgccattcgtgaacggcagcttcttcgagcacgacgggcagccctactgtgaggtgcactaccacgagcggcgcggctcgctgtgttctggctgccagaagcccatcaccggccgctgcatcaccgccatggccaagaagttccaccccgagcacttcgtctgtgccttctgcctcaagcagctcaacaagggcaacttcaaggagcagaacgacaagccttactgtcagaactgcttactcaagctcttctgc

SEQ ID NO: 7 - DNA, 997 bp, Homo sapiens
tttaaatgggcccacactaaagttagagaaccacaggctcgctcacaaccctgacttctccatgtcagttccgatctttgcgaaccgcagacagggaaggtcttctctcaggggtcatgcccgcggccgccctccacggcgaggtccgcactcgcgcagccggccccgcggccgcctcacctggtcgcacactaccacgtcgaactcctcgtcggcgaggaacagcacgtagagcgccaggaaaaccatgcgcacgtaggcgcagacggcggcgccgcggccgccccagcccaggcctcgcggcagccagtccccggcacagcgcaccggtagctcgcggctctcggcgaaacagtggcccgggtcgtagtgcgctgtccagatcttcacgctacacccgcgcgcctgcagcgccagcgccgcgtccaacaccagccgctcagcgccgcccacgcccaggtctgggtggaggaacagcaccgacggcttgggaaccgagtcccgttcccggccctgctcctccgccatggccctggagccgcaactgcaccccgcaccctgatgggggtcttctgcgcaagctccgcgctcgtagctcccagctggccactgcgggccgaccccgccctgccgtacgtgcgtcagttaggccacatcagcgcaaatctgtgagggtctagtaactgcctgagaaaatatcttgtctgaccccggttatatttttccttcggtagggattggactttctgaaggacgttgtgatccaaaggaaggaggccggaggtctctacttcccatacagcaggtaactaagttgtctgtagcagactgtctacaggcatatcgtgagacgacccaggcgtccctggggtcagagaggaccttgcctgcaagtccgggggcggggcctgagtcagtctcgccagctgccggtctttcgggggctccgtaactttctatccgtccgcgtcagcgccttgccacactcatctccaat

SEQ ID NO: 8 - DNA, 999 bp, Homo sapiens
agccttggcagtcggcgccggtgaacgagagcaacgcttctgaccctgccggagctcctcggagatgaaagccatgacgcgccttgcagaaaatgcattccgccttccgtgggaacaacgccgaggcacgcggtgacagccgtgaccatgctgtttgcccagtgaaggaaacaactgtcgggtatcggctctgccggcctttccagccgcactcatgcatggggctcaccccatgatgtgcgtggcttgtcgaggagcaagtggacaagtctcttaaggaaagctttggtgcacaggcgctttctccttgggggcgaattctgccagaccttggataaaaacaaacaggaagactcgcacggcagcggaaactgtcttccaagttacttgggttacccggcttttccttccgcgcttggggtcgggaccccggccgctcgtcccgccccctcccccgccgcggccccgccccctccccgcctcgcctcgcctcgcctcgtccagccccgcccccgccgggccgggcatgctcagtgggccgggccggcaggtttgcgtggccgctgagttgccggcgccggctgagccagcggacgccgcgttccttggcggccgccggttcccgggaagttacgtggcgaagccggcttccgaggagacgccgggaggccacgggtgctgctgacgggcgggcgaccgggcgaggccgacgtggccgggctgcgaaagctgcgggaggccgagtgggtggccgcgctcggagggaggtgccggtcgggcgcgccccgtggagaagacccgggcggggcgggcgcttcccggacttttgtccgagttgaattccctccccctgggccgggcccttccggccgcccccgcccgtgccccgctcgctctcgggagatgtttatttgggctgtggcgtgaggagcgggcgggccagcgccgcggagtttcgggtccgaggagttgcgcgcggcgctggagagagacaagatg

SEQ ID NO: 9 - DNA, 1000 bp, Homo sapiens
gatgataggaagtatttacagaactttatagttagtaactgactggttaatttttcaaactgatttttactcaactgaattagaaaaggactggaaagaaagtaaagatcccaacgacttgagggaacaagttggacaaccaaggactttgtctaaattgtttttatttagactaatgtggttctagttctagaggattcatactggaatcatcggtttaatattacgctatttgaaaggcagcatagtatagtacttttggaaaattggcctgagggtgatgtcttttggaatatttgtgaattcactatgaagcctaattccttaaaaatgacctccttcactcaattatcagtgttcttggtttgcctgggagtgaaaagagatcttaaaatctttttggttttagttacataattgactgatgtaatattatgtaatgatggctgtacacagtgtctcatgccctataatcctagcactcatttgagctcaggagttcaaggccagcctgggcaacatggtaaaaccctgtctgcaccaaaaataaaaaaaaaattagccgggcatggtggcatgagtctgtggttccagctactcaggaggctgaggtgggggaggatcgcttgagcccaggagtcagaggttgcagtgagctgagattgtgctactgcactccagcctgggttacagagaccccatctgaattaaaaacatatataatgtaatgatctgcctcctttgttaacttgacttttgaaatgggattgtcagtagtatgatcattgttttcttggatgccgactgtgtgtaaagtgttacattttgaattaaatgtcagaatgggtgaactttactaagattcaattctttgaatacaaagagcattttattttgaagttagaatactaattaaatgcttatgacactttaaaaaattattttttttttctttcagagaattgtaagtgcacagtccttggctgaagatgatgtggaa

SEQ ID NO: 10 - DNA, 1019 bp, Homo sapiens
gagtgttctttttttgatgaaagcaataagaggactgcggaagagctccctgtcaatgtaccgctctacaccagtgtattacgacagttcgtacacaacagtctgtagaggccacctgtctctccctgctgcgttaggaattcaggggagcaggtggtggcagtaagggattttgagggaacggaaatcggatcttgacccagatctgggccgccgataatctcctactgcgctcagactgctgtggaggtgttaggctgagcccgatgccggcaggcaagggaggatgggcggcttgggcagcgcctttgcagacgtggccatttcgtgcctctgcagcaccgccggggggcgcaagagcgcgcgcccggaattgctcattcatcctgtgccgcagagccccgccccttgtccctgcggacagacatttcttctgcgctggtctggccacgtgcttcctgtgctaggagctgcccggaaatgtgaccacctagtctaaagtgggcttctggggcctgagcgctggatggatgcccaccttcctgtcttggtcctccaaaggaggaagctgtgactgagctgtcttggtctggaaggaggccttcccggtttaggatgggaaggtaacattcattaaaagcaacgtagactatagtgtagctgttctcaaaagtagtacatcttagaaaaggatctttagaaaagatcgctttagaaaaggaaattcgttttcagattacgtgagtagcctaggtaacacagccagacctcatctccacaaaaaaaatgaaaaaattagccagcttggtggtctgtgcctgtggtcccagctgctccagaggctgaggtggggggatgactggagcctaggctgcagtgagcctagatggcatcactgcactcaagactgggcgacagaccttatctctaaaaaaataaagattgcatgagtattttgttccacttgacagtcatcaatagattggtttaaattgtgatatcttttttacttaccgcag

SEQ ID NO: 11 - DNA, 1000 bp, Homo sapiens
agtgtacacgtcggttgcctaacaaccggcagcggactcctttggctatggtgagtgctgccgctctgtcgcgccccacgcccagcggctgagcccttcccaggccccagctcggccacgccgcctctcctgcgcctttctcggctttagcgcaccagtctttcctttgagctctttcccgggagagtagggtacagacgctaaccaacctactgggcctgcgcttcgctgctgcctcctcttcacccacaacaggcccacacccgcccctcgggctccccttgcattccctgatcccctcggtgaacctccctgggcaaaaaagactcgaatccctcacaggcccctgactctctcagaccttggccccctggcctcctcattctgccaaaatttccacacacagctgggaaaaggtgtatcgtgttctgttacaatgttttttacaatttaaatacaggggcattcaggggatgctttagcttcttggagaaggcacgaaggtgccagatggattgagttttattactcggtactgacacaatatacatcttaacaacttactaatatttggccaaatctagaagtaattgggaagtcggagtacttactgggaaggggagtactgggagtgggcatcgtaagactgttttcagaaccaggatccccacgttcgggaagagctgattatctctagttattcacagtcatcttcttgactggctctcttttgggatgattttcagtaagaggtgtccttactgaacaagtgatctccctgcttctagaaacaacagtaaagaaaagggaggggtagaagttcatggaaagattcattgttcttgccagtaaaccctgttatctttctttttaagcaaagctttggaattggttatataaatgactttaaaaatacatatgggtgtctgtaactatgttatgtaaatatggactgtatgatatattattgtgcttgtgtattgcataacatataaaggcc

SEQ ID NO: 12 - DNA, 1006 bp, Homo sapiens
cggtttttcctacaaggaatccagttgaatacaattcttcctgacgccagaggtgagaacccacaatctctgcgagccccgcccccgcccccgcccgcgcccccagggtattctggagccactagacctctgtgtgtgttgcagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctggtgagtgtcaccgagggcagctgtcgcggggtgtggaggacgtgcttcagactccgcctgtggacgtttagtcgcttccgtgtgggctggggcgacgcccctgttcctctgcaaggagctgtttcttcttgccggtctgagattctagaggtaactccccctgctttagagaggcccagcgtgtctctcagctgggagcccctggtaccatttgagagtaagggaatcattttaagaaacagtggtggcgcttctcccatgaacgttgaatacaacactgtcatctgatgtgcacagaggccctggacgcagcacagctgtccggccacagcccctgattccagacgggggagagacgtgtgtggtttcctcgccgtgcagagagcaccagtctctgcagcggcttccccaagtgacaattccagctagagctggaaccttccgccagggttcctctttgccttttggctgatgtgggggagtggtggggagagaggcctgtttgcaggcccctgtgtgagcagagccctgacaccatccgtctgtcttggcagtggaggagtgtctgctggacgagaacagcatgctgatccccatcgctgtgggtggtgccctggcggggctggtcctcatcgtcctcatcgcctacctcgtcggcaggaagaggagtcacgcaggctatcagactatc

SEQ ID NO: 13 - DNA, 1000 bp, Homo sapiens
aaatgtacaattaaattattattgactatagtcacctgttgtgctagcaaatactaggtcttattcaaactatctatttttgtacctattaaccatccccaccttccccccgccactactcttcccagtagtctctggtaaccatccttctacctttatctccatgagttcaattgttttgatttttaggtccctcaaataagtgagaacatgcgatatttgtctttctgtgcctggtttgtttcacttagcagaatgacctccagttccatccatgttgttacaaacaacaaactctcattctttttgatggctgaatagtactgcattttgtataagtaccacattttctttatccatttatctgttgatggacatgtagcttgcttccaaatttaagacattatttgtaagaaccaaaaactagggcctggcacggtggctcacacctgtaatcccagcactttgggaggctgaggcgggcagatcacgaggtcaggggatcgagaccatcctggctaacatggtgaaaccccatctccactaaaaatacaaaaaaaaattagcctggtgtcgtggcgggtgcctctagtcccagctgctagggaggctgaggcaggagaatggcatgaactcgggaggcggagcttgcagtgagccgagatcgcgccactgcactccagcctgggcgacagagcgagactccgtctcaaaaaaaggaaagtactttgacaacagaagtctgtgttgaaatctaaaacctctattgcttgttcttatcaaagtagctatctaggtagcatgttctctgatgcaggagctgacttctgttttttcaaacctctttccctttagacgttttggaataatgggactctacaaaggccttgaagccaaactgctgcagacagtcctcactgctgctctcatgttccttgtttatgagaaactgacagctgccaccttcacagttatggggctgaagcgtgcacaccaacac

SEQ ID NO: 14 - DNA, 1000 bp, Homo sapiens
gttctgagcgcctgtagtctcagctactcaggaggttgaggcaggaggattgcttgagcccaggaggtctaggttgcagtgagctgtgaccacgccactgcactccagcctgggtgagagagcaaaaccctttctccaaaaaaatttttttaaaaatggcatgtatattgttcgtctaaagactacaaaaaagttacaattattgtaagtaaataagtcagtaataatcaccacatggagaacaactgttagcatttctgtgtatggtgtgcttgagcagatgaggaaacagttttaagtctgccgttaggcacacaaacctgggagactgaaaatatttgttgtccaaaattaccattaattaaatgaaaaatgcatcgtttaacagaaaacttcaaataatactaaattttaggatctgttctaaaagtttcccatttattaataacataatttttctcaaaacaatgttaatccaagtagcctggctacagaacttgaactcgagtcatctctcaggttgcctgggtttgaaactcggctctgtcacttctgtgacaataactgtgtgacctcaattaagcttcttaacatctctgtgcttgtttcctcatttataaggattgtaataatatctcattttataggattgttgagaagttaagtacttaaagcactgcctggcacaaacttgaagctttgaggttagcctttaaaaaaaaaagtaattttggactcacatcctatacctttgaatttcctttaaaagatagtgacataatctaaagggcattgaaggtctagtagagtttaagataatcatgctataggcaatatttaaagtgattaatagtaaatttgctttactgatttgtatatttaattcataattgtttctttacaggtttctttacctccagaaagaagaatattggccccttgaattctggaagttcattgaagagtctgaaattagggacttatttcaaatttggacatg

SEQ ID NO: 15 - DNA, 1000 bp, Homo sapiens
tacaatttaagggcgtaccccttaaaaactcagtcatcaagataaataatatttcactacagtatcttcagaaaacacaattaatgtaaaaattatgatgagtgaaatatcaagtatttaaataaagataggatcagtaacagtgctgtgcagagtttattggaacaatgctggccaggggcccccttcttggattttctctacatctcatcattcagactgtggagtgctcctgttccagctgtccctttgcccagctaagggaagaaaagtcttccctccccaacactgtttcatgcaaatgctataaggaaaaatctgctcccaaatagcaccatccaagaggttctgcaggatgtcacagtgctagaaagtgctaactacacatgatgccactgtctcctcaatcagctcagtaatcttgtcctctttctccaaataaattattccatggatacattcattggctgattgatggagtcatttaatctactagcatttagggaggatctattattcaccaggccctggcctaggtgctggggattcaaaggtgtataaccctcacacagccctgcatcctaaggaacactgctgggcttggcttgctgaaacttagattgggaatcacagtcataaatagaaactccagagggaaagctctccaaatttggggtcatgagctgctgaacccactgggcagagctctggggtgctggggtgggttgtcaggcatgactcacctctgctcccctctccaggtatcatcatcatgatgacgctgtgtgaccaggtggatatttatgagttcctcccatccaagcgcaagactgacgtgtgctactactaccagaagttcttcgatagtgcctgcacgatgggtgcctaccacccgctgctctatgagaagaatttggtgaagcatctcaaccagggcacagatgaggacatctacctgcttggaaaagccacactgcctggctttcgcacaattcactgc

SEQ ID NO: 16 - DNA, 1000 bp, Homo sapiens
gatgatgatatcgcagcgctcgtcgtcgacaacggctccggcatgtgcaaggccggcttcgcgggcgacgatgccccccgggccgtcttcccctccatcgtggggcgccccaggcaccaggtaggggagctggctgggtggggcagccccgggagcgggcgggaggcaagggcgctttctctgcacaggagcctcccggtttccggggtgggggctgcgcccgtgctcagggcttcttgtcctttccttcccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccaggtgagtggcccgctacctcttctggtggccgcctccctccttcctggcctcccggagctgcgccctttctcactggttctctcttctgccgttttccgtaggactctcttctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggcgcacagtaggtctgaacagactccccatcccaagaccccagcacacttagccgtgttctttgcactttctgcatgtcccccgtctggcctggctgtccccagtggcttccccagtgtgacatggtgtatctctgccttacagatcatgtttgagaccttcaacaccccagccatgtacgttgctatccaggctgtgctatccctgta

SEQ ID NO: 17 - DNA, 1000 bp, Homo sapiens
tagtagtcagttgcgagtggttgctataccttgacttcatttatatgaatttccactttattaaataatagaaaagaaaatcccggtgcttgcagtagagtgataggacattctatgcttacagaaaatatagccatgattgaaatcaaatagtaaaggctgttctggctttttatcttcttagctcatcttaaataagcagtacacttggatgcagtgcgtctgaagtgctaatcagttgtaacaatagcacaaatcgaacttaggatttgtttcttctcttctgtgtttcgatttttgatcaattctttaattttggaagcctataatacagttttctattcttggagataaaaattaaatggatcactgatattttagtcattctgcttctcatctaaatatttccatattctgtattaggagaaaattaccctcccagcaccagcccccctctcaaacccccaacccaaaaccaagcattttggaatgagtctcctttagtttcagagtgtggattgtataacccatatactcttcgatgtacttgtttggtttggtattaatttgactgtgcatgacagcggcaatcttttctttggtcaaagttttctgtttattttgcttgtcatattcgatgtactttaaggtgtctttatgaagtttgctattctggcaataaacttttagacttttgaagtgtttgtgttttaatttaatatgtttataagcatgtataaacatttagcatatttttatcataggtctaaaaatatttgtttactaaatacctgtgaagaaataccattaaaaaactatttggttctgaattcttactagaaggtggtcttttgaagttagtcctttcggtacttctcagatgcctgtcatgtacccgatggagtccttggaaagaaggcctgtgtaaagaggccagcctggaggtcaataacctgttctagtttattctggacattgagtaccaagtagcattggcaaa

SEQ ID NO: 18 - DNA, 1000 bp, Homo sapiens
tgaagttcagccctgagcggattgcgagagatgtgtgttgatactgttgcacgtgtgtttttctattaaaagactcatccgtctcccatgtctgctgctcattcctccccttgacctgctgacacagggagcacgcacccttggtcaattttgcggggttgggtaaattctcactcggtcacagagcgcatgctccgtttctagctgcctttgcgcagcggcagcctggatttcggttcttgggtgggattggtagctcgctgcgcatgcgtgcaggtaagcggccatctcgcgcaggcggagtgtcagtgtgggtcacgtgaggggagcggagagggagggatgggggcggagtccagggcgtgggggggccggtttgttgtggtcgccattttgctggttgcattactgggtaatcggggccctggcttgccgcgtccgccggataccctcagccagtgggcaggtctgagctcgggctccccgagcagtttgagtccccttgcccgctccttcaggtaacggcgcggggacgggtggggcggcaagcggtcgcagggaggtgggcaggacgggatccgccctgctcccgtcgccgtgagacttagcacgaggccaagggaggagaggaggggggtggcaggcaggtgcgggccctgcctggctattcatagttgaattcctggaaccggccaagcccgaggaagcagttgcaggagggaggctgggagggggtagccgggccccactcccgccctttgtttgggctcagctccgcgggccgcttcttcgtcgcctagcaacagctgccctaggctgtgattggctgagctcttggcaccagcgaccaatggtacagttgttgccatggcaggtgccgattgccaagctcagtcgggccccgccttccggtctcagcaggcccaggagggcctcctgggtggggggcgggacgccgggtccctaggggctggtggtcactcagggtggggcgtgtcgc

SEQ ID NO: 19 - DNA, 997 bp, Homo sapiens
gcgactgcgacccccgtgccgccgcggatgggcagccgcgctggcggccccaccacgccgctgagccccacgcgcctgtcgcggctccaggagaaggaggagctgcgcgagctcaatgaccggctggcggtgtacatcgacaaggtgcgcagcctggagacggagaacagcgcgctgcagctgcaggtgacggagcgcgaggaggtgcgcggccgtgagctcaccggcctcaaggcgctctacgagaccgagctggccgacgcgcgacgcgcgctcgacgacacggcccgcgagcgcgccaagctgcagatcgagctgggcaagtgcaaggcggaacacgaccagctgctcctcaagtgagtgctagctggcggccgcgttagcgccaaggaggggcgggggcgcaaccgcggcgaccagctcaccgggttctgccgtggggagggagcagaggccaggatgcacgcgtccttctgaaggaacagggtctcggtctccggaaaggagaaagaatctagagttcatagcggagcaggggtcgcggagggggctcgagctgtagcgctggggggccgtgatgcccatttctagattttggatacccgctgggacgtggtaagtgcgcgcctgggactgccgagaaggagctcccgctttcgcactcgaatccggggagccggcgcggagaggcggcccctcaggccccaggtgcggggagctggagcgcgagcgcgcgctcgcgtgcgcgccccagtttccggccggcgcgagacaaagcgtctagcggatttgcagtgccgggatgggcggccggggaggactggcagcccgcctctagaatgaatgagcttcgcgcgggcagagagaggaaggggagggaccttcccgcagcatccgcgtctcctgggggtgggtcccgctttggcgcgctcagtcttggccctgtgacgttttgcgaagattctacgcctgctttaggcgggagagagaggcggag

SEQ ID NO: 20 - DNA, 1000 bp, Homo sapiens
gcacaaaggacagggctggaggatccagagaggtacctgttcgtcgatcgtgctgtcatctacaaccctgccactcaagctgattggacagctaaaaagctagtgtggattccatcagaacgccatggttttgaggcagctagtatcaaagaagaacggggagatgaagttatggtggagttggcagagaatggaaagaaagcaatggtcaacaaagatgatattcagaagatgaacccacctaagttttccaaggtggaggatatggcagaattgacatgcttgaatgaagcttccgttttacataatctgaaggatcgctactattcaggactaatctatgtaagtatttcttccaaataatcatgtgaagtggtagctaggaattaatgtaaattatacatcttgtcataatcaaatgagaatgtggaatacccaaactctctgtttaacatttctatttctctttaagatagaaagatttgttgcttgcttacccatgtcttgcttttctttgaatcttaacacattaagtttaaataatacaggctgcaattacatataataaaatggcatttgaagacttttgtagtggtcttctggagcataataaggtgggagagagcatgtaacaggaagaccagaaggtttaataaggtaaagagagttgcattaattggacgcagacagcaaaacggtcaaaaatcaagtgcatacccaagagtaaagtggaggggctgtaagctgagaaatttctgtggacagcatgaacagcttcactggatgtagtagggaagtaggaaagatgaatgctgaggtttttaagaggaaacaattagggtaagattgaggctggctggggtcgtcctgtggttagcagctgacatgaatgttggagtcaccgactttgtcactgaccatgtagaagaagttattgaaaatcataagggataatgtagagagggataatgtagagaggaaaaatgtaagccagatacta

SEQ ID NO: 21 - DNA, 999 bp, Homo sapiens
gtgccctgcccctgtctctgccccccttccccagccagcatcaccaactgcgactgtgacctagagacttcacccgggggtgaaggggtaaacccgactgaaactggaacccttgtcctccgctggtgcgggatggacagagggccgtgaggggtccccctgcttgtcttcacccctgccagagcctctgggccccctcctccctcctgtagctctccctaggctgcccactctccatcctccccaggggtagaggctgggggctccaccccagcccatgtacgtccccacgaactggcctggccagcaccccacactggagccatctcttcctcatatttcagcagtgcagccggggggcagggaagggcaggcagggtctgttggggtctctttttatccttattcctcccccgacctaattgtctttgttctgtgattattgggggacacccggctccctccagacaatgccagcataaatccatccatccaaaggcagagaaccaaaggggccatggaaggttctctgtgctcctcctacccttccagtgccctaggcctggcgactgcccctgccttttagacccgccctccccttttatacctgctcttgttctactgagaaaagcctctccagcaataatgttttctagtcacttcctccgtctccgggacggcgtgcctggacactgtaccgactttgatagatttctacactgaggtttgaattcatatcgcctgagttgcttttacttctctatacaaaatgattttgaagagattttaaagacgttcccttttgtattctcttcctcatccaccgccactgggcctgtcactgatggtggctctggtgtgaagtttgctttgtactgagggttggggtggggaagcaatttgtattttattgtttcttagcacaagcaggtgaactgggagcagctctgtgactccccctctttcacttcatagctcaccaggactgttttataaact

SEQ ID NO: 22 - DNA, 951 bp, Homo sapiens
atggtatggcggcccttccatgatccccgcctctcccagaagccctgactcctcctgctttgcgccgtgcttttcctctgtagctcccttgcttcccccagcctcgggtgtgggtgtctaggccggggttctggggcaggcctgccgcgctcacccgtctgtctgcttgtctccctctacagcctggtccgacccccagtggcactaacgtgggatcctcagggcgctctcccagcaaagcagtggccgcccgggcggcgggatccactgtccggcagaggtaaggaaccctgcagttcgttcgcttccagactcggagataggacccagaacctcgctgattctggggtggagaccctagcatgtgaagattgacaaaggcaaaatgagcttctagtgacgtggccgtgggagtagttaaaggccttttgggaggaaggcgacattttttttctcgttgctcagtttagggcactactcttaaaaaaggaaagttaacaaactggaatagagtcagagataactttgagaaaaccgatgtcattaaactggtgtctctggacctgaggtttgcactcacatttccatctggcggccccataagcaatctgtcctacagataactcgtcctacacaaaacttagtctcttttcagctcagctctctcactctcaattatatctccttacttccatatggcactgttgtacactcatttactcagagccagaaacgtcagcgtcatcttggatttttcttatgctctttctctctctagtcatatgccagactttaaactctgcttgaaagctttctcataagctctttccttttccctttctactgctttgcatttgctacttaacccttttcttcaggctgtttgctttccagtccatcgttcgctctgctgttactcttctgcgtagtttctgttacttgttgctgaac

SEQ ID NO: 23 - DNA, 1001 bp, Homo sapiens
agtgccagagctgcggccgcaaaggtgagaacctccgcggccgccagggccagaccgggccgaccgtcgccgcccgcccaccggcatctggcccgcgtcccgccctccctcgctggcggctgtctgggccccggggcggcggggtgggcagggctggcgcggggccgcgggccgcgggccgcgggccgcggggagcccctcgggcgggggcggcgcgggccgcactgggggcggccggggagggggctgcgggcgcccggccgccgtactgggcaggtgcatagctgccggcgcctgtgcctggctgcggctcgctgagggcggggacacgcaacaggtccctcgcggagaaactcggctccagtgagggttcgggggctggaagccggctctcagcgggtcggggcttggggtgccacctcctgctggccgggagctgctgtctttggaggagtggttggtccccggcgaaaccctgtagtttcgatctgatgtcactccctgcggtatgcgcacgccagcgataaggctttgagactgcaaaacactccactcagcctgtgaggcgtagtaggtcgggttttctttcatgctgtattacttatttaaggtaactttgaaaataacctctttaacatttaataatttaacttgaattaaactttcacaagtaatacaaagtattcctacgaatggacaataagatgagcacttaaaaattagtaaaggccggtgagttcagccgaaaaaagtaacgtttttcctgttacttttcctatgtgctctgaaatattattgcattttcccattgctttgaaactaacttgtgtattacattaaaaagccaaagttcctgaaaaacagctaggatgctcctcccattttgtatattaattttttcatcataaaatagtacttgttatttcaaacaaaggaatacagaaatgtgaggagtaaaaaatctcccctttaaagaatatcaattcattacttcaaatagt

SEQ ID NO: 24 - DNA, 1000 bp, Homo sapiens
tgagaaacaaatgtcaacataataaaatctcagttaaaaatattttaaaaattcttggtagttgagcagctctggggtaataagggcaaatatgcttgttatgaactacactgaaatctaccaaagttaatgtttactttgtgtagatccatttgtctattttatttatttttcccagtgaaaagtgtattttgatagagaacttttcattctataaatacactatgagttactaaaatatcatggattttgtttattcctgaaacatagttacatagttaaactgtacatatgacatggcttatgttaaaaatacccagtgctcagttttgaaagataggcaaaaaaaaaaaagtataggagaaactgaagaatgtacacttttttagagggcacattttgctgtaaatctggaaatttgatagacttgactgtgtttgtgaaaactgagcattaaaggttttgattgatcctttctttccatttaatctctgagacgtaaatatgtgaggtgtgctgctgtgctgggttaacagcttccttccctttctgtgtagcagtcttgaaatgttctgtttaaatcagtaggcttaatgtgttctgggtatttatctccttgtattttaaatatatgtagttgcaaatagcaccaggaattagatttctgtacacccctaatctagccttgtgagcttcgctagttaatgtgtgctcactttccctccatttgttacgtgagagaatgcgtctgctgatcactgaagtgtcccttttagcttctgattcattgggttctgttgggcatctttaaatccaccttaacctgaggaatgtatgtgggcaaccaggccctgcatttttttatattctgaattttgcatgcttgcctgacttagtatttctgaattgatgttttttttaatggtataactatcttgattttcactgaaattatatggttctgtcactactctgtaaattaatccgaaacttttaaggt

SEQ ID NO: 25 - DNA, 999 bp, Homo sapiens
gagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattggtgagttgacctcagtaacctgagatcccaggatgctgggacaggaggtctgtccaggggcttctcttgtcactcactcactccctccgtccttctctccctcctccagatgaagttcgcactggcacctaccgccagctcttccaccctgagcagctcatcacaggcaaggaagatgctgccaataactatgcccgagggcactacaccattggcaaggagatcattgaccttgtgttggaccgaattcgcaagctggtaagcaccacatataaatatgcatttaatgtggtgtgatagttccagtgcaagttgggtggagtgactgacatcattcattctttggcacctaccaaaatgtggaataggctgcttgctatattaattggacttctaaatcagatagtccctaggttatggacagtttgtggatatgtctgttttgccaattccttgtgcttacatcagtgagatatggttcgtaatctaaaaagttgaaatagaaattctaagataatgtgtcctggcattaaaatattacatttttttattcccctacaggctgaccagtgcaccggtcttcagggcttcttggttttccacagctttggtgggggaactggttctgggttcacctccctgctcatggaacgtctctcagttgattatggcaagaagtccaagctggagttctccatttacccagcaccccaggtttccacagctgtagttgagccctacaactccatcctcaccacccacaccaccctggagcactc

SEQ ID NO: 26 - DNA, 1000 bp, Homo sapiens
caggtcaccctttcctttttaacaatgtaactcgagtctagacttgcggcaagtgtcaggaagcccattttggcctcaaagaaacaccaggcaagccaggcactccatccaaggtgacgtttgctacctatcgctatatctctccctatcccatatctatttcctaactgatttggaattctgtgggaagaggtatctgcttggaagtcttgagaaagaagcacaagggcttgttttgggagcccaaagtaacatgatgggagggcaggcctcctgatgcagttgctacctggactgctctgtggtcacatgactgttttaacaggaagacgccatggacctctgcagatgactggggctccataccatgataattgtcttagctacttaccattttctgggtcatcacagttaaaaagtcaccaaagttcatttttcctgtcccttccttatcaatttcacttatcattttcttaatttcttctttcttgggttcaaagcccagggccctcattgccacctataaagaaaacaggatcgtctcaataagcacatctaaagtagcaggagcaaaaataacagtgttgagagcattaagccagcatctgatgtggtcatgttaatatgattggggaacactatgacattgatctaattgatatttgcaatcaattttattgaataatactcaaagtaagtaaagacaggaaaacagcacagtacaacctcccctgttacttactgcagggagaagagggcaaaaggggtagagaggtgatagggagatgacaaggaagaggcagaggaaaggaagggcagtacggcaggaatgcacagagcttgccttcagttctttaacatctatggtgccagttccatccgcatcgaaaagatcaaaagcttcccggatctcctgcttttgctcttcagtaagctcaggcttaggactcattctttttcgctgagaacttgatgccatgtttgcctttttgaaattgga

SEQ ID NO: 27 - DNA, 1000 bp, Homo sapiens
tagcctggtgcacgcagccacagcagctgcaggggcctctgttcctttctctgggcttagggtcctgtcgaaggggaggcacactttctggcaaacgtttctcaaatctgcttcatccaatgtgaagttcatcttgcagcatttactatgcacaacagagtaactatcgaaatgacggtgttaattttgctaactgggttaaatattttgctaactggttaaacattaatatttaccaaagtaggattttgagggtgggggtgctctctctgagggggtgggggtgccgctgtctctgaggggtgggggtgccgctgtctctgaggggtgggggtgccgctctctctgagggggtgggggtgccgctttctctgagggggtgggggtgccgctctctctgagggggtgggggtgctgctctctccgaggggtggaatgccgctgtctctgaggggtgggggtgccgctctaaattggctccatatcatttgagtttagggttctggtgtttggtttcttcattctttactgcactcagatttaagccttacaaagggaaagcctctggccgtcacacgtaggacgcatgaaggtcactcgtggtgaggctgacatgctcacacattacaacagtagagagggaacatcctaagacagaggaactccagagatgagtgtctggagcgcttcagttcagctttaaaggccaggacgggccacacgtggctggcggcctcgttccagtggcggcacgtccttgggcgtctctaatgtctgcagctcaagggctggcacttttttaaatataaaaatgggtgttatttttatttttatttgtaaagtgatttttggtcttctgttgacattcggggtgatcctgttctgcgctgtgtacaatgtgagatcggtgcgttctcctgatgttttgccgtggcttggggattgtacacgggaccagctcacgtaatgcattgcctgtaacaatgtaataaaaagcctc

SEQ ID NO: 28 - DNA, 1000 bp, Homo sapiens
tgagacggcttgccatgaaaaattccgaagatgctcaagagggaggtttcctcctgagtgaagagaagtgattctcccttgactctggctcctgccaccacaaatgttaccctcattggcttgaaaagcatccaagggtgcacagggagtatggccaactggacctgttgtcaccttaattgtcatgctggctggttggattttggggtggcagttggactaatgtgaaaaaaacattgctgaaaacctaaaaatgaaagtttgtgagtgtttattggttttcttaagagaaatggactattttgctctcatgtgtaatgttttctatttaaatctttcttaaatataccagctgttctctttccctgaactctcccccaggttctaggacaaatttaataacatgtaattctcctcaaatacttttgtatgtctcagtgttggtgttttcctccctaaaactaacattagggctgtgccacgggcatgactttatttttgttgggcttttttttccctgcttaaggagaggtgtcttttttggatatgagctatttattttgtgaaatgaaaattgttcacccaaatgattctcttataaactatttgtaaatgtcacttattcattagtgtttgacataatttttagaatatttattttgaatcaatcctttcattacgaaagacttgaagttttgtgtccattcttacaagccctggtcagtcaagtcccaataaatggtcagcacaaaaaagatattcttgaaaattgctctttattaaggtattacgttgagtttgcaaccagatgggaaaaatcacaaaaatgagaaggggagcagatatcttgttgaggtctggatattatctccctttataaacttggtgtaggccgtatattttgaaaataaacatctggttgaggttatttcatttggaaaccttccttagagtcctgttgctcagtgtggtccatcagacggtagtactgccacaagct

SEQ ID NO: 29 - DNA, 1000 bp, Homo sapiens
gctagtcgaggcgccacacggccaaacgggccaaatacgggaaataaaatatgccagttcaaactagtacttctgggagagtccgctgttggcaaatcaagcctagtgcttcgttttgtgaaaggccaatttcatgaatttcaagagagtaccattgggggtgagattttcttttttccctgcttcatttaatcgaatcatacattgacagaaaatattttgatgttccaattgtgtgattatgaatagctaaataagtcatagagtgaagaaaagagctgttcagcttaccttagctttaaaacttgcctttatctttgttaaaatgatgtgtttttatatttgattgtttggttaatcccttgtggttatattgtagctaaaatacagataaaaaacatgagtagaatcagtgtctcaagtaataacatttgacctgatacctatttctagctaacaactagaagcttcttcaaattaatacagtacatgtttgtgctcaattatattggtttaaaatgaatagtaaaaaaggtaaccttaaattctgatctatataactgaagtatttgcaaataattatacttacttgtaaaattaaatgtaattaaatactgctagatgtacttttttttggtatttaagagatgggatcttgctgtcgcctaggctggagtgcagtgtccggatcatagctcactgcagactgcagcctgaaactctaggctcaaatgatcttcttgccttagcctcccaccaagtagttggacctaagggtgcatgccaccatgccaggcaagttttttttttttttttttaagagtcagagtctcgctgtgtttccaggttggtcttgaactcctggcctcaagtgagcctccagcctcaacctcccaggtagctgtgattacaggtgctccctgctgagatgcatatgtaagaatgctcctaagctgagggaagagaaaaagtgattaatggaaagggacataacaggaaa

SEQ ID NO: 30 - DNA, 1000 bp, Homo sapiens
taagcacaccctcctcactcttctccatcaggcattaaatgaatggtctcttggccaccccagcctgggaagaacattttcctgaacaattccagcctgctccttttactctaggggcctctgtcagcaagaccatggggacttcaagagcctgtggtcaggaaatcaggtccagccttccctgtagccagacagtttatgagcccagagcctcctgccacacacatgcacacatatctagcattctttccagacagcatcctccccgccttccaccttggtagatgcaaggtctatctctcccatcagggctgccaaagctgggctttgtttttcccagcagaatgatgccattctcacaaaccaatgctctatattgcttgaagtctgcatctaaatattgatttcacgttttaaagaaattctcttaaattacaattgtgcccaatgcagggtggctctggggggcaagtaggtggtacaggggattggaaacatgctccgcgcctccagagaaaagttgctcccgaggtccatgcccctggaacgtgttcctatcactctggctggttgggctggtccttagactgggtgcttatgattaaagggtcttggttagcccactttccctctccatgtggagatggaaggtagagaaggatacagtgtctatcctcaagttgctacggttcagtgagagaggcagacatctgaacaggcaggtaggattcagtgtgctcagtgcactggggatttggagagagatgggcttgctctctctgtgcacccaggagggccacgcacttaaaactgtgtttgtggatcagagaaggctttatagcacagggggcattcagatgagtcttagaggaagagaagaaacatggcaagcagattacatctgagccgtttgaattgtgtttttctttcttcccatgtttattttctaagatctacctgaacttagagactcaagatatttttttaggaaacctcctac

SEQ ID NO: 31 - DNA, 2740 bp, Artificial Sequence
  Made in Lab - synthesized insert sequence
cctctgcctgctgagttccagtgattctcccgcctcagcctcccaagtagctgagattacaggcacacgcccccatgcctggctaattttttgtattttcagtagagacggggtttcaccatgttggccaagctggtcttgaactcctgacctcaggtgatccgcctcccttggcctcccaaagtgctgggattacaagtgtgagccacctcacccggcccctctcagagccttttctacctatatgtgatgtgaatctccaatgagaatctaggaggcagagtttgactacagaccagtgtcacacctgtgtttctgggaacactgttacagccacctggctaagtgctcaggagtcagacctgtgtatgaatccaggctgtgacctcagtagctgcatgaccctgggcaagttacttcacctgtgtgcttcagttgcctcccctgttgggagaactaaataatcccagccctgtgggaggccgaggtgggaggattgcaggaggccacatttgaccagcatgggcaatatagtgagacccccatctctacaaaaaaaatttatttaataaaataaaaatgaaaaatgagcgtttaggacaacacggcacatgggaaacgcctagcaagtaggagtcactccgagcgtgccgactatgcccacctcggccccatcacacagggtgcagctctagcccgaggggcagctccctgagcccctctctccgcctggcaggaatgcttcacgccattcgtgaacggcagcttcttcgagcacgacgggcagccctactgtgaggtgcactaccacgagcggcgcggctcgctgtgttctggctgccagaagcccatcaccggccgctgcatcaccgccatggccaagaagttccaccccgagcacttcgtctgtgccttctgcctcaagcagctcaacaagggcaacttcaaggagcagaacgacaagccttactgtcagaactgcttactcaagctcttctgcggtaccagcggcggaagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgataggtgccctgcccctgtctctgccccccttccccagccagcatcaccaactgcgactgtgacctagagacttcacccgggggtgaaggggtaaacccgactgaaactggaacccttgtcctccgctggtgcgggatggacagagggccgtgaggggtccccctgcttgtcttcacccctgccagagcctctgggccccctcctccctcctgtagctctccctaggctgcccactctccatcctccccaggggtagaggctgggggctccaccccagcccatgtacgtccccacgaactggcctggccagcaccccacactggagccatctcttcctcatatttcagcagtgcagccggggggcagggaagggcaggcagggtctgttggggtctctttttatccttattcctcccccgacctaattgtctttgttctgtgattattgggggacacccggctccctccagacaatgccagcataaatccatccatccaaaggcagagaaccaaaggggccatggaaggttctctgtgctcctcctacccttccagtgccctaggcctggcgactgcccctgccttttagacccgccctccccttttatacctgctcttgttctactgagaaaagcctctccagcaataatgttttctagtcacttcctccgtctccgggacggcgtgcctggacactgtaccgactttgatagatttctacactgaggtttgaattcatatcgcctgagttgcttttacttctctatacaaaatgattttgaagagattttaaagacgttcccttttgtattctcttcctcatccaccgccactgggcctgtcactgatggtggctctggtgtgaagtttgctttgtactgagggttggggtggggaagcaatttgtattttattgtttcttagcacaagcaggtgaactgggagcagctctgtgactccccctctttcacttcatagctcaccaggactgttttataaact

SEQ ID NO: 32 - DNA, 2804 bp, Artificial Sequence
  Made in Lab - synthesized insert sequence
cgccaagcttgcatgatagcactttggagctggaaggatcctcaaaccatttagttgtagtggctaattttgtagaggaggaaaactagggcccagctaggggcctggtctcccaacttttggcttgacagtggcagctctaccctccctcaaactggttcccagggcactctggcagaggtagggcatggtgcagggcgactaccttgtgggtcagagcccccaaatgagctctcctggatcttgaagctcctaaaagaagcagcttccaagtagacctatcatgagcaaacctgggagaaggtagatacccaggctggagctgcttgcatggggcaaggcccgcatttctgcaaagctgcttaacatagcctggccatatttgggaagcagtgatgcccaaacatggatggtgttcttaatctgctggagggctcattaaaattagactgtctgtccgaggcgtccagagagctcagttgagtgcgtgtggggtctccccaggaacctatacgctccaccagagtatctgctgatactgctccagcacgcaattattgcagactagtgtgattcagaagatgattataggtgtaattggcacatggaattagattggaaagtggcttacaaagtaggtactgaagtggaaaacctgagagaattttaaaaatacatgttgtgtatgctggtatgtacatgtgtggatatttacgtccgtacatcctgtagttacaggagggaggcaaaatgcaaacggcacatgctgccttcacgccgccaggctttgcggctgtgtcgggccgcgccgctttacggctgctggactttgcggccttcctgaccactctctcctcgcagaactacattaccatggacgagctgcgccgcgagctgccacccgaccaggctgagtactgcatcgcgcggatggccccctacaccggccccgactccgtgccaggtgctctggactacatgagcttctccacggcgctgtacggcgagagtgacctcaagcttcgaattctgcagtcgacggtaccgcgggcccgggatccaccggtcgccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaatccaccccgaccgtccgccctcgtcttgtgcgccgtgccctgccttgcacctccgccgtcgcccatctcctgcctgggttcggtttcagctcccagcctccacccgggtgagctggggcccacgtggcatcgatcctccctgcccgcgaagtgacagtttacaaaattattttctgcaaaaaagaaaaaaaagttacgttaaaaaccaaaaaactacatattttattatagaaaaagtattttttctccaccagacaaatggaaaaaaagaggaaagattaactatttgcaccgaaatgtcttgttttgttgcgacataggaaaataaccaagcacaaagttatattccatcctttttactgatttttttttcttctatctgttccatctgctgtattcatttctccaatctcatgtccattttggtgtgggagtcggggtagggggtactcttgtcaaaaggcacattggtgcatgtgtgtttgctagctcacttgtccatgaaaatattttatgatattaaagaaaatcttttgaaatggctgttttttaaggaagagaatttatgtggcttctcatttttaaatcccctcagaggtgtgactagtctctttatcagcacacacttaaaaaatttttaatattgtctattaaaaataggacaaacttggagagtatggacaactttgatattgcttggcacagatggtattaaaaaaaaccacactcctatgacagctctttgcctgctccttgcttgatgttttctggaagtgtgtgtggggtcaaggatggtgtggggcacctgtatctactactaatgtttttgaccctgctgtgttcaatcttcttacacttcagtttcttcattttaaaaaggaaaaggtaatttcttcacagtctcctatgagttggacagaaagtcatggatgtgtaaaggacttcttgatgataagccccaccttcatttttccacggcaccaaacagccctgtgacaaggatccccgggtac

SEQ ID NO: 33 - DNA, 2807 bp, Artificial Sequence
  Made in Lab - synthesized insert sequence
cgccaagcttgcatgatagcactttggagctggaaggatcctcaaaccatttagttgtagtggctaattttgtagaggaggaaaactagggcccagctaggggcctggtctcccaacttttggcttgacagtggcagctctaccctccctcaaactggttcccagggcactctggcagaggtagggcatggtgcagggcgactaccttgtgggtcagagcccccaaatgagctctcctggatcttgaagctcctaaaagaagcagcttccaagtagacctatcatgagcaaacctgggagaaggtagatacccaggctggagctgcttgcatggggcaaggcccgcatttctgcaaagctgcttaacatagcctggccatatttgggaagcagtgatgcccaaacatggatggtgttcttaatctgctggagggctcattaaaattagactgtctgtccgaggcgtccagagagctcagttgagtgcgtgtggggtctccccaggaacctatacgctccaccagagtatctgctgatactgctccagcacgcaattattgcagactagtgtgattcagaagatgattataggtgtaattggcacatggaattagattggaaagtggcttacaaagtaggtactgaagtggaaaacctgagagaattttaaaaatacatgttgtgtatgctggtatgtacatgtgtggatatttacgtccgtacatcctgtagttacaggagggaggcaaaatgcaaacggcacatgctgccttcacgccgccaggctttgcggctgtgtcgggccgcgccgctttacggctgctggactttgcggccttcctgaccactctctcctcgcagaactacattaccatggacgagctgcgccgcgagctgccacccgaccaggctgagtactgcatcgcgcggatggccccctacaccggccccgactccgtgccaggtgctctggactacatgagcttctccacggcgctgtacggcgagagtgacctcaagcttcgaattctgcagtcgacggtaccgcgggcccgggatccaccggtcgccaccgtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcactcacggcatggacgagctgtacaagtaataatccaccccgaccgtccgccctcgtcttgtgcgccgtgccctgccttgcacctccgccgtcgcccatctcctgcctgggttcggtttcagctcccagcctccacccgggtgagctggggcccacgtggcatcgatcctccctgcccgcgaagtgacagtttacaaaattattttctgcaaaaaagaaaaaaaagttacgttaaaaaccaaaaaactacatattttattatagaaaaagtattttttctccaccagacaaatggaaaaaaagaggaaagattaactatttgcaccgaaatgtcttgttttgttgcgacataggaaaataaccaagcacaaagttatattccatcctttttactgatttttttttcttctatctgttccatctgctgtattcatttctccaatctcatgtccattttggtgtgggagtcggggtagggggtactcttgtcaaaaggcacattggtgcatgtgtgtttgctagctcacttgtccatgaaaatattttatgatattaaagaaaatcttttgaaatggctgttttttaaggaagagaatttatgtggcttctcatttttaaatcccctcagaggtgtgactagtctctttatcagcacacacttaaaaaatttttaatattgtctattaaaaataggacaaacttggagagtatggacaactttgatattgcttggcacagatggtattaaaaaaaaccacactcctatgacagctctttgcctgctccttgcttgatgttttctggaagtgtgtgtggggtcaaggatggtgtggggcacctgtatctactactaatgtttttgaccctgctgtgttcaatcttcttacacttcagtttcttcattttaaaaaggaaaaggtaatttcttcacagtctcctatgagttggacagaaagtcatggatgtgtaaaggacttcttgatgataagccccaccttcatttttccacggcaccaaacagccctgtgacaaggatccccgggtac

SEQ ID NO: 34 - DNA, 2750 bp, Artificial Sequence
  Made in Lab - synthesized insert sequence
gagtgttctttttttgatgaaagcaataagaggactgcggaagagctccctgtcaatgtaccgctctacaccagtgtattacgacagttcgtacacaacagtctgtagaggccacctgtctctccctgctgcgttaggaattcaggggagcaggtggtggcagtaagggattttgagggaacggaaatcggatcttgacccagatctgggccgccgataatctcctactgcgctcagactgctgtggaggtgttaggctgagcccgatgccggcaggcaagggaggatgggcggcttgggcagcgcctttgcagacgtggccatttcgtgcctctgcagcaccgccggggggcgcaagagcgcgcgcccggaattgctcattcatcctgtgccgcagagccccgccccttgtccctgcggacagacatttcttctgcgctggtctggccacgtgcttcctgtgctaggagctgcccggaaatgtgaccacctagtctaaagtgggcttctggggcctgagcgctggatggatgcccaccttcctgtcttggtcctccaaaggaggaagctgtgactgagctgtcttggtctggaaggaggccttcccggtttaggatgggaaggtaacattcattaaaagcaacgtagactatagtgtagctgttctcaaaagtagtacatcttagaaaaggatctttagaaaagatcgctttagaaaaggaaattcgttttcagattacgtgagtagcctaggtaacacagccagacctcatctccacaaaaaaaatgaaaaaattagccagcttggtggtctgtgcctgtggtcccagctgctccagaggctgaggtggggggatgactggagcctaggctgcagtgagcctagatggcatcactgcactcaagactgggcgacagaccttatctctaaaaaaataaagattgcatgagtattttgttccacttgacagtcatcaatagattggtttaaattgtgatatcttttttacttaccgcaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagggaggttcaggaggcagcgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattggtgagttgacctcagtaacctgagatcccaggatgctgggacaggaggtctgtccaggggcttctcttgtcactcactcactccctccgtccttctctccctcctccagatgaagttcgcactggcacctaccgccagctcttccaccctgagcagctcatcacaggcaaggaagatgctgccaataactatgcccgagggcactacaccattggcaaggagatcattgaccttgtgttggaccgaattcgcaagctggtaagcaccacatataaatatgcatttaatgtggtgtgatagttccagtgcaagttgggtggagtgactgacatcattcattctttggcacctaccaaaatgtggaataggctgcttgctatattaattggacttctaaatcagatagtccctaggttatggacagtttgtggatatgtctgttttgccaattccttgtgcttacatcagtgagatatggttcgtaatctaaaaagttgaaatagaaattctaagataatgtgtcctggcattaaaatattacatttttttattcccctacaggctgaccagtgcaccggtcttcagggcttcttggttttccacagctttggtgggggaactggttctgggttcacctccctgctcatggaacgtctctcagttgattatggcaagaagtccaagctggagttctccatttacccagcaccccaggtttccacagctgtagttgagccctacaactccatcctcaccacccacaccaccctggagcactc

SEQ ID NO: 35 - DNA, 2784 bp, Artificial Sequence
  Made in Lab - synthesized insert sequence
tgcatgcctgcaggtcgactgagtgttctttttttgatgaaagcaataagaggactgcggaagagctccctgtcaatgtaccgctctacaccagtgtattacgacagttcgtacacaacagtctgtagaggccacctgtctctccctgctgcgttaggaattcaggggagcaggtggtggcagtaagggattttgagggaacggaaatcggatcttgacccagatctgggccgccgataatctcctactgcgctcagactgctgtggaggtgttaggctgagcccgatgccggcaggcaagggaggatgggcggcttgggcagcgcctttgcagacgtggccatttcgtgcctctgcagcaccgccggggggcgcaagagcgcgcgcccggaattgctcattcatcctgtgccgcagagccccgccccttgtccctgcggacagacatttcttctgcgctggtctggccacgtgcttcctgtgctaggagctgcccggaaatgtgaccacctagtctaaagtgggcttctggggcctgagcgctggatggatgcccaccttcctgtcttggtcctccaaaggaggaagctgtgactgagctgtcttggtctggaaggaggccttcccggtttaggatgggaaggtaacattcattaaaagcaacgtagactatagtgtagctgttctcaaaagtagtacatcttagaaaaggatctttagaaaagatcgctttagaaaaggaaattcgttttcagattacgtgagtagcctaggtaacacagccagacctcatctccacaaaaaaaatgaaaaaattagccagcttggtggtctgtgcctgtggtcccagctgctccagaggctgaggtggggggatgactggagcctaggctgcagtgagcctagatggcatcactgcactcaagactgggcgacagaccttatctctaaaaaaataaagattgcatgagtattttgttccacttgacagtcatcaatagattggtttaaattgtgatatcttttttacttaccgcagcgtgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaggttcaggaggcagcgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattggtgagttgacctcagtaacctgagatcccaggatgctgggacaggaggtctgtccaggggcttctcttgtcactcactcactccctccgtccttctctccctcctccagatgaagttcgcactggcacctaccgccagctcttccaccctgagcagctcatcacaggcaaggaagatgctgccaataactatgcccgagggcactacaccattggcaaggagatcattgaccttgtgttggaccgaattcgcaagctggtaagcaccacatataaatatgcatttaatgtggtgtgatagttccagtgcaagttgggtggagtgactgacatcattcattctttggcacctaccaaaatgtggaataggctgcttgctatattaattggacttctaaatcagatagtccctaggttatggacagtttgtggatatgtctgttttgccaattccttgtgcttacatcagtgagatatggttcgtaatctaaaaagttgaaatagaaattctaagataatgtgtcctggcattaaaatattacatttttttattcccctacaggctgaccagtgcaccggtcttcagggcttcttggttttccacagctttggtgggggaactggttctgggttcacctccctgctcatggaacgtctctcagttgattatggcaagaagtccaagctggagttctccatttacccagcaccccaggtttccacagctgtagttgagccctacaactccatcctcaccacccacaccaccctggagcactcgtaccgagctcgaattcact

SEQ ID NO: 36 - DNA, 2680 bp, Artificial Sequence
  Made in Lab - synthesized insert sequence
tttaaatgggcccacactaaagttagagaaccacaggctcgctcacaaccctgacttctccatgtcagttccgatctttgcgaaccgcagacagggaaggtcttctctcaggggtcatgcccgcggccgccctccacggcgaggtccgcactcgcgcagccggccccgcggccgcctcacctggtcgcacactaccacgtcgaactcctcgtcggcgaggaacagcacgtagagcgccaggaaaaccatgcgcacgtaggcgcagacggcggcgccgcggccgccccagcccaggcctcgcggcagccagtccccggcacagcgcaccggtagctcgcggctctcggcgaaacagtggcccgggtcgtagtgcgctgtccagatcttcacgctacacccgcgcgcctgcagcgccagcgccgcgtccaacaccagccgctcagcgccgcccacgcccaggtctgggtggaggaacagcaccgacggcttgggaaccgagtcccgttcccggccctgctcctccgccatggccctggagccgcaactgcaccccgcaccctgatgggggtcttctgcgcaagctccgcgctcgtagctcccagctggccactgcgggccgaccccgccctgccgtacgtgcgtcagttaggccacatcagcgcaaatctgtgagggtctagtaactgcctgagaaaatatcttgtctgaccccggttatatttttccttcggtagggattggactttctgaaggacgttgtgatccaaaggaaggaggccggaggtctctacttcccatacagcaggtaactaagttgtctgtagcagactgtctacaggcatatcgtgagacgacccaggcgtccctggggtcagagaggaccttgcctgcaagtccgggggcggggcctgagtcagtctcgccagctgccggtctttcgggggctccgtaactttctatccgtccgcgtcagcgccttgccacactcatctccaatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggcctgagaagcatggtatggcggcccttccatgatccccgcctctcccagaagccctgactcctcctgctttgcgccgtgcttttcctctgtagctcccttgcttcccccagcctcgggtgtgggtgtctaggccggggttctggggcaggcctgccgcgctcacccgtctgtctgcttgtctccctctacagcctggtccgacccccagtggcactaacgtgggatcctcagggcgctctcccagcaaagcagtggccgcccgggcggcgggatccactgtccggcagaggtaaggaaccctgcagttcgttcgcttccagactcggagataggacccagaacctcgctgattctggggtggagaccctagcatgtgaagattgacaaaggcaaaatgagcttctagtgacgtggccgtgggagtagttaaaggccttttgggaggaaggcgacattttttttctcgttgctcagtttagggcactactcttaaaaaaggaaagttaacaaactggaatagagtcagagataactttgagaaaaccgatgtcattaaactggtgtctctggacctgaggtttgcactcacatttccatctggcggccccataagcaatctgtcctacagataactcgtcctacacaaaacttagtctcttttcagctcagctctctcactctcaattatatctccttacttccatatggcactgttgtacactcatttactcagagccagaaacgtcagcgtcatcttggatttttcttatgctctttctctctctagtcatatgccagactttaaactctgcttgaaagctttctcataagctctttccttttccctttctactgctttgcatttgctacttaacccttttcttcaggctgtttgctttccagtccatcgttcgctctgctgttactcttctgcgtagtttctgttacttgttgctgaac

SEQ ID NO: 37 - DNA, 2747 bp, Artificial Sequence
  Made in Lab - synthesized insert sequence
gatgataggaagtatttacagaactttatagttagtaactgactggttaatttttcaaactgatttttactcaactgaattagaaaaggactggaaagaaagtaaagatcccaacgacttgagggaacaagttggacaaccaaggactttgtctaaattgtttttatttagactaatgtggttctagttctagaggattcatactggaatcatcggtttaatattacgctatttgaaaggcagcatagtatagtacttttggaaaattggcctgagggtgatgtcttttggaatatttgtgaattcactatgaagcctaattccttaaaaatgacctccttcactcaattatcagtgttcttggtttgcctgggagtgaaaagagatcttaaaatctttttggttttagttacataattgactgatgtaatattatgtaatgatggctgtacacagtgtctcatgccctataatcctagcactcatttgagctcaggagttcaaggccagcctgggcaacatggtaaaaccctgtctgcaccaaaaataaaaaaaaaattagccgggcatggtggcatgagtctgtggttccagctactcaggaggctgaggtgggggaggatcgcttgagcccaggagtcagaggttgcagtgagctgagattgtgctactgcactccagcctgggttacagagaccccatctgaattaaaaacatatataatgtaatgatctgcctcctttgttaacttgacttttgaaatgggattgtcagtagtatgatcattgttttcttggatgccgactgtgtgtaaagtgttacattttgaattaaatgtcagaatgggtgaactttactaagattcaattctttgaatacaaagagcattttattttgaagttagaatactaattaaatgcttatgacactttaaaaaattattttttttttctttcagagaattgtaagtgcacagtccttggctgaagatgatgtggaaggcggtagcggggatccaccggtcgccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaatgagaaacaaatgtcaacataataaaatctcagttaaaaatattttaaaaattcttggtagttgagcagctctggggtaataagggcaaatatgcttgttatgaactacactgaaatctaccaaagttaatgtttactttgtgtagatccatttgtctattttatttatttttcccagtgaaaagtgtattttgatagagaacttttcattctataaatacactatgagttactaaaatatcatggattttgtttattcctgaaacatagttacatagttaaactgtacatatgacatggcttatgttaaaaatacccagtgctcagttttgaaagataggcaaaaaaaaaaaagtataggagaaactgaagaatgtacacttttttagagggcacattttgctgtaaatctggaaatttgatagacttgactgtgtttgtgaaaactgagcattaaaggttttgattgatcctttctttccatttaatctctgagacgtaaatatgtgaggtgtgctgctgtgctgggttaacagcttccttccctttctgtgtagcagtcttgaaatgttctgtttaaatcagtaggcttaatgtgttctgggtatttatctccttgtattttaaatatatgtagttgcaaatagcaccaggaattagatttctgtacacccctaatctagccttgtgagcttcgctagttaatgtgtgctcactttccctccatttgttacgtgagagaatgcgtctgctgatcactgaagtgtcccttttagcttctgattcattgggttctgttgggcatctttaaatccaccttaacctgaggaatgtatgtgggcaaccaggccctgcatttttttatattctgaattttgcatgcttgcctgacttagtatttctgaattgatgttttttttaatggtataactatcttgattttcactgaaattatatggttctgtcactactctgtaaattaatccgaaacttttaaggt

SEQ ID NO: 38 - DNA, 2738 bp, Artificial Sequence
  Made in Lab - synthesized insert sequence
tgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccgggctcccgctccggctcccgctcgggatctcgctccggatctcgctccgggtcccgcagtgggtcccggagaggaagctttgacgccacagggaattcttcctactcttattcatactcatttagcagtagtagcattgggcaccacgacccccccgttgctacggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaatagtagtcagttgcgagtggttgctataccttgacttcatttatatgaatttccactttattaaataatagaaaagaaaatcccggtgcttgcagtagagtgataggacattctatgcttacagaaaatatagccatgattgaaatcaaatagtaaaggctgttctggctttttatcttcttagctcatcttaaataagcagtacacttggatgcagtgcgtctgaagtgctaatcagttgtaacaatagcacaaatcgaacttaggatttgtttcttctcttctgtgtttcgatttttgatcaattctttaattttggaagcctataatacagttttctattcttggagataaaaattaaatggatcactgatattttagtcattctgcttctcatctaaatatttccatattctgtattaggagaaaattaccctcccagcaccagcccccctctcaaacccccaacccaaaaccaagcattttggaatgagtctcctttagtttcagagtgtggattgtataacccatatactcttcgatgtacttgtttggtttggtattaatttgactgtgcatgacagcggcaatcttttctttggtcaaagttttctgtttattttgcttgtcatattcgatgtactttaaggtgtctttatgaagtttgctattctggcaataaacttttagacttttgaagtgtttgtgttttaatttaatatgtttataagcatgtataaacatttagcatatttttatcataggtctaaaaatatttgtttactaaatacctgtgaagaaataccattaaaaaactatttggttctgaattcttactagaaggtggtcttttgaagttagtcctttcggtacttctcagatgcctgtcatgtacccgatggagtccttggaaagaaggcctgtgtaaagaggccagcctggaggtcaataacctgttctagtttattctggacattgagtaccaagtagcattggcaaa

SEQ ID NO: 39 - DNA, 2743 bp, Artificial Sequence
  Made in Lab - synthesized insert sequence
taaaggctggtacttggaacctgcaagccgtgcatttggaacctcggactcaagtgcctattacgtaattccacagcgtcccggcctccaggccgtttcccgagccctccagcggagcgggggataaggttaccacgcccgcggtggccggggacactctgagtttcgcgtgtggcttttagggacgtttatatttgaatttccctgaaccgccgagtgtgggcggtggcgcagatccgtcccggaaacctccgggctccttcccgcctttctcaggcccggcccctccaaggggtccccgcggggcggcgggagggccctgggcccagagccgcgcgggtgggcagtcccaggcgtccttccttacagccctgagcctggtccgggaaccgcccagccgggagggccgagctgacggttgcccaagggccagattttaaatttacaggcccggcccccgaaccgccgaagcgcgctgcctgctccccattggcccatggtagtcacgtggaggcgccggggcgtgccggccatgttggggagtgcggcgccgcggcccgcgccacctccgccccccgcggcttgcctccagcccgcccctcccggccctcctccccccgcccgccgctccgtgcagcctgagaggaaacaaagtgctgcgagcaggagacggcggcggcgcgaaccctgctgggcctccagtcaccctcgtcttgcattttcccgcgtgcgtgtgtgagtgggtgtgtgtgttttcttacaaagggtatttcgcgatcgatcgattgattcgtagttcccccccgcgcgcctttgccctttgtgctgtaatcgagctcccgccatcccaggtgcttctccgttcctctaaacgccagcgtctggacgtgagcgcaggtcgccggtttgtgccttcggtccccgcttcgccccctgccgtcccctccttatcacggtcccgctcgcggcctcgccgccccgctgtctccgccgcccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggcctgagaagcagagcccaggccagcgcgactgcgacccccgtgccgccgcggatgggcagccgcgctggcggccccaccacgccgctgagccccacgcgcctgtcgcggctccaggagaaggaggagctgcgcgagctcaatgaccggctggcggtgtacatcgacaaggtgcgcagcctggagacggagaacagcgcgctgcagctgcaggtgacggagcgcgaggaggtgcgcggccgtgagctcaccggcctcaaggcgctctacgagaccgagctggccgacgcgcgacgcgcgctcgacgacacggcccgcgagcgcgccaagctgcagatcgagctgggcaagtgcaaggcggaacacgaccagctgctcctcaagtgagtgctagctggcggccgcgttagcgccaaggaggggcgggggcgcaaccgcggcgaccagctcaccgggttctgccgtggggagggagcagaggccaggatgcacgcgtccttctgaaggaacagggtctcggtctccggaaaggagaaagaatctagagttcatagcggagcaggggtcgcggagggggctcgagctgtagcgctggggggccgtgatgcccatttctagattttggatacccgctgggacgtggtaagtgcgcgcctgggactgccgagaaggagctcccgctttcgcactcgaatccggggagccggcgcggagaggcggcccctcaggccccaggtgcggggagctggagcgcgagcgcgcgctcgcgtgcgcgccccagtttccggccggcgcgagacaaagcgtctagcggatttgcagtgccgggatgggcggccggggaggactggcagcccgcctctagaatgaatgagcttcgcgcgggcagagagaggaaggggagggaccttcccgcagcatccgcgtctcctgggggtgggtcccgctttggcgcgctcagtcttggccctgtgacgttttgcgaagattctacgcctgctttaggcgggagagagaggcggag

SEQ ID NO: 40 - DNA, 2734 bp, Artificial Sequence
  Made in Lab - synthesized insert sequence
taaaggctggtacttggaacctgcaagccgtgcatttggaacctcggactcaagtgcctattacgtaattccacagcgtcccggcctccaggccgtttcccgagccctccagcggagcgggggataaggttaccacgcccgcggtggccggggacactctgagtttcgcgtgtggcttttagggacgtttatatttgaatttccctgaaccgccgagtgtgggcggtggcgcagatccgtcccggaaacctccgggctccttcccgcctttctcaggcccggcccctccaaggggtccccgcggggcggcgggagggccctgggcccagagccgcgcgggtgggcagtcccaggcgtccttccttacagccctgagcctggtccgggaaccgcccagccgggagggccgagctgacggttgcccaagggccagattttaaatttacaggcccggcccccgaaccgccgaagcgcgctgcctgctccccattggcccatggtagtcacgtggaggcgccggggcgtgccggccatgttggggagtgcggcgccgcggcccgcgccacctccgccccccgcggcttgcctccagcccgcccctcccggccctcctccccccgcccgccgctccgtgcagcctgagaggaaacaaagtgctgcgagcaggagacggcggcggcgcgaaccctgctgggcctccagtcaccctcgtcttgcattttcccgcgtgcgtgtgtgagtgggtgtgtgtgttttcttacaaagggtatttcgcgatcgatcgattgattcgtagttcccccccgcgcgcctttgccctttgtgctgtaatcgagctcccgccatcccaggtgcttctccgttcctctaaacgccagcgtctggacgtgagcgcaggtcgccggtttgtgccttcggtccccgcttcgccccctgccgtcccctccttatcacggtcccgctcgcggcctcgccgccccgctgtctccgccgcccgccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtccggtctgcgatctcgagcacaggcatccgcgactgcgacccccgtgccgccgcggatgggcagccgcgctggcggccccaccacgccgctgagccccacgcgcctgtcgcggctccaggagaaggaggagctgcgcgagctcaatgaccggctggcggtgtacatcgacaaggtgcgcagcctggagacggagaacagcgcgctgcagctgcaggtgacggagcgcgaggaggtgcgcggccgtgagctcaccggcctcaaggcgctctacgagaccgagctggccgacgcgcgacgcgcgctcgacgacacggcccgcgagcgcgccaagctgcagatcgagctgggcaagtgcaaggcggaacacgaccagctgctcctcaagtgagtgctagctggcggccgcgttagcgccaaggaggggcgggggcgcaaccgcggcgaccagctcaccgggttctgccgtggggagggagcagaggccaggatgcacgcgtccttctgaaggaacagggtctcggtctccggaaaggagaaagaatctagagttcatagcggagcaggggtcgcggagggggctcgagctgtagcgctggggggccgtgatgcccatttctagattttggatacccgctgggacgtggtaagtgcgcgcctgggactgccgagaaggagctcccgctttcgcactcgaatccggggagccggcgcggagaggcggcccctcaggccccaggtgcggggagctggagcgcgagcgcgcgctcgcgtgcgcgccccagtttccggccggcgcgagacaaagcgtctagcggatttgcagtgccgggatgggcggccggggaggactggcagcccgcctctagaatgaatgagcttcgcgcgggcagagagaggaaggggagggaccttcccgcagcatccgcgtctcctgggggtgggtcccgctttggcgcgctcagtcttggccctgtgacgttttgcgaagattctacgcctgctttaggcgggagagagaggcggag

SEQ ID NO: 41 - DNA, 3454 bp, Artificial Sequence
  Made in Lab - synthesized insert sequence
taaaggctggtacttggaacctgcaagccgtgcatttggaacctcggactcaagtgcctattacgtaattccacagcgtcccggcctccaggccgtttcccgagccctccagcggagcgggggataaggttaccacgcccgcggtggccggggacactctgagtttcgcgtgtggcttttagggacgtttatatttgaatttccctgaaccgccgagtgtgggcggtggcgcagatccgtcccggaaacctccgggctccttcccgcctttctcaggcccggcccctccaaggggtccccgcggggcggcgggagggccctgggcccagagccgcgcgggtgggcagtcccaggcgtccttccttacagccctgagcctggtccgggaaccgcccagccgggagggccgagctgacggttgcccaagggccagattttaaatttacaggcccggcccccgaaccgccgaagcgcgctgcctgctccccattggcccatggtagtcacgtggaggcgccggggcgtgccggccatgttggggagtgcggcgccgcggcccgcgccacctccgccccccgcggcttgcctccagcccgcccctcccggccctcctccccccgcccgccgctccgtgcagcctgagaggaaacaaagtgctgcgagcaggagacggcggcggcgcgaaccctgctgggcctccagtcaccctcgtcttgcattttcccgcgtgcgtgtgtgagtgggtgtgtgtgttttcttacaaagggtatttcgcgatcgatcgattgattcgtagttcccccccgcgcgcctttgccctttgtgctgtaatcgagctcccgccatcccaggtgcttctccgttcctctaaacgccagcgtctggacgtgagcgcaggtcgccggtttgtgccttcggtccccgcttcgccccctgccgtcccctccttatcacggtcccgctcgcggcctcgccgccccgctgtctccgccgcccgccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagagcggcctgagaagcagagcccaggccagcgcgactgcgacccccgtgccgccgcggatgggcagccgcgctggcggccccaccacgccgctgagccccacgcgcctgtcgcggctccaggagaaggaggagctgcgcgagctcaatgaccggctggcggtgtacatcgacaaggtgcgcagcctggagacggagaacagcgcgctgcagctgcaggtgacggagcgcgaggaggtgcgcggccgtgagctcaccggcctcaaggcgctctacgagaccgagctggccgacgcgcgacgcgcgctcgacgacacggcccgcgagcgcgccaagctgcagatcgagctgggcaagtgcaaggcggaacacgaccagctgctcctcaagtgagtgctagctggcggccgcgttagcgccaaggaggggcgggggcgcaaccgcggcgaccagctcaccgggttctgccgtggggagggagcagaggccaggatgcacgcgtccttctgaaggaacagggtctcggtctccggaaaggagaaagaatctagagttcatagcggagcaggggtcgcggagggggctcgagctgtagcgctggggggccgtgatgcccatttctagattttggatacccgctgggacgtggtaagtgcgcgcctgggactgccgagaaggagctcccgctttcgcactcgaatccggggagccggcgcggagaggcggcccctcaggccccaggtgcggggagctggagcgcgagcgcgcgctcgcgtgcgcgccccagtttccggccggcgcgagacaaagcgtctagcggatttgcagtgccgggatgggcggccggggaggactggcagcccgcctctagaatgaatgagcttcgcgcgggcagagagaggaaggggagggaccttcccgcagcatccgcgtctcctgggggtgggtcccgctttggcgcgctcagtcttggccctgtgacgttttgcgaagattctacgcctgctttaggcgggagagagaggcggag

SEQ ID NO: 42 - DNA, 2756 bp, Artificial Sequence
  Made in Lab - synthesized insert sequence
tttatttttatttttatttttttgagatggagtcttactctgtcaccaggttggagtgcagtggtacgatctcggctcactgcaacctccagttcctgggttcaagcgattctcctgcgtcagcttcccgagtaggtgggactacaggtgtgcgccaccacacccgactaatttttgtatttttagtagagatagggtttcaccgtgttggccaggatggtctcaatctcctgatttcgtgattgagccacctcggcctcccaaagtgctgggattacaggcgtgagccaccacgcccagccttagactgggtaatttataatgaatggaaatttatttggctcccagttccaaaggctggaaagtccaagattggaggtctgaatctggcgagggccttcttgctgtcatccattggcagaagggtgagagcaagatagaaagggggcataatcatccttttaatcagcaacccactcttgtgataatagcattactctattcaggaaggcagaggcctcatgacctgaatcatctctcgaaggtcccacctctcaactcttgcatttaagggttacgtttccaacacatgaactttgggggacacactagaaccatagcactgagttttacttgaattaataatgaaaacatctggtttaaagagcacacaagagaaaaacagcccaaagccctgttgtagacattagtcctttctcctctttaggccaactgcattgactccacagcctcagccgaggccgtgtttgcctccgaagtgaaaaagatgcaacaggagaacatgaagccgcaggagcagttgacccttgagccatatgaaagagaccatgccgtggtcgtgggagtgtacaggtgagcaggggcccagcaatacaccaagacagacatctctgtcccttgcaccccgagtgccatgatcctggggaccctccttcatcacctatcttcctctcacaggccacctcctaaagtgaagaacaagcccaacagcgccgtggacggcaccgccggccccggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaatgaagttcagccctgagcggattgcgagagatgtgtgttgatactgttgcacgtgtgtttttctattaaaagactcatccgtctcccatgtctgctgctcattcctccccttgacctgctgacacagggagcacgcacccttggtcaattttgcggggttgggtaaattctcactcggtcacagagcgcatgctccgtttctagctgcctttgcgcagcggcagcctggatttcggttcttgggtgggattggtagctcgctgcgcatgcgtgcaggtaagcggccatctcgcgcaggcggagtgtcagtgtgggtcacgtgaggggagcggagagggagggatgggggcggagtccagggcgtgggggggccggtttgttgtggtcgccattttgctggttgcattactgggtaatcggggccctggcttgccgcgtccgccggataccctcagccagtgggcaggtctgagctcgggctccccgagcagtttgagtccccttgcccgctccttcaggtaacggcgcggggacgggtggggcggcaagcggtcgcagggaggtgggcaggacgggatccgccctgctcccgtcgccgtgagacttagcacgaggccaagggaggagaggaggggggtggcaggcaggtgcgggccctgcctggctattcatagttgaattcctggaaccggccaagcccgaggaagcagttgcaggagggaggctgggagggggtagccgggccccactcccgccctttgtttgggctcagctccgcgggccgcttcttcgtcgcctagcaacagctgccctaggctgtgattggctgagctcttggcaccagcgaccaatggtacagttgttgccatggcaggtgccgattgccaagctcagtcgggccccgccttccggtctcagcaggcccaggagggcctcctgggtggggggcgggacgccgggtccctaggggctggtggtcactcagggtggggcgtgtcgc

SEQ ID NO: 43 - DNA, 2744 bp, Artificial Sequence
  Made in Lab - synthesized insert sequence
gcgcgggactggtcgggggcccaggccttgtggctggggccaactatggccatgtgtacgcggtgtttgaaacagtgagtgccggtggcagtgccgcggcttgccgcctttctcctgttcacagacttagtgaggcacactcatcccatccggttagcccgttcaaaatgcatgatttagtagttttttggatatccagggttgtactgccgtcaccaccaggtcattccagaacattttcaccaccccacgaggaaagcctgtccccataagctatctgtccccacaccttcccccaacccctggcacccgctggtgggcttttgtccctgtggagtggcttcttctggacgtgtcacaggagcagagtcactgcccggcatccttctgccctccacccactctccttcgtcttgcctccccaggctacgaggaacaccggcaagagtatcgccaataagaacatcgccaaccccacggccaccctgctggccagctgcatgatgctggaccacctcaagtgagtggctcccagtgccccgcaagcccctggccagccctccgggccccagggactgaaggctggctgtcccatcctctaggctgcactcctatgccacctccatccgtaaggctgtcctggcatccatggacaatgagaatgtgaggttcccctcgcaccctaccctgctgccccgcccagtctccccctgcagcctcctgtagcccgcctcatgcactgaccacagcccccaggggaggatggtactggatctggccccatcttctggcccttagctggtgccacttccctcctgtggcaatcccagtcctgcacgtcacaggagacagcggggggtggcagggcctggtgtgtgccgagcctgcctgtgtcctacagatgcacactccggacatcgggggccagggcacaacatctgaagccatccaggacgtcatccgccacatccgcgtcatcaacggccgggccgtggaggccgtggaccccagggtgcccgtggccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtgagctggccctaggaccttcttggtttgctccttggattccccttcccactccagcaccccagccagcctggtacgcagatcccagaataaagcaccttctccctagacttggcgtgggcctccctcactccaggcaccgatgttcagtccacactttattaagaaaggaatacacacactttctccgagagccctcacgggggtctcaggcagaacacgctctgagagccgggcctggctccagcgtctctctgccctgtggttgtggagaactggcgcccagcagggtggtgggagctgtcccgcttttcccttaaggcacggagagctggaggtggagccatagccgggcctaggggttgagccaggggtgctggaggcagtcagcggcactggcccgcttttcggggatgtactccatcatgggcagcagaaaggcgctgaactgtgtggcctgctctaggggccactcgtacttttccatgagtacctcgtacaggccccagtgcttgagattgtggatgtgccgcagctctcctgcgggcaggccagcgggcgtcaggctctgtccctgtctctggacctgccttctgcttaggccctggctcctgggacccagtgttccctctgttgcagaatccaaggtccccgtcccaccctccctgcagccacatggcctgaggctgcccgggccctcacctctccggttgaagaactcccgggaatagcggcctgagagggcgaaggctggggggatgtcccccagaagctccactatgtgagcgatgtggtctgtgggcagaaagaggaggttgggggcaggcaggccgggaggccgggaggctgagcccagagccctcaccccttaccctcatcacgactgtagtcttctccagaatgcggctcgaacaggtagtcaccagtggccagctcgaaggcctggaagggggaggaggtgaggctgctggctggtgggctgctggccctgggcacagggcagc

SEQ ID NO: 44 - DNA, 2729 bp, Artificial Sequence
  Made in Lab - synthesized insert sequence
gctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctattctcgcaactgacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaaggccggctccggtaccgatgatgatatcgcagcgctcgtcgtcgacaacggctccggcatgtgcaaggccggcttcgcgggcgacgatgccccccgggccgtcttcccctccatcgtggggcgccccaggcaccaggtaggggagctggctgggtggggcagccccgggagcgggcgggaggcaagggcgctttctctgcacaggagcctcccggtttccggggtgggggctgcgcccgtgctcagggcttcttgtcctttccttcccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccaggtgagtggcccgctacctcttctggtggccgcctccctccttcctggcctcccggagctgcgccctttctcactggttctctcttctgccgttttccgtaggactctcttctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggcgcacagtaggtctgaacagactccccatcccaagaccccagcacacttagccgtgttctttgcactttctgcatgtcccccgtctggcctggctgtccccagtggcttccccagtgtgacatggtgtatctctgccttacagatcatgtttgagaccttcaacaccccagccatgtacgttgctatccaggctgtgctatccctgta

SEQ ID NO: 45 - DNA, 2765 bp, Artificial Sequence
  Made in Lab - synthesized insert sequence
ccaacaggccctgtcctagcctttctctcttccccactgccccaggagctaccctttgtggaccccaaggcgcggcgctcagggactggcacagagtgggcgactttcttgctgacttgctctccaccctccctctgcctttggcttctgccaaagagaagccaaagggggactgtgccctgagcgctggccgggtccctgtctcactgtcctatcaggtggccagcgtgaggaagaccacggtcctggatgtcatgaggcggctgctgcagcccaagaacgtgatggtgtccacaggccgagaccgccagaccaaccactgctacatcgccatcctcaacatcatccagggagaggtggaccccacccaggtaggggaggccccttcatcccacaccctggacctgcaggggtagaggagaggccacctccactgctcctatgcccaccccaggtccacaagagcttgcagaggatccgggaacggaagttggccaacttcatcccgtggggccccgccagcatccaggtggccctgtcgaggaagtctccctacctgccctcggcccaccgggtcagcgggctcatgatggccaaccacaccagcatctcctcggtgagtctcaaagtttgcaccttttttccctgaatcagtttcctgactatacctcacctctctgcatctgctggccctgcttctagcttttttgctgtgggcatagcccagccttggttccccagctttctgggccacgttattctttgaagttctttgtaacccctgttttctgcacaccccaagctcttcgagagaacctgtcgccagtatgacaagctgcgtaagcgggaggccttcctggagcagttccgcaaggaggacatgttcaaggacaactttgatgagatggacacatccagggagattgtgcagcagctcatcgatgagtatcatgcggctactagaccagactacatctcctggggcacccaggagcagagaatcctgcagagcacagtgcccagagccagggatccacctgtggccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtgagtcccccaggacagggaccctcatctgacttactggttggcccaagccctgcctgactgaccaccccctcagagcacagatcagggacctcacgcatctctttctcatatacatggactctctgttggcctgcaaacacatttacttctcctcttatgagactatttatctttaataaagcactggatataaatcaagtcactgctccctttaaagcctttgggttctggagatgcggtggggatgcctgttctttctccatcattctgacagggtcctcacccacttccagcatcttcaattctgaaccaacagcttcccctaggtttttcctctgcctagttttggagaacgctggtggggtttgacccacctgaaattcctttccagggtaaaaaggcccagagatattactttgttctctgtctgaccctttctgggtccccgttccctcccaccctcactgtatcaggtgaatattccaccagcctcatgtttccacatcagagtgtctgattaatcacaacctgaggcccaaggccaccaccactgccactaggccatgaacaagcaatggagtccaacccaatacctttaagcttctagcaagcagtccaggagagccgctcgtttcaggcagttggagtctagcagcatcaagtcagcgaccaaatctaaaacctagactgtaagcaggatcataagtggttccatagctaaggcaccgtaattctcttctctagtggttgccagtaatttaaatggccagtgccttctacttctggtggaagattgcagggaggtagtaaatacataggggcctaagtcttcaggattgtttttttttttttttgagatagggtcttgctctgtcgctttgtcacccaggctggaatgcagtggcataatcacggttcactgcagcctcgacctccctggctcaggcgatcctcctgtctcagcctcttaagtagctgggatcacaggcacacaccaccta

SEQ ID NO: 46 - DNA, 2756 bp, Artificial Sequence
  Made in Lab - synthesized insert sequence
ccattggcagctgcacgctcattactaccctccgctcctgcgctctgccactgtccggaaattcatggttggctacgaaatgcttgctcaggctcagagggacctcacccctgagcaggtcaggactcagaacagtctggcgtctccagactctcacatgcagtatgtgcaggcacctgatacttctgttgcccttgtgctccagtcattgcacaaggcagaaaacagctctggcaggaagggactgccaaagttaggagccctagggcctggaaggagagtatggtcctcagatcccccttctctcctgcttcctccagggaacccaacagtcatgaccctgatagtttcccataacaacctgggcattccttgggactcaggagctgctaaactctttcatcccctggtggcttcagcagtccttatcaccagcctcacaatcccacaggcccacccccagtgggcctgtggcattcatatttcatattcatatttcaaaccacaatatccagcaaaatgtctcctgagcacccagaactccataccatcggccgggtgtggtggctcatgccttaatcccagcactttgggaggtcaagatgggaggattgcttgagcccagaagttcgagactagcctgggaaacataggaagccctcgtctctacaaaaaaaatttaaaaagttagccaggtatggtggcatatacttgtggtcccagatacttgggaggctgagataggatcacttgggcccaggagtttgaggctgcagtgagccatcatcatggcatcattgcattccagcctgggcaacagagcaagacctcgtctcaaaaaaaaaaaaaaaatgaagtccatgccaccattcttggcagcccagcccttatcctccttaattgctccctgtcccttttccaggctgcagagagactaagggcacttcctgaggttcactatcatctggggcagaaggacagggagacagcaaccatcgccagcaccgtgcccagagccagagacccaccagtggccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaccacggagaccccagggccttgaatccttttttgttttcaacagtcttgctgaattaagcagaaagggccttgaatcctggcctggaatttgggcagatatagcattaataaaactgtgcatctcaaacttttatcacatactctaatatcagaggagtgtgaaccttcagagatctagggttaaaagctaaaggcatagctccagctacaacttttctttgtctacagatgtgtaaaatcttgatgcaaaaatctaccccaaatttctgaacaaaattaatattcagtattatatctagcctatagattctcttactcttggtgtaatgacagcattcccagtttaggaaactgttttaaaatcttgtgtcttggttgtggctgaggctttgggaaggccagagccccctttgccaccacccctagggtgtcagctccaagcctgggcctgagggtttgatggggagcgggtgagctggatcctgatcccagtggtcagtggccagatcctgggctgctggccagaaccctggctttgtgagtggctgcagcttggctctgctccagcccacaggggctaaaggggcgggtagggagtgtgtagggagcgtgtacgtggctttgtgtccttggctcctccagactctcacatgtgcgggcaccatatacttctgctgcccttgtgccccaatcattgcacaaacagaaacagctctgacagagaagggactgctagagttaggaaccctagggcctggaaggaaggtatggctctcagagtgacctggggtctattcagtcccctctggacctgtttccctggcaacgctgtgggaacaacagtctatgcaacacaggagacctgatgccactggtgtcctccaaattttaaggcgaaggcaggcattgtgaatgatgcagggatggccagaatggtttttgccttcgttctttccccaggggaaccaagcctgtgggtggggaaatcacactaag

SEQ ID NO: 47 - DNA, 3235 bp, Artificial Sequence
  Made in Lab - synthesized insert sequence
caaaaagtccgcacattcgagcaaagacaggctttagcgagttattaaaaacttaggggcgctcttgtcccccacagggcccgaccgcacacagcaaggcgatggcccagctgtaagttggtagcactgattaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcggtgagtcaccgccggtgactaagcgaccccacccctctccctcgggctttcctctgccaccgccgtctcgcaactcccgccgtccgaagctggactgagcccgttaggtccctcgacagaacctcccctccccccaacatctctccgccaaggcaagtcgatggacagaggcgcgggccggagcagcccccctttccaagcgggcggcgcgcgaggctgcggcgaggcctgagccctgcgttcctgcgctgtgcgcgcccccaccccgcgttccaatctcaggcgctctttgtttctttctccgcgacttcagatctgagggattccttactctttcctcttcccgctcctttgcccgcgggtctccccgcctgaccgcagccccgagaccgccgcgcacctcctcccacgcccctttggcgtggtgccaccggacccctctggttcagtcccaggcggacccccccctcaccgcgcgaccccgcctttttcagcaccccagggtgagcccagctcagactatcatccggaaagcccccaaaagtcccagcccagcgctgaagtaacgggaccatgcccagtcccaggccccggagcaggaaggctcgagggcgcccccaccccacccgcccaccctccccgcttctcgctaggtccctattggctggcgcgctccgcggctgggatggcagtgggaggggaccctctttcctaacggggttataaaaacagcgccctcggcggggtccagtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggactgagaagcggaagcggaggaggaagcgcgagcggaggcagcggaagcagtaccaggtccgtgtcctcgtccagttaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaagtaaggctgcgcccatgcaagtagctgggcctcgggagggggctggagggagaggggaacgcccccccggcccccgcgagagctgccacgcccttggggatgtggccggggggaggcctgccagggagacagcggagagcggggctgtggctgtggtggcgcagccccgcccagaacccagaccttgcagttcgcatttcctcctctgtccccacacattgcccaaggacgctccgtttcaagttacagatttcttaaaactaccactttgtgtgcagttgaaggcccttgggcacaatgagagccagtcctccaaactttcagaaagtttcctgccccttctggcaggctgccaatcaccgggcgggagaaggaaggaggggaaggcggtggagggagcgagacaaagggatggtccctcgggggcggggatggcggggctgtcctgtaggtctgtgcggccaccgtgattgcccctctgcgcggtgcccgaagtcccgctgaaacctgccgagggcagcaggtctgaaagctgcaggcgctagttgcgcggaggtggcgcagctgctctggaggcgcagagcgaatacgtggtgtttgggtgtggccgccccgcccctggcggtttcctcgttcccctttggttaatgcgcaactgtttcagattgcaggaggagatgcttcagagagaggaa

SEQ ID NO: 48 - DNA, 2777 bp, Artificial Sequence
  Made in Lab - synthesized insert sequence
cggtttttcctacaaggaatccagttgaatacaattcttcctgacgccagaggtgagaacccacaatctctgcgagccccgcccccgcccccgcccgcgcccccagggtattctggagccactagacctctgtgtgtgttgcagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctggtgagtgtcaccgagggcagctgtcgcggggtgtggaggacgtgcttcagactccgcctgtggacgtttagtcgcttccgtgtgggctggggcgacgcccctgttcctctgcaaggagctgtttcttcttgccggtctgagattctagaggtaactccccctgctttagagaggcccagcgtgtctctcagctgggagcccctggtaccatttgagagtaagggaatcattttaagaaacagtggtggcgcttctcccatgaacgttgaatacaacactgtcatctgatgtgcacagaggccctggacgcagcacagctgtccggccacagcccctgattccagacgggggagagacgtgtgtggtttcctcgccgtgcagagagcaccagtctctgcagcggcttccccaagtgacaattccagctagagctggaaccttccgccagggttcctctttgccttttggctgatgtgggggagtggtggggagagaggcctgtttgcaggcccctgtgtgagcagagccctgacaccatccgtctgtcttggcagtggaggagtgtctgctggacgagaacagcatgctgatccccatcgctgtgggtggtgccctggcggggctggtcctcatcgtcctcatcgcctacctcgtcggcaggaagaggagtcacgcaggctatcagactatcgaatttggcagcaccggcagcaccggcagcaccggcgcggatccgccggtggcgaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtagcctggtgcacgcagccacagcagctgcaggggcctctgttcctttctctgggcttagggtcctgtcgaaggggaggcacactttctggcaaacgtttctcaaatctgcttcatccaatgtgaagttcatcttgcagcatttactatgcacaacagagtaactatcgaaatgacggtgttaattttgctaactgggttaaatattttgctaactggttaaacattaatatttaccaaagtaggattttgagggtgggggtgctctctctgagggggtgggggtgccgctgtctctgaggggtgggggtgccgctgtctctgaggggtgggggtgccgctctctctgagggggtgggggtgccgctttctctgagggggtgggggtgccgctctctctgagggggtgggggtgctgctctctccgaggggtggaatgccgctgtctctgaggggtgggggtgccgctctaaattggctccatatcatttgagtttagggttctggtgtttggtttcttcattctttactgcactcagatttaagccttacaaagggaaagcctctggccgtcacacgtaggacgcatgaaggtcactcgtggtgaggctgacatgctcacacattacaacagtagagagggaacatcctaagacagaggaactccagagatgagtgtctggagcgcttcagttcagctttaaaggccaggacgggccacacgtggctggcggcctcgttccagtggcggcacgtccttgggcgtctctaatgtctgcagctcaagggctggcacttttttaaatataaaaatgggtgttatttttatttttatttgtaaagtgatttttggtcttctgttgacattcggggtgatcctgttctgcgctgtgtacaatgtgagatcggtgcgttctcctgatgttttgccgtggcttggggattgtacacgggaccagctcacgtaatgcattgcctgtaacaatgtaataaaaagcctc

SEQ ID NO: 49 - DNA, 2720 bp, Artificial Sequence
  Made in Lab - synthesized insert sequence
agtgtacacgtcggttgcctaacaaccggcagcggactcctttggctatggtgagtgctgccgctctgtcgcgccccacgcccagcggctgagcccttcccaggccccagctcggccacgccgcctctcctgcgcctttctcggctttagcgcaccagtctttcctttgagctctttcccgggagagtagggtacagacgctaaccaacctactgggcctgcgcttcgctgctgcctcctcttcacccacaacaggcccacacccgcccctcgggctccccttgcattccctgatcccctcggtgaacctccctgggcaaaaaagactcgaatccctcacaggcccctgactctctcagaccttggccccctggcctcctcattctgccaaaatttccacacacagctgggaaaaggtgtatcgtgttctgttacaatgttttttacaatttaaatacaggggcattcaggggatgctttagcttcttggagaaggcacgaaggtgccagatggattgagttttattactcggtactgacacaatatacatcttaacaacttactaatatttggccaaatctagaagtaattgggaagtcggagtacttactgggaaggggagtactgggagtgggcatcgtaagactgttttcagaaccaggatccccacgttcgggaagagctgattatctctagttattcacagtcatcttcttgactggctctcttttgggatgattttcagtaagaggtgtccttactgaacaagtgatctccctgcttctagaaacaacagtaaagaaaagggaggggtagaagttcatggaaagattcattgttcttgccagtaaaccctgttatctttctttttaagcaaagctttggaattggttatataaatgactttaaaaatacatatgggtgtctgtaactatgttatgtaaatatggactgtatgatatattattgtgcttgtgtattgcataacatataaaggccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagcggcctgagaagctccaatttcaaaaaggcaaacatggcatcaagttctcagcgaaaaagaatgagtcctaagcctgagcttactgaagagcaaaagcaggagatccgggaagcttttgatcttttcgatgcggatggaactggcaccatagatgttaaagaactgaaggcaagctctgtgcattcctgccgtactgcccttcctttcctctgcctcttccttgtcatctccctatcacctctctaccccttttgccctcttctccctgcagtaagtaacaggggaggttgtactgtgctgttttcctgtctttacttactttgagtattattcaataaaattgattgcaaatatcaattagatcaatgtcatagtgttccccaatcatattaacatgaccacatcagatgctggcttaatgctctcaacactgttatttttgctcctgctactttagatgtgcttattgagacgatcctgttttctttataggtggcaatgagggccctgggctttgaacccaagaaagaagaaattaagaaaatgataagtgaaattgataaggaagggacaggaaaaatgaactttggtgactttttaactgtgatgacccagaaaatggtaagtagctaagacaattatcatggtatggagccccagtcatctgcagaggtccatggcgtcttcctgttaaaacagtcatgtgaccacagagcagtccaggtagcaactgcatcaggaggcctgccctcccatcatgttactttgggctcccaaaacaagcccttgtgcttctttctcaagacttccaagcagatacctcttcccacagaattccaaatcagttaggaaatagatatgggatagggagagatatagcgataggtagcaaacgtcaccttggatggagtgcctggcttgcctggtgtttctttgaggccaaaatgggcttcctgacacttgccgcaagtctagactcgagttacattgttaaaaaggaaagggtgacctg

SEQ ID NO: 50 - DNA, 3440 bp, Artificial Sequence
  Made in Lab - synthesized insert sequence
agtgtacacgtcggttgcctaacaaccggcagcggactcctttggctatggtgagtgctgccgctctgtcgcgccccacgcccagcggctgagcccttcccaggccccagctcggccacgccgcctctcctgcgcctttctcggctttagcgcaccagtctttcctttgagctctttcccgggagagtagggtacagacgctaaccaacctactgggcctgcgcttcgctgctgcctcctcttcacccacaacaggcccacacccgcccctcgggctccccttgcattccctgatcccctcggtgaacctccctgggcaaaaaagactcgaatccctcacaggcccctgactctctcagaccttggccccctggcctcctcattctgccaaaatttccacacacagctgggaaaaggtgtatcgtgttctgttacaatgttttttacaatttaaatacaggggcattcaggggatgctttagcttcttggagaaggcacgaaggtgccagatggattgagttttattactcggtactgacacaatatacatcttaacaacttactaatatttggccaaatctagaagtaattgggaagtcggagtacttactgggaaggggagtactgggagtgggcatcgtaagactgttttcagaaccaggatccccacgttcgggaagagctgattatctctagttattcacagtcatcttcttgactggctctcttttgggatgattttcagtaagaggtgtccttactgaacaagtgatctccctgcttctagaaacaacagtaaagaaaagggaggggtagaagttcatggaaagattcattgttcttgccagtaaaccctgttatctttctttttaagcaaagctttggaattggttatataaatgactttaaaaatacatatgggtgtctgtaactatgttatgtaaatatggactgtatgatatattattgtgcttgtgtattgcataacatataaaggccgtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagagcggcctgagaagctccaatttcaaaaaggcaaacatggcatcaagttctcagcgaaaaagaatgagtcctaagcctgagcttactgaagagcaaaagcaggagatccgggaagcttttgatcttttcgatgcggatggaactggcaccatagatgttaaagaactgaaggcaagctctgtgcattcctgccgtactgcccttcctttcctctgcctcttccttgtcatctccctatcacctctctaccccttttgccctcttctccctgcagtaagtaacaggggaggttgtactgtgctgttttcctgtctttacttactttgagtattattcaataaaattgattgcaaatatcaattagatcaatgtcatagtgttccccaatcatattaacatgaccacatcagatgctggcttaatgctctcaacactgttatttttgctcctgctactttagatgtgcttattgagacgatcctgttttctttataggtggcaatgagggccctgggctttgaacccaagaaagaagaaattaagaaaatgataagtgaaattgataaggaagggacaggaaaaatgaactttggtgactttttaactgtgatgacccagaaaatggtaagtagctaagacaattatcatggtatggagccccagtcatctgcagaggtccatggcgtcttcctgttaaaacagtcatgtgaccacagagcagtccaggtagcaactgcatcaggaggcctgccctcccatcatgttactttgggctcccaaaacaagcccttgtgcttctttctcaagacttccaagcagatacctcttcccacagaattccaaatcagttaggaaatagatatgggatagggagagatatagcgataggtagcaaacgtcaccttggatggagtgcctggcttgcctggtgtttctttgaggccaaaatgggcttcctgacacttgccgcaagtctagactcgagttacattgttaaaaaggaaagggtgacctg

SEQ ID NO: 51 - DNA, 2744 bp, Artificial Sequence
  Made in Lab - synthesized insert sequence
agtgtacacgtcggttgcctaacaaccggcagcggactcctttggctatggtgagtgctgccgctctgtcgcgccccacgcccagcggctgagcccttcccaggccccagctcggccacgccgcctctcctgcgcctttctcggctttagcgcaccagtctttcctttgagctctttcccgggagagtagggtacagacgctaaccaacctactgggcctgcgcttcgctgctgcctcctcttcacccacaacaggcccacacccgcccctcgggctccccttgcattccctgatcccctcggtgaacctccctgggcaaaaaagactcgaatccctcacaggcccctgactctctcagaccttggccccctggcctcctcattctgccaaaatttccacacacagctgggaaaaggtgtatcgtgttctgttacaatgttttttacaatttaaatacaggggcattcaggggatgctttagcttcttggagaaggcacgaaggtgccagatggattgagttttattactcggtactgacacaatatacatcttaacaacttactaatatttggccaaatctagaagtaattgggaagtcggagtacttactgggaaggggagtactgggagtgggcatcgtaagactgttttcagaaccaggatccccacgttcgggaagagctgattatctctagttattcacagtcatcttcttgactggctctcttttgggatgattttcagtaagaggtgtccttactgaacaagtgatctccctgcttctagaaacaacagtaaagaaaagggaggggtagaagttcatggaaagattcattgttcttgccagtaaaccctgttatctttctttttaagcaaagctttggaattggttatataaatgactttaaaaatacatatgggtgtctgtaactatgttatgtaaatatggactgtatgatatattattgtgcttgtgtattgcataacatataaaggccgtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagagcggcctgagaagctccaatttcaaaaaggcaaacatggcatcaagttctcagcgaaaaagaatgagtcctaagcctgagcttactgaagagcaaaagcaggagatccgggaagcttttgatcttttcgatgcggatggaactggcaccatagatgttaaagaactgaaggcaagctctgtgcattcctgccgtactgcccttcctttcctctgcctcttccttgtcatctccctatcacctctctaccccttttgccctcttctccctgcagtaagtaacaggggaggttgtactgtgctgttttcctgtctttacttactttgagtattattcaataaaattgattgcaaatatcaattagatcaatgtcatagtgttccccaatcatattaacatgaccacatcagatgctggcttaatgctctcaacactgttatttttgctcctgctactttagatgtgcttattgagacgatcctgttttctttataggtggcaatgagggccctgggctttgaacccaagaaagaagaaattaagaaaatgataagtgaaattgataaggaagggacaggaaaaatgaactttggtgactttttaactgtgatgacccagaaaatggtaagtagctaagacaattatcatggtatggagccccagtcatctgcagaggtccatggcgtcttcctgttaaaacagtcatgtgaccacagagcagtccaggtagcaactgcatcaggaggcctgccctcccatcatgttactttgggctcccaaaacaagcccttgtgcttctttctcaagacttccaagcagatacctcttcccacagaattccaaatcagttaggaaatagatatgggatagggagagatatagcgataggtagcaaacgtcaccttggatggagtgcctggcttgcctggtgtttctttgaggccaaaatgggcttcctgacacttgccgcaagtctagactcgagttacattgttaaaaaggaaagggtgacctg

SEQ ID NO: 52 - DNA, 2750 bp, Artificial Sequence
  Made in Lab - synthesized insert sequence
agccttggcagtcggcgccggtgaacgagagcaacgcttctgaccctgccggagctcctcggagatgaaagccatgacgcgccttgcagaaaatgcattccgccttccgtgggaacaacgccgaggcacgcggtgacagccgtgaccatgctgtttgcccagtgaaggaaacaactgtcgggtatcggctctgccggcctttccagccgcactcatgcatggggctcaccccatgatgtgcgtggcttgtcgaggagcaagtggacaagtctcttaaggaaagctttggtgcacaggcgctttctccttgggggcgaattctgccagaccttggataaaaacaaacaggaagactcgcacggcagcggaaactgtcttccaagttacttgggttacccggcttttccttccgcgcttggggtcgggaccccggccgctcgtcccgccccctcccccgccgcggccccgccccctccccgcctcgcctcgcctcgcctcgtccagccccgcccccgccgggccgggcatgctcagtgggccgggccggcaggtttgcgtggccgctgagttgccggcgccggctgagccagcggacgccgcgttccttggcggccgccggttcccgggaagttacgtggcgaagccggcttccgaggagacgccgggaggccacgggtgctgctgacgggcgggcgaccgggcgaggccgacgtggccgggctgcgaaagctgcgggaggccgagtgggtggccgcgctcggagggaggtgccggtcgggcgcgccccgtggagaagacccgggcggggcgggcgcttcccggacttttgtccgagttgaattccctccccctgggccgggcccttccggccgcccccgcccgtgccccgctcgctctcgggagatgtttatttgggctgtggcgtgaggagcgggcgggccagcgccgcggagtttcgggtccgaggagttgcgcgcggcgctggagagagacaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggcctgagaagcagagccctggagagagacaagagtgccagagctgcggccgcaaaggtgagaacctccgcggccgccagggccagaccgggccgaccgtcgccgcccgcccaccggcatctggcccgcgtcccgccctccctcgctggcggctgtctgggccccggggcggcggggtgggcagggctggcgcggggccgcgggccgcgggccgcgggccgcggggagcccctcgggcgggggcggcgcgggccgcactgggggcggccggggagggggctgcgggcgcccggccgccgtactgggcaggtgcatagctgccggcgcctgtgcctggctgcggctcgctgagggcggggacacgcaacaggtccctcgcggagaaactcggctccagtgagggttcgggggctggaagccggctctcagcgggtcggggcttggggtgccacctcctgctggccgggagctgctgtctttggaggagtggttggtccccggcgaaaccctgtagtttcgatctgatgtcactccctgcggtatgcgcacgccagcgataaggctttgagactgcaaaacactccactcagcctgtgaggcgtagtaggtcgggttttctttcatgctgtattacttatttaaggtaactttgaaaataacctctttaacatttaataatttaacttgaattaaactttcacaagtaatacaaagtattcctacgaatggacaataagatgagcacttaaaaattagtaaaggccggtgagttcagccgaaaaaagtaacgtttttcctgttacttttcctatgtgctctgaaatattattgcattttcccattgctttgaaactaacttgtgtattacattaaaaagccaaagttcctgaaaaacagctaggatgctcctcccattttgtatattaattttttcatcataaaatagtacttgttatttcaaacaaaggaatacagaaatgtgaggagtaaaaaatctcccctttaaagaatatcaattcattacttcaaatagt

SEQ ID NO: 53 - DNA, 2747 bp, Artificial Sequence
  Made in Lab - synthesized insert sequence
aagtgatggagcttcctctgctagccctttgtgagccaatggtaaatgggtgctaaataaaacaactaggtcttgagatacattaattgtaaatgtcacagaaccagtactttcctcaatgtggctaagatagttgatggttcctttttcttctgcactggtcagaccatatctgggctatgatgtttgcttctgggccacacactttagagggaagacaagcagcatgttggagtctgtttaggcgagagatccggcaggggaaggagtcttggtgaatgaggggtggaggagctgcaggatgggaatagaggcctgaactgctaccatgacatattcaaaaggctgccgtgtgaagccaagttatgcttgtcttttgtggtcccagttgatcacattaagacctcatggggccatagaaaagctagagggagactgatttgggttattcataagaagaactttaagtctgttatctgagggtagaatgagaggcatgttttagattctttagattctttactcttctgacaatcatgtgtttttgtagctgtttccttgtggtcatattaattctggtaccacttcatgaaccttttattacccatctttgttttctttttttttttcccttcttaactccctgtttaatttgtggtgagggtgaaagaggagataaagaaaaaaaagggtcaacttgtaactttgccttttcttttcttttcttttcttttttttgccctcagtaactgagggcaaacccatcagacaaccagagccataatttgtggtcaccgctgaaatttaccttggaaactcggttagtatggctgtgaagaggtataccccagctccttaacacagagttaatgcttaatctaaggttttaagtttcttagaaaagaaaaacgtgtacattcttttgtttcttaaacatctaattttgccctcctcctcttctcttagaggcaattgcttttggatcgttccatttacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtacagcgatctggagctgaagctgcggatccctgcacaaaggacagggctggaggatccagagaggtacctgttcgtcgatcgtgctgtcatctacaaccctgccactcaagctgattggacagctaaaaagctagtgtggattccatcagaacgccatggttttgaggcagctagtatcaaagaagaacggggagatgaagttatggtggagttggcagagaatggaaagaaagcaatggtcaacaaagatgatattcagaagatgaacccacctaagttttccaaggtggaggatatggcagaattgacatgcttgaatgaagcttccgttttacataatctgaaggatcgctactattcaggactaatctatgtaagtatttcttccaaataatcatgtgaagtggtagctaggaattaatgtaaattatacatcttgtcataatcaaatgagaatgtggaatacccaaactctctgtttaacatttctatttctctttaagatagaaagatttgttgcttgcttacccatgtcttgcttttctttgaatcttaacacattaagtttaaataatacaggctgcaattacatataataaaatggcatttgaagacttttgtagtggtcttctggagcataataaggtgggagagagcatgtaacaggaagaccagaaggtttaataaggtaaagagagttgcattaattggacgcagacagcaaaacggtcaaaaatcaagtgcatacccaagagtaaagtggaggggctgtaagctgagaaatttctgtggacagcatgaacagcttcactggatgtagtagggaagtaggaaagatgaatgctgaggtttttaagaggaaacaattagggtaagattgaggctggctggggtcgtcctgtggttagcagctgacatgaatgttggagtcaccgactttgtcactgaccatgtagaagaagttattgaaaatcataagggataatgtagagagggataatgtagagaggaaaaatgtaagccagatacta

SEQ ID NO: 54 - DNA, 2729 bp, Artificial Sequence
  Made in Lab - synthesized insert sequence
tatgactgccctttcacagatgagtctgagactcaaaaactgtcactagcgtaaggttgtccagcgagtttagtggctgggaggctgaggggtagacagggcggctagcgatgtggggcaggcctggcggtcgccacagacgacctaacggtaggaaaatcttacagccaccaggagagttccaggcgccgcggcagggggactgggagaggggactgcgcccagaatgaaggctcgggacaaaagcagttgcgcaaacgcgccaaggctgggcgtcgagtgaccgcgggcggaggtcaccagcggccgctccccggaagccacccacggaccacgcgcgcccctgcacgcagagggggccagggctccacgggcgagcggcgaccctgcctccgggagacggcgcggcctgccctgcgcgcctcagccccgggtgccggcgtctcgggcagcaccaccaagtctctctggaggggaaaggatggtcggatttgccccatgtcccttcctctgacccctccctcaagagtgccccgggacaccccgcctgtggctcagcctcccccgccccgcgctgccatctcctcagggccgggcagcaggctcccgagcgcccacagacccggggtgcggcccagcccacaaccgtcacctcaggggcctcaggcgcccagcggtgctgggcggggctggggcacgaccgggagcatgcgcagagcgcgcgtttcgcccatcgcgcacgcgcacacacctgctccgcccccacgctgcgtgccgctgctgggttccgccacgcccgtcatggcggcggccccggccggctctggccccgcccctcggtgacgcgtcgcgagtcacctgaccaggctgcgggctgaggagatacaagggaagtggctatcgccagagtcggattcgccgccgcagcagccgccgcccccgggagccgccgggaccctcgcgtcgtcgccgccgtagccgcccagatccccgcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggcctgagaagcccgtcggagaagaccttcaagcagcggcgcaccttcggtgagtgtcgccgcgagggcggcgggtgcggcggggccggggtccgagctgtggagggcggcagggcctgggacgccgtgaggggtcggggccgagccgggaggccgagcggggccggtggctgccggccggcggggccgagggatgcggggcccggggccccgtgagggacccaggccggggccgagccgggagggccaggggctggtctgggccggggcggcctgcggacccctcggaggcctgggaggaggcagccggcggagggcgggcgggggccggtgcgctcggggccccgggcctgctgaatcaccccgcgccctccgcggcgtggtgcctgccgtaccccggccaccgccgcccccgcaagcgcgctgcgggcgagggtcgcgcttctggggcccaacagcccccggggccgcgctgggccgttcggctctgaagcggggctgcgccgggacccagcgcgtcaccttcagggctggttttcccgtcgagggagcccgcccgggtgcgagtgcctcttacagacctcagtgcctcggtcgagaggaggggaatgtgctgggccccggcctcgccgcgcccgggacagcccacagcggtgttgccagttcctgtctgtagcccaggagctggggtgggagacttctcttgggtttgcgattacgttctcaccttggcaccaggaataccgaatacatcccttttctgccggttcagactggaaacccagcttaggtgtgctggcatttggttaaacgtgcaggtgtcaccatcacaatatttactccacgcagatgatgcgaagtgattatttcccgcttgagcaagagaacgccgcagatccaggtatctgttgttgcctaagttccagacaccggtttggaagggaagcaccgtgttcatcgcggtggactgcgaatgcctgccgccatcctcgtcaggagcccacacagtggt

SEQ ID NO: 55 - DNA, 2759 bp, Artificial Sequence
  Made in Lab - synthesized insert sequence
tacaatttaagggcgtaccccttaaaaactcagtcatcaagataaataatatttcactacagtatcttcagaaaacacaattaatgtaaaaattatgatgagtgaaatatcaagtatttaaataaagataggatcagtaacagtgctgtgcagagtttattggaacaatgctggccaggggcccccttcttggattttctctacatctcatcattcagactgtggagtgctcctgttccagctgtccctttgcccagctaagggaagaaaagtcttccctccccaacactgtttcatgcaaatgctataaggaaaaatctgctcccaaatagcaccatccaagaggttctgcaggatgtcacagtgctagaaagtgctaactacacatgatgccactgtctcctcaatcagctcagtaatcttgtcctctttctccaaataaattattccatggatacattcattggctgattgatggagtcatttaatctactagcatttagggaggatctattattcaccaggccctggcctaggtgctggggattcaaaggtgtataaccctcacacagccctgcatcctaaggaacactgctgggcttggcttgctgaaacttagattgggaatcacagtcataaatagaaactccagagggaaagctctccaaatttggggtcatgagctgctgaacccactgggcagagctctggggtgctggggtgggttgtcaggcatgactcacctctgctcccctctccaggtatcatcatcatgatgacgctgtgtgaccaggtggatatttatgagttcctcccatccaagcgcaagactgacgtgtgctactactaccagaagttcttcgatagtgcctgcacgatgggtgcctaccacccgctgctctatgagaagaatttggtgaagcatctcaaccagggcacagatgaggacatctacctgcttggaaaagccacactgcctggctttcgcacaattcactgcctccagagcaccgtgccacgggctcgggacccacctgtggccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagcacaccctcctcactcttctccatcaggcattaaatgaatggtctcttggccaccccagcctgggaagaacattttcctgaacaattccagcctgctccttttactctaggggcctctgtcagcaagaccatggggacttcaagagcctgtggtcaggaaatcaggtccagccttccctgtagccagacagtttatgagcccagagcctcctgccacacacatgcacacatatctagcattctttccagacagcatcctccccgccttccaccttggtagatgcaaggtctatctctcccatcagggctgccaaagctgggctttgtttttcccagcagaatgatgccattctcacaaaccaatgctctatattgcttgaagtctgcatctaaatattgatttcacgttttaaagaaattctcttaaattacaattgtgcccaatgcagggtggctctggggggcaagtaggtggtacaggggattggaaacatgctccgcgcctccagagaaaagttgctcccgaggtccatgcccctggaacgtgttcctatcactctggctggttgggctggtccttagactgggtgcttatgattaaagggtcttggttagcccactttccctctccatgtggagatggaaggtagagaaggatacagtgtctatcctcaagttgctacggttcagtgagagaggcagacatctgaacaggcaggtaggattcagtgtgctcagtgcactggggatttggagagagatgggcttgctctctctgtgcacccaggagggccacgcacttaaaactgtgtttgtggatcagagaaggctttatagcacagggggcattcagatgagtcttagaggaagagaagaaacatggcaagcagattacatctgagccgtttgaattgtgtttttctttcttcccatgtttattttctaagatctacctgaacttagagactcaagatatttttttaggaaacctcctac

SEQ ID NO: 56 - DNA, 2735 bp, Artificial Sequence
  Made in Lab - synthesized insert sequence
aaatgtacaattaaattattattgactatagtcacctgttgtgctagcaaatactaggtcttattcaaactatctatttttgtacctattaaccatccccaccttccccccgccactactcttcccagtagtctctggtaaccatccttctacctttatctccatgagttcaattgttttgatttttaggtccctcaaataagtgagaacatgcgatatttgtctttctgtgcctggtttgtttcacttagcagaatgacctccagttccatccatgttgttacaaacaacaaactctcattctttttgatggctgaatagtactgcattttgtataagtaccacattttctttatccatttatctgttgatggacatgtagcttgcttccaaatttaagacattatttgtaagaaccaaaaactagggcctggcacggtggctcacacctgtaatcccagcactttgggaggctgaggcgggcagatcacgaggtcaggggatcgagaccatcctggctaacatggtgaaaccccatctccactaaaaatacaaaaaaaaattagcctggtgtcgtggcgggtgcctctagtcccagctgctagggaggctgaggcaggagaatggcatgaactcgggaggcggagcttgcagtgagccgagatcgcgccactgcactccagcctgggcgacagagcgagactccgtctcaaaaaaaggaaagtactttgacaacagaagtctgtgttgaaatctaaaacctctattgcttgttcttatcaaagtagctatctaggtagcatgttctctgatgcaggagctgacttctgttttttcaaacctctttccctttagacgttttggaataatgggactctacaaaggccttgaagccaaactgctgcagacagtcctcactgctgctctcatgttccttgtttatgagaaactgacagctgccaccttcacagttatggggctgaagcgtgcacaccaacacagagacccccctgtcgccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtgagacggcttgccatgaaaaattccgaagatgctcaagagggaggtttcctcctgagtgaagagaagtgattctcccttgactctggctcctgccaccacaaatgttaccctcattggcttgaaaagcatccaagggtgcacagggagtatggccaactggacctgttgtcaccttaattgtcatgctggctggttggattttggggtggcagttggactaatgtgaaaaaaacattgctgaaaacctaaaaatgaaagtttgtgagtgtttattggttttcttaagagaaatggactattttgctctcatgtgtaatgttttctatttaaatctttcttaaatataccagctgttctctttccctgaactctcccccaggttctaggacaaatttaataacatgtaattctcctcaaatacttttgtatgtctcagtgttggtgttttcctccctaaaactaacattagggctgtgccacgggcatgactttatttttgttgggcttttttttccctgcttaaggagaggtgtcttttttggatatgagctatttattttgtgaaatgaaaattgttcacccaaatgattctcttataaactatttgtaaatgtcacttattcattagtgtttgacataatttttagaatatttattttgaatcaatcctttcattacgaaagacttgaagttttgtgtccattcttacaagccctggtcagtcaagtcccaataaatggtcagcacaaaaaagatattcttgaaaattgctctttattaaggtattacgttgagtttgcaaccagatgggaaaaatcacaaaaatgagaaggggagcagatatcttgttgaggtctggatattatctccctttataaacttggtgtaggccgtatattttgaaaataaacatctggttgaggttatttcatttggaaaccttccttagagtcctgttgctcagtgtggtccatcagacggtagtactgccacaagct

SEQ ID NO: 57 - DNA, 2746 bp, Artificial Sequence
  Made in Lab - synthesized insert sequence
aaagcaataagaggactgcggaagagctccctgtcaatgtaccgctctacaccagtgtattacgacagttcgtacacaacagtctgtagaggccacctgtctctccctgctgcgttaggaattcaggggagcaggtggtggcagtaagggattttgagggaacggaaatcggatcttgacccagatctgggccgccgataatctcctactgcgctcagactgctgtggaggtgttaggctgagcccgatgccggcaggcaagggaggatgggcggcttgggcagcgcctttgcagacgtggccatttcgtgcctctgcagcaccgccggggggcgcaagagcgcgcgcccggaattgctcattcatcctgtgccgcagagccccgccccttgtccctgcggacagacatttcttctgcgctggtctggccacgtgcttcctgtgctaggagctgcccggaaatgtgaccacctagtctaaagtgggcttctggggcctgagcgctggatggatgcccaccttcctgtcttggtcctccaaaggaggaagctgtgactgagctgtcttggtctggaaggaggccttcccggtttaggatgggaaggtaacattcattaaaagcaacgtagactatagtgtagctgttctcaaaagtagtacatcttagaaaaggatctttagaaaagatcgctttagaaaaggaaattcgttttcagattacgtgagtagcctaggtaacacagccagacctcatctccacaaaaaaaatgaaaaaattagccagcttggtggtctgtgcctgtggtcccagctgctccagaggctgaggtggggggatgactggagcctaggctgcagtgagcctagatggcatcactgcactcaagactgggcgacagaccttatctctaaaaaaataaagattgcatgagtattttgttccacttgacagtcatcaatagattggtttaaattgtgatatcttttttacttaccgcaggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagggaggttcaggaggcagcgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattggtgagttgacctcagtaacctgagatcccaggatgctgggacaggaggtctgtccaggggcttctcttgtcactcactcactccctccgtccttctctccctcctccagatgaagttcgcactggcacctaccgccagctcttccaccctgagcagctcatcacaggcaaggaagatgctgccaataactatgcccgagggcactacaccattggcaaggagatcattgaccttgtgttggaccgaattcgcaagctggtaagcaccacatataaatatgcatttaatgtggtgtgatagttccagtgcaagttgggtggagtgactgacatcattcattctttggcacctaccaaaatgtggaataggctgcttgctatattaattggacttctaaatcagatagtccctaggttatggacagtttgtggatatgtctgttttgccaattccttgtgcttacatcagtgagatatggttcgtaatctaaaaagttgaaatagaaattctaagataatgtgtcctggcattaaaatattacatttttttattcccctacaggctgaccagtgcaccggtcttcagggcttcttggttttccacagctttggtgggggaactggttctgggttcacctccctgctcatggaacgtctctcagttgattatggcaagaagtccaagctggagttctccatttacccagcaccccaggtttccacagctgtagttgagccctacaactccatcctcaccacccacaccaccctggagcactc

SEQ ID NO: 58 - DNA, 2758 bp, Artificial Sequence
  Made in Lab - synthesized insert sequence
taaaggctggtacttggaacctgcaagccgtgcatttggaacctcggactcaagtgcctattacgtaattccacagcgtcccggcctccaggccgtttcccgagccctccagcggagcgggggataaggttaccacgcccgcggtggccggggacactctgagtttcgcgtgtggcttttagggacgtttatatttgaatttccctgaaccgccgagtgtgggcggtggcgcagatccgtcccggaaacctccgggctccttcccgcctttctcaggcccggcccctccaaggggtccccgcggggcggcgggagggccctgggcccagagccgcgcgggtgggcagtcccaggcgtccttccttacagccctgagcctggtccgggaaccgcccagccgggagggccgagctgacggttgcccaagggccagattttaaatttacaggcccggcccccgaaccgccgaagcgcgctgcctgctccccattggcccatggtagtcacgtggaggcgccggggcgtgccggccatgttggggagtgcggcgccgcggcccgcgccacctccgccccccgcggcttgcctccagcccgcccctcccggccctcctccccccgcccgccgctccgtgcagcctgagaggaaacaaagtgctgcgagcaggagacggcggcggcgcgaaccctgctgggcctccagtcaccctcgtcttgcattttcccgcgtgcgtgtgtgagtgggtgtgtgtgttttcttacaaagggtatttcgcgatcgatcgattgattcgtagttcccccccgcgcgcctttgccctttgtgctgtaatcgagctcccgccatcccaggtgcttctccgttcctctaaacgccagcgtctggacgtgagcgcaggtcgccggtttgtgccttcggtccccgcttcgccccctgccgtcccctccttatcacggtcccgctcgcggcctcgccgccccgctgtctccgccgcccgccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagagcggcctgagaagcagagcccaggccagcgcgactgcgacccccgtgccgccgcggatgggcagccgcgctggcggccccaccacgccgctgagccccacgcgcctgtcgcggctccaggagaaggaggagctgcgcgagctcaatgaccggctggcggtgtacatcgacaaggtgcgcagcctggagacggagaacagcgcgctgcagctgcaggtgacggagcgcgaggaggtgcgcggccgtgagctcaccggcctcaaggcgctctacgagaccgagctggccgacgcgcgacgcgcgctcgacgacacggcccgcgagcgcgccaagctgcagatcgagctgggcaagtgcaaggcggaacacgaccagctgctcctcaagtgagtgctagctggcggccgcgttagcgccaaggaggggcgggggcgcaaccgcggcgaccagctcaccgggttctgccgtggggagggagcagaggccaggatgcacgcgtccttctgaaggaacagggtctcggtctccggaaaggagaaagaatctagagttcatagcggagcaggggtcgcggagggggctcgagctgtagcgctggggggccgtgatgcccatttctagattttggatacccgctgggacgtggtaagtgcgcgcctgggactgccgagaaggagctcccgctttcgcactcgaatccggggagccggcgcggagaggcggcccctcaggccccaggtgcggggagctggagcgcgagcgcgcgctcgcgtgcgcgccccagtttccggccggcgcgagacaaagcgtctagcggatttgcagtgccgggatgggcggccggggaggactggcagcccgcctctagaatgaatgagcttcgcgcgggcagagagaggaaggggagggaccttcccgcagcatccgcgtctcctgggggtgggtcccgctttggcgcgctcagtcttggccctgtgacgttttgcgaagattctacgcctgctttaggcgggagagagaggcggag

SEQ ID NO: 59 - DNA, 5708 bp, Artificial Sequence
  Made in Lab - synthesized insert sequence
caccctctgccctaccagctttccccttgtcaacccctcacatggcctcaggctggcccaggagctcctgttggagaccagtcagcttgacagacagaacattctagaatgctttgggagaccagtgtgggaagaaagaaagaagatttgctcccttccttcaggctgttcccagtctgacgaagaaactaggacataaagaaagctagaccccagacggagaaatgaagtaaaaatcttgttcccagtgtgaagataatgtggagatttagggtaatggtggagatgactgcctggtcaccacagggaaaggagacagcagctcatcttggttatagccacacacgagcatagacattagagaactgagttcaaccacccagcaacaaattattcaaagttggttgaattcacagtatatgtgccccaaattgataggctctgtaaggaacagggatgaaggaacagtctctggcccaagaagcttcagaggagaagagataagccagacaatatactgaggaaatggaaaagcagccgcatttgcaggggaagacttcttggaagaggtggccttgagcctgctgagtgccagaccaggagggtggcatggagccttggaggaggttttgctgaggtgcactgaggtgggaactagccccatagctgccactttcacccagtgctcccaagccacacagcttcctttttcctgtggggcctcggcttggcctgtgtccacctgcctggctgagtcctaggaccttaccagctggggaggggcatcaggatttcttatcatccgaggagactggtttcctatcacccagcttctcttgagctggggggccgtccagcctggtaagtccctgatgtgtgtgccacttgtctacaggagcggcctgtggaggtgggtgactggaggaagaacgtggaggccatgtctggcatggaaggccggaagaagatgtttgatgccgccaagtctccgacctcacaatccgggtccgggtcccccggagaggttcccgacataccaataacttcgtatagtgtaggatatacgaagttatattagtattccgataacttgtttattgcagcttataatggttacaaataaagcaatagcatcacaaatttcacaaataaagcatttttttcactgcattctagttgtggtttgtccaaactcatcaatgtatcttatcatgtctggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccggggatccaccggagcttaccatggtctccaaaggagaagaagataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggaatggatgaactctataaatgaggtactcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaataacttcgtatagtgtaggatatacgaagttatataggtatgtcgggaacctctccgggtccggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtagaggtacctagagaccagctcccttctctggggtccatcagagtctagaggataatgagggagtccacaatgggcaggcgggttgtacttagtgtggtgcacggatgcacggactaaggcaccgatgagactgatttgagtcggctctcctgggctcagtcctggctccagcaccatgatacttgggcatgtcatttctgtttggcggccctttttgttcccattatctatgaaattgggggctgtatcaaatcagtggttctccaagtgtggtcctgggaccagcagcgtctacatcacctgggatgcaaatgatcagcccccaccccagatccactaaattggaaactctggggtgaggccagccttctaggtgattctgatgcacatgagagtttgagaaccactaggttagagggtccctgggatcccttcatctctgactttctgagaatctatgcacttttaaatacctcctggtgatgtcctttatttgtgctccctgcgaagtacccagcactcacagtacctgttcccaaggagctaccactcacagaagctaagacttcccaccatctgaggaaatagctggagaaactgggcaaagcaaggaaatcaggccacgtaaaaccctctacggaagtgctgagagaatgtgcaatagggcaggcatccaccagacagcactggtcagagcaggctttctggagaggaggagctctctgaggatgcgacgagcgagtgttagctgataaagggaagggcatgttgaagggctgatggtgcaacagaagcatggaggtgggaattaagcagccacagagtagcatggcaaggctaggtaagagctgagggccgcagtgtggaggagcggtgtgaggggtcccaaaaggggagcaactgtgcccgagggccgagaagctgaggttttgtaggagcccttatgaatgaagatccagtccggggtgcgctagggctgagaagccctggggactgca

SEQ ID NO: 60 - DNA, 4717 bp, Artificial Sequence
  Made in Lab - synthesized insert sequence
ccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtgggccaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtagacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 61 - DNA, 4828 bp, Artificial Sequence
  Made in Lab - synthesized insert sequence
ccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtgggccaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtccggactcagatctcgagctcaagcttcgaattcaaagatgagcaaagatggtaaaaagaagaaaaagaagtcaaagacaaagtgtgtaattatgtagacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 62 - DNA, 4487 bp, Artificial Sequence
  Made in Lab - synthesized insert sequence
tagccgttcagtcagtaaaaaattggcaagcttttcattacatgtagcctagaccttcaggaggagtttaattgccagttcagaattgtaatattttaccccccaaacaataaaaacatctctatgtataaattcaatttgggacaggattattttaccctgttttctggctgagaggataatgaacattaccacaaggaggttagcatgctaaaacacagtaacagagccatatttattaccttaaaaatcagataagaccaactatttttaaaataatgttttctttaaaatgaatcatttacccatggggtgagggtggatgggaaatagcccagctgatttagaaggtaagaaaccatggctcctccaaccccactaagaaacttctactataaattatataatatgcaaactataatgatataaattataatgtgatatttgagatttacttattttgacttttaccaaccagactatttggctggaattgtcctatttcccactgaactttttttttaaaagcttcatcttttctggtatgaaatgcagatcatagtacgtatcctcgcattgtggctcagtttgagaactactagtaatgctccatttgcctttatgaagcatatcacccttcctgctggccaggccgcttctagatacgctcctacaagtaaaacttggctttctgttggttgtctggtactactatgccaatagactccctattctttagtccttttaaaaaattaaacagatgcaagaaatatgtaagtattaagactgtttatgttgtggtgtttctgcaactgactgcaaacacgtgtgtattttttcccagccatacatcctggcggaggagctgcgtcgggagctgcccccggatcaggcccagtactgcatcaagaggatgcccgcctactcgggcccaggcagtgtgcctggtgcactggattacgctgcgttctcttccgcactgtacggcgagagcgatctggtcgacgggactgctgggcccggagaggttcccgacataccaataacttcgtatagtgtaggatatacgaagttatattagtattccgataacttgtttattgcagcttataatggttacaaataaagcaatagcatcacaaatttcacaaataaagcatttttttcactgcattctagttgtggtttgtccaaactcatcaatgtatcttatcatgtctgattggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatggtctccaaaggagaagaagataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggaatggatgaactctataaatgaggtactcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaataacttcgtatagtgtaggatatacgaagttatataggtatgtcgggaacctctccgggcccggatccattgctactgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtgatgctgagcttctgtaatcactcatggcatcagaatgcaataaaagcggaagtcacagtttgtttcctggaaactttgacaagctttattaagttgagagagagagaggggaaaaaaaaaagcctttcgtagttcagtaattgccagcaatataacacggctaaaatgaagtttttacagtatatgacatagtgcgcttcataaataggtttatttctgagtttttagcaaaatgtaatgaaatatcaggttgatttctttgattaaacagaacaaattacttgagtaataggaaattaggaggatctagggacagaaggaaagtgaaaaatgtgaaaatacaaaatacccaagatttaagaccggggggaaaaaaccacaaattggtaaataaaggtttgctatttgtaaaaaatttcatttatctctaatatgcttatgtgattgcccctaggggagtatatttgggattctaatgttttattttcatgcttatccaaagattactattgtatcttcaaatgaatttaatattgtgagatggaactgccggggattaaaaagactacccaaaagatttttggcacttacaatttttaaaatagtttatgtcatctcttcattatttagggctggatggtcaactcagtcagtgattttttgatgcttctcttatcctccagaatagagacctaaggacacgtggaagtcagtttaattgccagagagaaggatgcaatcactaggtaaaatgaggtttttaggattatttattgattccaggttcccatgctttttgttagagcttattagtacaggttctcaagagatgaccacataaaagtgctctgtttataaataagcaggtttctgtagtactgactggttcatcacaaggcaagtcagaaaccagtatccttctagctctccagtcaggacttccttatgcctctagttttatgaccggttaaggagaagccagagttagagtaggagag

SEQ ID NO: 63 - DNA, 3335 bp, Artificial Sequence
  Made in Lab - synthesized insert sequence
gattaaagatagaaggaaactgttaagattggtttgtgagtggattgaaaaaggttcttcaaatattcactaattccttagagattaaaaattagcatagaagtcgccattcaggctagatttctttaatgtgttgtttaaataaaaaataatggcctgcaggctgggcgtggtggctcacacctgtaatcccagcactttgcgaggctgaggcaggaggattgcttgagcccgagagttcaggatcatcctgggcaacatagcagttaagtgtctctaccaaaaaattagcctggcatggttctgagcgcctgtagtctcagctactcaggaggttgaggcaggaggattgcttgagcccaggaggtctaggttgcagtgagctgtgaccacgccactgcactccagcctgggtgagagagcaaaaccctttctccaaaaaaatttttttaaaaatggcatgtatattgttcgtctaaagactacaaaaaagttacaattattgtaagtaaataagtcagtaataatcaccacatggagaacaactgttagcatttctgtgtatggtgtgcttgagcagatgaggaaacagttttaagtctgccgttaggcacacaaacctgggagactgaaaatatttgttgtccaaaattaccattaattaaatgaaaaatgcatcgtttaacagaaaacttcaaataatactaaattttaggatctgttctaaaagtttcccatttattaataacataatttttctcaaaacaatgttaatccaagtagcctggctacagaacttgaactcgagtcatctctcaggttgcctgggtttgaaactcggctctgtcacttctgtgacaataactgtgtgacctcaattaagcttcttaacatctctgtgcttgtttcctcatttataaggattgtaataatatctcattttataggattgttgagaagttaagtacttaaagcactgcctggcacaaacttgaagctttgaggttagcctttaaaaaaaaaagtaattttggactcacatcctatacctttgaatttcctttaaaagatagtgacataatctaaagggcattgaaggtctagtagagtttaagataatcatgctataggcaatatttaaagtgattaatagtaaatttgctttactgatttgtatatttaattcataattgtttctttacaggtttctttacctccagaaagaagaatattggccccttgaattctggaagttcattgaagagtctgaaattagggacttatttcaaatttggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggcctgagaagcagggccgctagtcgaggcgccacacggccaaacgggccaaatacgggaaataaaatatgccagttcaaactagtacttctgggagagtccgctgttggcaaatcaagcctagtgcttcgttttgtgaaaggccaatttcatgaatttcaagagagtaccattgggggtgagattttcttttttccctgcttcatttaatcgaatcatacattgacagaaaatattttgatgttccaattgtgtgattatgaatagctaaataagtcatagagtgaagaaaagagctgttcagcttaccttagctttaaaacttgcctttatctttgttaaaatgatgtgtttttatatttgattgtttggttaatcccttgtggttatattgtagctaaaatacagataaaaaacatgagtagaatcagtgtctcaagtaataacatttgacctgatacctatttctagctaacaactagaagcttcttcaaattaatacagtacatgtttgtgctcaattatattggtttaaaatgaatagtaaaaaaggtaaccttaaattctgatctatataactgaagtatttgcaaataattatacttacttgtaaaattaaatgtaattaaatactgctagatgtacttttttttggtatttaagagatgggatcttgctgtcgcctaggctggagtgcagtgtccggatcatagctcactgcagactgcagcctgaaactctaggctcaaatgatcttcttgccttagcctcccaccaagtagttggacctaagggtgcatgccaccatgccaggcaagttttttttttttttttttaagagtcagagtctcgctgtgtttccaggttggtcttgaactcctggcctcaagtgagcctccagcctcaacctcccaggtagctgtgattacaggtgctccctgctgagatgcatatgtaagaatgctcctaagctgagggaagagaaaaagtgattaatggaaagggacataacaggaaaatggaacctgaagaaaaagatgaaagaagaagggaaagagtaggcactgacaagggtggttatggtgggacagagatgaggggaaggtaagatgcagggtgaaaaggagacaacaaacagtacagtattgtcaagagaggacgtgaaatgggaaaattgaattttagctggacatggtggtgcattcctgtagtcccaggtatttggaggctgaggcaggattattttagcttgggagttcaagtctagcctgggcaacataatgagaacccatctcttaaaaaaaattgactttaatca

SEQ ID NO: 64 - DNA, 4472 bp, Artificial Sequence
  Made in Lab - synthesized insert sequence
cactgggagactccatctcaaaacaaaacaaaacagaacaaaagttagctgggcatggtggcacacacctgtggtcccagctcctcaggagtctgaggtgagaggatggcttgagcccaggaagttgaggctgcagtgagccgagattgcacgactgcactccagcttggatgaggcagccagaccctgtctcaaataataataataataaaatagaacaattataacagattgtaataaaactcatgaatgtggtctctttctcaaaatatcttatagcactgtaatcacccttcttttccttgtgatgtaaaatgacagtgcctatgtgctgagatgaggtgaggtggatgacataggcattatgacctggcgttaggctactattgacctgagaatccatcaaacttatgaattgtttatttctggaattttccatttaatatttttgggccatggtttacctcaggtaactgaaaccacacaaagtaaaattgcagaaaaggagggactactataataagaagagaaggaaggagacaggaagggcataaggcagacaggaagtggaggggaaagataggaagtgcaggaaggagacaggaagtggaggggaaagacaggaagtgcatgagggagacaggaagtggatggggaaagacagaaagtacatgagggagacaggaagtgctggggaaagacaggaagtgcatgagggggacaggaagtgcatgagggagacaggaagtgcatggggaaaattggcagggattatcttgaaaagacaggaagtgctccagaactagatacttaggcatccagggtagagtggccccacaggctggagggaagacagggattcttgagagactggagaccaagaagagaccctaacctctgactcattgccattctgcaggaaaaccgggaggtgggagactggcgcaagaacatcgacgctctgtctgggatggaggggcgcaagaaaaagtttgagagctccgggtccgggtcccccggagaggttcccgacataccaataacttcgtatagtgtaggatatacgaagttatattagtattccgataacttgtttattgcagcttataatggttacaaataaagcaatagcatcacaaatttcacaaataaagcatttttttcactgcattctagttgtggtttgtccaaactcatcaatgtatcttatcatgtctgattggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatggtctccaaaggagaagaagataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggaatggatgaactctataaatgaggtactcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaataacttcgtatagtgtaggatatacgaagttatataggtatgtcgggaacctctccgggtccggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtgagccttcctgcctactgcccctgccctgaggagggccctgaggaataaagcttctctctgagctgaaagtgactccatgtctattacccagggcttaggcaggagacagatgggaagactgcaggtggggctcccccaaagccacacagcaggttggggaccagatgggtctcccatgtgaagcactcttggctgtgttattgaaaagaatcccggggttcatgaatttggagagcggagctttgtttcttaagaagcggatcacaacctgaagaccagaagcatggcttcttgccaaaaaacaaaagcaggcactttaagggagggaagggcaaggcaggaatttatgctgagtgggttagctaagtgcacgtattcaactggttatagaaggagctatgaatattcatggacaggtggacacatggacacacgcatgtgtgacaagcaaacactcatttttttttttttttgagacggagtcttgctctgtcacccaggctggagtgcagtggcacgatctcagctcactgcaacctctgcgtcctgggttcaagccattctcctgcctcagcctctccagtagctgggattacaggcatgcaccaccacgcccagctaatttttgtgtttttcgtagagacggggtttcaccatgttggccaggctggtctcgatttcctgacctcgtgatccgccctccttggcctcccaaagtgctgggattacaggcgtgagccaccgcgactggccgcaaacacattttacatgcatcccaccttcacttggtggtggaggcttaacatttaagtgcattccattgaatgcattcgtatcgaaacacgaagcagggatatgaagacactcagtgcacagcctctgtcaacagccagaaccagtccgtggtctgcggcatcttatcaggagaaagttactgaaatcagtctcttgttcaactaatgctgtagttatgccttgtggaacagggggttcagcta

SEQ ID NO: 65 - DNA, 2723 bp, Artificial Sequence
  Made in Lab - synthesized insert sequence
cgcagcccgggcgaccgagggcgaggaggcgagccaaggacatcagcccgagggcgcctcgagacgccccgcgtggaccgcgctcccagctcctcggcctcgccttccaaccatccgcccaccggccccagagcagcgtgcccactgtgagcgccccaccctgcgtctgcaggtgggtgggtcagagaaccgcaggcacagaagagggtacccagcttcccctccgccagccccgcgaccgcggcgcgcgcggcctcgatccgggttcctaggggcggcgcgcgggagggggcggggcctgcgcggcagcgtgggcgccaggcgcgcgggaggagggagccgggaggagggggcggggccgcgccgcccgcgccgcgctgggcgctctcggccaatgagcggcgtccacatgccgcggcggcggcgaaaggggaggcagcggccgataaatgctattagagcagccgccgcggagccgtccccgacgccacctccttttccttcgccgcagtttcctccgccgctgtcgggcgtgcggcgctgagggacccgggcgagcgcgccgcgcaccgccccgccggctcgcctccctcgccgcgttccgccctcagtggtctgccgggcgccccctcctccggcccgggcggggcctctgatcgcctcaagagagcggggagggggctcgggggccgcggcctgccctcccggcgggcggctgagggcgagggaggccctcccttctggcgaggggagggagggtgggtcaggagcccccaacccgccctgcggagctcggggccgcgcgaggggcggttgtctgggggagggggcgcggggtgattcagcgcccggcgaggcggaagcggccgcaagaggaggaggggagagcccgtccgcgcctgggctcccggggtggcacgagcccgcggccggagtgcgaggcggaggcgaggaggccgcggggacgggaggcgaggccggccgggcccccgaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagggcagcgctgagaacgcgcacaccaagacggtggaggaggtgctgggacacttcggcgtcaacgagagtacggggctgagcctggaacaggtcaagaagcttaaggagagatggggctccaacggtaggtgcagggcgctccgctgcaggggcccggcgcggccgggagagccagggaagatggctgaccgggctccacctcgtgggcttcggctccgcgcccgccgacagctgcgggcggagggtcgggccagcgcgccggccccgcgggagagaaaggggctgcggtcctcgcctcgccttccctggacctcttcgcttctcgggccctcgaccttttcggcgcgcaaggctcggaggcttctctccagcagcagccggccccggtggagggagggacgtggcttctgcagctaggttgagcccggcaagacgttttctcgtcccctgccgatttatgaggagtcgaggtcgttggaaggcctctgaccgttcttgtcctacccaaagttacacatctggcagaagtgatgacatcgctgaaaccactcctaggttcaggagcccgaagtgatttcacgcttagggctagacctcaggccattgattacaaagagtgcatgcttccttgagatatcttcgtggtatgccctctgctaaaaaaatcggatattcttatagctaggtctgtgttttaaagatattgatgcttagaattgtagcagttctttttagaaaaacgaagtgctaaaatgtctctctctttttttttttaacctccctcttgacacattgcttgacgaatttctacattctacagagttaccggctgaagaaggtaatcttaacatgctgtttctgttttttttcctctgttggtgtgctgatggtaagatgacagttaaaacacatgtgtttgtttcttacaggaaaaaccttgctggaacttgtgattgagcagtttgaagacttgctagttaggattttattactggcagcatgtatatct

SEQ ID NO: 66 - DNA, 5959 bp, Artificial Sequence
  Made in Lab - synthesized insert sequence
ccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtgggccaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtccggactcagatctcgagctcaagcttcgaattcaaagatgagcaaagatggtaaaaagaagaaaaagaagtcaaagacaaagtgtgtaattatgtaaacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 67 - DNA, 3033 bp, Artificial Sequence
  Made in Lab - synthesized insert sequence
gcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcgcccagacgacctggagatcgaccctcctgtggccacagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtagatacaggcttgaaagcatcaagattccactcaattgtgcagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttc

SEQ ID NO: 68 - DNA, 2753 bp, Artificial Sequence
  Made in Lab - synthesized insert sequence
caaactcctgacctcaagtgatccgcccgcctcggcctcccaaagtgctgggatgataggcgtgagacaccgcgccctcccttctgctttgtacttcttcatcgtacttatcacaacttgagcattacacgtatttgtctgtctcctctctctaaaaggtaacttctataagggtggatccccaggaacatggagtgtgctcaacaaacatctgaactaacctgattggcgtatcctctcagtgtctccttttagtctcgacaattgtgtttcacactttctcagtatgcaaatatctttctcggtctgggtcatgcctttcggtttctgccccagtgcagaggcctctaagtttatcagcgtgtggaccacagattcgcagaggcctgagctgcggcgctgccgggatatggcgcgtcgtcgggcctcaagcacagcccggggtccctcgctggctctgttcggaccctgctcggtcgctcgcctgggccccagccatctctcacttcagcccatgagttctgccccacgtctgtctccctgggtgtcttaagagacgcgacccgcaccgacgcccccgcgaatgcaccgcgaatgcacgtgaccgcgcctagtggcgccccctcagtcaacgccgtcgcagtgcccctcccgaagcccgccgcttccgccggcccgcgcggccccgcccccagccgcgaggaaccaatccgcatgcacacttcttgtccccgccccggcaccctcctcggcgcgcgcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggtcagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggcagaacccagatcagcaggtgctgcgctgctaacgcggcggcggcggcggcgggcgcgggaccggaaatggtgcgcgggcaggtgttcgacgtggggccgagatataccaacctctcgtacatcggcgagggcgcctacggcatggtgtggtgagtgtccgcgaccggccacgcccggcccccccaccgcccggggccgcagcggacgcagcctcggcctcgcccgcgcctcggcctcaggcgccgcggcgacctcgaacccggacttcaccgcggtccgcgcgccagggctcgggcgcggccccgggccggttgaggtttaggtgttgttgctcgcggttcccgggccttcggtcgctcgggaaggggctcagggagtggcgcggggggcgtaggccggtactagtggacccttggccccgggcgcagcgcggcctttaaagtctggaccctcgggaaggcaacggaagggtccccacccctcaatttgctagtatcatggccttcttttggactttcgattgtaaaactgtgaattccatcagatgcttgtcggtgttggcggcggtggctgctgaggtcctgaaagcgttttgcctccgcgtgtgtgcagaacaccgaaataatcgtttttctctcttcgcctctgtttgcagccaacggctttcattaaatatcctcagcggtgctaatggtggtctccgcgcaggctttctaaaatggattactgacgccagtgttggtggcatatttccgtcttgaagtttttacttctaagtggattttaatatcgtactgctggcgtttcaggaaggataaactaacattactctcttgatttgttaaagatccgtttagagttgggcctaatttgactttttttttagcccccaagtgagctcttttaccagcataaatgctcccagatgttttagcccaattgctatggactcctgttaatgatttagtggtcattatgtatgcagccttaatggagttcttgcagaatcacacgtagactccctcatcttgat

SEQ ID NO: 69 - DNA, 5709 bp, Artificial Sequence
  Made in Lab - synthesized insert sequence
ctatatgactaatcatttttctcctagtgattcctagcacctgcatgtaatgtaatgatagaaatataaggattcatgttttggagacattgagctagctcttgacaaatgtatgtatatatgtatttgcttatgcttcaacagcaaattgtttgggttatgctgctgtgtgtgtgttgaagtttccatttctgtaagtcagcgtcatatgtcatcaattaaccagatgtcattgatgttcactgtgtatctgctatgctccacagctctagttgaagaaccttccagagaggtagtattgagaacaagtggtgacacaagcttgcaaggaagcttctcgtctcagtcagtccaaatgtctgcctccaagcaggaggcctccttcagcagtttcagcagcagcagtgctagcagcatgactgagatgaaatttgcaagcatgtctgcccaaagcatgtcctccatgcaagagtcctttgtagaaatgagttccagcagctttatgggaatatctaatatgacacaactggaaagctcaactagtaaaatgcttaaagcaggcataagaggtgagcaataaaattactggtagaggtagacaagccaagtaacatggtgtagtctttagtacacacaattcagtagaaagcaaatctttatttttttcagtgattctaagaccttttttttgtcattgttccataaaggaattccgcctaaaattgaagctcttccatctgatatcagcattgatgaaggcaaagttctaacagtagcctgtgctttcacgggtgagcctaccccagaagtaacatggtcctgtggtggaagaaaaatccacagtcaagaacaggggaggttccacattgaaaacacagatgacctgacaaccctgatcatcatggacgtacagaaacaagatggtggactttataccctgagtttagggaatgaatttggatctgactctgcaactgtgaatatacatattcgatccatttccgggtccgggtcccccggagaggttcccgacataccaataacttcgtatagtgtaggatatacgaagttatattagtattccgataacttgtttattgcagcttataatggttacaaataaagcaatagcatcacaaatttcacaaataaagcatttttttcactgcattctagttgtggtttgtccaaactcatcaatgtatcttatcatgtctggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccggggatccaccggagcttaccatggtctccaaaggagaagaagataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggaatggatgaactctataaatgaggtactcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaataacttcgtatagtgtaggatatacgaagttatataggtatgtcgggaacctctccgggtccggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagagggcctctccacttatactctacactcattcttaacttttcgcaaacgtttcacacggactaatctttctgaactgtaaatatttaaagaaaaaaaaagtagttttgtatcaacctaaatgagtcaaagttcaaaaatattcatttcaatcttttcataattgttgacctaagaatataatacatttgctagtgacatgtacatactgtatatagccggattaacggttataaagttttgtaccatttattttatgacattttacaatgtaagttttgaaactaactgttggtaggagaaagtttcttatggaacgaataccctgctcaacatttaatcaatctttgtgcctcaacatactgttgatgtctaagtatgcctcagtgggttgagaaaatccccattgaagatgtcctgtccacctaaaagagaatgatgctgtgcatatcacttgatatgtgcaccaatacctactgaatcagaaatgtaaggcattggtgatgtttgcatttaccctcctgtaagcaacactttaacgtcttacattttctctgatgatgtcacacaaaattatcatgacaaatattaccagagcaaagtgtaacggccaacactttgttcgctcattttacgctgtctctgacataaggagtgcctgaatagcttggaaaagtaacatctcctggccatcccttcatttaaccaagctattcaagtattcctatgccagagcagtgccaactcttggaggtcccagagtgcagccaatgcctttgtgtggtagttctaaattttaattgcacctgaaaaacctgggcacctaagcaatgagccacagcaaaaagtaaagaacaacaacaaaataaagctgttgttaaattttaaacaatattactaattgcccaaaatgtcaatttgatgtagttcttttcatgcaagtataaattcaattgttagttataattgttggacctccttgagatagtaacaacaaaa

SEQ ID NO: 70 - DNA, 5705 bp, Artificial Sequence
  Made in Lab - synthesized insert sequence
ctcctgcgggggacctgggatgctggagacatggatgctcacctggctgcctcggccttccagggacagaccccgaggaagccatcctgagtgccttccgcatgtttgaccccagcggcaaaggggtggtgaacaaggatgagtaagtatgggcccagccagatgaggagcaccgtggtggaagcagagagcggggtgaggcccctagtgaggggggctgcctgtgcttcggggccttacactgctctttggggtgcagccaacccttccctgcgccatgggagcctccgtacccaccttccctgtgcagtcactcccccgcagtctcctgctcagaccctcctcaccccccaggttcaagcagcttctcctgacccaggcagacaagttctctccagctgaggtgaggctgcccagccccttcaatactcatccccagcaccttctctgggccttcacccatgacccagagcccagtaccagtgaggcagttgctggaagggtgagccgagggcccttctggaggaggtgccatctctgttgagacctagagggtaaagatgtggagtcagaaaagagggcagggtgcgccaggcagggagactgtgcacagacctggggggaagtggatagggagaggtttcgtacactcggggtgggcctgtgcctgtggctggaggggcgtcctttgccccttggcccacatttgcactgactcctcactctgcccagagtcagccaagagaaaaacattaacccagagtctggggtctagggttgaaaagctaaggcaaaaagcacagatgcagggggcagacagaaaggccacaggactcaggtgaggtctctgccgggctgggccaggagccaggggactgccactcaccagtgtcccctgcaggtggagcagatgttcgccctgacacccatggatttggcggggaacatcgactacaagtcactgtgctacattattacacatggagacgagaaagaggaggggtccgggtcccccggagaggttcccgacataccaataacttcgtatagtgtaggatatacgaagttatattagtattccgataacttgtttattgcagcttataatggttacaaataaagcaatagcatcacaaatttcacaaataaagcatttttttcactgcattctagttgtggtttgtccaaactcatcaatgtatcttatcatgtctggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccggggatccaccggagcttaccatggtctccaaaggagaagaagataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggaatggatgaactctataaatgaggtactcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaataacttcgtatagtgtaggatatacgaagttatataggtatgtcgggaacctctccgggtccggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtgaggggcagggccaggcccacgggggggcacctcaataaactctgttgcaaaattggaattgctgtggtgtcttgtctgtgacagatgcgttggggaccagccaagggggatcccagggtctcagtgcgcacatcaccatgatcatggccaccatctacctcctgggagctggcccctcgccagctcaccttgattcactcccatgatgccaagtgaagtgtgaactatgatcatgcctagtttacagatgaggacactgaggcccagaaagtgtgagcatcttaccaaggccagccctctagaagaggagatggtgggatttacaccacctccaccaagcccaggaatgagccacaaagtgggcactgcccagctacttggggctgtgcagagaagaggctgcttgctgggcactcagcaaactctgcccaacagcccagcgggtgggcagcagccctgggacccccacacccaaccacacagcctcccctggcccactgctcgcaccccatctcaatacactggcttgggtgcctccctgcatgggccctttgtgaaaggcagagaggtacccatttgaaacacaaccagcttctcattgcaaatacaggcaaggcactaagacatgaggaacatggacaccaaagcaggggccaggtaacatgcaaatttctagaggaaatgcccagaacctggcatcatgcctcctgagcccctcatgcgccgtgaggggtaagagggtcagacagctggagtgtagggagacgacttctcaggagagaatagttagtgctcccgtcacccttcatctgagaacccaagagctagaggagaaagtgatcctcatgagtaccagaggagcagcaggggacatccaaagcaccagagagagaaacagagacagagagacaggcagtgacagctcaaacctcagccagatccagagcatacaaagtctcctgcctacaggacagcccagtaagagctctcagcttgcc

SEQ ID NO: 71 - DNA, 2774 bp, Artificial Sequence
  Made in Lab - synthesized insert sequence
ctcaaaaaaaaacaaaacatagttcacacttaaatattttattccatatctttacatacccaatatgttaatttatagttcaagatgaacttgtttgggacagattttgtaataaaggaaatcgtgttattagaaatatctagaggccatgagcccttaaactgttctaatttgcaagtagttccctgtgtgatgcagtttttttcaatattgcacaataaaggcaaaatacggacaaattagatgataagatttatataaatttttaaaatattgatcaaaatatgtatccatattggtaatatttgtatttataataaatcattgctgtaaatttgaacttagaaaaattttactaataaaggtgcttttgtgttgcaaactttcatttgaaaagtaatttttctttgtaccaaaaaatctaaaattcgctattctagtcaccaaaatttgctttatgaaaaataatttttgatggcactatatcagaaaacaacttgttaaagaaaatgtggagtttttaaaatcccactgtacctctgttatccaaaggggatctgtgaatttttctgtgaaaggttaaaaaaggagagacctttaggaattcagagagcagctgatttttgaatagtgttttcccctccctggcttttattattacaactctgtgctttttcatcaccatcctgaatatctataattaatatttatactattaataaaaagacatttttggtaaggaggagttttcactgaagttcagcagtgatggagctgtggttgaggtgtctggaggagaccatgaggtctgcgtttcactaacctggtaaaagaggatatgggttttttttgtgggtgtaatagtgacatttaacaggtatcccagtgacttaggagtattaatcaagctaaatttaaatcctaatgacttttgattaactttttttagggtatttgaagtataccatacaactgttttgaaaacccggtgtggacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggcctgagaagcagagcccaggccagcaacagcgccgtggacggaaccgcagcaaccgctactcaaggtttgtgtcattaaatctttagttactgaattggggctctgcttcgttgccattaagccagtctggctgagatccccctgctttcctctctccctgcttacttgtcaggctaccttttgctccattttctgctcactcctcctaatggcttggtgaaatagcaaacaagccaccagcaggaatctagtctggatgactgcttctggagcctggatgcagtaccattcttccactgattcagtgagtaactgttaggtggttccctaagggattaggtatttcatcactgagctaaccctggctatcattctgcttttcttggctgtctttcagatttgactttatttctaaaaatatttcaatgggtcatatcacagattctttttttttaaattaaagtaacatttccaatctactaatgctaatactgtttcgtatttatagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctggtaagagtattatttttcattgccttactgaaagtcagaatgcagttttgagaactaaaaagttagtgtataatagtttaaataaaatgttgtggtgaagaaaagagagtaatagcaatgtcacttttaccatttaggatagcaaatacttaggtaaatgctgaactgtggatagtgagtgttgaattaaccttttccagatattgatggacagtatgcaatgactcgagctcagagggtacgagctgctatgttccctgagacattagatgagggcatgcagatcccatctacacagtttgatgctgctcatcccac

SEQ ID NO: 72 - DNA, 2726 bp, Artificial Sequence
  Made in Lab - synthesized insert sequence
ccagtgctcccccacttacttgctgcctcccgactgctgtaattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcaggttagggaggctgggaaggccttttggggatgggggtgatttgtccaacggctgggggaggtgggaatggggaggtgagcaaggcagcagctctcagggcctggctgttgcgggtggtggtggcaggggctggaggctctaagcctagaataaggagaggcccaggtccagggaactgtgttcaattacatggatttgacacttggcagccctgagtgttttggggagagggaaggcaggcgggcagatgggggtcagagagcttagagggatggcagcccacctgggaaggcaggtgcgggtggagcccccaggcacgtgcagtgggtctctggctcacccagggcgaggagctgcccttagccaggcgtggcctcacattcagcttcctttgcttctcccagaggctgtggccaggccagctgggctcggggagcgccagcctgagaggagcgcgtgagcgtcgcgggagcctccggcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagaatgcacatgtccgacgtggctattgtgaaggagggttggctgcacaaacgaggttagtacccgctgccagggctgggcctggggagggagagacaggggtagtagccccagggtctgtgagtgcctgtgccctgctgggtgggaggggctgcctcccctggggctcctgggctggcctagggtgagtgtcctgggccccatgtcttcccatggcatggagggcacggcctcggggaggccaggaggatgaggccctgtgcagggccggccatctgagtggcttcctggtgtgggccctggcaggcgggggagctgctgccagcctctctcctggctttcctggaaggcagcgaggacactgggctgttccagggtctggcacgctggcggcccagggcactgcccaagggctcctgagggcagggcagggctgcttggcttggcctggtgcctgcttcaggcctgagctttgggggtggggggaatccgtaccgcctgggcacagggcatgtttgccttggagctgcagagctggctgagccaagtcccacagcccctgtgtccccctcctcactcatttggccttgcagcctatggaggaggtgctgcccgaggggcctggccagcagctgggtgggatgggagctggggacatagccccaccccttccaaccctgtgcaggcccttctgcctcacccaccctccgttgctcagcggggtgcggtggagtctgggtggctgttttgctcctctgtcctgctagggtgggcttcctgttcaggtccaggtcctctggccaagtcactctcttctgccccaggcggaatcggtccgccctctcctgacttctttttccagacttgttccctcctaagttctagtgatctcatgccagcagccccccggctttacgtacaccctctagcagatgggtttcacatctggtagtggggagaccccaaacacagctggagcagacagggaggtgtggcaaaagtaggtgtcaca

SEQ ID NO: 73 - DNA, 5708 bp, Artificial Sequence
  Made in Lab - synthesized insert sequence
ctaaaagggctgccttcactttccacctgctgtgtgctttgaacaagctccttaacctcactgcacctcaatttcctcatctgtaaagtgggaataataatagaagctacctcataggagattgcgaggattcgacatgttaatgtggggaaagtgtgaacttgtggcaagggcaatataagtgctagctatgaatagccagtatgttttaaaaaaataataataataactagccaatgctttttaaatggggaaattttgtgtaaaaacaaaaatctagatttctgacaccctggaccctgtggtcacttcatgtcacctttttagatggccacgtgtctccatttagccataggacctatcctgctcccttgcataacctgcctggcccctcggaacccttgagtttgtgatcccagtggaaggctgggtttccagggacatctctgcatgatgtttacttgggagggcccttggggtcagggaaggaagaacagggaagggcaagaaaggaagaacagggaagggcaaggaaggaaaagtgcatgaaggaagaagttgagccaagatttaggcccaaagatcggcactgctgactacccagggagggagcttattctacagctctcctgagaggtaccaagttgggcgagatggccgtgcctttatacccccacaccaatcccatccgctggctgtgggctgccccagaaagggcatgaccttgggcgagggggccctgcagcagctggggaagtccttccccggaggaccctctgagtggcccatgttcccatcccccaccccccagctatccgtccactcaggccccgcccacagccccccaccctccgtctcagttcccctcccctcgtccttagcacgtgttgctggctcattgcaggttgaccagatgttcgccgccttcccccctgacgtgactggcaacctcgactacaagaacctcgtgcacatcatcacacacggagaagagaaggactccgggtccgggtcccccggagaggttcccgacataccaataacttcgtatagtgtaggatatacgaagttatattagtattccgataacttgtttattgcagcttataatggttacaaataaagcaatagcatcacaaatttcacaaataaagcatttttttcactgcattctagttgtggtttgtccaaactcatcaatgtatcttatcatgtctggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccggggatccaccggagcttaccatggtctccaaaggagaagaagataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggaatggatgaactctataaatgaggtactcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaataacttcgtatagtgtaggatatacgaagttatataggtatgtcgggaacctctccgggtccggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaggagggggctcgctgctgcgcgctgggctcgtctttgcagagtggtccctgccctcatctctctcccccgagtaccgcctctgtccctaccttgtctgttagccatgtggctgccccatttatccacctccatcttctttgcagcctgggtggctatgggtacttcgtggccgcacatcctacagttggaaatccatccagaggccatgttccaataaacaggaggtcgtgtatttggtcacgacatttctctgacaaacagtgccttgtgtaaggaaggcaaaggcagatgagctagtgacagccttgcagtcattattattgaatcttggtgactctttatttaaggtagacctttgcagccactcagaactgtttctatcaaggatcacgttttcctggaatgttacctggatgaccaaagccctctcttccaaagagctcaagcatttagagggccttgcttccatgacatctgtcccttgggatgccatcggaatgcctctcctgccagtgacagtgacaagaaggaagcggactgacacatggtaggaacatagctcgttcaacagtaatcacacctgctaatcctaagagggtgacttctagtttcagataaagtagaagttgtctgtacttcttccctagcaaactgagggagtggtactgacttctgtctctgtctggggcccaggcctggatgtgagttgacctttgattttgaatgaccacagtcacggttgaagcattcaggtctgttatggctgcaagagtcccagtgtaagaaacaaatagggccaggtgtggtggctcacgcctgtgatgctagcgctttgagaggctgaggcaggaggatcacttgagcccaaaagtttgagaccagcctaggcaacatagtgagatccaatctctataaaaaataaatagccaggcatggtggtgtgcacctgtggtctcagctacccaggaggctgcagtgggaagatcgcttgagccca

SEQ ID NO: 74 - DNA, 2765 bp, Artificial Sequence
  Made in Lab - synthesized insert sequence
atttaatattggcattcggtattcagttaccttgtacctgagaacccattggctgtgaaacagtgacagctgagagaatcctgagtcatctcatttctagttcttggtgaacttctggacttttcttcagaaccaccttgccatgttggccaggctggtcttgaactcctgacctctcaggtgatccaacaccttggcctcttaaagtgctgggattacaggcatgagccaccatgcctggccagctgttttttttgttggtttgttttttgttttggttacccatctgtagtgtgatcttggctcactgcaacctctgcctcttgggctcaggcagtcctcccacctcagcctcctgagtagctgggcctcctgtagttgcacaccaccaagcctggctaatttttgcatttttagtagacagggtttcaccatgttgcccaggctggtctcaaattcctgagctgaagtgatctgcccgcctcagtctcccaaagtgtagggattacaggcgtgagccaccatgcctagcctcagcatatagttttttctaaatgtacacatgcccaggcacacatgcacaggcaattcagaataagtttctggtgtttatgtaactttatttgccaaatctggccaactctaaagctgatctcgggagatgaagttggaagtaacattggccatatgggtctctgttctttctgttgatttccttaagtaaataatgctaaactattaaataattattagtatattgttcacatttttatgactgattaaagtgtttggaattaaattacatctgagtataaattttcttggagtcatatctttatctagagttaactctctggtggtagaatgaaaaatagatgttgaactatgcaaagagacatttaatttattgatgtctatgaagtgttgtcgttccttaaccacatttctttttttttttttctaggctattcaagatctctcgcagtggaggaagtctcttaagcccaacagcgccgtggacggcaccgctggccctggcagcatcgccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagtagcacggtttctattgacttatttaactgctttatactttgtcaaagaaataattaatgtagttaggaatggcaaatagtcttgtaaaattctatgagaatgtccctgccctccccttcaatattctctctggagctaaccactttttcatcataaggatttagtgctgtgttcccacctcctgatgatagttaacaattattataactatgcaacatgtttccaaatgttccattagacctcctatctgcctattctagcctcacttgcaaagaaaatgtggcatgttaaaacagcttaaaagcagcctttcaacctgtatggttttttcccctaggctggagtgcagtggcacaatctcagcttattgcagcttctgcttcttgggttcaagcaggtctcctgcctcagcctcccaagtagctgggattacaggtgtgagccaccagcccggctaatttttgtatttttagtagagatggggtttcaccttgttggccaggctggtctcgaactcctgagctcaagtgatctgcccacctcggcttcctaaagtgctaggattataggtgtgagcaactgcacctgacctcaacctggattccttatctgaatcaaagcaattatttcagtgactatttgctcaagttgcctagtacttcttacagatgtataggag

SEQ ID NO: 75 - DNA, 5708 bp, Artificial Sequence; identical to SEQ ID NO: 59
  Made in Lab - synthesized insert sequence

SEQ ID NO: 76 - DNA, 2732 bp, Artificial Sequence
  Made in Lab - synthesized insert sequence
ctttgcccagcagcttgttgagctcctcgtcgttgcggatggccagttggagatgacgcgggatgatgcgggtcttcttgttgtcgcgggccgcgttgcccgccagttccaggatctcggcggtcaggtactccagcaccgctgccagatacaccggcgcgccggccccgacccgctcggcatagttgcctttgcggagcaggcggtgcactcggcccacggggaactggagaccggccctagaagagcgagtcttggccttagcgcgggctttgcctccctgcttgccacgtccagacatagcgagcgcaactcactacgagcaaccacaaagtgaacgggaaaggcggcgctttttataaacactattgggcgcgaaaaagaagacgtgttgttggttagggctgcagtttaatttcaaccaatagtagtgcgtcttctggatttgcgaatcctgattgggcagacctgacctctgacgttaccctgaataactaccaatcagacacaagacttcaactcttcaccttatttgcataagcgattctatataaaagcgccttgtcataccctgctcacgctgtttttccttttcgttggcgctttatagctacacagtgctatgccagagccagcgaagtctgctcccgccccgaaaaagggctccaagaaggcggtgactaaggcgcagaagaaagacggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccgtcaccaagtacaccagcgctaaggaccctcctgtggccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaaacagtgagttggttgcaaactctcaatcctaacgcctcttttaagagccacccatgttctcaaagaaagagctggtgcttgtattcctcctctgctggccactgacaaacccttgtaacttgctactgtgttttttggtctgaagtagagcagttatttaactaatccttagtgactttttttttttagatctgccattctaatcttagagttaagtaaggagatgggaaattttctattataagttcgaaaccaattaaaatacgttagaaaccaattaaaatactcgtcggtcccccgtcggttagtgatttggaacagtgccaagttgcagcggttgtcagtttgaatttgcccgggcaacgcccgcccttcctctgcatcctgattggttgttgtatccaggaagtccacggtcggcttgggtgcgccaaaggaatccaaaccccgcctttatttggccttccttacaaactcagtaaactgtcagtttctcattctttagtattttatcgctttcgggaaggagatttatttctaattagttttcgttggatagagcgtttttttttttaaagggaccctttatggcaacataatcagtttttttttttttgagaaggagacttcctctgtcgcccagactggagtgcggtggcgcgatctcagctcactgcagcctccgcctcccgggttccagcgattctcctgcttcacccttccgtgtagctgggattacaggcatgcgccaccatgcccggataatttttgtatttttagtagagatggggtttcaccatgttggctaggctggtctcgaactgacttcaggtgatccactagcctcagccttaaagtgctaggattacaggcatgagccatcaagcccggctacaacggaatttgaatgctgtagttttcagcctgagaaagatcaatgctttatttctgattagaatactattttccctttatggtatatctatataagcaaagg

SEQ ID NO: 77 - DNA, 2750 bp, Artificial Sequence
  Made in Lab - synthesized insert sequence
taggcggtggagggtaaaccgcgaggcttccatgaagaagagtggcctgggaagtgaggaggacagcgaagccccaaacctcaccttgggatacgggaagtgaagcaggttccagagaaggggacaagataatgacttagggtaggataaatttcagacaattgcagagcatcacattagatgtgttcagaagagtggaaataaagttctgtgcctaggccctcatgcaggattctgttgctagaagtcagtgctggccgcagggagaccggagatcacagtggaggcaccagcgatccgagagtcctatgacgaatccctgggatccccacaatgtgtggagacagaagccaaccaaagagcgagaaagcatctccaaaccagcagaatcaggggcaggaggcccgccaaatgcacagcaagtcagccttggtagtttgcaaatggaaagatttctttgacccgctggagacttcagctcaggctggatgtcgctaattcacaccatctcaccaaaaggttctttccctgccctttggtgaatcgaggcagaagtggctggtggataactgggacccaagagttatctcagcgtcgggcaggcatcgacagctccaggagccctttccctgcaggcgggctggcgggtgagcgattccctcccttcccaggccttcccgggaatggaacgtgggcgcccgcgcccgggctccggttccacttggaacgcggactcgggagccgccgggcgagcgcgcagaccgcgggggcggctccctcgcacctccccgctcagcgcgccgcggcggatgggcgaggcggggcgaggccagagggaggcgggccaaaggggcgggcgagaggacgcgggaccgcggggaggtcgggggcggggagcaggcggcggcgggcgccgggaagaaaggaacatggctcctgaggcgcacagcgccgagcgcggcgccgcgcacccgcgcgcaggacgccagtgaccgcgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagagcggaggaggaggaaccggaggaggaagcggaggagtgaactcaagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtaagagcgcgcgacccgcagcggcagatgcacgaaccagaacggccggcgccggccggggccatcgcccgctgcggcagctccccgggctccatctcgcatcccctctgcgttccgcctcccttggaagcgcattccccacctccgctagtgctgccctatttccggtacccagcgcggaattccactgctcttttgttggtgcacatttattggatacctccttcttcaggatatgtcaccatagtcttttttactgaaaattagtgaaagcctaattagagtgaaagagtacatctgggttttgtttttttttttcttgtagaggaaaaaatgaacattacttgtgtaactgatggtagttgcaactgcatatttgccaatgtcacaaaatctaaaggaaaatgt

SEQ ID NO: 78 - DNA, 7135 bp, Artificial Sequence
  Made in Lab - synthesized insert sequence
ccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtgggccaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgggtaccgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggaccctcctgtggccaccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcagtggagagggcagaggaagtctgctaacatgcggtgacgtcgaggagaatcctggcccagtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtccggactcagatctcgagctcaagcttcgaattcaaagatgagcaaagatggtaaaaagaagaaaaagaagtcaaagacaaagtgtgtaattatgtaaacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 79 - DNA, 6410 bp, Artificial Sequence
  Made in Lab - synthesized insert sequence
taaaactatgggacatagacgaaaaggcaagccataaagtaagtctcagattatcttactctaaatctgaattttgctacgaagtacccagggacattgtgctgttctttttctccgtgctgggagattcgtgctaggtcgtgctgcattattagtaaaaggctattgtgatagttaaccctcatttaggaggagctctaagatggattcacaaaatacccaaatgcatacaatacatgtgggtcaccttataattagggtctagagtaatttggaatgtaaagcaatttttattctgaggtttcctaattcaaatgttaatagtgcaccaaaaggtcaattcaagagtttattattattatttttcaacccaagtaaaagcagagagaaaatagccacctccaccatagcctcagaagcaagccaacagcctgaaacagctttgaaatgaaaagttggtgtggcggtgatggtggcagtgataatggtgacgatggttgggtgctggtgatggtagtggtagttgtgaaggtggtgatggtggtttgattgatagtaaaaaaaatgttcgttaatacaagtagagagtaagtaatcaatcaatcactcatagccaaggtggaaaagatgtatcccatcatggaatattcctgttctgatagaaatcttgtgcttatctatggaattcttttgatatatatttacattgggaacctgaatgtagcttgacatttttccatgtaaacaccagtagcctgatccaacattaagctgatactaacaaacaacgtgtaatggcttcattaataaggctttgcttcttcctggaaactggtgaaaaatcaaaccttgttgtgtacaccctcgatgcagcttctgtgttgtcttcacccagaaatggggaatgatttcccaaatggcaaagaaacagagtgatgctatctatctgcaccttttgtaaagtctgtctttctttctctttgttttccaggacacaatgcttgaagccgattacaaggatgatgatgatcccggagaggttcccgacataccaataacttcgtatagtgtaggatatacgaagttatattagtattccgataacttgtttattgcagcttataatggttacaaataaagcaatagcatcacaaatttcacaaataaagcatttttttcactgcattctagttgtggtttgtccaaactcatcaatgtatcttatcatgtctggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccggggatccaccggagcttaccatggtctccaaaggagaagaagataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggaatggatgaactctataaagaattcggcagtggagagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtactcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaataacttcgtatagtgtaggatatacgaagttatataggtatgtcgggaacctctccggggatgatgatgataagagatctgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaggaagtctttttcacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacagga

SEQ ID NO: 80 - DNA, 2753 bp, Artificial Sequence
  Made in Lab - synthesized insert sequence
ggatggtctcgatctcctgacctccggtaatctgcccgcctcagcctcccaaagtgctgggattacaggcgtgagccaccgtgcccggcctggcctgataattctttgtggtggaggactgacctgtgtcttggaggatgtttaatagcatctctagctttcacccacgagataacagtagcacatcccagcccagtagtgacaaccaaaaatgtgttagccaaatgttcctagaggcaaagtcaccccaggctgagaactactgatctactggttctccctccaatcagtcttcccttgtattcttcttgttatctatttgcagaagaacctgagtataatcttgcagtgtttccctcggtatggatttcactgatttaatccctgtagtataatttaacatcctgagtcaccaatttcctgttaattgggttggatctagagacttgattggtttcagagttttggagtttgtttgacaaaattgcattataggtagtggttttttctttttcttttttttttttttttttttgagatggagtcttgctctgtcccccacgctggagcgcagtggtgcaatctcggctcactgcaagctccgcctcctggattcaagctattcttctgcctcagcctcccgagtagctgggactacaggcgcccgccaccatgcctggctaattttttttgtatttttagtagagacggggtttcaccgtgttagccaggatgttctcgatctcctgacctcgtgatccgcccgcctcagcctcccaaagtgctgggattacaggcgtgagccactgcacctggccagtagtggttgtttctttcatcaagaggcacatgtctgttgtgtcttttttaatattaacaaccattgatgcctaattcattcaccaaagggtctttttgttttaaaatgtatatttttatttagacatgctttgctttaaataacaatctgtgttctcccttaataaaggaaggggaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtccggcagaacccagatcagctccagcagtttcgagtttgaaggtgatgcagtcgaagcaattgtcgaggagtccgaaacttttattaaaggaaaggagagaaagacttaccagagacgccgggaagggggccaggaagaagatgcctgccacttaccccagaaccagacggatgggggtgaggtggtccaggatgtcaacagcagtgtacagatggtgatgatggaacagctggaccccacccttcttcagatgaagactgaagtaatggagggcacagtggctccagaagcagaggctgctgtggacgatacccagattataactttacaggttgtaaatatggaggaacagcccataaacataggagaacttcagcttgttcaagtacctgttcctgtgactgtacctgttgctaccacttcagtagaagaacttcagggggcttatgaaaatgaagtgtctaaagagggccttgcggaaagtgaacccatgatatgccacaccctacctttgcctgaagggtttcaggtggttaaagtgggggccaatggagaggtggagacactagaacaaggggaacttccaccccaggaagatcctagttggcaaaaagacccagactatcagccaccagccaaaaaaacaaagaaaaccaaaaagagcaaactgcgttatacagaggagggcaaagatgtagatgtgtctgtctacgattttgaggaagaacagcaggagggtctgctatcagaggttaatgcagagaaagtggttggtaatatgaagcctccaaagccaacaaaaattaaaaagaaaggtaaaacgagtttatccatagtggtttcataaaaccattttgggataagcatacaacacagtgcatatgcaagttgttttatattaaccgtatttgtaaaaggtcgttatgtgggtaccgttctttaaaaccagtctaaaataagttttttccagattgaatgctctttttttaatcccaaagaagaaggaaatgtattagtgacatgagataattatgact

SEQ ID NO: 81 - DNA, 2741 bp, Artificial Sequence
  Made in Lab - synthesized insert sequence
ctactggttccctcccagctacaagccagcccccttcttcgtcctggatgagattgatgctgccttggataacaccaacattggcaaggtgggtgcaaagaaagtggggcctgggagggatgcccccaggtcggggttgggtttcagggagcaactcctgagcgtgccctagccgttcctgctgcctgctctctgtgcctgggaggctgggcccagagtcatccatgctgtggtagtcagtagtctcctctcaacctctcttttccccaccttcctctccctaccctacctcttctgccttctggttgtggctggaaaatactggggactgttaaggtttgagccttgctaagtgcctggcggcctttcccacaggtggcaaattacatcaaggagcagtcgacttgcaacttccaggccatcgtcatctctctcaaggaggagttctacaccaaggccgagagcctcattggagtctatcctgaggtagggcgggcctggctcaggagccagtcctactccctgcctgatttcccagggcaggaaaggaaggctgcagtggaggggaaaggaggtgggggagcatagggaatcaggtcctgaatggccatttgggcttgttggagccctgcctctggaggcttagccagcccagccaggcagggccattgcatttggggacaggtggaaaggcaggcagaggcagccagacagtggagaaaggaggagagtacagaagtaggcaaggagcgaaggcacatgggactgagcatatcctctcacacccagagagaaccattgactgggctttgggcaggtatgtagggaggagggtttgaggcccctggtcctggggcactagacccctcttaattctctccttacaattttatttttctttttctccctcctccttcagcaaggggactgtgtgatcagcaaagtcctgaccttcgacctcaccaagtacccagatgccaaccccaaccccaatgagcagctgagctccagcggcccctcaggcagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtagcagtatttttgtcctccggccctgtctggatccctaagctgtccctctcccaatctctggatatttgactcccaaccttccccctacctcctggccctttttggtgtagtcatgggatttaggcactgctaatcaagcatgaagaggaacagaggtgatgttaggtctggagcaaaaattcctgaacgacagggagtattctggcctctgaaaggaggtgctgagctgaacagggccatctgttcatcacacacacccccttcctccccctcatcacccataatcgtgggccccttgggcctcttgcccactgtgtgtgtgggtatgtatgtgtgtatgtatgtatccgcatgtgtgcatgtgagtatgtttgcaaaataataaaggatattggagacctgttttagaaggagcctaggctgaatttgattccaagagagcttaggatgacagcacccctgagctgggcaaaggtactcaggacctcataggagtcttaggcagttacctgaaactgccttcattcactcatttgtgtattcattcatttatgtattcatcagacacataccgaacaccctctatttgtcaggctctgtgcttggaatacagagttgaatcagacatgatctctaccctcctagtaaggagatacagtgggttcatgaatgactatagttagctgaatgtcatatgtactttgaatttgagaagtgggtgatcccctctaggcttcctggaggtcacatttaagctagaccttgacaaattggtaggatttggtcaggcactaggagtggagcatgagctctggggacagacagttatgggttctggtcccactttttatcacttactagttgtttgaccttgggcaagtcatttgaccttctgtgcctcagtttcctcatctgtaaaatggggctaacaatattacctacctcataggatttaatgatgtcaagctcctcactggaggccttatcccttcgtggag

SEQ ID NO: 82 - DNA, 2729 bp, Artificial Sequence
  Made in Lab - synthesized insert sequence
aacctttatatgtttaaagaaacattaatgaagccgtatatattttctaattctaaggatcatggactttatttttcaaccacagatttttttttcctccttcttaaatcattagctcttgtctttgattttactgtctgcttacttcctgcctacttaaccaaccgacagggagaaggagaatgggaagaggttactgtttttatgttaagtgtcttttcaaagtctaatttacagccaagaacagcaaaagtaccccttggctttgtgcctctgcggcgccccggagatctccttccccgtccctcttcctttccgagaagccgcgttccgttgttgaactacgtttcccagcgtgccgcgcggccgagagcgtggactcgaccagcccttcctctttgcctatcagctcgtcgcagcatgatgatgcaaatgcgtcgggcgccgatgagtgacgacattctggctcgcgaccgcggcagccgccttagcaggggtaataggcgcaacggcggcggaggctgcagggacgacgacgacgacggcggcggggcgggcgctgtgcgcacaggggaggggtaggcgggcgtcgccagcggcttcaactgcggcagctacggctagggcgacggaagaactcccaccagtcggcggtcgagttaggcctcagcaccgcggggaactgttcgtgctgtcctctgcgggagatctccatagagaccgtgacacacacagaggcgggggtccgcagaggcggccggaccggagcctcctcggcctctgcggccgccacccccttccccgctgccagcactcaccctctctccgctcctgctcgcaacctcgcggttcctggggtgcttgtgcccactgtgtggacagcgcgggcggacttttgggccggggccgggcggcgggggaggctctctaaggcctccgcctctgcctctcccgcccccttacccgccccggagcgggaagcggcggaggctccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtacagcgacctggaggcctcaggggcaggaggagtcggagggggcggtggcggcaagatccggacgcggcgttgccaccaggggccaattaagccttaccagcaggggcgacaacagcatcaggtatgggaccccgacgccgcggtggcctggcgggtggggaaactcggctggaggggacgggcggttcgaggccccggcgccccacgtggagcagacaggagggcctgaggaaaggcccgagtgaggctccgggggagctcaggccgttgggggtggcgacgggaagtgggctgagacaggcccagccttgcggcgcacacgtgtggtttgggccgcctttcccccaactctctgggtcaggggctttcccaggcaggctctgggcgggaaaagaaattgtaggcccatgatgcgcgcgtttctttattttttggcggggatttggaatttgagggtattactcatgttctctcgagggagaaaggaaaagatgtggttgatggcccttatacttgctgttttctggtgtcccttctttagtgtgtttgaggagggggggctgccccttggaatcttaactctggggcagacggggagttgtggggggcgctgttacatggaaagtggaataacttagtctcagcctatgagttaattctcggtttcccctgcctgtggccctcctcgtcttgagagcattggcttagattttattaaccaggttatagttaaccgtggcactggggcattttgaaagtccctctccacccttacgtcttttctctgctgcgcgactcccctccccccccccaacaccgccacccccgcctttattttggaggctggaattggccattctccacagttgaccgggactccagctctgatctcatacacaaaatattttaatattatgaggcgtattctagaagtatagttacttttgacactccaggaatgttggtggtcttatagtttagccactgtatgaactcctggatgtttaaaatta

SEQ ID NO: 83 - DNA, 2726 bp, Artificial Sequence
  Made in Lab - synthesized insert sequence
ttgtatttttagtagggacagggtctcgccatgttggccaggatggtcttgaacccctgacctcaggtgatctgcccgcctcggcctcccaaagtgctagaattataggtgtgagccactgcacccagcctttcaatttaatgcaatattttagctaccgtgctcttttacaacggtttttttgtgaagatggagtcttgctgtgttgcctaggctggtcacacactactgggcttcaggaattctcccacttgggccttccaaaatgctgggattgcagatgtgagcctgcacgcctggccctagctaccatgctttatggtaaaaggagtattgggagccttttaaaattaccaagaatgggtataaaagttttaaatggatacacaagaataaagaagtttgctgtgaaggccaggcgtggtggctcatgcctgtaattccaacactttgggaggccgaggcaggtggatcacctgaggtcaggagttcgagaccaacctggccaacacatagtgaaatcccgtctctactaaaaaaaaaaaaaaaacaaaaacaaaaaaaaaaacaacaaaaattagctgggcttggtggcacacgcctgtagtcccagctgcttgggaagctgaggcaggagaatcgcttgaacccaggaggcggaggttgcagtaagccgagattgcgccactttgcactccagcctgggcaacagagcaagactccgtctctcaaaaaaaaaaaaaaagtttgctgtaagattaattggggctgttttatgagctttctggctggacatatcagaaaactattttgtatcgagtgtgagaagaagcctaacacttactataagcagcatacaatattatcacctgtcatgtccctggttttcttaccatccagatattatctaaccaataagagtgctccctaatgccctttttatttcatttatcattttagcagcgtcagcctttacaccagaaagctggcgggcactatgctggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagaagggcagcgggaaaaaacaaaacaagaagaaagtggaggaggtgctagaagaggaggaagaggaatatgtggtggaaaaagttctcgaccgtcgagtggtaaagggcaaagtggagtacctcctaaagtggaagggattctcagagtaagtttcactgacacaggtaaatgtagccaccacgtgtttatcaggagtctagagatgtatgagacctccagtgctatgatggatacagtgtgactcagtccccaccttcatggcttattgtttaattaggaatatggcctactgcttataatacaaagcagaatgacttctttttaaaaattatttcttttgagacgaggtctcgctctgtcacccaggctggagtacagtggtgcagtcatagctcgctgcagcctcaacctcccaggctcaaggtatcctcccacctcagccttctaagtagctggtaccacaggcatgcaccaccatgtctcgcttagttgcccaggtttgtctcttaactcctggcctcaagcagtcctcccacctgggcctcccaaagtgttgggactacaagcatgagccactgtacccagcccatagtgattgctgagaaacagttttgtgtgtggctatagaccaatttaaattgacaaataaaagtcatttgcctttgaaggaagccagtttcttagctttctatttatactaaaaagcttccccattccccaagaatttcagcttttccatagacatctgaggtcttgtcctcttacgattatccttagtatgagtatacttatttgagtgaaagtgtgacttgctgtgccctctggtttcagtgaggacaacacatgggagccagaagagaacctggattgccccgacctcattgctgagtttctgcagtcacagaaaacagcacatgagacagataaatcagagggaggcaagcgcaaagctgattctgattctgaagataagggagaggagagcaaaccaaagaagaagaaagaaga

SEQ ID NO: 84 - DNA, 2726 bp, Artificial Sequence
  Made in Lab - synthesized insert sequence
gccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggcggaagcggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtc

SEQ ID NO: 85 - DNA, 20 bp, Artificial Sequence
  Made in Lab - synthetic crRNA
cttgtcgttctgctccttga

SEQ ID NO: 86 - DNA, 20 bp, Artificial Sequence
  Made in Lab - synthetic crRNA
cttgtcgttctgctccttga

SEQ ID NO: 87 - DNA, 20 bp, Artificial Sequence
  Made in Lab - synthetic crRNA
gcacctagcagaagagcttg

SEQ ID NO: 88 - DNA, 20 bp, Artificial Sequence
  Made in Lab - synthetic crRNA
gcacctagcagaagagcttg

SEQ ID NO: 89 - DNA, 20 bp, Artificial Sequence
  Made in Lab - synthetic crRNA
tctaggtcacagtcgcagtt

SEQ ID NO: 90 - DNA, 20 bp, Artificial Sequence
  Made in Lab - synthetic crRNA
tctaggtcacagtcgcagtt

SEQ ID NO: 91 - DNA, 20 bp, Artificial Sequence
  Made in Lab - synthetic crRNA
ccctcatctccaatatggta

SEQ ID NO: 92 - DNA, 20 bp, Artificial Sequence
  Made in Lab - synthetic crRNA
cactcatctccaatatggta

SEQ ID NO: 93 - DNA, 20 bp, Artificial Sequence
  Made in Lab - synthetic crRNA
gccataccatattggagatg

SEQ ID NO: 94 - DNA, 20 bp, Artificial Sequence
  Made in Lab - synthetic crRNA
gccataccatattggagatg

SEQ ID NO: 95 - DNA, 20 bp, Artificial Sequence
  Made in Lab - synthetic crRNA
aattgtaagtgctcagagct

SEQ ID NO: 96 - DNA, 20 bp, Artificial Sequence
  Made in Lab - synthetic crRNA
aattgtaagtgcacagtcct

SEQ ID NO: 97 - DNA, 20 bp, Artificial Sequence
  Made in Lab - synthetic crRNA
tggtagttgagcagctctgg

SEQ ID NO: 98 - DNA, 20 bp, Artificial Sequence
  Made in Lab - synthetic crRNA
tggtagttgagcagctctgg

SEQ ID NO: 99 - DNA, 20 bp, Artificial Sequence
  Made in Lab - synthetic crRNA
gatgcactcacgctgcggga

SEQ ID NO: 100 - DNA, 17 bp, Artificial Sequence
  Made in Lab - synthetic crRNA
gatgcactcctgcggta

SEQ ID NO: 101 - DNA, 20 bp, Artificial Sequence
  Made in Lab - synthetic crRNA
agagataaggtctgtcgccc

SEQ ID NO: 102 - DNA, 20 bp, Artificial Sequence
  Made in Lab - synthetic crRNA
agagataaggtctgtcgccc

SEQ ID NO: 103 - DNA, 20 bp, Artificial Sequence
  Made in Lab - synthetic crRNA
ggggtcgcagtcgccatggc

SEQ ID NO: 104 - DNA, 17 bp, Artificial Sequence
  Made in Lab - synthetic crRNA
ggggtcgcagtcgcggc

SEQ ID NO: 105 - DNA, 20 bp, Artificial Sequence
  Made in Lab - synthetic crRNA
gtcgcagtcgccatggcggg

SEQ ID NO: 106 - DNA, 17 bp, Artificial Sequence
  Made in Lab - synthetic crRNA
gtcgcagtcgcggcggg

SEQ ID NO: 107 - DNA, 20 bp, Artificial Sequence
  Made in Lab - synthetic crRNA
aactgaagttcagcgctgtc

SEQ ID NO: 108 - DNA, 20 bp, Artificial Sequence
  Made in Lab - synthetic crRNA
aactgaagttcagccctgag

SEQ ID NO: 109 - DNA, 20 bp, Artificial Sequence
  Made in Lab - synthetic crRNA
cagttcttcaccttgggggg

SEQ ID NO: 110 - DNA, 20 bp, Artificial Sequence
  Made in Lab - synthetic crRNA
cagttcttcactttaggagg

SEQ ID NO: 111 - DNA, 20 bp, Artificial Sequence
  Made in Lab - synthetic crRNA
gctattctcgcagctcacca

SEQ ID NO: 112 - DNA, 20 bp, Artificial Sequence
  Made in Lab - synthetic crRNA
gctattctcgcaactgacaa

SEQ ID NO: 113 - DNA, 20 bp, Artificial Sequence
  Made in Lab - synthetic crRNA
gccgttgtcgacgacgagcg

SEQ ID NO: 114 - DNA, 20 bp, Artificial Sequence
  Made in Lab - synthetic crRNA
gccgttgtcgacgacgagcg

SEQ ID NO: 115 - DNA, 20 bp, Artificial Sequence
  Made in Lab - synthetic crRNA
tcatttagcagtagttctat

SEQ ID NO: 116 - DNA, 20 bp, Artificial Sequence
  Made in Lab - synthetic crRNA
tcatttagcagtagtagcat

SEQ ID NO: 117 - DNA, 20 bp, Artificial Sequence
  Made in Lab - synthetic crRNA
agaactactgctaaatgagt

SEQ ID NO: 118 - DNA, 20 bp, Artificial Sequence
  Made in Lab - synthetic crRNA
gctactactgctaaatgagt

SEQ ID NO: 119 - DNA, 20 bp, Artificial Sequence
  Made in Lab - synthetic crRNA
cttggcggccgcagctctgg

SEQ ID NO: 120 - DNA, 20 bp, Artificial Sequence
  Made in Lab - synthetic crRNA
ctttgcggccgcagctctgg

SEQ ID NO: 121 - DNA, 20 bp, Artificial Sequence
  Made in Lab - synthetic crRNA
tctctctccagcgccgcgcg

SEQ ID NO: 122 - DNA, 20 bp, Artificial Sequence
  Made in Lab - synthetic crRNA
tctctctccagcgccgcgcg

SEQ ID NO: 123 - DNA, 20 bp, Artificial Sequence
  Made in Lab - synthetic crRNA
ggccgcggaggcgctcacct

SEQ ID NO: 124 - DNA, 20 bp, Artificial Sequence
  Made in Lab - synthetic crRNA
ggccgcggaggttctcacct

SEQ ID NO: 125 - DNA, 20 bp, Artificial Sequence
  Made in Lab - synthetic crRNA
tttacaatggcgcagagaac

SEQ ID NO: 126 - DNA, 20 bp, Artificial Sequence
  Made in Lab - synthetic crRNA
tttacaatggcacaaaggac

SEQ ID NO: 127 - DNA, 20 bp, Artificial Sequence
  Made in Lab - synthetic crRNA
ggcgcagagaactggactcg

SEQ ID NO: 128 - DNA, 19 bp, Artificial Sequence
  Made in Lab - synthetic crRNA
ggcacaaaggcagggctgg

SEQ ID NO: 129 - DNA, 20 bp, Artificial Sequence
  Made in Lab - synthetic crRNA
gttctctgcgccattgtaaa

SEQ ID NO: 130 - DNA, 20 bp, Artificial Sequence
  Made in Lab - synthetic crRNA
gtcctttgtgccattgtaaa

SEQ ID NO: 131 - DNA, 20 bp, Artificial Sequence
  Made in Lab - synthetic crRNA
cgcctgaccacgccgaccac

SEQ ID NO: 132 - DNA, 20 bp, Artificial Sequence
  Made in Lab - synthetic crRNA
cgcctgaccacggagacccc

SEQ ID NO: 133 - DNA, 20 bp, Artificial Sequence
  Made in Lab - synthetic crRNA
tcaaggccctgtggtcggcg

SEQ ID NO: 134 - DNA, 20 bp, Artificial Sequence
  Made in Lab - synthetic crRNA
tcaaggccctggggtctccg

SEQ ID NO: 135 - DNA, 20 bp, Artificial Sequence
  Made in Lab - synthetic crRNA
agtctggccgtgtggccgca

SEQ ID NO: 136 - DNA, 20 bp, Artificial Sequence
  Made in Lab - synthetic crRNA
agtctggtctagtagccgca

SEQ ID NO: 137 - DNA, 20 bp, Artificial Sequence
  Made in Lab - synthetic crRNA
ggagatgtagtctggccgtg

SEQ ID NO: 138 - DNA, 20 bp, Artificial Sequence
  Made in Lab - synthetic crRNA
ggagatgtagtctggtctag

SEQ ID NO: 139 - DNA, 20 bp, Artificial Sequence
  Made in Lab - synthetic crRNA
agggcttgggccaaccagta

SEQ ID NO: 140 - DNA, 20 bp, Artificial Sequence
  Made in Lab - synthetic crRNA
agggcttgggccaaccagta

SEQ ID NO: 141 - protein, 6 aa, Artificial Sequence
  Made in Lab - linker sequence
GTSGGS

SEQ ID NO: 142 - protein, 6 aa, Artificial Sequence
  Made in Lab - linker sequence
GGSGGS

SEQ ID NO: 143 - protein, 10 aa, Artificial Sequence
  Made in Lab - linker sequence
SGLRSRAQAS

SEQ ID NO: 144 - protein, 19 aa, Artificial Sequence
  Made in Lab - linker sequence
KLRILQSTVPRARDPPVAT

SEQ ID NO: 145 - protein, 10 aa, Artificial Sequence
  Made in Lab - linker sequence
GGSGDPPVAT

SEQ ID NO: 146 - protein, 7 aa, Artificial Sequence
  Made in Lab - linker sequence
HDPPVAT

SEQ ID NO: 147 - protein, 5 aa, Artificial Sequence
  Made in Lab - linker sequence
SGLRS

SEQ ID NO: 148 - protein, 17 aa, Artificial Sequence
  Made in Lab - linker sequence
KPNSAVDGTAGPGSIAT

SEQ ID NO: 149 - protein, 5 aa, Artificial Sequence
  Made in Lab - linker sequence
AGSGT

SEQ ID NO: 150 - protein, 11 aa, Artificial Sequence
  Made in Lab - linker sequence
YSDLELKLRIP

SEQ ID NO: 151 - protein, 18 aa, Artificial Sequence
  Made in Lab - linker sequence
SGLRSGSGGGSASGGSGS

SEQ ID NO: 152 - protein, 12 aa, Artificial Sequence
  Made in Lab - linker sequence
SGLRSRALERDK

SEQ ID NO: 153 - protein, 15 aa, Artificial Sequence
  Made in Lab - linker sequence
LQSTVPRARDPPVAT

SEQ ID NO: 154 - protein, 19 aa, Artificial Sequence
  Made in Lab - linker sequence
EFGSTGSTGSTGADPPVAT

SEQ ID NO: 155 - protein, 7 aa, Artificial Sequence
  Made in Lab - linker sequence
RDPPVAT

SEQ ID NO: 156 - protein, 7 aa, Artificial Sequence
  Made in Lab - linker sequence
SGLRSRA

SEQ ID NO: 157 - protein, 6 aa, Artificial Sequence
  Made in Lab - linker sequence
DPPVAT

SEQ ID NO: 158 - protein, 13 aa, Artificial Sequence
  Made in Lab - linker sequence
SGRTQISRCCAAN

SEQ ID NO: 159 - protein, 4 aa, Artificial Sequence
  Made in Lab - linker sequence
RMHM

SEQ ID NO: 160 - protein, 20 aa, Artificial Sequence
  Made in Lab - linker sequence
SGLRSRAQASNSAVDGTAAT

SEQ ID NO: 161 - protein, 17 aa, Artificial Sequence
  Made in Lab - linker sequence
KPNSAVDGTAGPGSIAT

SEQ ID NO: 162 - protein, 12 aa, Artificial Sequence
  Made in Lab - linker sequence
SGGGGTGGGSGG

SEQ ID NO: 163 - protein, 7 aa, Artificial Sequence
  Made in Lab - linker sequence
SGSGSSG

SEQ ID NO: 164 - protein, 10 aa, Artificial Sequence
  Made in Lab - linker sequence
VDGTAGSIAT

SEQ ID NO: 165 - protein, 7 aa, Artificial Sequence
  Made in Lab - linker sequence
SGSGSSG

SEQ ID NO: 166 - protein, 11 aa, Artificial Sequence
  Made in Lab - linker sequence
GGGGGGVPVEK

SEQ ID NO: 167 - protein, 9 aa, Artificial Sequence
  Made in Lab - linker sequence
VDPRVPVAT

SEQ ID NO: 168 - protein, 17 aa, Artificial Sequence
  Made in Lab - linker sequence
SGLRSRAQASNSAVDAT

SEQ ID NO: 169 - protein, 13 aa, Artificial Sequence
  Made in Lab - linker sequence
STVPRARDPPVAT

SEQ ID NO: 170 - protein, 13 aa, Artificial Sequence
  Made in Lab - linker sequence
SGRTQISSSSFEF

SEQ ID NO: 171 - protein, 9 aa, Artificial Sequence
  Made in Lab - linker sequence
LSSSGPSGS

SEQ ID NO: 172 - protein, 5 aa, Artificial Sequence
  Made in Lab - linker sequence
YSDLE

SEQ ID NO: 173 - protein, 4 aa, Artificial Sequence
  Made in Lab - linker sequence
GGSG

SEQ ID NO: 174 - DNA, 23 bp, Artificial Sequence
  Made in Lab - synthetic crRNA
aattgtaagtgctcagagcttgg

SEQ ID NO: 175 - DNA, 23 bp, Artificial Sequence
  Made in Lab - synthetic crRNA
  misc_feature (21)..(21): n is a, c, g, or t
aattgtaagtgctcagagctngg

SEQ ID NO: 176 - DNA, 23 bp, Artificial Sequence
  Made in Lab - synthetic crRNA
  misc_feature (21)..(21): n is a, c, g, or t
aattgtaagtgctcagagctnag

SEQ ID NO: 177 - DNA, 22 bp, Artificial Sequence
  Made in Lab - synthetic crRNA
  misc_feature (20)..(20): n is a, c, g, or t
aatttaagtgctcagagctngg

SEQ ID NO: 178 - DNA, 22 bp, Artificial Sequence
  Made in Lab - synthetic crRNA
  misc_feature (20)..(20): n is a, c, g, or t
aatttaagtgctcagagctnag

SEQ ID NO: 179 - DNA, 22 bp, Artificial Sequence
  Made in Lab - synthetic crRNA
  misc_feature (20)..(20): n is a, c, g, or t
aatttaaatgctcagagctngg

SEQ ID NO: 180 - DNA, 22 bp, Artificial Sequence
  Made in Lab - synthetic crRNA
  misc_feature (20)..(20): n is a, c, g, or t
aatttaaatgctcagagctnag

SEQ ID NO: 181 - DNA, 23 bp, Artificial Sequence
  Made in Lab - synthetic crRNA
  misc_feature (21)..(21): n is a, c, g, or t
aattgtaagtgctcggagctngg

SEQ ID NO: 182 - DNA, 23 bp, Artificial Sequence
  Made in Lab - synthetic crRNA
  misc_feature (21)..(21): n is a, c, g, or t
aattgtaagtgctcggagctnag

SEQ ID NO: 183 - DNA, 23 bp, Artificial Sequence
  Made in Lab - synthetic crRNA
  misc_feature (21)..(21): n is a, c, g, or t
aattataagtgctaagagctngg

SEQ ID NO: 184 - DNA, 23 bp, Artificial Sequence
  Made in Lab - synthetic crRNA
  misc_feature (21)..(21): n is a, c, g, or t
aattataagtgctaagagctnag

SEQ ID NO: 185 - DNA, 24 bp, Artificial Sequence
  Made in Lab - synthetic crRNA
  misc_feature (22)..(22): n is a, c, g, or t
aattatacagtgctaagagctngg

SEQ ID NO: 186 - DNA, 24 bp, Artificial Sequence
  Made in Lab - synthetic crRNA
  misc_feature (22)..(22): n is a, c, g, or t
aattatacagtgctaagagctnag

SEQ ID NO: 187 - DNA, 19 bp, Artificial Sequence
  Made in Lab - primer
gccgacaagcagaagaacg

SEQ ID NO: 188 - DNA, 20 bp, Artificial Sequence
  Made in Lab - primer
gggtgttctgctggtagtgg

SEQ ID NO: 189 - DNA, 21 bp, Artificial Sequence
  Made in Lab - synthesized oligonucleotide probe
agatccgccacaacatcgagg

SEQ ID NO: 190 - DNA, 21 bp, Artificial Sequence
  Made in Lab - primer
tttccgtgtcgcccttattcc

SEQ ID NO: 191 - DNA, 21 bp, Artificial Sequence
  Made in Lab
atgtaacccactcgtgcaccc

SEQ ID NO: 192 - DNA, 23 bp, Artificial Sequence
  Made in Lab - synthesized oligonucleotide probe
tgggtgagcaaaaacaggaaggc

SEQ ID NO: 193 - DNA, 21 bp, Artificial Sequence
  Primer
tgtgcagtggcacgatcttgg

SEQ ID NO: 194 - DNA, 20 bp, Artificial Sequence
  Primer
acttcagggtcagcttgccg

SEQ ID NO: 195 - DNA, 17 bp, Artificial Sequence
  Primer
aagaccccaacgagaag

SEQ ID NO: 196 - DNA, 20 bp, Artificial Sequence
  Primer
tcagtgaagagcttgctggc

SEQ ID NO: 197 - DNA, 20 bp, Artificial Sequence
  Primer
tatctacctcggaatcaccc

SEQ ID NO: 198 - DNA, 21 bp, Artificial Sequence
  Primer
aagtcgatgcccttcagctcg

SEQ ID NO: 199 - DNA, 21 bp, Artificial Sequence
  Primer
gtgagcaagggcgaggagctg

SEQ ID NO: 200 - DNA, 21 bp, Artificial Sequence
  Primer
tgggcgacagagtgagattcc

SEQ ID NO: 201 - DNA, 23 bp, Artificial Sequence
  Primer
agcgtgtctgttacaagtgtttg

SEQ ID NO: 202 - DNA, 21 bp, Artificial Sequence
  Primer
aagtcgatgcccttcagctcg

SEQ ID NO: 203 - DNA, 21 bp, Artificial Sequence
  Primer
gtgagcaagggcgaggagctg

SEQ ID NO: 204 - DNA, 20 bp, Artificial Sequence
  Primer
cccacctgctccactctttt

SEQ ID NO: 205 - DNA, 17 bp, Artificial Sequence
  Primer
gactagggctacagggc

SEQ ID NO: 206 - DNA, 20 bp, Artificial Sequence
  Primer
gcagatgaacttcagggtca

SEQ ID NO: 207 - DNA, 17 bp, Artificial Sequence
  Primer
aagaccccaacgagaag

SEQ ID NO: 208 - DNA, 20 bp, Artificial Sequence
  Primer
ttagtgtaggttgggcgctc

SEQ ID NO: 209 - DNA, 21 bp, Artificial Sequence
  Primer
ttcaagacgcacagatctcac

SEQ ID NO: 210 - DNA, 21 bp, Artificial Sequence
  Primer
aagtcgatgcccttcagctcg

SEQ ID NO: 211 - DNA, 21 bp, Artificial Sequence
  Primer
gtgagcaagggcgaggagctg

SEQ ID NO: 212 - DNA, 21 bp, Artificial Sequence
  Primer
acacatttccccagagaaagc

SEQ ID NO: 213 - DNA, 21 bp, Artificial Sequence
  Primer
attacaggcacgagccactgc

SEQ ID NO: 214 - DNA, 21 bp, Artificial Sequence
  Primer
aagtcgatgcccttcagctcg

SEQ ID NO: 215 - DNA, 21 bp, Artificial Sequence
  Primer
gtgagcaagggcgaggagctg

SEQ ID NO: 216 - DNA, 20 bp, Artificial Sequence
  Primer
acgcgggggaagagtagagc

SEQ ID NO: 217 - DNA, 20 bp, Artificial Sequence
  Primer
agaagtccaccgagtcctgc

SEQ ID NO: 218 - DNA, 21 bp, Artificial Sequence
  Primer
aagtcgatgcccttcagctcg

SEQ ID NO: 219 - DNA, 21 bp, Artificial Sequence
  Primer
gtgagcaagggcgaggagctg

SEQ ID NO: 220 - DNA, 19 bp, Artificial Sequence
  Primer
gtgaagctgtagcgcgctc

SEQ ID NO: 221 - DNA, 20 bp, Artificial Sequence
  Primer
accctcaggaagcgtagagt

SEQ ID NO: 222 - DNA, 20 bp, Artificial Sequence
  Primer
ttgccgtgctccttgaagtc

SEQ ID NO: 223 - DNA, 20 bp, Artificial Sequence
  Primer
gagcaaagaccccaacgaga

SEQ ID NO: 224 - DNA, 20 bp, Artificial Sequence
  Primer
tgccaatgctttgttgtcgg

SEQ ID NO: 225 - DNA, 20 bp, Artificial Sequence
  Primer
ggtctaatgtggggtgtggg

SEQ ID NO: 226 - DNA, 20 bp, Artificial Sequence
  Primer
aagtcgatgccttcagctcg

SEQ ID NO: 227 - DNA, 21 bp, Artificial Sequence
  Primer
gtgagcaagggcgagcagctg

SEQ ID NO: 228 - DNA, 20 bp, Artificial Sequence
  Primer
ttctcccagccagcaaacaa

SEQ ID NO: 229 - DNA, 20 bp, Artificial Sequence
  Primer
gggccattgtgcccagaagt

SEQ ID NO: 230 - DNA, 19 bp, Artificial Sequence
  Primer
gacacgctgaacttgtggc

SEQ ID NO: 231 - DNA, 17 bp, Artificial Sequence
  Primer
aagaccccaacgagaag

SEQ ID NO: 232 - DNA, 20 bp, Artificial Sequence
  Primer
caccgttccaaccctgtggc

SEQ ID NO: 233 - DNA, 20 bp, Artificial Sequence
  Primer
gtgacctcagtagctgcatg

SEQ ID NO: 234 - DNA, 20 bp, Artificial Sequence
  Primer
caggggttgaagacaagcag

SEQ ID NO: 235 - DNA, 21 bp, Artificial Sequence
  Primer
tgtgcactggcacgatcttgg

SEQ ID NO: 236 - DNA, 20 bp, Artificial Sequence
  Primer
tcagtgaagagcttgctggc

SEQ ID NO: 237 - DNA, 20 bp, Artificial Sequence
  Primer
tcagttaggccacatcagcg

SEQ ID NO: 238 - DNA, 20 bp, Artificial Sequence
  Primer
gtgccctaaactgagcaacg

SEQ ID NO: 239 - DNA, 20 bp, Artificial Sequence
  Primer
tatctacctcggaatcaccc

SEQ ID NO: 240 - DNA, 21 bp, Artificial Sequence
  Primer
tgggcgacagagtgagattcc

SEQ ID NO: 241 - DNA, 23 bp, Artificial Sequence
  Primer
tctgcctcctttgttaacttgac

SEQ ID NO: 242 - DNA, 23 bp, Artificial Sequence
  Primer
tgctcagttttcacaaacacagt

SEQ ID NO: 243 - DNA, 22 bp, Artificial Sequence
  Primer
agcgtgtctgttacaagtgttg

SEQ ID NO: 244 - DNA, 20 bp, Artificial Sequence
  Primer
gccacctgctccactctttt

SEQ ID NO: 245 - DNA, 20 bp, Artificial Sequence
  Primer
gtcttggtctggaaggaggc

SEQ ID NO: 246 - DNA, 20 bp, Artificial Sequence
  Primer
caagagaagcccctggacag

SEQ ID NO: 247 - DNA, 17 bp, Artificial Sequence
  Primer
gactagggctacagggc

SEQ ID NO: 248 - DNA, 20 bp, Artificial Sequence
  Primer
ttagtgtaggttgggcgctc

SEQ ID NO: 249 - DNA, 20 bp, Artificial Sequence
  Primer
ctcgtcttgcattttcccgc

SEQ ID NO: 250 - DNA, 20 bp, Artificial Sequence
  Primer
gaccgagaccctgttccttc

SEQ ID NO: 251 - DNA, 21 bp, Artificial Sequence
  Primer
ttcaagacgcacagatctcac

SEQ ID NO: 252 - DNA, 21 bp, Artificial Sequence
  Primer
acacatttccccagagaaagc

SEQ ID NO: 253 - DNA, 20 bp, Artificial Sequence
  Primer
gccaactgcattgactccac

SEQ ID NO: 254 - DNA, 20 bp, Artificial Sequence
  Primer
agcaaaatggcgaccacaac

SEQ ID NO: 255 - DNA, 21 bp, Artificial Sequence
  Primer
attacaggcacgagccactgc

SEQ ID NO: 256 - DNA, 20 bp, Artificial Sequence
  Primer
acgcgggggaagagtagagc

SEQ ID NO: 257 - DNA, 20 bp, Artificial Sequence
  Primer
ctgggactcaaggcgctaac

SEQ ID NO: 258 - DNA, 20 bp, Artificial Sequence
  Primer
cgatggggtacttcagggtg

SEQ ID NO: 259 - DNA, 20 bp, Artificial Sequence
  Primer
agaagtccaccgagtcctgc

SEQ ID NO: 260 - DNA, 19 bp, Artificial Sequence
  Primer
gtgaagctgtagcgcgctc

SEQ ID NO: 261 - DNA, 21 bp, Artificial Sequence
  Primer
aggtcttgttgacccggaagt

SEQ ID NO: 262 - DNA, 22 bp, Artificial Sequence
  Primer
acgcactgcatccaagtgtact

SEQ ID NO: 263 - DNA, 20 bp, Artificial Sequence
  Primer
accctcaggaagcgtagagt

SEQ ID NO: 264 - DNA, 20 bp, Artificial Sequence
  Primer
tgccaatgctttgttgtcgg

SEQ ID NO: 265 - DNA, 20 bp, Artificial Sequence
  Primer
ccgagttgaattccctcccc

SEQ ID NO: 266 - DNA, 20 bp, Artificial Sequence
  Primer
ctatgcacctgcccagtacg

SEQ ID NO: 267 - DNA, 20 bp, Artificial Sequence
  Primer
ggtctaatgtggggtgtggg

SEQ ID NO: 268 - DNA, 20 bp, Artificial Sequence
  Primer
ttctcccagccagcaaacaa

SEQ ID NO: 269 - DNA, 21 bp, Artificial Sequence
  Primer
tgtggtgagggtgaaagagga

SEQ ID NO: 270 - DNA, 22 bp, Artificial Sequence
  Primer
agacatgggtaagcaagcaaca

SEQ ID NO: 271 - DNA, 20 bp, Artificial Sequence
  Primer
gggccattgtgcccagaagt

SEQ ID NO: 272 - DNA, 20 bp, Artificial Sequence
  Primer
caccgttccaaccctgtggc

SEQ ID NO: 273 - DNA, 15 bp, Artificial Sequence
  Made in Lab - synthesized linker sequence
gccggctccggtacc

SEQ ID NO: 274 - DNA, 21 bp, Artificial Sequence
  Made in Lab - synthesized linker sequence
cacgacccccccgttgctacg

SEQ ID NO: 275 - DNA, 39 bp, Artificial Sequence
  Made in Lab - synthesized linker sequence
aagcccaacagcgccgtggacggcaccgccggccccggc

SEQ ID NO: 276 - DNA, 30 bp, Artificial Sequence
  Made in Lab - synthesized linker sequence
agcggcctgagaagcagagcccaggccagc

SEQ ID NO: 277 - DNA, 33 bp, Artificial Sequence
  Made in Lab - synthesized linker sequence
tacagcgatctggagctgaagctgcggatccct

SEQ ID NO: 278 - DNA, 18 bp, Artificial Sequence
  Made in Lab - synthesized linker sequence
ggtaccagcggcggaagc

SEQ ID NO: 279 - DNA, 15 bp, Artificial Sequence
  Made in Lab - synthesized linker sequence
agcggcctgagaagc

SEQ ID NO: 280 - DNA, 36 bp, Artificial Sequence
  Made in Lab - synthesized linker sequence
agcggcctgagaagcagagccctggagagagacaag

SEQ ID NO: 281 - DNA, 30 bp, Artificial Sequence
  Made in Lab - synthesized linker sequence
ggcggtagcggggatccaccggtcgccacc

SEQ ID NO: 282 - DNA, 18 bp, Artificial Sequence
  Made in Lab - synthesized linker sequence
ggaggttcaggaggcagc

SEQ ID NO: 283 - DNA, 15 bp, Artificial Sequence
  Made in Lab - synthesized linker sequence
agcggcctgagaagc

SEQ ID NO: 284 - DNA, 57 bp, Artificial Sequence
  Made in Lab - synthesized linker sequence
gaatttggcagcaccggcagcaccggcagcaccggcgcggatccgccggtggcgacc

SEQ ID NO: 285 - DNA, 21 bp, Artificial Sequence
  Made in Lab - synthesized linker sequence
agagacccccctgtcgccacc

SEQ ID NO: 286 - DNA, 21 bp, Artificial Sequence
  Made in Lab - synthesized linker sequence
agcggcctgagaagcagggcc

SEQ ID NO: 287 - DNA, 45 bp, Artificial Sequence
  Made in Lab - synthesized linker sequence
ctccagagcaccgtgccacgggctcgggacccacctgtggccacc

(19) United States
(12) Patent Application Publication
(10) Pub. No.: US 2019/0365818 A1
(43) Pub. Date: Dec. 5, 2019

(54) GENETICALLY-TAGGED STEM CELL LINES AND METHODS OF USE

(71) Applicant: ALLEN INSTITUTE, Seattle, WA (US)

(72) Inventors: Ruwanthi GUNAWARDANE, Seattle, WA (US); Brock ROBERTS, Seattle, WA (US); Amanda HAUPT, Burien, WA (US); Andrew TUCKER, Edmonds, WA (US)

(21) Appl. No.: 16/483,540

(22) PCT Filed: Feb. 9, 2018
(86) PCT No.: PCT/US2018/017708

§ 371 Date: Aug. 5, 2019

Related U.S. Application Data
(60) Provisional application No. 62/457,088, filed on Feb. 9, 2017.
(60) Provisional application No. 62/519,045, filed on Jun. 13, 2017.
(60) Provisional application No. 62/546,237, filed on Aug. 16, 2017.
(60) Provisional application No. 62/552,185, filed on Aug. 30, 2017.
(60) Provisional application No. 62/556,115, filed on Sep. 8, 2017.
(60) Provisional application No. 62/570,081, filed on Oct. 9, 2017.
(60) Provisional application No. 62/582,295, filed on Nov. 6, 2017.

(51) Int. Cl. A61K 35/28; G01N 33/50; C12N 5/0797; C12N 15/90; C12N 9/22; C12N 15/62

(52) U.S. Cl. CPC A61K 35/28; G01N 33/5073; C12N 5/0623; C12N 2310/20; C12N 9/22; C12N 15/62; C07K 2319/60; C12N 15/907

(57) ABSTRACT

The present invention provides stably tagged stem cells and methods for producing stem cells comprising one or more tagged proteins using a gene editing system. The methods described herein enable the insertion of large fluorescent tags into a plurality of genomic loci to generate stem cells that are phenotypically and functional similar to the un-modified parent population. Stem cells produced by the methods described herein additionally retain the capacity to self-renew and differentiate into specialized cell types and can be used in assays and visualization of three-dimensional live cell imaging.

### REFERENCE TO RELATED APPLICATIONS

This application claims priority to U.S. Provisional Application Nos. 62/457,088, filed Feb. 9, 2017; 62/519,045, filed Jun. 13, 2017; 62/546,237, filed Aug. 16, 2017; 62/552,185, filed Aug. 30, 2017; 62/556,115, filed Sep. 8, 2017; 62/570,081, filed Oct. 9, 2017; and 62/582,295, filed Nov. 6, 2017, the contents of which are each incorporated herein by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: AIBS_005_07WO_ST25.txt; date recorded: Feb. 9, 2018; file size 326 kilobytes)

### FIELD OF THE INVENTION

The present disclosure relates to the fields of stem cell biology, genetics, and genetic engineering. In particular aspects, the present disclosure relates to methods of genetically engineering stem cells to express one or more fluorescently-tagged structural or other proteins. In further embodiments, the methods described herein allow for the generation of genetically-engineered, fluorescently-tagged stem cells, wherein the endogenous functions of the stem cells remain un-altered (e.g., pluripotency and genomic stability). In further embodiments, the methods allow for three-dimensional live cell imaging of intracellular proteins. In further embodiments, the methods allow for use of the cells for screening, observing cellular dysplasia, disease staging, monitoring disease progression or improvement, or cellular stress in response to a test agent

### BACKGROUND OF THE INVENTION

Conventional methods of live-cell protein imaging utilize protein fusion constructs, wherein a detectable marker (e.g., a fluorescent protein) is fused to the protein of interest, transduced or transfected into a cell. As such, these systems result essentially in the production of a cell that overexpresses the transduced protein. Although these systems have enabled the probing and analysis of protein localization and cellular dynamics in a wide range of cell types and assays, they fail to allow for the analysis and characterization of a target protein in an un-altered, endogenous state. For example, fusion constructs often result in unpredictable and artificial expression levels of the tagged protein, either as a result of transient expression of transfected constructs, or as a result of copy number variation with transduced constructs. These realities hinder the interpretation of experiments and in turn the study of pathogenesis and drug discovery.

The limitations of exogenous fusion construct systems are further exacerbated in the context of cells that are difficult to transfect or transduce, such as stem cells. In such cells, variation in the expression level of the construct may be especially problematic, as levels of transduction/transfection efficiency may be particularly low to begin with. Accordingly, there is a need in the art for methods that enable tagging of endogenous proteins such that the endogenous expression levels, function, and localization of the protein remain unaltered.

### SUMMARY OF THE INVENTION

In stem cells, and other cells that are particularly difficult to transfect or transduce, engineering of the endogenous genomic sequence to insert a protein tag overcomes the challenges of variable expression and allows for dynamic study of the endogenously-regulated, targeted gene product. These systems are enabled by the CRISPR (Clustered Regularly Interspaced Short Palindromic Repeats)/Cas (CRISPR Associated) nuclease system, which allows for the precise targeting of a genomic locus, and with the insertion of a fluorescent protein (FP) tag under the endogenous regulatory control of the target locus.

CRISPR/Cas9 eliminates many of the challenges associated with genetic engineering and an ever-growing number of studies illuminate the power of this approach. The system is most commonly used in loss-of-function studies, wherein one or more genes are mutated or deleted to generate genetic knock-outs. Less common is the use of the system to introduce exogenous genetic sequences into a target locus. In this instance, homology-directed repair (HDR) mediates the insertion of a repair template into the target locus and can be used to correct an existing mutation in the genomic sequence or to insert exogenous nucleic acid sequences (e.g., a nucleic acid sequence encoding a fluorescent protein). Although HDR has a low error-rate, it is an inherently inefficient process, with rates of less than 10% in normal cells. As such, until now it has been difficult to reproduce HDR-mediated protein tagging across multiple targets to enable systematic use of this process in the study of endogenous protein dynamics particularly in view of the unpredictability of how the introduction of large fluorescent tags may affect endogenous gene function as well as stem cell viability, pluripotency, and chromosomal stability.

The methods provided herein utilize CRISPR/Cas9-mediated gene editing to introduce fluorescent tags via HDR into the genomic loci of target proteins, into the genomic safe harbor location, or other locations in the genome. These methods result in the production of isogenic hiPSC clones expressing detectable endogenously-regulated fusion proteins unique to each cell line, and do not substantially modify or alter stem cell pluripotency or function.

In some embodiments, the present invention provides a method for producing a stem cell comprising at least one tagged endogenous protein comprising: (a) providing a ribonucleoprotein (RNP) complex comprising a Cas protein, a CRISPR RNA (crRNA) and a trans-activating RNA (tracrRNA), wherein the crRNA is specific for a target genomic locus; (b) providing a donor plasmid comprising a first polynucleotide sequence encoding a detectable tag, a second polynucleotide sequence encoding a 5′ homology arm, and a third polynucleotide sequence encoding a 3′ homology arm, wherein the 5′ homology arm and 3′ homology arms are at least about 1 kb in length; and (c) transfecting the complex of (a) and the donor plasmid of (b) into a stem cell such that the polynucleotide sequence encoding the detectable tag is inserted into a target genomic locus to generate a tagged endogenous protein, thereby producing a stem cell comprising at least one tagged endogenous protein.

In some embodiments, the polynucleotide sequence encoding the detectable tag further comprises a polynucleotide sequence encoding a flexible linker. In some embodiments, the polynucleotide sequence encoding the detectable tag comprises at least about 20 nucleotides in length. In some embodiments, the encoded detectable tag comprises at least about 8 amino acids in length. In some embodiments, the polynucleotide sequence encoding the detectable tag comprises at least about 300 nucleotides in length. In some embodiments, the encoded detectable tag comprises at least about 100 amino acids in length. In some embodiments, the detectable tag is a fluorescent protein, a luminescent protein, a photoactivatable protein, a FLAG tag, a SNAP tag, or a Halo tag. In some embodiments, the fluorescent protein is selected from the group comprising green fluorescent protein (GFP), blue fluorescent protein, cyan fluorescent protein, yellow fluorescent protein, or red fluorescent protein.

In some embodiments, the RNP comprises a crRNA, tracrRNA, and Cas9 protein complexed at a ratio of 1:1:1. In some embodiments, the Cas protein is a wild-type Cas9 protein or a Cas9-nickase protein. In some embodiments, the crRNA sequence is selected to minimize off-target cleavage of genomic DNA sequences and/or insertion of the detectable tag. In some embodiments, the off-target cleavage of genomic DNA sequences and/or insertion of the detectable tag is less than 1.0%. In some embodiments, transfecting the CRISPR/Cas9 RNP and the donor plasmid into a stem cell results in a double stranded break at the target genomic locus. In some embodiments, the double stranded break is repaired by homology directed repair (HDR). In some embodiments, the polynucleotides encoding 5′ homology arm, the detectable tag, and the 3′ homology arm act as a repair template during HDR In some embodiments, protospacer adjacent motif (PAM) sequences are removed from the polynucleotide backbone of the donor plasmid. In some embodiments, the donor plasmid further comprises an antibiotic-resistance gene. In some embodiments, the antibiotic-resistance gene confers resistance to ampicillin and/or kanamycin.

In some embodiments, the stem cell is an induced pluripotent stem cell (iPSC) derived from a healthy donor. In some embodiments, the iPSC is a WTC cell or a WTB cell.

In some embodiments, transfecting the CRISPR/Cas9 RNP and the donor plasmid occurs by electroporating the stem cells. In some embodiments, the stem cells are electroporated using a Neon® transfection system or an Amaxa Nucleofector® system. In some embodiments, the stem cells are electroporated for at least 1 pulse. In some embodiments, the pulse is at least about 15 ms at a voltage of at least about 1300 V. In some embodiments, the stem cells are electroporated for 1-5 pulses. In some embodiments, the stem cells are electroporated for at least 2 pulses.

In some embodiments, the target genomic locus is a locus within a gene encoding a structural protein. In some embodiments, the structural protein is selected from paxillin, alpha tubulin, lamin B1, Tom20, desmoplakin, beta actin, Sec61B, fibrillarin, myosin, centrin2, ZO-1, Safe-harbor-GFP, ST6Gal1, vimentin, LAMP1, LC3, Safe harbor-CAAX, and PMP34.

In some embodiments, a plurality of detectable tags are inserted into a plurality of target loci. In some embodiments, a plurality polynucleotides encoding a plurality of detectable tags are inserted into one donor plasmid. In some embodiments, two or more polynucleotides encoding two or more detectable tags are inserted into one donor plasmid. In some embodiments, a first plurality of polynucleotides encoding two or more detectable tags are inserted into a first donor plasmid and a second plurality of polynucleotides encoding two or more detectable tags are inserted into a second donor plasmid. In some embodiments, a first polynucleotide encoding a first detectable tag is inserted into a first donor plasmid and a second polynucleotide encoding a second detectable tag is inserted into a second donor plasmid. In some embodiments, the first and second donor plasmid are introduced to the cell at the same time. In some embodiments, the first and second donor plasmid are introduced to the cell sequentially.

In some embodiments, 10 polynucleotides each encoding a unique detectable tag and each inserted into one of about 10 different donor plasmids. In some embodiments, the 10 different donor plasmids are introduced to the cell at the same time. In some embodiments, the 10 different donor plasmids are introduced to the cell sequentially.

In some embodiments, between 2 and 10 detectable tags are inserted into between 2 and 10 target loci. In some embodiments, between 3 and 5 detectable tags are inserted into between 3 and 5 target loci.

In some embodiments, the methods described herein further comprise selecting the stem cells that comprise at least one tagged protein. In some embodiments, selecting the stem cells comprises selecting the stem cells that are positive for the detectable tag using fluorescence activated cell sorting (FACS). In some embodiments, at least about 0.1% of the stem cells are positive for the detectable tag.

In some embodiments, the methods described herein further comprise screening of the stem cells comprises genetic screening to determine at least two or more of the following: (a) insertion of the detectable tag sequence; (b) stable integration of the plasmid backbone; and/or (c) relative copy number of the detectable tag sequence. In some embodiments, the genetic screen is performed by droplet digital PCR (ddPCR), by tile junction PCR, or both.

In some embodiments, selecting clones comprising an insertion of the detectable tag comprises selecting clones that have the detectable tag sequence inserted into one or both alleles of the target genomic locus and do not have stable integration of the plasmid backbone.

In some embodiments, the methods described herein further comprise sequencing clones comprising an insertion of the detectable tag to identify clones comprising a precise insertion of the detectable tag. In some embodiments, clones comprising a precise insertion are identified by: (a) amplifying the genomic sequences across the junction between the inserted detectable tag and the 5′ and 3′ distal genomic regions to generate tiled-junction amplification products. (b) sequencing the tiled-junction amplification products of (a); and (c) comparing the sequence of the tiled-junction amplification products with a reference sequence.

In some embodiments, the stem cell comprising at least one tagged endogenous protein expresses at least one protein associated with pluripotency. In some embodiments, the protein associated with pluripotency is selected from the group comprising Oct3/4, Sox2, Nanog, Tra-160, and Tra-181, SSEA3/4. In some embodiments, expression level of the at least one protein associated with pluripotency is comparable to the expression level of the same protein in an unmodified stem cell. In some embodiments, the stem cell comprising at least one tagged protein maintains a differentiation potential that is comparable to an unmodified stem cell. In some embodiments, the stem cell comprising at least one tagged protein is capable of differentiating into mesoderm, endoderm, or ectoderm.

In some embodiments, the expression of the at least one tagged protein is maintained in a differentiated cell derived from the stem cell comprising at least one tagged protein. In some embodiments, the morphology, viability, potency, and endogenous cellular functions of the stem cells comprising at least one tagged protein and/or differentiated cells derived from stem cells comprising at least one tagged protein are not substantially changed compared to unmodified stem cells and differentiated cells thereof.

In some embodiments, the present invention provides a method for screening the effects of one or more test agents on one or more cellular structures in one or more cell types comprising: providing one or more cultures of one or more stem cells and/or differentiated cells derived therefrom produced by the methods described herein, wherein the stem cells or differentiated cells derived therefrom comprise a tagged endogenous protein; adding one or more test agent to one or more of the cultures; assaying the culture at one or more time points before and/or after the addition of the one or more test agent, and determining the effects of the one or more test agent on one or more cellular structures in the one or more cell types.

In some embodiments, the effect of the one or more test agents are determined by visualization of the cells. In some embodiments, the tagged endogenous protein comprises at least about 100 amino acids in length. In some embodiments, the tagged endogenous protein is a fluorescent protein, a luminescent protein, a photoactivatable protein, a FLAG tag, a SNAP tag or a Halo tag. In some embodiments, the fluorescent protein is selected from the group comprising green fluorescent protein (GFP), blue fluorescent protein, cyan fluorescent protein, yellow fluorescent protein or red fluorescent protein. In some embodiments, the tagged endogenous protein is a structural protein. In some embodiments, the structural protein is selected from paxillin, alpha tubulin, lamin B1, Tom20, desmoplakin, beta actin, Sec61B, fibrillarin, myosin, centrin2, ZO-1, Safe-harbor-GFP, ST6Gal1, vimentin, LAMP, LC3, Safe harbor-CAAX, and PMP34.

In some embodiments, the methods provided herein for determining the effect of one or more test agents comprises providing two or more cultures of stem cells and/or one or more differentiated cells derived therefrom. In some embodiments, the two or more cultures each comprise a different differentiated cell type and/or a different tagged endogenous structure. In some embodiments, the two or more cultures each comprise a different differentiated cell type and a different tagged endogenous structure.

In some embodiments, the methods described herein comprise microscopy of the one or more cultures of one or more stem cells and/or one or more differentiated cells derived therefrom at one or more time points before and/or after addition of the one or more test agent. In some embodiments, the microscopy is confocal microscopy.

In some embodiments, determining effects on one or more cellular structures comprises comparing one or more variables selected from subcellular morphology, localization and/or dynamics of tagged structure(s), viability and cellular morphology from one or more cultures of one or more stem cells and/or one or more differentiated cells derived therefrom at one or more time points after treatment with the same variable prior to treatment.

In some embodiments, the determining effects on one or more cellular structures comprises comparing one or more variables selected from subcellular morphology, localization and/or dynamics of tagged structure(s), viability and cellular morphology from one or more cultures of one or more stem cells and/or one or more differentiated cells derived therefrom at one or more time points after treatment with one or more cultures of one or more stem cells and/or one or more differentiated cells derived therefrom treated with a control agent.

In some embodiments, the present invention provides kits comprising an array of stem cells or differentiated cells derived therefrom comprising at least one tagged endogenous protein. In some embodiments, the kit comprises stem cells or differentiated cells derived therefrom comprising at least one tagged endogenous protein made according to the methods described herein. In some embodiments of the kits, the detectable tag comprises at least about 100 amino acids in length. In some embodiments of the kits, the detectable tag is a fluorescent protein, a luminescent protein, a photoactivatable protein, a FLAG tag, a SNAP tag or a Halo tag. In some embodiments of the kits, the fluorescent protein is selected from the group comprising green fluorescent protein (GFP), blue fluorescent protein, cyan fluorescent protein, yellow fluorescent protein or red fluorescent protein. In some embodiments of the kits, the tagged protein is a structural protein. In some embodiments of the kits, the structural protein is selected from paxillin, alpha tubulin, lamin B1, Tom20, desmoplakin, beta actin, Sec61B, fibrillarin, myosin, centrin2, ZO-1, Safe-harbor-GFP, ST6Gal1, vimentin, LAMP1, LC3, Safe harbor-CAAX, and PMP34.

In some embodiments, the present invention provides a method for visualizing a stem cell produced by the method of claim 1, comprising: (a) plating the stem cells on plates; and (b) imaging the cells by microscope. In some embodiments, the imaging is live-cell imaging. In some embodiments, the imaging is in three dimensions. In some embodiments, the imaging involves co-localization with antibodies.

In some embodiments, the present invention provides a donor polynucleotide comprising a first polynucleotide sequence encoding a detectable tag, a second polynucleotide sequence encoding a 5′ homology arm, and a third polynucleotide sequence encoding a 3′ homology arm, wherein the 5′ homology arm and 3′ homology arm are each about 1 kb in length. In some embodiments, the donor polynucleotide further comprises a flexible linker sequence. In some embodiments, the polynucleotide sequence encoding the detectable tag comprises at least about 20 nucleotides in length. In some embodiments, the polynucleotide sequence encoding the detectable tag comprises between about 300 nucleotides in length and 3,000 nucleotides in length. In some embodiments, the polynucleotide sequence encoding the detectable tag is greater than 3000 nucleotides. In some embodiments, the polynucleotide sequence encoding the detectable tag encodes a detectable tag that comprises at least about 8 amino acids in length. In some embodiments, the polynucleotide sequence encoding the detectable tag encodes a detectable tag that comprises between about 8 and about 100 amino acids in length.

In some embodiments, at least two detectable tags are encoded by the donor polynucleotide. In some embodiments, the detectable tag is selected from the group consisting of a fluorescent protein, a luminescent protein, a photoactivatable protein, a FLAG tag, a SNAP tag, and a Halo tag. In some embodiments, the fluorescent protein is selected from the group consisting of green fluorescent protein (GFP), blue fluorescent protein, cyan fluorescent protein, yellow fluorescent protein, and red fluorescent protein. In some embodiments, the fluorescent protein is selected from the group consisting of mCherry, tdTomato, mNeonGreen, and mTagRFPt. In some embodiments, n the donor polynucleotide is a plasmid.

In some embodiments, the present invention provides a use of a donor polynucleotide of any of claims 91 to 92 to produce a stem cell using a gene editing system selected from the group consisting of: (a) a CRISPR/Cas9 ribonucleoprotein (RNP) complex comprising a Cas9 protein, a CRISPR RNA (crRNA) and a trans-activating RNA (tracrRNA), wherein the crRNA is specific for a target genomic locus; (b) a polynucleotide encoding a Cas nuclease, a CRISPR RNA (crRNA) and a trans-activating RNA (tracrRNA), wherein the crRNA is specific for a target genomic locus; (c) a TALEN; and (d) a zinc finger nuclease.

In some embodiments, the present invention provides use a of the donor polynucleotide described herein for imaging one or more proteins in one or more cells. In some embodiments, the one or more cells are tissue. In some embodiments, the one or more cells are living. In some embodiments, the imaging is three dimensional imaging.

In some embodiments, the present invention provides a stably tagged stem cell clone produced by the methods described herein.

In some embodiments, the present invention provides a purified preparation of the stably tagged stem cell clones described herein.

In some embodiments, the present invention provides a method of generating a signature for a test agent comprising: (a) admixing the test agent with one or more stably tagged stem cell clones produced by the methods described herein; (b) detecting a response in the one or more stem cell clone; (c) detecting a response in a control stem cell; (d) detecting a difference in the response in the one or more stem cell clones from the control stem cell; and (e) generating a data set of the difference in the response.

In some embodiments, the present invention provides a stably tagged stem cell clone produced by the methods described herein in an activity selected from the group consisting of: (a) determining toxicity of a test agent on the stably tagged stem cell clone; (b) determining the stage of disease in a stably tagged stem cell clone; (c) determining the dose of a test agent or drug for treatment of disease; (d) monitoring disease progression in a stably tagged stem cell clone; and (e) monitoring effects of treatment of a test agent or drug on the stably tagged stem cell clone.

In some embodiments, the present invention provides use of a stably tagged stem cell clone produced by the methods described herein for monitoring progression of disease or effect of a test agent on a disease wherein the disease is selected from the group consisting of aberrant cell growth, wound healing, inflammation, and neurodegeneration.

In some embodiments, the present invention provides a differentiated cell or group of differentiated cells derived from a stably tagged stem cell clone described herein. In some embodiments, the differentiated cell or group of differentiated cells are selected from the group consisting of cardiomyocytes, differentiated kidney cells, and differentiated fibroblasts.

In some embodiments, the present invention provides a stably tagged stem cell clone comprising a CRISPR/Cas9 ribonucleoprotein (RNP) complex. In some embodiments, the stably tagged stem cell clone comprises a donor polynucleotide, wherein in the donor polynucleotide comprises a first polynucleotide sequence encoding a detectable tag, a second polynucleotide sequence encoding a 5′ homology arm, and a third polynucleotide sequence encoding a 3′ homology arm, wherein the 5′ homology arm and 3′ homology arm are about 1 kb in length.

In some embodiments, the present invention provides a stably tagged stem cell clone comprising a donor polynucleotide, wherein in the donor polynucleotide comprises a first polynucleotide sequence encoding a detectable tag, a second polynucleotide sequence encoding a 5′ homology arm, and a third polynucleotide sequence encoding a 3′ homology arm, wherein the 5′ homology arm and 3′ homology arm are about 1 kb in length.

In some embodiments, the methods described here further comprise microscopy of the one or more cultures of one or more stem cells and/or one or more differentiated cells derived therefrom at one or more time points before and/or after addition of the one or more test agent. In some embodiments, the microscopy is confocal microscopy.

In some embodiments, the present invention provides a kit comprising an array of stem cells or differentiated cells derived therefrom for visualizing or screening the effects of one or more test agents on one or more cellular structures in one or more cell types comprising at least one tagged protein.

### BRIEF DESCRIPTION OF THE DRAWINGS

FIG. 1A-FIG. 1D provide schematics of illustrative gene editing and clone selection protocols. FIG. 1A shows a schematic illustrating design features important for genome editing experiments. FIG. 1B illustrates a schematic of donor plasmids for N-terminal tagging of LMNB1 and C-terminal tagging of DSP. FIG. 1C illustrates a schematic depicting the genome editing process. FIG. 1D shows a schematic overview of the clone isolation, genetic screening, and quality control workflow.

FIG. 2A-FIG. 2D illustrate comparisons of gene editing efficiency. FIG. 2A shows flow cytometry plots displaying GFP intensity (y-axis) 3-4 days after editing. FIG. 2B shows a comparison of genome editing efficiency, as defined by FACS, shown as a percentage of GFP+ cells within the gated cell population in each panel of FIG. 2A. FIG. 2C shows estimated percentage of cells in the FACS-enriched populations expressing GFP, as determined by live microscopy. FIG. 2D shows a representative image of the LMNB1 Crl FACS-enriched population showing an enrichment of GFP+ cells. Scale bars are 10 μm.

FIG. 3A-FIG. 3C show a schematic illustrating the sequential process for identifying precisely tagged clones. In step 1 (FIG. 3A), ddPCR was used to identify clones with GFP insertion (normalized genomic GFP copy number ˜1 or ˜2) and no plasmid integration (normalized genomic plasmid backbone copy number <0.2). Hypothetical example of a typical editing experiment is shown with examples for pass and fail criteria. In step 2 (FIG. 3B), junctional PCR amplification of the tagged allele was used to determine precise on-target GFP insertion. In step 3 (FIG. 3C), the untagged allele of a clone with monoallelic GFP insertion is amplified. The amplicon was then sequenced to ensure that no mutations have been introduced to this allele.

FIG. 4A-FIG. 4E shows results of genetic assays to screen for precise genome editing in clones. FIG. 4A shows ddPCR screening data from five experiments representative of experimental outcome categories. FIG. 4B shows examples of ddPCR screening data from experiments representative of the range of outcomes observed. Each data point represents one clone. FIG. 4C shows the rates of clonal confirmation by junctional tiled PCR following selection by ddPCR FIG. 4D shows the rates of clonal confirmation by junctional tiled PCR when ddPCR was not used as an initial screening criterion. FIG. 4E shows the rate of clonal confirmation by untagged allele amplification and sequencing.

FIG. 5A-FIG. 5E shows additional results of genetic assays to screen for precise genome editing in clones. FIG. 5A shows percentage of clones confirmed by ddPCR to have incorporated the GFP tag but not the plasmid backbone. FIG. 5B shows percentage of clones confirmed in step 1 that also had correctly sized junctional PCR amplicons. FIG. 5C shows percentage of clones confirmed to have wild type untagged alleles by PCR amplification and Sanger sequencing following steps 1 and 2. FIG. 5D shows the percentage of clones in each experiment with KAN/AMP copy number ≥0.2 is displayed on the y-axis. Stacked bars represent 3 observed subcategories of rejected clones. FIG. 5E shows fragment analysis of complete junctional allele amplification.

FIG. 6A-FIG. 6C show amplification of complete junctional (non-tiled) PCR products to demonstrate presence of the allele anticipated from tiled junctional PCR product data. FIG. 6A shows junctional PCR primers complementary to sequences flanking the homology arms in the distal genome were used together to co-amplify tagged and untagged alleles. FIG. 6B shows an assay served to rule out anticipated DNA repair outcomes where tiled junctional PCR data leads to a misleading result because the GFP tag sequence has been duplicated during HDR, as indicated by the schematic. FIG. 6C shows molecular weight markers are as indicated (kb).

FIG. 7 illustrates the morphology of final candidate clones with GFP-tagged PXN.

FIG. 8A-FIG. 8K show live-cell imaging of final 10 edited clonal lines. Scale bars in all panels are as indicated.

FIG. 9A-FIG. 9C show cell biological assays to evaluate co-expression of tagged and untagged protein forms and their relative contributions to cellular proteome and structure. FIG. 9A shows comparison of labeled structures in edited cells and unedited WTC parental cells. FIG. 9B shows lysate from ACTB cl. 184 (left), TOMM20 cl. 27 (middle), and LMNB1 cl. 210 (right) are compared to unedited WTC cell lysate by western blot. FIG. 9C shows quantification of the Western blot analyses in FIG. 9B.

FIG. 10A-FIG. 10F show an assessment of stem cell quality after genome editing. FIG. 10A shows representative phase contrast images depicting cell and colony morphology of the unedited WTC line and several GFP-tagged clones (LMNB1, ACTB, TOMM20, and PXN). FIG. 10B shows representative flow cytometry plots ofgene-edited LMNB1 cl. 210 cells and unedited WTC cells immunostained for indicated pluripotency markers (Nanog, Oct3/4, Sox2, SSEA-3, TRA-1-60) and a marker of differentiation (SSEA-1). FIG. 10C shows representative flow cytometry plots of differentiated unedited WTC cells or gene-edited LMNB1 cl. FIG. 10D shows cardiomyocytes differentiated from unedited WTC cells and stained with cardiac Troponin T (cTnT) antibody to label cardiac myofibrils. FIG. 10E shows representative flow cytometry plots showing cTnT expression in unedited WTC control cells and several gene edited cell lines (LMNB1 cl. 210, ACTB cl. 184, and TOMM20 cl. 27). FIG. 10F shows a quantitative assessment of pluripotency and cardiomyocte differentiation markers for final clones

FIG. 11A-FIG. 11E illustrate results of phenotypic validation of candidate clones.

FIG. 12 illustrates expression levels of the 12 genes attempted for genome editing in the WTC parental cell line.

FIG. 13A-FIG. 13E illustrate predicted genome wide CRISPR/Cas9 alternative binding sites, categorized according to sequence profile and location with respect to genes. FIG. 13A shows predicted alternative CRISPR/Cas9 binding sites (SEQ ID NOs: 174-186) categorized for each crRNA used. FIG. 13B shows predicted off-target sequence breakdown based on sequence profile. FIG. 13C shows breakdown of sequenced off-target sites by sequence profile. FIG. 13D shows all predicted off-target sites were additionally categorized according to their location with respect to annotated genes. FIG. 13E shows breakdown of sequenced off-target sites by genomic location with respect to annotated genes.

FIG. 14A-FIG. 14B illustrate ddPCR screening data. FIG. 14A shows ddPCR screening data for all experiments. FIG. 14B shows a dilution series of the donor plasmid used for the PXN-EGFP tagging experiment was used to confirm equivalent amplification of the AMP and GFP sequences in two-channel ddPCR assays.

FIG. 15 illustrates comparison of unedited versus edited cells by immunofluorescence.

FIG. 16 illustrates comparison of GFP tag localization and endogenous protein stain in edited cell lines.

FIG. 17 shows live cell imaging comparison of transiently transfected cells and genome edited cells. Top panels depict transiently transfected WTC cells and bottom panels depict gene edited clonal lines. Left: WTC transfected with EGFP-tagged alpha tubulin construct compared to the TUBA1B-mEGFP edited cell line. Images are a single apical frame. Middle: WTC transfected with EGFP-tagged desmoplakin construct compared to the DSP-mEGFP edited cell line. Images are maximum intensity projections of apical 4 z-frames. Right: WTC transfected with mCherry-tagged Tom20 construct compared to the TOMM20-mEGFP edited cell line. Images are single basal frames of the cell.

FIG. 18A-FIG. 18B shows Western blot analysis of all 10 edited clonal lines.

FIG. 19A-FIG. 19B show editing experiments testing the feasibility of biallelic editing of the LMNB1 and TUBA1B loci. FIG. 19A shows final clones LMNB1-mEGFP and TUBA 1B-mEGFP were transfected using the standard editing protocol with a donor cassette targeting the untagged allele of the tagged locus, encoding mTagRFP-T (sequential delivery, top row). FIG. 19B shows the sorted population from FIG. 19A (indicated by asterisk) revealed similar subcellular localization of GFP and mTagRFP-T signal to the nuclear envelope in the majority of cells, suggesting successful biallelic tagging.

FIG. 20A-FIG. 20B show live imaging analysis at two culture time points of TUBA 1B-mEGFP edited cells and the four final edited clones that displayed a low abundance of tagged protein.

FIG. 21A-FIG. 21C show Western blot analysis of candidate clones at one culture time point and final clones at two culture time points from editing experiments that displayed a low abundance of tagged protein.

FIG. 22A-FIG. 22D show flow cytometry analysis of GFP tag expression stability, flow cytometry analysis of cell cycle dynamics, microscopy analysis of mitotic index, and culture growth assays. FIG. 22A shows endogenous GFP signal in final edited clones was compared in otherwise identical cultures separated by four passages (14 days) of culturing time (indicated). FIG. 22B shows propidium iodide staining and flow cytometry were used to quantify numbers of cells in G1 (indicated), S phase (indicated) and G2/M phase (indicated) in final edited clones. FIG. 22C shows DAPI staining of colonies from each of the same five clonal lines was additionally used to quantify the numbers of mitotic cells per colony, as indicated. FIG. 22D shows ATP quantitation was used as an indirect measure of cell growth.

FIG. 23 illustrates PCR primers (SEQ ID NOs: 193-272) used in experiments. All primers are listed in 5′ to 3′ orientation.

FIG. 24A-FIG. 24B illustrates antibodies used in western blot, immunofluorescence, and flow cytometry experiments.

FIG. 25 illustrates a workflow overview and strategy for building predictive models of the dynamic organization and behavior of cells using image-based 3D data sets of fluorescently tagged structures in human induced pluripotent stem cells (hiPSC).

FIG. 26A-FIG. 26C illustrate image-based feature extraction: colony growth and fluorescent texture quantification to sort and select drug-induced end point phenotypes.

FIG. 27 illustrates high resolution 3D images reveal drug signatures on target and non-target cell structures as well as the morphological spectrum of each structure

FIG. 28A-FIG. 28C illustrate fluorescence quantification of 3D images to analyze drug-induced Golgi reorganization.

FIG. 29A-FIG. 29F illustrate relative fluorescence quantification of 3D images and z-axis intensity profiling to analyze drug-induced cytoskeleton reorganization.

FIG. 30 illustrates Z-axis intensity profiling of 3D images to analyze drug-induced cell junction reorganization.

FIG. 31 illustrates Z-axis intensity profiling of 3D images to analyze drug-induced cell junction reorganization.

FIG. 32 illustrates exemplary factors for producing differentiated cell types from human iPSCs.

### DETAILED DESCRIPTION OF THE INVENTION

The present invention provides methods for producing stem cells comprising one or more tagged proteins using the CRISPR/Cas9 gene editing system. The methods described herein enable the insertion of fluorescent tags into a target genomic loci or plurality of target genomic loci to generate stem cells that are phenotypically and functional similar to the un-modified parent population. Stem cells produced by the methods described herein additionally retain the capacity to self-renew and differentiate into specialized cell types.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more detectable tags).

### I. Stem Cells

In some embodiments, the present invention provides for methods of producing a stem cell comprising at least one tagged endogenous protein. In certain embodiments, the endogenous protein is a wild-type protein, whereas in other embodiments, the endogenous protein comprises one or more naturally-occurring mutations and/or one or more introduced mutations. Examples of mutations include but are not limited to amino acid insertions, deletions and substitutions.

The term “stem cell,” as used herein, refers to a multipotent, non-specialized cell with the capacity to self-renew and to differentiate into at least one differentiated cell lineage (e.g., potency). The “stemness” of a stem cell include the characteristics of self-renewal and multipotency. Self-renewal refers to the proliferation of a stem cell to generate one (asymmetric division) or two (symmetric division) daughter cells with development potentials that are indistinguishable from those of the mother cell. Self-renewal results in an expanded population of stem cells, each of which maintains an undifferentiated state and the ability to differentiate into specialized cells. Typically, an expanded population of stem cells retains the stemness characteristics of the parent cell.

Potency refers to the ability of a stem cell to differentiate into at least one type of specialized cell. The greater the number of different specialized cell types a stem cell can differentiate into, the greater its potency. In some embodiments, a stem cell may be a totipotent cell, and able to differentiate into any specialized cell type (e.g., a zygote). In some embodiments, a stem cell may be pluripotent and able to differentiate into cell types of any of the three germ layers (endoderm, mesoderm, or ectoderm) (e.g., an embryonic stem cell or an induced pluriopotent stem cell (iPSC)). In some embodiments, the stem cell may be multipotent and have the capacity to differentiate into multiple cell types of a particular cell lineage (e.g., a hematopoietic stem cell). Multipotent stem cells may also be referred to as progenitor cells. In certain embodiments, stem cells may be obtained from a donor, or they may be generated from a non-stem cell. Non-limiting examples of stem cells include embryonic stem cells and adult stem cells. Stem cells include, but are not limited to, mesenchymal stem cells, adipose tissue-derived stem cells, hematopoietic stem cells, and umbilical cord-derived stem cells.

In some embodiments, the stem cells described herein are human iPSCs. iPSCs are derived from differentiated adult cells and have been modified to express transcription factors and proteins responsible for the induction and/or maintenance of a pluripotent state (e.g., Oct 3/4, Sox family transcription factors, Klf family transcription factors, and Nanog). In some embodiments, the iPSCs described herein are derived from a normal, healthy human donor. In some embodiments, the iPSC is a WTC or a WTB cell line (Kreitzer et al, American Journal of Stem Cells, 2:119-31, 2013; Miyaoka et al., Nature Methods, 11:291-3, 2013). In some embodiments, the iPSC is derived from a human donor that has been diagnosed with a disease or disorder. For example, in some embodiments the iPSC may be derived from a patient diagnosed with a cardiomyopathy (e.g., arrhythmogenic right ventricular cardiomyopathy, dialated cardiomyopathy, hypertrophic cardiomyopathy, left ventricular non-compaction cardiomyopathy, or restrictive cardiomyopathy), a heritable disease (e.g., deficiency of acyl-CoA dehydrogenase, very long chain (ACADVL), Barth syndrome (BTHS), carnitine-acylcarnitine translocase deficiency (CACTD), congenital disorder of deglycosylation (CDDG), muscular dystrophies (including Emery-Dreifuss muscular dystrophy (EDMD1), autosomal dominant Emery-Dreifuss muscular dystrophy (EDMD2), Duchenne's muscular dystrophy, and chronic granulomatous disease), Friedreich ataxia 1 (FRDA), glycogen storage disease II, Hurler-Scheie syndrome, isobutyryl-CoA dehydrogenase deficiency, Kearn-Sayre syndrome (KSS), Leigh syndrome, leprechaunism, long chan 3-hydroxyacyl-CoA dehydrogenase deficiency, mitochondrial DNA depletion syndrome 12 (cardiomyopathic type), mucolipidosis mIa, myoclonus epilepsy associated with ragged-red fibers (MERFF), centronuclear myopathy 1 (CNMI), Preader-Willi syndrome (PWS), adult-onset progeria, propionic academia, Vici syndrome (VICIS), or Werner syndrome), or a disease caused by or associated with a chromosomal abnormality (e.g., chromosome 1P36 deletion syndrome, Duchenne's muscular dystrophy, and Prader-Willi syndrome).

“Stem cell markers” as used herein are defined as gene products (e.g. protein, RNA, glycans, glycoproteins, etc.) that are specifically or predominantly expressed by stem cells. Cells may be identified as a particular type of stem cell based on their expression of one or more of the stem cell markers using techniques commonly available in the art including, but not limited to, analysis of gene expression signatures of cell populations by microarray, qPCR, RNA-sequencing (RNA-Seq), Next-generation sequencing (NGS), serial analysis of gene expression (SAGE), and/or analysis of protein expression by immunohistochemistry, western blot, and flow cytometry. Stem cell markers may be present in the nucleus (e.g., transcription factors), in the cytosol, and/or on the cell membrane (e.g., cell-surface markers). In some embodiments, a stem cell marker is a gene product that directly and specifically supports the maintenance of stem cell identity and/or stem cell function. In some embodiments, a stem cell marker is gene that is expressed specifically or predominantly by stem cells but does not necessarily have a specific function in the maintenance of stem cell identity and/or stem cell function. Examples of stem cell markers include, but are not limited to, Oct 3/4, Sox2, Nanog, Tra-160, Tra-181, and SSEA3.

In some embodiments, the present invention provides genetically engineered stem cells. Herein, the terms “genetically engineered stem cells” or “modified stem cells” or “edited stem cells” refer to stem cells that comprise one or more genetic modifications, such as one or more tags inserted into a locus of one or more endogenous target genes. “Genetic engineering” refers to the process of manipulating a genomic DNA sequence to mutate or delete one or more nucleic acids of the endogenous sequence or to introduce an exogenous nucleic acid sequence into the genomic locus. The genetically-engineered or modified stem cells described herein comprise a genomic DNA sequence that is altered (e.g., genetically engineered to express a tag) compared to an un-modified stem cell or control stem cell. As used herein, an un-modified or control stem cell refers to a cell or population of cells wherein the genomes have not been experimentally manipulated (e.g., stem cells that have not been genetically engineered to express a tag).

In some embodiments, the stem cells described herein are derived from a donor (e.g., a healthy donor) and comprise one or more genetic mutations associated with a particular disease or disorder introduced into the iPSC genome. Such embodiments are referred to herein as “mutant stem cells.” Introduction of mutations into an iPSC derived from a health donor can mimic the genetic state of a particular disease or disorder, while maintaining the isogenic relationship between the mutant stem cell and the normal iPSC from which it is derived. This allows direct comparisons between the two cell types to be made when assessing the effect of a particular mutation on cellular structure, cellular function, protein localization, protein function, and/or protein expression. For example, mutations may be introduced into the PKD1 and/or PKD2 genes of an iPSC derived from a healthy donor to produce a PC 1-mutant stem cell, a PC2-mutant stem cell, or a PC1/PC2-mutant stem cell. These mutant stem cells and the corresponding normal stem cells from which they are derived can then be further engineered to express one or more detectable markers in one or more endogenous target genomic loci. In some embodiments, these cells are assayed according to the methods described herein to determine the effect of a particular mutation on cellular structure, cellular function, protein localization, protein function, and/or protein expression, and can elucidate the role of a protein in different diseases, such as polycystic kidney disease.

In some embodiments, the present invention provides populations of genetically engineered stem cells that have been modified to express one or more tagged endogenous proteins. Herein, a “population” of cells (e.g., stem cells) refers to any number of cells greater than 1, e.g., at least 1×10³ cells, at least 1×10⁴ cells, at least 1×10⁵ cells, at least 1×10⁶ cells, at least 1×10⁷ cells, at least 1×10⁸ cells, at least 1×10⁹ cells, or at least 1×10¹⁰ or more cells.

### II. Methods of Producing Genetically-Engineered Stem Cells

In some embodiments, the present invention provides methods of producing genetically-engineered stem cells comprising at least one tagged endogenous protein. In some embodiments, the method comprises (a) providing a gene-editing system capable of producing double or single stranded DNA breaks at a target endogenous locus; (b) providing a repair template comprising a polynucleotide sequence encoding a detectable tag: (c) introducing the gene-editing system and the repair template into a stem cell such that the polynucleotide sequence encoding the detectable tag is inserted into an endogenous target genomic locus to generate the tagged endogenous protein. In certain embodiments, during step (c), the cells are cultured under conditions that allow insertion of the sequence encoding the detectable tag into the target genomic locus, such as any of those disclosed herein. In particular embodiments, the cells produced in step (c) are cultured under conditions suitable for expression of the tagged endogenous protein. In various embodiments of any of the methods disclosed herein, the stem cell is an iPSC, and the methods further comprise generating the iPSC. In particular embodiments, the iPSCs are generated from cells obtained from a donor, such as a normal, healthy donor or a diseased donor.

In some embodiments, the methods described herein are used to produce a genetically-engineered stem cell comprising one tagged endogenous protein. In some embodiments, the methods described herein are used to produce a genetically-engineered stem cell comprising two, three, four, five, six, seven, eight, nine, ten, or more tagged endogenous proteins. In some embodiments, the repair template comprises a 5′ homology arm and a 3′ homology arm, each of about 1 kb in length, or each more than 1 kb in length.

### A. Gene-Editing Systems

Herein, the term “gene-editing system” refers to a protein, nucleic acid, or combination thereof that is capable of modifying a target locus of an endogenous DNA sequence when introduced into a cell. Numerous gene editing systems suitable for use in the methods of the present invention are known in the art including, but not limited to, zinc-finger nuclease systems, TALEN systems, and CRISPR/Cas systems.

In some embodiments, the gene editing system used in the methods described herein is a CRISPR (Clustered Regularly Interspaced Short Palindromic Repeats)/Cas (CRISPR Associated) nuclease system, which is an engineered nuclease system based on a bacterial system that can be used for mammalian genome engineering. Generally, the system comprises a CRISPR-associated endonuclease (for example, a Cas endonuclease) and a guide RNA (gRNA). The gRNA is comprised of two parts; a crispr-RNA (crRNA) that is specific for a target genomic DNA sequence, and a trans-activating RNA (tracrRNA) that facilitates endonuclease binding to the DNA at the targeted insertion site. In some embodiments, the crRNA and tracrRNA may be present in the same RNA oligonucleotide, referred to as a single guide-RNA (sgRNA). In some embodiments, the crRNA and tracrRNA may be present as separate RNA oligonucleotides. In such embodiments, the gRNA is comprised of a crRNA oligonucleotide and a tracrRNA oligonucleotide that associate to form a crRNA:tracrRNA duplex. As used herein, the term “guide RNA” or “gRNA” refers to the combination of a tracrRNA and a crRNA, present as either an sgRNA or a crRNA:tracrRNA duplex.

In some embodiments, the CRISPR/Cas systems described herein comprise a Cas protein, a crRNA, and a tracrRNA. In some embodiments, the crRNA and tracrRNA are combined as a duplex RNA molecule to form a gRNA. In some embodiments, the crRNA:tracrRNA duplex is formed in vitro prior to introduction to a cell. In some embodiments, the crRNA and tracrRNA are introduced into a cell as separate RNA molecules and crRNA:tracrRNA duplex is then formed intracellularly. In some embodiments, polynucleotides encoding the crRNA and tracrRNA are provided. In such embodiments, the polynucleotides encoding the crRNA and tracrRNA are introduced into a cell and the crRNA and tracrRNA molecules are then transcribed intracellularly. In some embodiments, the crRNA and tracrRNA are encoded by a single polynucleotides. In some embodiments, the crRNA and tracrRNA are encoded by separate polynucleotides.

In some embodiments, a detectable tag is inserted into a target locus of an endogenous gene mediated by Cas-mediated DNA cleavage at or near a target insertion site. As such, the term “target insertion site” refers to a specific location within a target locus, wherein a polynucleotide sequence encoding a detectable tag can be inserted. In some embodiments, a Cas endonuclease is directed to the target insertion site by the sequence specificity of the crRNA portion of the gRNA, which requires the presence of a protospacer motif (PAM) sequence near the target insertion site. A variety of PAM sequences suitable for use with a particular endonuclease (e.g., a Cas9 endonuclease) are known in the art (See e.g., Nat Methods. 2013 November; 10(11): 1116-1121 and Sci Rep. 2014; 4: 5405). Exemplary PAM sequences suitable for use in the present invention are shown in Table 5. In some embodiments, the target locus comprises a PAM sequence within 50 base pairs of the target insertion site. In some embodiments, the target locus comprises a PAM sequence within 10 base pairs of the target insertion site. The genomic loci that can be targeted by this method are limited only by the relative distance of the PAM sequence to the target insertion site and the presence of a unique 20 base pair sequence to mediate sequence-specific, gRNA-mediated Cas9 binding. In some embodiments, the target insertion site is located at the 5′ terminus of the target locus. In some embodiments, the target insertion site is located at the 3′ end of the target locus. In some embodiments, the target insertion site is located within an intron or an exon of the target locus.

The specificity of a gRNA for a target loci is mediated by the crRNA sequence, which comprises a sequence of about 20 nucleotides that are complementary to the DNA sequence at a target locus. In some embodiments, the crRNA sequences used in the methods of the present invention are at least 90% complementary to a DNA sequence of a target locus. In some embodiments, the crRNA sequences used in the methods of the present invention are at least 95%, 96%, 97%, 98%, or 99% complementary to a DNA sequence of a target locus. In some embodiments, the crRNA sequences used in the methods of the present invention are 100% complementary to a DNA sequence of a target locus. In some embodiments, the crRNA sequences described herein are designed to minimize off-target binding using algorithms known in the art (e.g., Cas-OFF finder) to identify target sequences that are unique to a particular target locus or target gene. In some embodiments, the crRNA sequences used in the methods of the present invention are at least 90% identical to one of SEQ ID NOs: 85-140. In some embodiments, the crRNA sequences used in the methods of the present invention are at least 95%, 96%, 97%, 98%, or 99% identical to one of SEQ ID NOs: 85-140. In some embodiments, the crRNA sequences used in the methods of the present invention are 100% identical to one of SEQ ID NOs: 85-140. Exemplary crRNA sequences are shown in Table 5.

In some embodiments, the endonuclease is a Cas protein. In some embodiments, the endonuclease is a Cas9 protein. In some embodiments, the Cas9 protein is derived from Streptococcus pyogenes (e.g., SpCas9), Staphylococcus aureus (e.g., SaCas9), or Neisseria meningitides (NmeCas9). In some embodiments, the Cas endonuclease is a Cas9 protein or a Cas9 ortholog and is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the endonuclease is selected from the group consisting of C2C1, C2C3, Cpf1 (also referred to as Cas12a), Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the Cas9 is a wildtype (WT) Cas9 protein or ortholog. WT Cas9 comprises two catalytically active domains (HNH and RuvC). Binding of WT Cas9 to DNA based on gRNA specificity results in double-stranded DNA breaks that can be repaired by non-homologous end joining (NHEJ) or homology-directed repair (HDR). In some embodiments, Cas9 is fused to proteins that recruit DNA-damage signaling proteins, exonucleases, or phosphatases to further increase the likelihood or the rate of repair of the target sequence by one repair mechanism or another. In some embodiments, a WT Cas9 is co-expressed with a nucleic acid repair template to facilitate the incorporation of an exogenous nucleic acid sequence by homology-directed repair. In some embodiments, a WT Cas9 is co-expressed with an exogenous nucleic acid sequence encoding a detectable tag to facilitate the incorporation of the nucleic acid encoding the detectable tag into an endogenous target loci by homology-directed repair.

In some embodiments, the Cas9 is a Cas9 nickase mutant. Cas9 nickase mutants comprise only one catalytically active domain (either the HNH domain or the RuvC domain). The Cas9 nickase mutants retain DNA binding based on gRNA specificity, but are capable of cutting only one strand of DNA resulting in a single-strand break (e.g. a “nick”). In some embodiments, two complementary Cas9 nickase mutants (e.g., one Cas9 nickase mutant with an inactivated RuvC domain, and one Cas9 nickase mutant with an inactivated HNH domain) are expressed in the same cell with two gRNAs corresponding to two respective target sequences; one target sequence on the sense DNA strand, and one on the antisense DNA strand. This dual-nickase system results in staggered double stranded breaks and can increase target specificity, as it is unlikely that two off-target nicks will be generated close enough to generate a double stranded break. In some embodiments, a Cas9 nickase mutant is co-expressed with a nucleic acid repair template to facilitate the incorporation of an exogenous nucleic acid sequence by homology-directed repair. In some embodiments, a Cas9 nickase mutant is co-expressed with an exogenous nucleic acid sequence encoding a detectable tag to facilitate the incorporation of the nucleic acid encoding the detectable tag into an endogenous target loci by homology-directed repair.

### B. Repair Templates

In some embodiments, the components of a gene editing system (e.g., one or more gRNAs and a Cas9 protein, or nucleic acids encoding the same) are introduced into a population of stem cells with a repair template. In some embodiments, the repair template comprises a polynucleotide sequence encoding a detectable tag flanked on both the 5′ and 3′ ends by homology arm polynucleotide sequences. In such embodiments, the homology arm sequences and detectable tag sequences comprised within a repair template facilitate the repair of the Cas9-induced double-stranded DNA breaks at an endogenous target loci by homology-directed repair (HDR). In such embodiments, repair of the double-stranded breaks by HDR results in the insertion of the polynucleotide sequence encoding the detectable tag into the endogenous target locus. In some embodiments, the repair template comprises a nucleic acid sequence that is at least about 90% identical to a sequence selected from SEQ ID NOs: 31-84. In some embodiments, the repair template comprises a nucleic acid sequence that is at least about 95%, 96%, 97%, 98%, or 99% identical to a sequence selected from SEQ ID NOs: 31-84. In some embodiments, the repair template comprises a nucleic acid sequence that is 100% identical to a sequence selected from SEQ ID NOs: 31-84.

1. Homology Arms

In some embodiments, each of the 5′ and 3′ homology arms is at least about 500 base pairs long. For example, the homology arm sequences may be at least 500, 510, 520, 530, 540, 550, 560, 570, 580, 590, 600, 650, 700, 750, 800, 850, 900, 950, 1000, 2000, 3000 or more base pairs long. In some embodiments, the homology arm sequences are at least about 1000 base pairs long. In some embodiments, the 5′ homology arm polynucleotide sequence is at least about 90% identical to an endogenous nucleic acid sequence located 5′ to a particular endogenous target locus. In some embodiments, the 5′ homology arm polynucleotide sequence is at least about 95%, 96%, 97%, 98%, or 99% identical to an endogenous nucleic acid sequence located 5′ to a particular endogenous target locus. In some embodiments, the 5′ homology arm polynucleotide sequence is 1⁰⁰% identical to an endogenous nucleic acid sequence located 5′ to a particular endogenous target locus. In some embodiments, the 5′ homology arm polynucleotide sequence is at least about 90% identical to a sequence selected from SEQ ID NOs: 1-15. In some embodiments, the 5′ homology arm polynucleotide sequence is at least about 95%, 96%, 97%, 98%, or 99% identical to a sequence selected from SEQ ID NOs: 1-15. In some embodiments, the 5′ homology arm polynucleotide sequence is 100% identical to a sequence selected from SEQ ID NOs: 1-15.

In some embodiments, the 3′ homology arm polynucleotide sequence is at least about 90% identical to an endogenous nucleic acid sequence located 3′ to a particular endogenous target locus. In some embodiments, the 3′ homology arm polynucleotide sequence is at least about 95%, 96%, 97%, 98%, or 99% identical to an endogenous nucleic acid sequence located 3′ to a particular endogenous target locus. In some embodiments, the 3′ homology arm polynucleotide sequence is 1⁰⁰% identical to an endogenous nucleic acid sequence located 3′ to a particular endogenous target locus. In some embodiments, the 3′ homology arm polynucleotide sequence is at least about 90% identical to a sequence selected from SEQ ID NOs: 16-30. In some embodiments, the 3′ homology arm polynucleotide sequence is at least about 95%, 96%, 97%, 98%, or 99% identical to a sequence selected from SEQ ID NOs: 16-30. In some embodiments, the 3′ homology arm polynucleotide sequence is 100% identical to a sequence selected from SEQ ID NOs: 16-30.

In some embodiments, the 5′ homology arm polynucleotide sequence is at least about 90% identical to a sequence selected from SEQ ID NOs: 1-15 and the 3′ homology arm polynucleotide sequence is at least about 90% identical to a sequence selected from SEQ ID NOs: 16-30. In some embodiments, the 5′ homology arm polynucleotide sequence is at least about 95%, 96%, 97%, 98%, or 99% identical to a sequence selected from SEQ ID NOs: 1-15 and the 3′ homology arm polynucleotide sequence is at least about 95%, 96%, 97%, 98%, or 99% identical to a sequence selected from SEQ ID NOs: 16-30. In some embodiments, the 5′ homology arm polynucleotide sequence is 100% identical to a sequence selected from SEQ ID NOs: 1-15 and the 3′ homology arm polynucleotide sequence is 100% identical to a sequence selected from SEQ ID NOs: 16-30.

In some embodiments, the 5′ homology arm polynucleotide sequence is at least about 90% identical to SEQ ID NO: 1 and the 3′ homology arm polynucleotide sequence is at least about 90% identical to SEQ ID NO: 16. In some embodiments, the 5′ homology arm polynucleotide sequence is at least about 95%, 96%, 97%, 98%, or 99% identical to SEQ ID NO: 1 and the 3′ homology arm polynucleotide sequence is at least about 95%, 96%, 97%, 98%, or 99% identical to SEQ ID NO: 16. In some embodiments, the 5′ homology arm polynucleotide sequence is 100% identical to SEQ ID NO: 1 and the 3′ homology arm polynucleotide sequence is 1000% identical to SEQ ID NO: 16.

In some embodiments, the 5′ homology arm polynucleotide sequence is at least about 90 identical to SEQ ID NO: 2 and the 3′ homology arm polynucleotide sequence is at least about 90% identical to SEQ ID NO: 17. In some embodiments, the 5′ homology arm polynucleotide sequence is at least about 95%, 96%, 97%, 98%, or 99% identical to SEQ ID NO: 2 and the 3′ homology arm polynucleotide sequence is at least about 95%, 96%, 97%, 98%, or 99% identical to SEQ ID NO: 17. In some embodiments, the 5′ homology arm polynucleotide sequence is 100% identical to SEQ ID NO: 2 and the 3′ homology arm polynucleotide sequence is 100% identical to SEQ ID NO: 17.

In some embodiments, the 5′ homology arm polynucleotide sequence is at least about 90% identical to SEQ ID NO: 3 and the 3′ homology arm polynucleotide sequence is at least about 90% identical to SEQ ID NO: 18. In some embodiments, the 5′ homology arm polynucleotide sequence is at least about 95%, 96%, 97%, 98%, or 99% identical to SEQ ID NO: 3 and the 3′ homology arm polynucleotide sequence is at least about 95%, 96%, 97%, 98%, or 99% identical to SEQ ID NO: 18. In some embodiments, the 5′ homology arm polynucleotide sequence is 100% identical to SEQ ID NO: 3 and the 3′ homology arm polynucleotide sequence is 100% identical to SEQ ID NO: 18.

In some embodiments, the 5′ homology arm polynucleotide sequence is at least about 90% identical to SEQ ID NO: 4 and the 3′ homology arm polynucleotide sequence is at least about 90% identical to SEQ ID NO: 19. In some embodiments, the 5′ homology arm polynucleotide sequence is at least about 95%, 96%, 97%, 98%, or 99% identical to SEQ ID NO: 4 and the 3′ homology arm polynucleotide sequence is at least about 95%, 96%, 97%, 98%, or 99% identical to SEQ ID NO: 19. In some embodiments, the 5′ homology arm polynucleotide sequence is 100% identical to SEQ ID NO: 4 and the 3′ homology arm polynucleotide sequence is 100% identical to SEQ ID NO: 19.

In some embodiments, the 5′ homology arm polynucleotide sequence is at least about 90% identical to SEQ ID NO: 5 and the 3′ homology arm polynucleotide sequence is at least about 90% identical to SEQ ID NO: 20. In some embodiments, the 5′ homology arm polynucleotide sequence is at least about 95%, 96%, 97%, 98%, or 99% identical to SEQ ID NO: 5 and the 3′ homology arm polynucleotide sequence is at least about 95%, 96%, 97%, 98%, or 99% identical to SEQ ID NO: 20. In some embodiments, the 5′ homology arm polynucleotide sequence is 100% identical to SEQ ID NO: 5 and the 3′ homology arm polynucleotide sequence is 100% identical to SEQ ID NO: 20.

In some embodiments, the 5′ homology arm polynucleotide sequence is at least about 90% identical to SEQ ID NO: 6 and the 3′ homology arm polynucleotide sequence is at least about 90% identical to SEQ ID NO: 21. In some embodiments, the 5′ homology arm polynucleotide sequence is at least about 95%, 96%, 97%, 98%, or 99% identical to SEQ ID NO: 6 and the 3′ homology arm polynucleotide sequence is at least about 95%, 96%, 97%, 98%, or 99% identical to SEQ ID NO: 21. In some embodiments, the 5′ homology arm polynucleotide sequence is 100% identical to SEQ ID NO: 6 and the 3′ homology arm polynucleotide sequence is 100% identical to SEQ ID NO: 21.

In some embodiments, the 5′ homology arm polynucleotide sequence is at least about 90% identical to SEQ ID NO: 7 and the 3′ homology arm polynucleotide sequence is at least about 90% identical to SEQ ID NO: 22. In some embodiments, the 5′ homology arm polynucleotide sequence is at least about 95%, 96%, 97%, 98%, or 99% identical to SEQ ID NO: 7 and the 3′ homology arm polynucleotide sequence is at least about 95%, 96%, 97%, 98%, or 99% identical to SEQ ID NO: 2. In some embodiments, the 5′ homology arm polynucleotide sequence is 100% identical to SEQ ID NO: 7 and the 3′ homology arm polynucleotide sequence is 100% identical to SEQ ID NO: 22.

In some embodiments, the 5′ homology arm polynucleotide sequence is at least about 90% identical to SEQ ID NO: 8 and the 3′ homology arm polynucleotide sequence is at least about 90% identical to SEQ ID NO: 23. In some embodiments, the 5′ homology arm polynucleotide sequence is at least about 95%, 96%, 97%, 98%, or 99% identical to SEQ ID NO: 8 and the 3′ homology arm polynucleotide sequence is at least about 95%, 96%, 97%, 98%, or 99% identical to SEQ ID NO: 23. In some embodiments, the 5′ homology arm polynucleotide sequence is 100% identical to SEQ ID NO: 8 and the 3′ homology arm polynucleotide sequence is 100% identical to SEQ ID NO: 23.

In some embodiments, the 5′ homology arm polynucleotide sequence is at least about 90% identical to SEQ ID NO: 9 and the 3′ homology arm polynucleotide sequence is at least about 90% identical to SEQ ID NO: 24. In some embodiments, the 5′ homology arm polynucleotide sequence is at least about 95%, 96%, 97%, 98%, or 99% identical to SEQ ID NO: 9 and the 3′ homology arm polynucleotide sequence is at least about 95%, 96%, 97%, 98%, or 99% identical to SEQ ID NO: 24. In some embodiments, the 5′ homology arm polynucleotide sequence is 100% identical to SEQ ID NO: 9 and the 3′ homology arm polynucleotide sequence is 100% identical to SEQ ID NO: 24.

In some embodiments, the 5′ homology arm polynucleotide sequence is at least about 90% identical to SEQ ID NO: 10 and the 3′ homology arm polynucleotide sequence is at least about 90% identical to SEQ ID NO: 25. In some embodiments, the 5′ homology arm polynucleotide sequence is at least about 95%, 96%, 97%, 98%, or 99% identical to SEQ ID NO: 10 and the 3′ homology arm polynucleotide sequence is at least about 95%, 96%, 97%, 98%, or 99% identical to SEQ ID NO: 25. In some embodiments, the 5′ homology arm polynucleotide sequence is 100% identical to SEQ ID NO: 10 and the 3′ homology arm polynucleotide sequence is 100% identical to SEQ ID NO: 25.

In some embodiments, the 5′ homology arm polynucleotide sequence is at least about 90% identical to SEQ ID NO: 11 and the 3′ homology arm polynucleotide sequence is at least about 90% identical to SEQ ID NO: 26. In some embodiments, the 5′ homology arm polynucleotide sequence is at least about 95%, 96%, 97%, 98%, or 99% identical to SEQ ID NO: 11 and the 3′ homology arm polynucleotide sequence is at least about 95%, 96%, 97%, 98%, or 99% identical to SEQ ID NO: 26. In some embodiments, the 5′ homology arm polynucleotide sequence is 100% identical to SEQ ID NO: 11 and the 3′ homology arm polynucleotide sequence is 100% identical to SEQ ID NO: 26.

In some embodiments, the 5′ homology arm polynucleotide sequence is at least about 90% identical to SEQ ID NO: 12 and the 3′ homology arm polynucleotide sequence is at least about 90% identical to SEQ ID NO: 27. In some embodiments, the 5′ homology arm polynucleotide sequence is at least about 95%, 96%, 97%, 98%, or 99% identical to SEQ ID NO: 12 and the 3′ homology arm polynucleotide sequence is at least about 95%, 96%, 97%, 98%, or 99% identical to SEQ ID NO: 27. In some embodiments, the 5′ homology arm polynucleotide sequence is 100% identical to SEQ ID NO: 12 and the 3′ homology arm polynucleotide sequence is 100% identical to SEQ ID NO: 27.

In some embodiments, the 5′ homology arm polynucleotide sequence is at least about 90% identical to SEQ ID NO: 13 and the 3′ homology arm polynucleotide sequence is at least about 90% identical to SEQ ID NO: 28. In some embodiments, the 5′ homology arm polynucleotide sequence is at least about 95%, 96%, 97%, 98%, or 99% identical to SEQ ID NO: 13 and the 3′ homology arm polynucleotide sequence is at least about 95%, 96%, 97%, 98%, or 99% identical to SEQ ID NO: 28. In some embodiments, the 5′ homology arm polynucleotide sequence is 100% identical to SEQ ID NO: 13 and the 3′ homology arm polynucleotide sequence is 100% identical to SEQ ID NO: 28.

In some embodiments, the 5′ homology arm polynucleotide sequence is at least about 90% identical to SEQ ID NO: 14 and the 3′ homology arm polynucleotide sequence is at least about 90% identical to SEQ ID NO: 29. In some embodiments, the 5′ homology arm polynucleotide sequence is at least about 95%, 96%, 97%, 98%, or 99% identical to SEQ ID NO: 14 and the 3′ homology arm polynucleotide sequence is at least about 95%, 96%, 97%, 98%, or 99% identical to SEQ ID NO: 29. In some embodiments, the 5′ homology arm polynucleotide sequence is 100% identical to SEQ ID NO: 14 and the 3′ homology arm polynucleotide sequence is 100% identical to SEQ ID NO: 29.

In some embodiments, the 5′ homology arm polynucleotide sequence is at least about 90% identical to SEQ ID NO: 15 and the 3′ homology arm polynucleotide sequence is at least about 90% identical to SEQ ID NO: 30. In some embodiments, the 5′ homology arm polynucleotide sequence is at least about 95%, 96%, 97%, 98%, or 99% identical to SEQ ID NO: 15 and the 3′ homology arm polynucleotide sequence is at least about 95%, 96%, 97%, 98%, or 99% identical to SEQ ID NO: 30. In some embodiments, the 5′ homology arm polynucleotide sequence is 100% identical to SEQ ID NO: 15 and the 3′ homology arm polynucleotide sequence is 100% identical to SEQ ID NO: 30.

### C. Introduction of Gene-Editing Systems

The components of the gene-editing system (e.g., a CRISPR/Cas system comprising a Cas, tracrRNA, and crRNA) can be intracellularly delivered to a population of cells by any means known in the art. In some embodiments, the Cas component of a CRISPR/Cas gene editing system is provided as a protein. In some embodiments, the Cas protein may be complexed with a crRNA:tracrRNA duplex in vitro to form an CRISPR/Cas RNP (crRNP) complex. In some embodiments, the crRNP complex is introduced to a cell by transfection. In some embodiments, the Cas protein may be introduced to a cell before or after a gRNA is introduced to the cell. In some embodiments, the Cas protein is introduced to a cell by transfection before or after a gRNA is introduced to the cell.

In some embodiments, a nucleic acid encoding a Cas protein is provided. In some embodiments, the nucleic acid encoding the Cas protein is an DNA nucleic acid and is introduced to the cell by transduction. In some embodiments, the Cas9 and gRNA components of a CRISPR/Cas gene editing system are encoded by a single polynucleotide molecule. In some embodiments, the polynucleotide encoding the Cas protein and gRNA component are comprised in a viral vector and introduced to the cell by viral transduction. In some embodiments, the Cas9 and gRNA components of a CRISPR/Cas gene editing system are encoded by a different polynucleotide molecules. In some embodiments, the polynucleotide encoding the Cas protein is comprised in a first viral vector and the polynucleotide encoding the gRNA is comprised in a second viral vector. In some aspects of this embodiment, the first viral vector is introduced to a cell prior to the second viral vector. In some aspects of this embodiment, the second viral vector is introduced to a cell prior to the first viral vector. In such embodiments, integration of the vectors results in sustained expression of the Cas9 and gRNA components. However, sustained expression of Cas9 may lead to increased off-target mutations and cutting in some cell types. Therefore, in some embodiments, an mRNA nucleic acid sequence encoding the Cas protein may be introduced to the population of cells by transfection. In such embodiments, the expression of Cas9 will decrease over time, and may reduce the number of off target mutations or cutting sites.

In some embodiments, each of the Cas9, tracrRNA, crRNA, and repair template components are introduced to a cell by transfection alone or in combination (e.g., transfection of a crRNP). Transfection may be performed by any means known in the art, including but not limited to lipofection, electroporation (e.g., Neon® transfection system or an Amaxa Nucleofector®), sonication, or nucleofection. In such embodiments, the gRNA components can be transfected into a population of cells with a plasmid encoding the Cas9 nuclease. In such embodiments, the expression of Cas9 will decrease over time, and may reduce the number of off target mutations or cutting sites.

### D. Detectable Tags

In some embodiments, the repair templates described herein comprise a polynucleotide sequence encoding a “detectable tag”, “tag,” or “label.” These terms are used interchangeably herein and refer to a protein that is capable of being detected and is linked or fused to a heterologous protein (e.g., an endogenous protein). Herein, the detectable tag serves to identify the presence of the heterologous protein. Insertion of a polynucleotide sequence encoding a detectable tag into an endogenous target loci results in the expression of a tagged version of the endogenous protein. Examples of detectable tags include but are not limited to, FLAG tags, poly-histidine tags (e.g. 6×His), SNAP tags, Halo tags, cMyc tags, glutathione-S-transferase tags, avidin, enzymes, fluorescent molecules, luminescent proteins, chemiluminescent proteins, bioluminescent proteins, and phosphorescent proteins.

In some embodiments, the detectable tag is a fluorescent protein such as green fluorescent protein (GFP), blue fluorescent protein, cyan fluorescent protein, yellow fluorescent protein, or red fluorescent protein. In some embodiments, the detectable tag is GFP. Additional examples of detectable tags suitable for use in the present methods and compositions include mCherry, tdTomato, mNeonGreen, eGFP, Emerald, mEGFP (A208K mutation), mKate, and mTagRFPt. In some embodiments the fluorescent protein is selected from the group consisting ofbBlueiUV proteins (such as TagBFP, mTagBFP2, Azurite, EBFP2, mKalamal, Sirius, Sapphire, and T-Sapphire); cyan proteins (such as ECFP, Cerulean, SCFP3A, mTurquoise, mTurquoise2, monomeric Midoriishi-Cyan, TagCFP, and mTFP1); green proteins (such as: EGFP, Emerald, Superfolder GFP, Monomeric Azami Green, TagGFP2, mUKG, mWasabi, Clover, and mNeonGreen); yellow proteins (such as EYFP, Citrine, Venus, SYFP2, and TagYFP); orange proteins (such as Monomeric Kusabira-Orange, mKOκ, mKO2, mOrange, and mOrange2); red proteins (such as mRaspberry, mCherry, mStrawberry, mTangerine, tdTomato, TagRFP, TagRFP-T, mApple, mRuby, and mRuby2); far-red proteins (such as mPlum, HcRed-Tandem, mKate2, mNeptune, and NirFP); near-infrared proteins (such as TagRFP657, IFP1.4, and iRFP): long stokes shift proteins (such as mKeima Red, LSS-mKate1, LSS-mKate2, and mBeRFP); photoactivatible pProteins (such as PA-GFP, PAmCherryl, and PATagRFP); photoconvertible proteins (such as Kaede (green), Kaede (red), KikGRI (green), KikGRI (red), PS-CFP2, PS-CFP2, mEos2 (green), mEos2 (red), mEos3.2 (green), mEos3.2 (red), PSmOrange, and PSmOrange); and photoswitchable proteins (such as Dronpa). In some embodiments, the detectable tag can be selected from AmCyan, AsRed, DsRed2, DsRed Express, E2-Crimson, HcRed, ZsGreen, ZsYellow, mCherry, mStrawberry, mOrange, mBanana, mPlum, mRasberry, tdTomato, DsRed Monomer, and/or AcGFP, all of which are available from Clontech.

In some embodiments, the polynucleotide sequence encoding the detectable tag is at least about 20 base pairs long. In some embodiments, the polynucleotide sequence encoding the detectable tag is at least 100 base pairs long. For example, the polynucleotide sequence encoding the detectable tag may be about 100, 110, 120, 130, 140, 150, 160, 170, 180, 190, 200, 250, 300, 350, 400, 450, 500, 550, 600, 650, 700, 750, 800, 850, 900, 950, 1000, 2000, 3000, 4000, 5000 or more base pairs long. In some embodiments, the polynucleotide sequence encoding the detectable tag comprises at least about 300 base pairs long. In some embodiments, the polynucleotide sequence encoding the detectable tag comprises at least about 500 base pairs long. In further embodiments, the polynucleotide sequence encoding the detectable tag is about 700 to about 750 base pairs long. For example, the polynucleotide sequence encoding the detectable tag may be about 701, 702, 703, 704, 705, 706, 707, 708, 709, 710, 711, 712, 713, 7114, 715, 716, 717, 718, 719, 720, 721, 722, 723, 724, 725, 726, 727, 728, 729, 730, 740, or about 750 base pairs long. In some embodiments, the polynucleotide sequence encoding the detectable tag is between 710 and 730 base pairs long. The polynucleotide sequence can encode a full-length detectable tag or a portion or fragment thereof. In some embodiments, the polynucleotide sequence encodes a full-length detectable tag. In some embodiments, insertion of the detectable tag into the target locus does not significantly alter the expression or function of either the endogenous protein or the encoded detectable tag.

The insertion of the detectable tag sequence into an endogenous gene results in the production of a tagged endogenous protein. In some embodiments, the tag is directly fused to the endogenous protein. The term “directly fused” refers to two or more amino acid sequences connected to each other (e.g., by peptide bonds) without intervening or extraneous sequences (e.g., two or more amino acid sequences that are not connected by a linker sequence). In some embodiments, the polynucleotide sequence encoding the detectable tag further comprises a linker sequence such that the detectable tag is attached (or linked) to the endogenous protein by a linker sequence. In such embodiments, the attachment may be by covalent or non-covalent linkage. In some embodiments, the attachment is covalent. In some embodiments, the linker sequence is a flexible linker sequence. In some embodiments, the tag is directly fused, or attached by a linker, to the C-terminal or N-terminal end of an endogenous protein. In some embodiments, the linker sequence is selected from the group consisting of sequences shown in Tables 3 and 4.

In some embodiments, the donor polynucleotide further comprises a polynucleotide sequence encoding a selectable marker that allows for the selection of cells comprising the donor polynucleotide. Selectable markers are known in the art and include antibiotic resistance genes. In some embodiments, the antibiotic resistance gene confers resistance to gentamycin, thymidine kinase, ampicillin, and/or kanamycin.

In some embodiments, the donor polynucleotide is a plasmid, referred to herein as a “donor plasmid.” In some embodiments, the donor plasmid comprises a repair template comprising (i) a 5′ homology arm sequence, (ii) a nucleic acid sequence encoding a detectable tag; and (iii) a 3′ homology arm sequence. In some embodiments, the repair template comprised within the donor plasmid further comprises a linker sequence located at the 5′ end or the 3′ end of the nucleic acid sequence encoding the detectable tag. In some embodiments, the repair template comprised within the donor plasmid further comprises an antibiotic resistance cassette located between the 5′ and 3′ homology arm sequences. In such embodiments, the antibiotic resistance cassette may be located 3′ to the 5′ homology arm sequence and 5′ to the nucleic acid sequence encoding the detectable tag. Alternatively, the antibiotic resistance cassette may be located 5′ to the 3′ homology arm sequence and 3′ to the nucleic acid sequence encoding the detectable tag. In some embodiments, the donor plasmid does not comprise a promoter. In such embodiments, the donor plasmid functions as a vehicle to deliver the tag sequence intracellularly to a cell and does not mediate transcription and/or translation of the tag sequence or any polynucleotide sequence comprised therein.

### E. Endogenous Target Loci.

In some embodiments, the present invention provides for methods of inserting one or more detectable tags into one or more endogenous target loci. In some embodiments, the target locus is located within an endogenous gene encoding a structural protein or a non-structural protein. Exemplary target genes are shown below in Tables 1 and 2. In some embodiments, the structural protein is selected from paxillin (PXN), tubulin-alpha 1b (TUBA1B), lamin B1 (LMNB1), actinin alpha 1 (ACTN1), translocase of outer mitochondrial membrane 20 (TOMM20), desmoplakin (DSP), Sec61 translocon beta subunit (SEC61B), fibrillarin (FBL), actin beta (ACTB), myosin heavy chain 10 (MYH10), vimentin (VIM), tight junction protein 1 (TJPI, also known as ZO-1), safe harbor locus, CAGGS promoter (AAVS1), microtubule-associated protein 1 light chain 3 beta (MAP1LC3B, also known as LC3), ST6 beta-galactoside alpha-2,6-sialyltransferase 1 (ST6GAL1), lysosomal associated membrane protein 1 (LAMP1), centrin 2 (CETN2), solute carrier family 25 member 17 (SLC25A17), RAB5A, member RAS oncogene family (RAB5A), gap junction protein alpha 1 (also known as connexin 43 (CX43)) (GJA1), mitogen-activated protein kinase 1 (MAPK1), ATPase sarcoplasmiciendoplasmic reticulum Ca2+ transporting 2 (ATP2A2), AKT serine/threonine kinase 1 (AKT1), catenin beta 1 (CTNNB1), nucleophosmin (NPM1), histone cluster 1 H2B family member j (HIST1H2BJ), Histone cluster 1 H2B family member j:2A:CAAX (CAGGS:HIST1H2BJ:2A:CAAX), polycystin 2, transient receptor potential cation channel (PKD2), dystrophin (DMD), desmin (DES), solute carrier family 25 member 17 (SLC25A17, also known as PMP34), Structural maintenance of chromosomes 1A (SMCIA), Nucleoporin 153 (NUP153), CCCTC-binding factor (CTCF), Chromobox 1 (CBXI), POU class 5 homeobox 1 (Oct4), Sex-determining region-box 2 (Sox2), and Nanog homeobox (Nanog). In certain embodiments, any of these target loci are tagged with a detectable tag, e.g., a fluorescent tag, such as GFP.

In some embodiments, the one or more detectable tags are inserted into an endogenous target locus in a gene encoding a structural protein or a non-structural protein, wherein the expression of the gene and/or the encoded protein is associated with a particular cell type or tissue type. For example, in some embodiments, the expression of the gene and/or the encoded protein is associated with cardiomyocytes, hepatocytes, renal cells, epithelial cells, endothelial cells, neurons, mucosal cells of the gut, lung, or nasal passages. In some embodiments, the expression of the gene and/or the encoded protein is associated with cardiac tissue including, but not limited to, troponin II, slow skeletal type (TNNII), actinin alpha 2 (ACTN2), troponin 13, cardiac type (TNN13), myosin light chain 2 (MYL2), myosin light chain 7 (MYL7), titin (TTN), SMAD family member 2 (SMAD), SMAD family member 5 (SMAD5), NK2 homeobox 5 (NKX2-5), Mesoderm posterior bHLH transcription factor 1 (MESP1), Mix paired-like homeobox (MIXL1), and ISL LIM homeobox 1 (ISL1).

In some embodiments, the expression of the gene and/or the encoded protein is associated with liver tissue including, but not limited to Cytochrome P450E1 (CYP2E1), Transferrin (TF), hemopexin (HPX), and albumin (ALB). In some embodiments, the expression of the gene and/or the encoded protein is associated with kidney tissue including, but not limited to Polycystic kidney disease 1 (PKD1) and Polycystic kidney disease 2 (PKD2). In some embodiments, the expression of the gene and/or the encoded protein is associated with epithelial tissue including, but not limited to keratin 5 (KRT5) and lamanin subunit gamma 2 (LAMC2). Exemplary genes associated with specific tissue and cell types are shown below in Table 2.

TABLE 1
Illustrative Target Genes and Corresponding Cell Structures

| Structure | Gene Name | Gene Symbol |
|---|---|---|
| Matrix adhesions | Paxillin | PXN |
| Microtubules | Tubulin-alpha 1b | TUBA1B |
| Nuclear envelope | Lamin B1 | LMNB1 |
| Actin bundles | Actinin alpha 1 | ACTN1 |
| Mitochondria | Translocase of outer mitochondrial membrane 20 | TOMM20 |
| Desmosomes | Desmoplakin | DSP |
| Endoplasmic reticulum | Sec61 translocon beta subunit | SEC61B |
| Nucleolus | Fibrillarin | FBL |
| Actin filaments | Actin beta | ACTB |
| Actomyosin bundles | Myosin heavy chain 10 | MYH10 |
| Intermediate filaments | Vimentin | VIM |
| Tight junctions | Tight junction protein 1 | TJP1 (also ZO-1) |
| Cytoplasm | Safe harbor locus, CAGGS promoter | AAVS1 |
| Autophagosomes | Microtubule associated protein 1 light | MAP1LC3B (also |
| | chain 3 beta | - LC3) |
| Golgi | ST6 beta-galactoside alpha-2,6-sialyltransferase 1 | ST6CIAL1 |
| Lysosome | Lysosomal associated membrane protein 1 | LAMP1 |
| Centrosome | Centrin 2 | CETN2 |
| Peroxisomes | Solute carrier family 25 member 17 | SLC25A17 |
| Endosomes | RAB5A, member RAS oncogene family | RAB5A |
| Gap junctions | Gap junction protein alpha 1 (also known as | GJA1 (also CX43) |
| | connexin 43) | |
| MAPK1/ERK2 | Mitogen-activated protein kinase 1 | MAPK1 |
| Plasma Membrane | Safe harbor locus. CAGGS promoter | AAVS1 |
| Sarcoplasmic reticulum | ATPase sarcoplasmiclendoplasmie reticulum | ATP2A2 |
| | Ca2+ transporting 2 | |
| PKB/AKT1 | AKT serine/threonine kinase 1 | AKT1 |
| Adherens junctions | Catenin beta 1 | CTNNB1 |
| Nucleolus | Nucleophosmin | NPM1 |
| Histone | Histone cluster 1 H2B family member) | HIST1H2BJ |
| Cation channel | Polycystin 2, transient receptor potential cation | PKD2 |
| | channel | |
| Plasma membrane | Histone cluster 1 H2B family member | CAGGS:HIST1H2 |
| | j:2A:CAAX | BJ:2A:CAAX |
| Cytoskeletal | Dystrophin | DMD |
| Intermediate filament | Desmin | DES |
| Peroxisomes | Solute carrier family 25 member 17 | SLC25A17 (also |
| | | PMP34) |
| chromosomal | Structural maintenance of chromosomes 1A | SMC1A |
| nuclear envelope | Nucleoporin 153 | NUP153 |
| chromosomal | CCCTC-binding factor | CTCF |
| Nucleus | Chromobox 1 | CBX1 |
| Nucleus | POU class 5 homeobox 1 | Oct4 |
| Nucleus | Sex-determining region-box 2 | Sox2 |
| Nucleus | Nanog homeobox | Nanog |

TABLE 2
Illustrative tissue-type and cell-type associated genes

| | | |
|---|---|---|
| Structure | Gene Name | Gene Symbol |

Cardiac-Specific Genes

| | | |
|---|---|---|
| Sarcomeric thin filament | Troponin I1, slow skeletal type | TNNI1 |
| Sarcomeric z-disk | Actinin alpha 2 | ACTN2 |
| Sarcomeric thick filament | Troponin 13, cardiac type | INNI3 |
| Sarcomeric thick filament | Myosin light chain 2 | MYL2 |
| Sarcomeric thick filaments | Myosin light chain 7 | MYL7 |
| Sarcomere | Titin | TTN |
| Sarcoplasmic recticulum | Ryanodine receptor 2 | RYR2 |
| Nucleus | SMAD family member 2 | SMAD2 |
| Nucleus | SMAD family member 5 | SMAD5 |
| Nucleus | NK2 homeobox 5 | NKX2-5 |
| Nucleus | Mesoderm posterior bHLH | MESP1 |
| | transcription factor 1 | |
| Nucleus | Mix paried-like homeobox | MIXL1 |
| Nucleus | ISL LIM homeobox 1 | ISL1 |

Kidney-Specific Genes

| | | |
|---|---|---|
| Cilia | Polycystic kidney disease 1 | PKD1 |
| Cilia | Polycystic kidney disease 2 | PKD2 |

Liver-Specific Genes

| | | |
|---|---|---|
| cellular membrane | Cytochrome P450E1 | CYP2E 1 |
| cellular membrane | Transferrin | TF |
| Endoplasmic reticulum and | hemopexin | HPX |
| microbodies | | |
| cytoplasm | albumin | ALB |

Epithelial-Specific Genes

| | | |
|---|---|---|
| cytoskeleton | Keratin 5 | KRT5 |
| extracellular matrix | lamanin subunit gamma 2 | LAMC2 |

In some embodiments, a plurality of detectable labels is inserted into a plurality of target loci. For example, one detectable label is inserted at one endogenous loci and a different detectable label is inserted at a different endogenous loci. In such embodiments, each of the individual detectable labels is selected such that the detection of one does not interfere, or minimally interferes with, the detection of another. In such embodiments, a unique crRNA is generated for each target locus. In further embodiments, a CRISPR ribonucleoprotein (crRNP), comprising a Cas protein complexed with a crRNA:tracrRNA duplex, is produced for each target locus. In some embodiments, the plurality of nucleic acid sequences encoding the plurality of detectable labels are comprised in a single donor plasmid and are flanked on the 5′ and 3′ ends by homology arms corresponding to genomic sequences within the target locus. For example, at least 2, 3, 4, 5, 6, 7, 8, 9, 10 or more detectable labels and their corresponding homology arms may be comprised within one donor polynucleotide.

In some embodiments, the plurality of nucleic acid sequences encoding the plurality of detectable labels and their corresponding homology arms are comprised within at least two different donor plasmids. For example, at least 2, 3, 4, 5, 6, 7, 8, 9, 10 or more donor plasmids may be used in the present methods. In some embodiments, a plurality of donor plasmids (e.g., at least 2, 3, 4, 5, 6, 7, 8, 9, 10 or more) each comprising one sequence encoding a detectable label and the corresponding homology arms may be used in the present methods. In some embodiments, a plurality of donor plasmids (e.g., at least 2, 3, 4, 5, 6, 7, 8, 9, 10 or more) each comprising a plurality of sequences encoding two or more detectable labels (e.g., at least 2, 3, 4, 5, 6, 7, 8, 9, 10 or more) and the corresponding homology arms may be used in the present methods. In some embodiments, the plurality of donor plasmids are introduced to a stem cell at the same time. In some embodiments, the plurality of donor plasmids are introduced to a stem cell sequentially.

### III. Stably-Tagged Stem Cell Clones

In some embodiments, the present disclosure provides edited stem cell clones that stably express one or more tagged endogenous proteins. In some embodiments, the stably tagged stem cell clones of the current invention are characterized by (i) mono- or biallelic insertion of a nucleic acid sequence encoding a detectable tag (e.g. GFP) into one or more endogenous proteins (e.g., structural, non-structural, or non-expressed proteins of the stem cell); (ii) pluripotency (e.g., the ability to differentiate into all three germ layers); and (iii) the lack of additional mutations or alternations in the endogenous stem cell genome. Such edited stem cell clones are herein referred to as “stably tagged stem cell clones.”

The stably tagged stem cell clones described herein phenotypically differ from non-engineered stem cell clones only by the expression of one or more endogenous proteins that have been tagged with a detectable tag and the incorporation of one or more antibiotic resistance cassettes into the one or more tagged endogenous loci. In some embodiments, the stably tagged stem cell clones of the current invention are characterized by (i) mono- or biallelic insertion of a nucleic acid sequence encoding a detectable tag (e.g. GFP) into one or more endogenous proteins (e.g., structural, non-structural, or non-expressed proteins of the stem cell); (ii) pluripotency (e.g., the ability to differentiate into all three germ layers); and (iii) the presence of one or more additional mutations or alternations in the endogenous stem cell genome. Such edited stem cell clones are herein referred to as “stably tagged mutant stem cell clones.” In some embodiments, the stably tagged mutant stem cell clones comprise one or more one or more additional mutations or alternations in the endogenous stem cell genome that are associated with a particular disease or disorder. Thus, the stably tagged mutant stem cell clones described herein phenotypically differ from non-engineered stem cell clones by the expression of one or more endogenous proteins that have been tagged with a detectable tag, the incorporation of one or more antibiotic resistance cassettes into the one or more tagged endogenous loci, and the presence of one or more mutations additional not found in the non-engineered stem cell clones. The stably tagged mutant stem cell clones described herein phenotypically differ from the corresponding stably tagged stem cell clones only by the presence of one or more additional mutations.

Provided herein are compositions comprising stably tagged stem cell clones made by the methods described herein. In some embodiments, the compositions comprise a stably tagged stem cell clone wherein one endogenous protein is tagged. For example, a composition may comprise a stably tagged stem cell clone expressing a tagged endogenous protein wherein the endogenous protein is one selected from Tables 1 and/or 2 (e.g., one of PXN, TUBA1B, LMNB1, ACTN1, TOMM20, DSP, SEC61B, FBL, ACTB, MYH10, VIM. TJPI (also known as ZO-1), AAVS1, MAP1LC3B (also known as LC3), ST6GAL1, LAMP, CETN2, SLC25A17 (also known as PMP34), RAB5A, GJA1 (also known as connexin 43 (CX43)), MAPK1, ATP2A2, AKT1, CTNNB1, NPM1, HIST1H2BJ, CAGGS:HIST1H2BJ:2A:CAAX, PKD2, DMD, DES, SLC25A17 (also known as PMP34), SMC1A, NUP153, CTCF, CBX1, Oct4, Sox2, Nanog, TNNI1, ACTN2, TNN13, MYL2, MYL7, TTN, SMAD, SMAD5, NKX2-5, MESP1, MIXL1, ISL1, CYP2E1, TF, HPX, ALB, PKD1, PKD2, KRT5, and LAMC2.

In some embodiments, the compositions described herein comprise a stably tagged stem cell clone wherein at least two endogenous proteins are tagged. For example, a composition may comprise a stably tagged stem cell clone wherein one endogenous loci is tagged with a detectable tag and wherein another endogenous loci is tagged with a different detectable tag. In such embodiments, either of the endogenous loci may be selected from Tables 1 and/or 2. For example, the endogenous proteins may be two or more of those listed in Tables 1 and 2 (e.g., two or more of PXN, TUBA1B, LMNB1, ACTN1, TOMM20, DSP, SEC61B, FBL, ACTB, MYH10, VIM, TJP1 (also known as ZO-1), AAVS1, MAP1LC3B (also known as LC3), ST6GAL1, LAMP1, CETN2, SLC25A17 (also known as PMP34), RAB5A, GJA1 (also known as connexin 43 (CX43)), MAPK1, ATP2A2, AKT1, CTNNB1, NPM1, HIST1H2BJ, CAGGS:HIST1H2BJ:2A:CAAX, PKD2, DMD, DES, SLC25A17 (also known as PMP34), SMCIA, NUP153, CTCF, CBX1, Oct4, Sox2, Nanog, TNNII, ACTN2, TNN13, MYL2, MYL7, TTN, SMAD, SMAD5, NKX2-5, MESP1, MIXL1, ISL1, CYP2E1, TF, HPX, ALB, PKD1, PKD2, KRT5, and LAMC2. In some embodiments, one detectable tag may be inserted into a target loci in TUBABI and a different detectable tag may be inserted into a target loci in LMNB1. In some embodiments, one detectable tag may be inserted into a target loci in SEC61B and a different detectable tag may be inserted into a target loci in LMNB1. In some embodiments, one detectable tag may be inserted into a target loci in TOMM20 and a different detectable tag may be inserted into a target loci in TUBABI. In some embodiments, one detectable tag may be inserted into a target loci in SEC61B and a different detectable tag may be inserted into a target loci in TUBAB1. In some embodiments, one detectable tag may be inserted into a target loci in TUBABI and a different detectable tag may be inserted into a target loci in CETN2. In some embodiments, one detectable tag may be inserted into a target loci in SEC61B and a different detectable tag may be inserted into a target loci in LMNB1. In some embodiments, one detectable tag may be inserted into a target loci in AAVS1 and a different detectable tag may be inserted into a target loci in CAGGS:HIST1H2BJ:2A:CAAX. In some embodiments, one detectable tag may be inserted into a target loci in TOMM20 and a different detectable tag may be inserted into a target loci in TUBAB1.

In some embodiments, the compositions described herein comprise a stably tagged stem cell clone wherein at least three endogenous proteins are tagged. For example, a composition may comprise a stably tagged stem cell clone wherein a first endogenous loci is tagged with a first detectable tag, a second endogenous loci is tagged with a second detectable tag, and a third endogenous loci is tagged with a third detectable tag. In such embodiments, any of the endogenous loci may be selected from Tables 1 and/or 2. For example, the endogenous proteins may be three or more of those listed in Tables 1 and 2 (e.g., three or more of PXN, TUBAlB, LMNB1, ACTN1, TOMM20, DSP, SEC61B, FBL, ACTB, MYH10, VIM, TJP1 (also known as ZO-1), AAVS1, MAP1LC3B (also known as LC3), ST6GAL1, LAMP1, CETN2, SLC25A17 (also known as PMP34), RAB5A, GJA1 (also known as connexin 43 (CX43)), MAPK1, ATP2A2, AKT1, CTNNB,I NPM1, HIST1H2BJ, CAGGS:HIST1H2BJ:2A:CAAX, PKD2, DMD, DES, SLC25A17 (also known as PMP34), SMCIA, NUP153, CTCF, CBX1, Oct4, Sox2, Nanog, TNNI1, ACTN2, TNN13, MYL2, MYL7, TTN, SMAD, SMAD5, NKX2-5, MESP1, MIXL1, ISL1, CYP2E1, TF, HPX, ALB, PKD1, PKD2, KRT5, and LAMC2.

In some embodiments, the compositions described herein comprise a stably tagged stem cell clone wherein at least four or five or more endogenous proteins are tagged. In such embodiments, the endogenous proteins may be three or more of those listed in Tables 1 and 2 (e.g., four, five, or more of PXN, TUBA1B, LMNB1, ACTN 1, TOMM20, DSP, SEC61B, FBL, ACTB, MYH10, VIM, TJP1 (also known as ZO-1), AAVS1, MAP1LC3B (also known as LC3), ST6GAL1, LAMP, CETN2, SLC25A17 (also known as PMP34), RAB5A, GJA1 (also known as connexin 43 (CX43)), MAPK1, ATP2A2, AKT1, CTNNB1, NPM1, HIST1H2BJ, CAGGS:HIST1H2BJ:2A:CAAX, PKD2, DMD, DES, SLC25A17 (also known as PMP34), SMC1A, NUP153, CTCF, CBX1, Oct4, Sox2, Nanog, TNNI1, ACTN2, TNNI3, MYL2, MYL7, TTN, SMAD, SMAD5, NKX2-5, MESP1, MIXL1, ISL1, CYP2E1, TF, HPX, ALB, PKD1, PKD2, KRT5, and LAMC2.

In some embodiments, the compositions described herein comprise two or more stably tagged stem cell clones, wherein each stably tagged stem cell clone expresses a tagged endogenous protein. In some embodiments, each stably tagged stem cell clone express a different tagged endogenous protein. In some embodiments, the compositions described herein comprise 2, 3, 4, 5, 6, 7, 8, 9, 10 or more stably tagged stem cell clones, wherein each stably tagged stem cell clone expresses a different tagged endogenous protein. In some embodiments, the compositions described herein comprise two or more stably tagged stem cell clones, wherein each stably tagged stem cell clone expresses two or more tagged endogenous proteins. In some embodiments, the compositions described herein comprise 2, 3, 4, 5, 6, 7, 8, 9, 10 or more stably tagged stem cell clones, wherein each stably tagged stem cell clone expresses two or more tagged endogenous proteins. In some embodiments, the compositions described herein comprise two or more stably tagged stem cell clones, wherein each stably tagged stem cell clone expresses 2, 3, 4, 5, 6, 7, 8, 9, 10 or more tagged endogenous proteins. In some embodiments, the compositions described herein comprise 2, 3, 4, 5, 6, 7, 8, 9, 10 or more stably tagged stem cell clones, wherein each stably tagged stem cell clone expresses 2, 3, 4, 5, 6, 7, 8, 9, 10 or more tagged endogenous proteins. In some embodiments, each stably tagged stem cell clone express a group of tagged endogenous proteins that are different from the tagged endogenous proteins expressed by another stem cell clone in the same composition. Exemplary endogenous proteins that can be tagged in these embodiments are shown in Tables 1 and 2, including but not limited to PXN, TUBAlB, LMNB1, ACTN1, TOMM20, DSP, SEC61B, FBL, ACTB, MYH10, VIM, TJPI (also known as ZO-1), AAVS1, MAP1LC3B (also known as LC3), ST6GAL1, LAMP1, CETN2, SLC25A17 (also known as PMP34), RAB5A, GJA1 (also known as connexin 43 (CX43)), MAPK1, ATP2A2, AKT1, CTNNB1, NPM1, HIST1H2BJ, CAGGS:HIST1H2BJ:2A:CAAX, PKD2, DMD, DES, SLC25A17 (also known as PMP34), SMCIA, NUP153, CTCF, CBX1, Oct4, Sox2, Nanog, TNNI1, ACTN2, TNNI3, MYL2, MYL7, TTN, SMAD, SMAD5, NKX2-5, MESP1, MIXL1, ISL1, CYP2E1, TF, HPX, ALB, PKD1, PKD2, KRT5, and LAMC2.

Exemplary stably tagged stem cell clones that can be produced by the methods and techniques are shown below in Tables 3 and 4. The association of any tag in the table with any structural protein in the table is for illustrative purposes only. In this regard, any tag (or fluorescent protein) in the Table can be associated with any structural gene in the table.

TABLE 3
Exemplary Embodiments of Stably Tagged Stem Cell Clones

| | Tagged | | | Tag | Linker | SEQ ID |
|---|---|---|---|---|---|---|
| Gene | alleles | Structure | Tag | Location | sequence | NO: |
| PXN | mono | Matrix adhesions | EGFP | C-term | GTSGGS | 141 |
| TUBA1IB | mono | Microtubules | mEGFP | N-term, | GC-SGGS | 142 |
| | | | | 2_{nd} exon | | |
| LMNB1 | mono | Nuclear envelope | mEGFP | N-term | SGLRSRAQAS | 143 |
| ACTN1 | mono | Actin bundles | mEGFP | C- | KLRILQSTVPR | 144 |
| | | | | terminus | ARDPPVAT | |
| TOMM20 | mono | Mitochondria | mEGFP | C- | GGSGDPPVAT | 145 |
| | | | | terminus | | |
| DSP | mono | Desmosomes | mEGFP | C- | HDPPVAT | 146 |
| | | | | terminus | | |
| SEC61B | mono | Endoplasmic | mEGFP | N-term | SGLRS | 147 |
| | | reticulum | | | | |
| FBL | mono | Nucleolus | mEGFP | C- | KPNSAVDGTAG | 148 |
| | | | | terminus | PGSIAT | |
| ACTB | mono | Actin filaments | mEGFP | N-term | AGSGT | 149 |
| MYH10 | mono | Actomyosin | mEGFP | N-term | YSDLELKLRIP | 150 |
| | | bundles | | | | |
| VIM | mono | Intermediate | mEGFP | N-term | SGLRSGSGGGS | 151 |
| | | filaments | | | ASGGSGS | |
| TJP1 | mono | Tight junctions | mEGFP | N-term | SSLRSRALERD | 152 |
| | | | | | K | |
| AAVS1 | mono | Cytoplasm | mEGFP | Internal | N/A | |
| MAP1LC3B | mono | Autophagosomes | mEGFP | N-term | SGLRS | 147 |
| ST6GAL1 | mono, bi | Golgi | mEGFP | C- | LQSTVPRARDP | 153 |
| | | | | terminus | PVAT | |
| LAMP1 | mono | Lysosome | mEGFP | C- | EFGSTGSTGST | 154 |
| | | | | terminus | GADPPVAT | |
| CETN2 | mono | Centrosome | mTagRFPt | N-term, | SGLRS | 147 |
| | | | | 2^{nd} exon | | |
| SLC25A17 | mono | Peroxisomes | mEGFP | C- | RDPPVAT | 155 |
| | | | | terminus | | |
| RAB5A | mono | Endosomes | mEGFP | N-term | SGLRSRA | 156 |
| GJA1 | mono | Gap junctions | mEGFP | C- | DPPVAT | 157 |
| | | | | terminus | | |
| MAPK1 | mono | MAPK1/ERK2 | mEGFP | N-term | SGRTQISRCCA | 158 |
| | | | | | AN | |
| AAVS1 | mono | Plasma | mTagRFPt | Internal | N/A | |
| | | Membrane | | | | |
| ATP2A2 | mono | Sarcoplasmic | mEGFP | N-term | GSA | |
| | | reticulum | | | | |
| AKT1 | | PKB/AKT1 | mEGFP | N-term | RMHM | 159 |
| CTNNB1 | | Adherens | mEGFP | N-term | SGLRSRAQASN | 160 |
| | | junctions | | | SAVDGTAAT | |
| NPM1 | | Nucleolus | mEGFP | C- | KPNSAVDGTAG | 161 |
| | | | | terminus | PGSIAT | |
| HIST1H2BJ | | Histone | mEGFP | C- | DPPVAT | 157 |
| | | | | terminus | | |
| PKD2 | | Cation channel | mEGFP | N-term | SGGGGTGGGSG | 162 |

Other FPS and linkers

| | Tagged | | | Tag | Linker | SEQ ID |
|---|---|---|---|---|---|---|
| Gene | alleles | Structure | Tag | Location | sequence | NO: |
| TUBA1B | mono | Microtubules | mTagRFPt | N-term, | GGSGGS | 142 |
| | | | | 2^{nd} exon | | |
| CETN2 | | Centrosome | tdTomato | N-term, | SGLRS | 147 |
| | | | | 2^{nd} exon | | |
| LMNB1 | | Nuclear envelope | tdTomato | N-term | SGLRSRAQAS | 143 |
| LMNB1 | | Nuclear envelope | mTagRFPt | N-term | SGLRSRAQAS | 143 |
| AAVS1 | | Cytoplasm | mEGFP | Internal | N/A | |
| AAVS1 | | Plasma | mTagRFPt | Internal | N/A | |
| | | Membrane | | | | |

Cardiac specific genes

| | Tagged | | | Tag | Linker | SEQ ID |
|---|---|---|---|---|---|---|
| Gene | alleles | Structure | Tag | Location | sequence | NO: |
| TNNI1 | | Sarcomeric thin | mEGFP | C-term | SGSGS-SG | 163 |
| | | | | filament | | |
| ACTN2 | | Sarcomeric z- | mEGFP | C-term | VDGTAG/SIAT | 164 |
| | | | | disk | ** | |
| TNMI3 | | Sarcomeric thick | mEGFP | C-term | SGSGS/SG** | 165 |
| | | filament | | | | |
| MYL2 | | Sarcomeric thick | mEGFP | C-term | GGGGGGVFVEK | 166 |
| | | filament | | | ** | |
| MYL7 | | Sarcomeric thick | mEGFP | C-term | GGGGGGVFVEK | 166 |
| | | filaments | | | ** | |
| TTN | | Sarcomere | mEGFP | C-term | Tia1L-CAGGS- | |
| | | | | | mCherry-Tia1L | |
| | | | | | excisable | |
| | | | | | element** | |
| DMD | | Sarcolemma | mEGFP | | | |
| DES | | Intermediate | mEGFP | | | |
| | | filament | | | | |

**variable based

TABLE 4
Exemplary Embodiments of Stably Dual-Tagged Stem Cell Clones

| | | Tagged | | | Tag | Linker | SEQ ID |
|---|---|---|---|---|---|---|---|
| Genes | | alleles | Structur | Tag | Location | sequence | NO: |
| TUBA1B/ | TUBA1B | | Microtubules | mEGFP | N-term, | GGSGGS | 142 |
| LMNB1 | | | | | 2^{nd} exon | | |
| | LMNB1 | | Nuclear | mTagRFPt | N-term | SGLRSR | 143 |
| | | | envelope | | | AQAS | |
| SEC61B/ | SEC61B | | Endoplasmic | mEGFP | N-term | SGLRS | 147 |
| LMNB1 | | | reticulum | | | | |
| | LMNB1 | | Nuclear | mCherry | N-term | SGLRSR | 143 |
| | | | envelope | | | AQAS | |
| TUBA1B/ | TUBA1B | mono | Microtubules | mEGFP | N-term, | GGSGGS | 142 |
| LMNB1 | | | | | 2^{nd} exon | | |
| | LMNB1 | analysis | Nuclear | tdTomato | N-term | SGLRSR | 143 |
| | | not | envelope | | | AQAS | |
| | | performed | | | | | |
| TOMM20/ | TOMM20 | mono | Mitochondria | mEGFP | C-term | GGSGDP | 145 |
| TUBA1B | | | | | | PVAT | |
| | TUBA1B | analysis | Microtubules | mTagRFPt | N-term, | GGSGGS | 142 |
| | | not | | | 2^{nd} exon | | |
| | | performed | | | | | |
| SEC61B/ | SEC6IB | | Endoplasmic | mEGFP | N-term | SGLRS | 147 |
| TUBA1B | | | reticulum | | | | |
| | TUBA1B | | Microtubules | mTagRFPt | N-term, | GGSGGS | 142 |
| | | | | | 2^{nd} exon | | |
| TUBA1B/ | TUBA1B | | Microtubules | mEGFP | N-term, | GGSGGS | 142 |
| CETN2 | | | | | 2^{nd} exon | | |
| | CETN2 | | Centrosome | mTagRFPt | N-term, | SGLRS | 147 |
| | | | | | 2^{nd} exon | | |
| SEC61B/ | SEC61B | | Endoplasmic | mEGFP | N-term | SGLRS | 147 |
| LMNB1 | | | reticulum | | | | |
| | LMNB1 | | Nuclear | mTagRFPt | N-term | SGLRSR | 143 |
| | | | envelope | | | AQAS | |
| AAVS1/ | AAVS1 | | Histone | mCherry | N-term | DPPVAT | 157 |
| CAGGS: | CAGGS: | | Plasma | mTagRFPt | internal, | N/A | |
| HIST1H2BJ: | HIST1H2BJ: | | membrane | | separated | | |
| 2A:CAAX | | | | | by 2A | | |
| | | | | | peptide | | |
| TOMM20/ | TOMM20 | | Mitochondria | mEGFP | C-term | GGSGDP | 145 |
| Tuba1B | | | | | | PVAT | |
| | TUBA1B | | Microtubules | mTagRFPt | N-term, | GGSGGS | 142 |
| | | | | | 2^{nd} exon | | |

### A. Validation Assays

In some embodiments, the present invention provides methods for selecting a stem cell that has been modified by the methods described herein to express a tagged endogenous protein. In some embodiments, the insertion of the tag sequence into the endogenous target loci does not result in additional genetic mutations or alterations in the endogenous target locus, or any other heterologous locus in the endogenous genome. In further embodiments, the insertion of the tag sequence into the endogenous target loci does not modify or alter the expression, function, or localization of the endogenous protein. In some embodiments, methods are provided herein for selecting stem cells modified by the methods described herein, wherein the identified stem cells comprise one or more of precise insertion of the nucleic acid sequence encoding a tag; pluripotency; maintained cell viability and function as compared to a non-modified stem cell; maintained levels of expression of the tagged endogenous protein as compared to a non-modified stem cell; maintained protein localization of the tagged endogenous protein as compared to a non-modified stem cell; maintained protein function of the tagged endogenous protein as compared to a non-modified stem cell; maintained expression of stem cell markers as compared to a non-modified stem cell; and/or maintained differentiation potential. In some embodiments, the properties of a selected stem cell are validated by one or more of several downstream assays.

In some embodiments, a population of edited stem cells (e.g., wherein a crRNP and a donor plasmid have been transfected into the cells) are sorted based on their relative expression of the detectable tag. In some embodiments, cells are sorted by fluorescence activated cell sorting (FACS). Cells that are positive for the inserted tag (e.g., express the tag at levels that are increased compared to non-edited population) are selected for further analysis. In some embodiments, the selected cells are expanded in a single colony expansion assay to produce individual clones of edited stem cells.

In some embodiments, edited clones are further analyzed by digital droplet PCR (ddPCR) to identify clones that have an inserted tag sequence and that do not have stable genomic incorporation of the plasmid backbone. In some embodiments, the clones are further analyzed to determine the copy number of the inserted tag sequence. In some embodiments, identified clones have monoallelic or biallelic insertion of the tag sequence.

In further embodiments, the modified cells are assessed for the functional expression of the one or more detectable tags. For example, live cell imaging may be used to observe localization, expression intensity, and persistence of expression of the tagged endogenous protein in the modified stem cells described herein. In some embodiments, the expression of one or more detectable tags does not substantially or does not significantly alter the endogenous expression, localization, or function of the tagged protein. In some embodiments, the precise insertion of the tag sequence is analyzed by sequencing the edited target locus or a portion thereof. In some embodiments, the junctions between the endogenous genomic sequence and the 5′ and 3′ ends of the tag sequence are amplified. The amplification products derived from the population of edited cells are sequenced and compared with sequences of the corresponding target locus derived from a population of non-edited cells. In some embodiments, potential off-target sites for the crRNA sequences are determined using algorithms known in the art (e.g., Cas-OFF finder). To determine the presence of off-target cutting or insertions, these predicted off-target sites and the surrounding genomic sequences can be amplified and sequenced to determine the presence of any mutations or inserted tag sequences. Sequencing can be performed by a number of methods known in the art, e.g., Sanger sequencing and Next-generation, high-throughput sequencing. In some embodiments, the edited populations of cells can be assessed for the expression of transcription factors, cell surface markers, and other proteins or genes associated with stem cells (e.g. Oct 3/4, Sox2, Nanog, Tra-160, Tra-181, and SSEA3). Protein expression can be determined by a number of means known in the art including flow cytometry, ELISA, Western blots, immunohistochemistry, or co-immunoprecipication. Gene expression can be determined by qPCR, microarray, and/or sequencing techniques (e.g., NGS, RNA-Seq, or CHIP-Seq). In some embodiments the edited populations of cells can be assessed for the presence of the CRISPR/Cas9 ribonucleoprotein (RNP) complex and/or the donor polynucleotide. In some embodiments, the edited stem cells are determined to be pluripotent according to the methods outlined above may be cryopreserved for later differentiation or use.

### B. Differentiation Assays

In some embodiments, the invention provides for methods of live-cell imaging in three dimensions using the stably tagged stem cell clones and methods described herein. In some embodiments, the present invention provides methods of assaying the differentiation potential of the edited stem cells and stably tagged clones thereof described herein. Such assays typically involve culturing edited stem cells or stably tagged clones thereof in media comprising one or more factors required for differentiation. Factors required for differentiation are referred to herein as “differentiation agents” and will vary according to the desired differentiated cell type. In some embodiments, the ability of the edited stem cells or stably tagged clones thereof described herein to differentiate into specialized cells is substantially similar to the ability of un-modified stem cells to differentiate into specialized cells. For example, in some embodiments, the edited stem cells and/or stably tagged clones thereof described herein are able to differentiate into substantially the same number of different types of specialized cells, differentiate at substantially the same rate (e.g., 1, 2, 3, 4, 5, 6, 7, 8, 9, 10, or more days to differentiated), and produce differentiated cells that are as viable and as function as un-modified stem cells.

In some embodiments, the methods of assaying the differentiation potential of the edited stem cells and stably tagged clones thereof described herein includes the addition of one or more test agents to a culture of edited stem cells or stably tagged clones thereof prior to, during, or after the addition of one or more differentiation agents. The edited stem cells or stably tagged clones thereof can then be visualized for changes in cellular morphology associated with the individual structural proteins tagged within each edited stem cells or stably tagged clones thereof. In some embodiments, these methods may be used to identify agents that promote differentiation into one or more cell lineages and therefore may be useful as differentiation agents. In some embodiments, these methods may be used to identify agents that disrupt or inhibit differentiation. In some embodiments, the stably tagged stem cells may be differentiated into any cell type, including but not limited to hematopoietic cells, neurons, astrocytes, dendritic cells, hepatocytes, cardiomyocytes, kidney cells, smooth muscle cells, skeletal muscle cells, epithelial cells, or endothelial cells.

C. Screening Assays with Stably-Tagged Stem Cells and Cells Derived Therefrom

In some aspects, the present invention provides methods for drug screening to identify candidate therapeutic agents, and methods of screening agents to determine the effects of agents on the stably-tagged stem cell clones described herein and cells derived therefrom produced by the methods of the present invention. The methods may be employed to identify an agent having a desired effect on the cells. The stably-tagged stems cells of the present invention enable changes across multiple cell types to be assayed with the built in control of the cell types all being derived from the same progenitor clone.

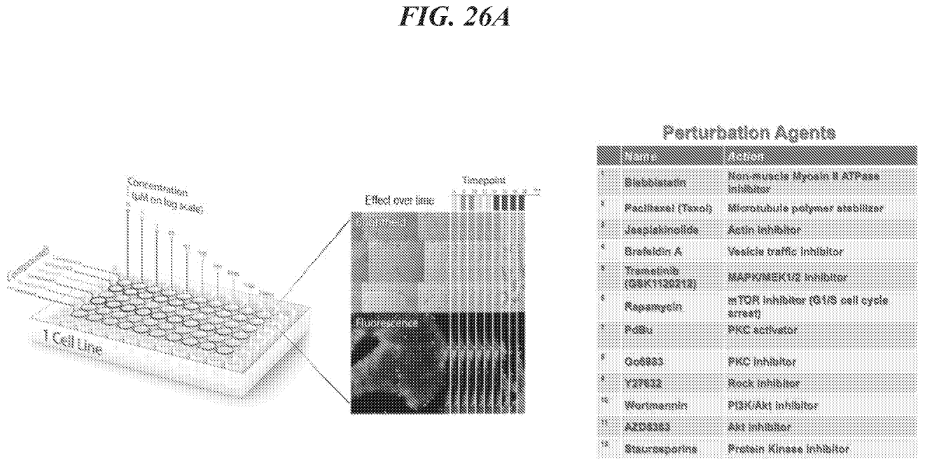

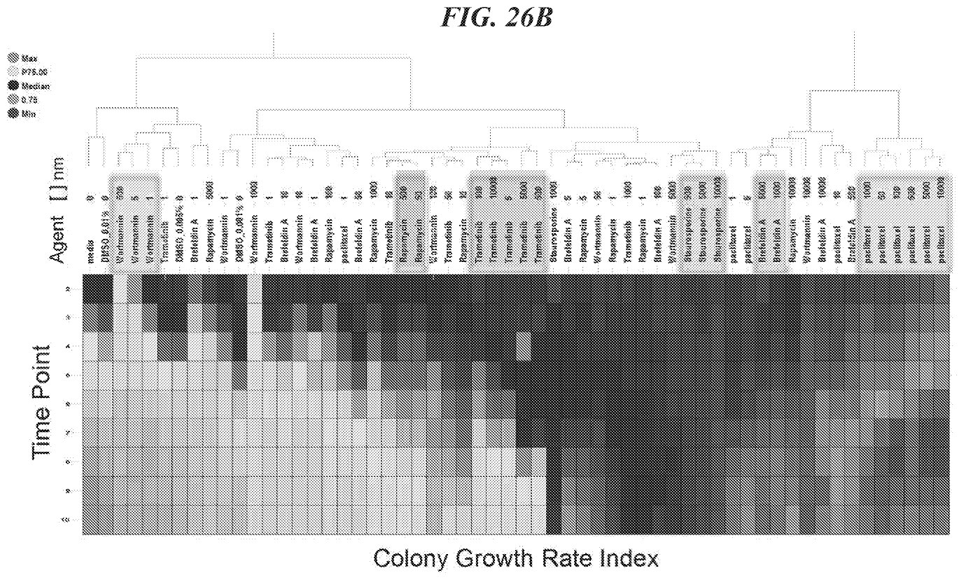

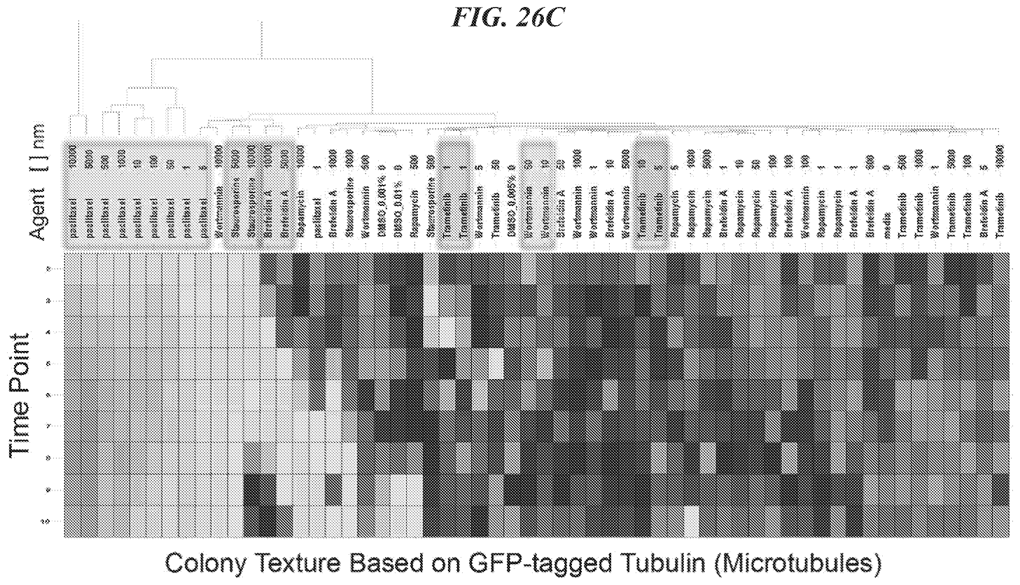

In some embodiments, methods are provided for determining the effect of agents including small molecules, proteins, nucleic acids, lipids or even physical or mechanical stress (i.e. UV light, temperature shifts, mechanical sheer, etc.) by culturing a population of the stably-tagged stem cell clones described herein and cells derived therefrom in the presence and absence of the test agent(s). In some embodiments, agents that disrupt, alter, or modulate various key cellular structures and processes, including but not limited to cell division, microtubule organization, actin dynamics, vesicle trafficking, cell signaling, DNA replication, calcium regulation, ion channel regulators, and/or statins are assayed by the present methods. In some embodiments, the agent exerts a biological effect on the cells, such as increased cell growth or differentiation, increased or reduced expression of one or more genes, or increased or reduced cell death or apoptosis, etc. In particular embodiments, the stably-tagged stem cell clones used to screen for agents having a particular effect comprise a tagged protein associated with the cellular structure, process or biological activity being examined, such as any of the combinations of genes and structures shown in tables 3 and 4. Exemplary agents are shown in FIG. 26A.

In a further embodiment, the method provides assaying the cells after the exposure period by any known method, including confocal microscopy in order to determine changes in the content, orientation or cellular composition of the tagged structural protein contained within the given cell population. In one embodiment, a comparison can be made between the treated cells and untreated controls. In a further embodiment, a positive control may also be utilized in such methods. In some embodiments, one or more positive control agents with known effects on targeted structures may be applied to differentiated cell cultures derived from stably tagged stem cell clones and imaged, for example by confocal microscopy. The data obtained from these positive control experiments may be used as a training set for data that would allow for the automated assaying of different cellular structures in different cell types based on machine learning.

In some embodiments, the data obtained from these experiments are used to generate a signature for a test agent. In some embodiments, the method of generating a signature for a test agent comprises (a) admixing the test agent with one or more stably tagged stem cell clones; (b) detecting a response in the one or more stem cell clones; (c) detecting a response in a control stem cell; (d) detecting a difference in the response in the one or more stem cell clones from the control stem cell; and (e) generating a data set of the difference in the response. In some embodiment the detected response in the stem cell clones and/or control cells is one or more of cell proliferation, microtubule organization, actin dynamics, vesicle trafficking, cell-surface protein expression, DNA replication, cytokine or chemokine production, changes in gene expression, and/or cell migration. In some embodiments, the control cell is a stably tagged stem cell clone that has not been exposed to the test agent or a control agent (e.g., a vehicle control). In some embodiments, the control cell is a stably tagged stem cell clone that has been exposed a control agent (e.g., a vehicle control). In some embodiments, these methods are used to determine the toxicity of a test agent and/or to determine the optimal dose of a test agent required to induce or inhibit a particular cell function or cell response. In such embodiments, the difference in the response in the one or more stem cell clones from the control stem cell are quantified and used to generate a data set of the difference in the response. This data-set can then be used as a training set for an algorithm to predict the effect of a related agent on a particular cellular function.

In some embodiments, stably tagged stem cell clones derived from diseased patients or stably tagged mutant stem cell clones can be differentiated into one or more differentiated cell types assayed by the methods described herein to generate a cell-type specific data-set related to a particular disease. In such embodiments, the cell proliferation, microtubule organization, actin dynamics, vesicle trafficking, cell-surface protein expression, DNA replication, cytokine or chemokine production, changes in gene expression, and/or cell migration of the differentiated cells can be determined at one or more time points during differentiation and maturation. Data sets derived from such assays can then be used as a training set for one or more disease-specific algorithms that can be applied to a cell sample derived from a patient to determine whether the patient has a disease, the stage of disease, and/or used to monitor the effects of a particular disease treatment. In some embodiments, the disease is selected from a disease characterized by aberrant cell growth, wound healing, inflammation, and/or neurodegeneration.

In some embodiments, methods are provided for live-cell imaging to observe intracellular protein localization, expression intensity, and persistence of expression in the modified stem cells or stably transfected stem cell clones described herein. In some embodiments, the expression of one or more detectable tags does not substantially or does not significantly alter the endogenous expression or localization of the tagged protein. In some embodiments, the invention provides for methods of live-cell imaging in three dimensions using the stably tagged stem cell clones and the cell culturing and plating and microscopy methods described herein.

### IV. Kits

In some embodiments, provided herein are kits comprising the stably tagged stem cell clones described herein. In some embodiments, the kits described herein comprise two or more stably tagged stem cell clones, wherein each stably tagged stem cell clone expresses a tagged endogenous protein. In some embodiments, each stably tagged stem cell clone express a different tagged endogenous protein. In some embodiments, the kits described herein comprise 2, 3, 4, 5, 6, 7, 8, 9, 10 or more stably tagged stem cell clones, wherein each stably tagged stem cell clone expresses a different tagged endogenous protein. In some embodiments, the kits described herein comprise two or more stably tagged stem cell clones, wherein each stably tagged stem cell clone expresses two or more tagged endogenous proteins. In some embodiments, the kits described herein comprise 2, 3, 4, 5, 6, 7, 8, 9, 10 or more stably tagged stem cell clones, wherein each stably tagged stem cell clone expresses two or more tagged endogenous proteins. In some embodiments, the kits described herein comprise two or more stably tagged stem cell clones, wherein each stably tagged stem cell clone expresses 2, 3, 4, 5, 6, 7, 8, 9, 10 or more tagged endogenous proteins. In some embodiments, the kits described herein comprise 2, 3, 4, 5, 6, 7, 8, 9, 10 or more stably tagged stem cell clones, wherein each stably tagged stem cell clone expresses 2, 3, 4, 5, 6, 7, 8, 9, 10 or more tagged endogenous proteins. In some embodiments, each stably tagged stem cell clone express a group of tagged endogenous proteins that are different from the tagged endogenous proteins expressed by another stem cell clone in the same composition. Exemplary endogenous proteins that can be tagged in these embodiments are shown in Tables 1 and 2, including but not limited to PXN, TUBA1B, LMNB1, ACTN1, TOMM20, DSP, SEC61B, FBL, ACTB, MYH10, VIM, TJP1 (also known as ZO-1), AAVS1, MAP1LC3B (also known as LC3), ST6GAL1, LAMP1, CETN2, SLC25A17 (also known as PMP34), RABSA, GJA1 (also known as connexin 43 (CX43)), MAPK1, ATP2A2, AKT1, CTNNB1, NPM1, HIST1H2BJ, CAGGS:HIST1H2BJ:2A:CAAX, PKD2, DMD, DES, SLC25A17 (also known as PMP34), SMC1A, NUP153, CTCF, CBX1, Oct4, Sox2, Nanog, TNNI1, ACTN2, TNNI3, MYL2, MYL7, TTN, SMAD, SMAD5, NKX2-5, MESP1, MIXL1, ISL1, CYP2E1, TF, HPX, ALB, PKD1, PKD2, KRT5, and LAMC2. In some embodiments, the kits also allow for building an entire “cell clinic” or reference set that comprises cell types from every major organ system, or those of interest, that allows for the interrogation of likely function of new genes and assaying of cellular toxicity.

In some embodiments, the present disclosure provides kits for assessing differentiation agents and/or the effect of compounds or drugs on the differentiation of stem cells. In some embodiments, the present disclosure provides a kit comprising one or more stably tagged stem cell clones expressing one or more tagged endogenous proteins. In some embodiments, the present disclosure provides a kit comprising a plurality of stably tagged stem cell clones expressing one or more tagged endogenous proteins. In some embodiments, the cells are provided as an array such that all cellular structures are tagged among a plurality of stably tagged stem cell clones.

In some embodiments, the kits described herein further comprise one or more agents known to elicit stem cell differentiation into one or more cell types. One of skill in the art would understand the appropriate media and agents for differentiation into various cell types. For example, a kit may include stably tagged stem cells and media containing Activin A for cardiomyocyte differentiation. Alternatively, a kit may include stably tagged stem cells and media containing factors described in Methods Mol Biol. 2014; 1210:131-41 or Biomed Rep. 2017 April; 6(4): 367-373 for hepatocyte differentiation. Alternatively, a kit may include stably tagged stem cells and media containing factors described in Methods Mol Biol. 2017, 1597:195-206 or Nat Commun. 2015 Oct 23; 6:8715 for renal cell differentiation. Alternatively, a kit may include stably tagged stem cells and media containing factors described in Mol Psychiatry. 2017 Apr. 18. doi: 10.1038/mp.2017.56 or Scientific Reports volume 7, Article number: 42367 (2017) for neuronal cell differentiation. Additional exemplary factors for producing differentiated cell types from human iPSCs are shown in FIG. 32. The stably tagged stem cells according to this embodiment may be provided in expanded form, for example, on a multi-well plate and ready for assay. Alternatively, the cells may be provided in a form that requires further expansion before plating and assaying.

In some embodiments, provided herein are kits comprising one or more differentiated cell types derived from one or more stably tagged stem cell clones. As used herein “derived from,” for example, one or more stably tagged stem cell clones refers to cells that are differentiated, from the stably tagged stem cell clones. In some embodiments, cells that are derived from stably tagged stem cell clones are terminally differentiated cells that are direct progeny of the stably tagged stem cell clones. Therefore, the differentiated cell types, like their stably tagged stem cell clone progenitors also express tagged (e.g. with a detectable marker, such as, for example, GFP and the like) structural or non-structural proteins. In one embodiment, the kits provided herein comprise one or more differentiated cell types. In some embodiments, kits provided herein contain differentiated cell types from all three germ layers. In some embodiments, kits are provided containing differentiated cells of substantially all major cell types of the body derived from stably tagged stem cell clones. In some embodiments, the kits are provided on multi-well plates in assay ready format. In some embodiments, the cells are provided in a form that requires thawing, culturing and/or expanding the cells. In some embodiments, the differentiated cells derived from stably tagged stem cells are provided in an array such that for each cell type member in the array, a tagged protein member is provided such that every structure being studied is tagged in each cell type being assayed.

### EXAMPLES

The following examples are for the purpose of illustrating various exemplary embodiments of the invention and are not meant to limit the scope of the present invention in any fashion. Alterations, modifications, and other changes to the described embodiments which are encompassed within the spirit of the invention as defined by the scope of the claims are specifically contemplated.

### Example 1—a Ribonudeoprotein (RNP)-Based CRISPR/Cas9 System to Create Fluorescent Protein-Tagged hiPSC Lines

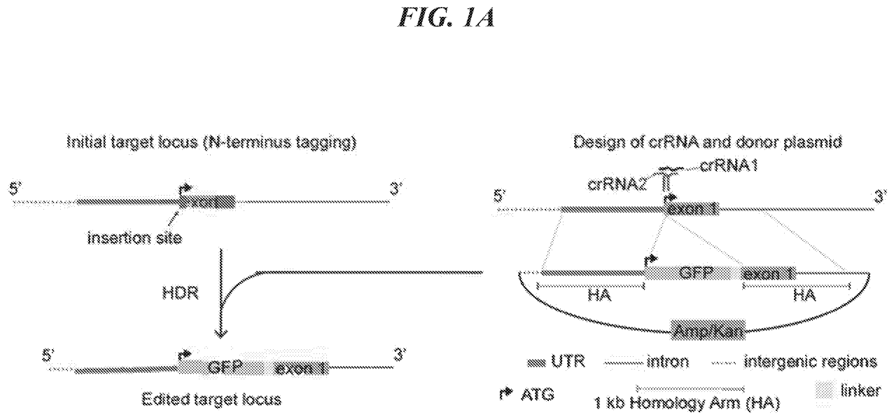

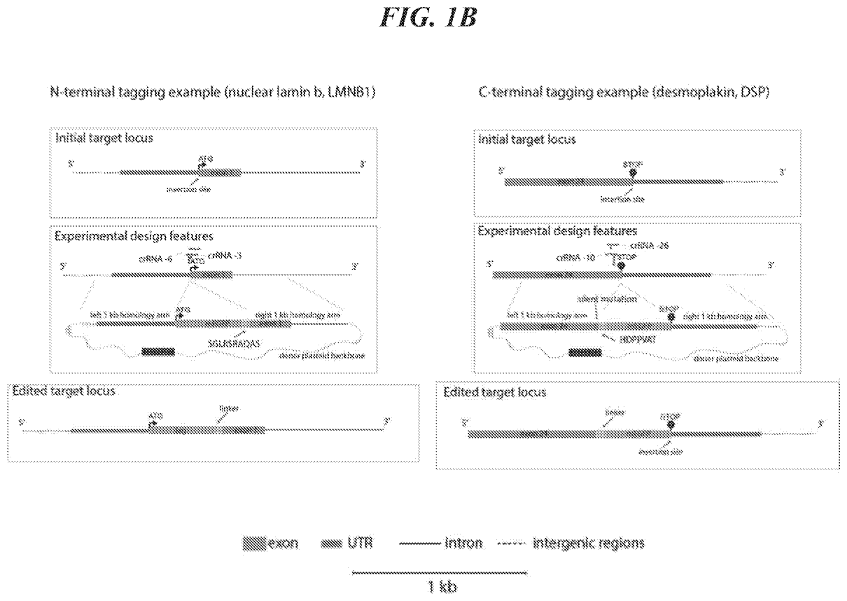

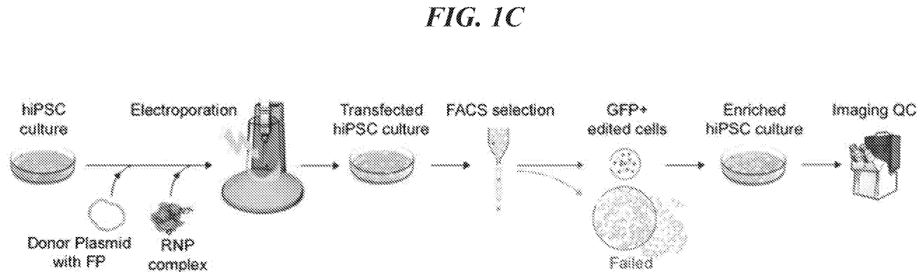

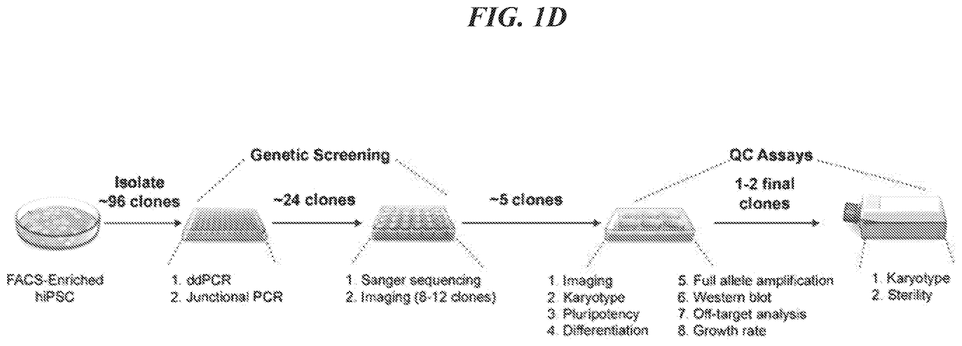

The CRISPR/Cas9 system was used to introduce a GFP tag into the genomic loci of various proteins by HDR-mediated incorporation. Exemplary proteins tagged by the methods described herein are shown in Tables 1 and 2 above. Experiments were designed to introduce GFP at the N- or C-terminus along with a short linker using a CRISPR/Cas9 RNP and a donor plasmid encoding the full length GFP protein (FIG. 1A). The donor plasmid contained 1 kb homology arms about 1 kb in length, on either side of the GFP operably linked to a linker sequence and a bacterial selection sequence in the backbone. The example in the schematic shows successful N-terminal tagging via HDR resulting in the tag and linker being inserted after the endogenous start codon (ATG) in frame with the first exon (FIG. 1A, right panel). FIG. 1B illustrates a schematic of donor plasmids for N-terminal tagging of LMNB1 and C-terminal tagging of DSP.

crRNA Design

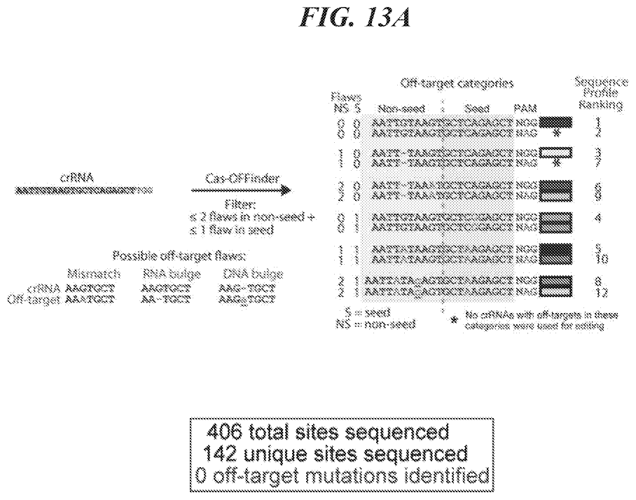

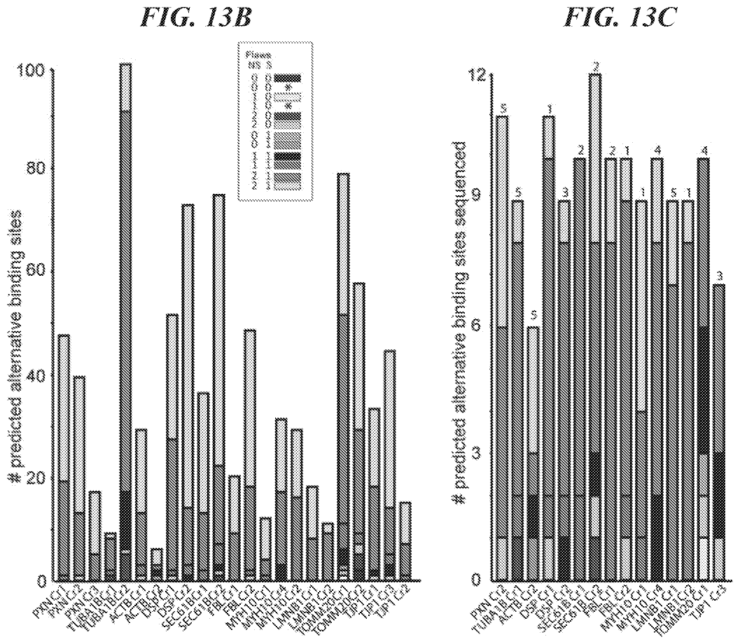

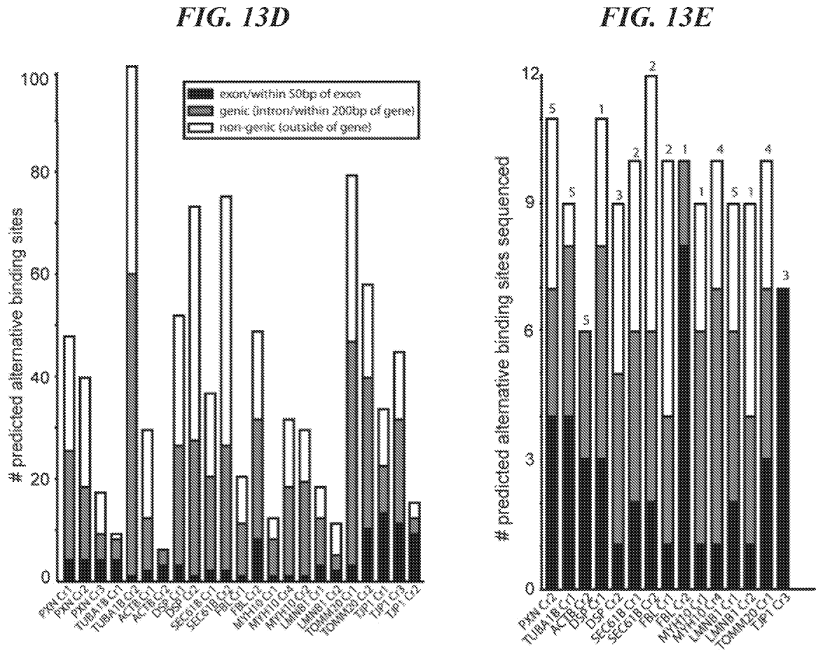

Custom synthetic crRNAs and their corresponding tracrRNAs were ordered from either IDT or Dharmacon. FIG. 13 shows the predicted genome wide CRISPR/Cas9 binding sites, categorized according to sequence profile and location with respect to genes. At least two independent crRNA sequences were used in each editing experiment in an effort to maximize editing success and elucidate the potential significance of possible off-target effects in the clonal cell lines generated (FIG. 13A). Predicted alternative CRISPR/Cas9 binding sites were categorized for each crRNA used and each predicted off-target sequence was categorized according to its sequence profile (the number of mismatches and RNA or DNA bulges it contains relative to the crRNA used in the experiment and their position relative to the PAM) (FIGS. 13B and 13C). Cas-OFFinder was used to discriminate between crRNA sequences with respect to their genome-wide specificity (Bae et al., (2014) Bioinformatics, 30(10): 1473-1475) by identifying all alternative sites genome-wide with ≤2 mismatches/bulges in the non-seed and/or ≤1 mismatch/bulge in the seed region, with an NGG or NAG PAM. As indicated in FIG. 13A, the seed and non-seed region of a crRNA binding sequence was defined with respect to its proximity to the PAM sequence. All predicted off-target sites were additionally categorized according to their location with respect to annotated genes (FIG. 13D). Genomic location was defined as follows:

(a) exon: inside exon or within 50 bp of exon;

(b) genic: in intron (but >50 bp from an exon) or within 200 bp of an annotated gene;

(c) non-genic: >200 bp from an annotated gene.

When possible, crRNAs targeting Cas9 to within 50 bp of the intended GFP integration site were used, with a strong preference for any crRNAs with binding sites within 1 Obp. A subset of CRISPR/Cas9 alternative binding sites identified by Cas-OFFinder were selected for sequencing and FIG. 13E shows the breakdown of sequenced off-target sites by genomic location with respect to annotated genes. Numbers above bars represent the number of clones sequenced for each experiment. All 406 sequenced sites were found to be wild type.

Only crRNAs unique within the human genome were used with one unavoidable exception (TOMM20, where the locus sequence restricted crRNA choice), and crRNAs whose alternative binding sites include mismatches in the “seed” region and are in non-genic regions were prioritized whenever possible. Table 5 below shows exemplary polynucleotide sequences of the crRNA sequences.

TABLE 5
Exemplary crRNA sequences

| | | | crRNA | SEQ ID | |
|---|---|---|---|---|---|
| Gene | Cell Line | crRNA | sequence (5′-3′) | NO: | PAM |
| PSN | AICS | Cr1 | CTTGTCGTTCTGCTCCTTGA | 85 | AGG |
| | | | CTTGTCGTTCTGCTCCTTGA | 86 | AGt |
| | | Cr2 | GCACCTA/GCAGAAGAGCTTG | 87 | AGG |
| | | | GCACCTA/GCAGAAGAGCTTG | 88 | AGt |
| | | Cr3 | TCTAGGTCACAGTCGCAGTT | 89 | GGG |
| | | | TCTAGGTCACAGTCGCAGTT | 90 | GGt |
| SEC61B | AICS-10 | Cr1 | CCCTCATCTCCAAT/ATGGTA | 91 | TGG |
| | | | CaCTCATCTCCAAT/ATGGTA | 92 | TGG |
| | | Cr2 | GCCATACCAT/ATTGGAGATG | 93 | AGG |
| | | | GCCATACCAT/ATTGGAGATG | 94 | AGt |
| TOMM20 | AICS-11 | Cr1 | AATTGTAAGTGCTCAGAGCT | 95 | TGG |
| | | | AATTGTAAGTGCaCAGtcCT | 96 | TGG |
| | | Cr2 | TGGTAGTTGAGCAGCTCTGG | 97 | GGG |
| | | | TGGTAGTTGAGCAGCTCTGG | 98 | GGt |
| TUBA1B | AICS-12 | Cr1 | GATGCACTCACG/CTGCGGGA | 99 | AGG |
| | | | GATGCACTC/CTGCGGtA | 100 | AGt |
| | | Cr2 | AGAGATAAGGTCTGTCGCCC | 101 | AGG |
| | | | AGAGATAAGGTCTGTCGCCC | 102 | AGt |
| LMNB1 | AICS-13 | Cr1 | GGGGTCGCAGTCGCCAT/GGC | 103 | GGG |
| | | | GGGGTCGCAGTCGC/GGC | 104 | GGG |
| | | Cr2 | GTCGCAGTCGCCAT/GGCGGG | 105 | CGG |
| | | | GTCGCAGTCGC/GGCGGG | 106 | CGG |
| FBL | AICS-14 | Cr1 | AAC/TGAAGTTCAGCGCTGTC | 107 | AGG |
| | | | AAC/TGAAGTTCAGCcCTGag | 108 | CGG |
| | | Cr2 | CA/GTTCTTCACCTTGGGGGG | 109 | TGG |
| | | | CA/GTTCTTCACtTTaGGaGG | 110 | TGG |
| ACTB | AICS-16 | Cr1 | GCTATTCTCGCAGCTCACCA | 111 | TG/G |
| | | | GCTATTCTCGCAaCTgACaa | 112 | TG/G |
| | | Cr2 | GCCGTTGTCGACGACGAGCG | 113 | CGG |
| | | | GCCGTTGTCGACGACGAGCG | 114 | CtG |
| DSP | AICS-17 | Cr1 | TCATTTAGCAGTAGTTCTAT | 115 | TGG |
| | | | TCATTTAGCAGTAGTagcAT | 116 | TGG |
| | | Cr2 | AGAACTACTGCTAAATGAGT | 117 | AGG |
| | | | cctACTACTGCTAAATGAGT | 118 | AtG |
| TJP1 | AICS-23 | Cr1 | CTTGGCGGCCGCAGCTCTGG | 119 | CGG |
| | | | CTTtGCGGCCGCAGCTCTGG | 120 | Cac |
| | | Cr2 | TCTCTCTCCAGCGCCGCGCG | 121 | AGG |
| | | | TCTCTCTCCAGCGCCGCGCG | 122 | caa |
| | | Cr3 | GGCCGCGGAGGCGCTCACCT | 123 | TGG |
| | | | GGCCGCGGAGGttCTCACCT | 124 | TtG |
| MYH10 | AICS-24 | Cr1 | TTTACAATG/GCGCAGAGAAC | 125 | TGG |
| | | | TTTACAATG/GCaCAaAGgAC | 126 | aGG |
| | | Cr2 | G/GCGCAGAGAACTGGACTCG | 127 | AGG |
| | | | G/GCaCAaAGgCaGGgCTGG | 128 | AGG |
| | | Cr4 | GTTCTCTGCGC/CATTGTAAA | 129 | TGG |
| | | | GTcCTtTGLGC/CATTGTAAA | 130 | TGG |
| GALT | AICS-19 | Cr1 | CGCC/TGACCACGCCGACCAC | 131 | AGG |
| | | | CGCC/TGACCACGgaGACCcC | 132 | AGG |
| | | Cr2 | TCAAGGCCCTGTGGTCGGCG | 133 | TGG |
| | | | TCAAGGCCCTGgGGTCtcCG | 134 | TGG |
| TUBG1 | AICS-18 | Cr1 | AGTCTGGCCGTGTGGCCGCA | 135 | TGG |
| | | | AGTCTGGtCtaGTaGCCGCA | 136 | TGa |
| | | Cr2 | GGAGATGTAGTCTGGCCGTG | 137 | TGG |
| | | | GGAGATGTAGTCTGGtCtaG | 138 | TaG |
| | | Cr3 | AGGGCTTGGGCCAACCAGTA | 139 | AGG |
| | | | AGGGCTTGGGCCAACCAGTA | 140 | AGt |

### Donor Plasmid Design

Donor plasmids were designed for each target locus and contained design features specific to each target and a GFP-encoding nucleic acid sequence (See e.g., FIG. 1A and FIG. 1B). Homology arms of about 1 kb in length and corresponding to the endogenous DNA regions located 5′ and 3′ to the target insertion site were designed from the hg38 reference genome and were corrected for known SNPs in WTC11 cells. Unique linkers for each locus were used and were inserted 5′ of the GFP sequence for C-terminal tagging of the endogenous protein or 3′ of the GFP sequence for N-terminal tagging of the endogenous protein. When necessary, mutations were introduced to the plasmid backbone to prevent crRNA binding and Cas9-mediated cleavage of the plasmid. Plasmids were initially created either by In-Fusion assembly of gBlock pieces (IDT) into a pUC19 backbone, or the plasmids were synthesized and cloned into a pU57 backbone by Genewiz. All plasmids were deposited in the Addgene database. Donor plasmids were diluted to working concentrations of 1 μg/μL in TE. In some experiments, higher concentrations of donor plasmid were used, but lower concentrations (<500 ng/μL) were avoided. Table 6 below illustrates nucleic acid sequences for exemplary plasmid inserts comprising GFP detectable tags, homology arms targeting the indicated genes, and linkers including:

(a) 5′ paxillin homology arm (SEQ ID NO: 6)—linker (SEQ ID NO: 278)—EGFP—3′ paxillin homology arm (SEQ ID NO: 21);

(b) 5′ SEC61B homology arm (SEQ ID NO: 7)—mEGFP—linker (SEQ ID NO: 279)—3′ SEC61B homology arm (SEQ ID NO: 22);

(c) 5′ TOMM20 homology arm (SEQ ID NO: 9)—linker (SEQ ID NO: 281)—mEGFP—3′ TOMM20 homology arm (SEQ ID NO: 24);

(d) 5′ TUBA1B homology arm (SEQ ID NO: 10)—mEGFP—linker (SEQ ID NO: 282)—3′ TUBA1B homology arm (SEQ ID NO: 25);

(e) 5′ LMNB1 homology arm (SEQ ID NO: 4)—mEGFP—linker (SEQ ID NO: 276)—3′ LMNB1 homology arm (SEQ ID NO: 19);

(f) 5′ FBL homology arm (SEQ ID NO: 3)—linker (SEQ ID NO: 275)—mEGFP—3′ FBL homology arm (SEQ ID NO: 18);

(g) 5′ ACTB homology arm (SEQ ID NO: 1)—mEGFP—linker (SEQ ID NO: 273)—3′ ACTB homology arm (SEQ ID NO: 16);

(h) 5′ DSP homology arm (SEQ ID NO: 2)—linker (SEQ ID NO: 274)—mEGFP—3′ DSP homology arm (SEQ ID NO: 17);

(i) 5′ TJP1 homology arm (SEQ ID NO: 8)—mEGFP—linker (SEQ ID NO: 280)—3′ TJPI homology arm (SEQ ID NO: 23); and

(j) 5′ MYH10 homology arm (SEQ ID NO: 5)—mEGFP—linker (SEQ ID NO: 277)—3′ MYH10 homology arm (SEQ ID NO: 20).

5′ homology arm sequences are shown in underlined text, linker sequences are shown in italic text, tag sequences are shown in regular text, and 3′ homology arm sequences are shown in bold text. Additional plasmid insert sequences are provided in SEQ ID NOs: 31-84.

TABLE 6
Exemplary plasmid insert sequences

| | | SEQ |
|---|---|---|
| Gene | | ID |
| Targeted | AA Sequence | NO: |
| Paxillin | CCTCTGCCTGCTGAGTTCCAGTGATTCTCCCGCCTCAGCCTCCCAAGTAGCTGAGATTACA | 31 |
| | GGCACACGCCCCCATGCCTGGCTAATTTTTTGTATTTTCAGTAGAGACGGGGTTTCACCAT | |
| | GTTGGCCAAGCTGGTCTTGAACTCCTGACCTCAGGTGATCCGCCTCCCTTGGCCTCCCAAA | |
| | GTGCTGGGATTACAAGTGTGAGCCACCTCACCCGGCCCCTCTCAGAGCCTTTTCTACCTAT | |
| | ATGTGATGTGAATCTCCAATGAGAATCTAGGAGGCAGAGTTTGACTACAGACCAGTGTCAC | |
| | ACCTGTGTTTCGGGGAACACTGTTACAGCCACCTGGCTAAGTGCTCAGGAGTCAGAGCTGT | |
| | GTATGAATCCAGGCTGTGACCTCAGTAGCTGCATGACCCTGGGCAAGTTACTTCACCTGTG | |
| | TGCTTCAGTTGCCTCCCCTGTTGGGAGAACTAAATAATCCCAGCCCTGTGGGAGGCGGAGG | |
| | TGGGAGGATTGCAGGAGGCCACATTTGACCAGCATGGGCAATATAGTGAGACCCCCATCTC | |
| | TACAAAAAAAATTTATTTAATAAAATAAAAATGAAAAATGAGCGTTTAGGACAACAGGGCA | |
| | CATGGGAAACGCCTAGCAAGTAGGAGGCACTCCGAGCGTGCCGACTAGGCCCACCGCGGCC | |
| | CCATCACACAGGGTGCAGCTCTAGCCCGAGGGGCAGCTCCCTGAGCCCCTCTCTCCGCCTG | |
| | GCAGGAATGCTTCACGCCATTCGTGAACGGCAGCTTCTTCGAGCACGACGGGCAGCCCTAC | |
| | TGTGAGGGGCACTACCACGAGCGGCGCGGCTCGCTGTGTTCTGGCTGCCAGAAGCCCATCA | |
| | CCGGCCGCTGCATCACCGCCATGGCCAAGAAGTTCCACCCCGAGCACTTCGTCTGTGCCTT | |
| | CTGCCTCAAGCAGCTCAACAAGGGCAACTTCAAGGAGCAGAACGACAAGCCTTACTGTCAG | |
| | AACTGCTTACTCAAGCTCTTCTGCGGTACCAGCGGCGGAAGCGTGAGCAAGGGCGAGGAGC | |
| | TGTTCACCGGGGTGGTGCCCATCCTGGTCGAGCTGGACGGCGACGTAAACGGCCACAAGTT | |
| | CAGCGTGTCCGGCGAGGGCGAGGGCGATGCCACCTACGGCAAGCTGACCCTGAAGTTCATC | |
| | TGCACCACCGGCAAGCTGCCCGTGCCCTGGCCCACCCTCGTGACCACCCTGACCTACGGCG | |
| | TGCAGTGCTTCAGCCGCTACCCCGACCACATGAAGCAGCACGACTTCTTCAAGTCCGCCAT | |
| | GCCCGAAGGCTACGTCCAGGAGCGCACCATCTTCTTCAAGGACGACGGCAACTACAAGACC | |
| | CGCGCCGAGGTGAAGTTCGAGGGCGACACCCTGGTGAACCGCATCGAGCTGAAGGGCATCG | |
| | ACTTCAAGGAGGACGGCAACATCCTGGGGCACAAGCTGGAGTACAACTACAACAGCCACAA | |
| | CGTCTATATCATGGCCGACAAGCAGAAGAACGGCATCAAGGTGAACTTCAAGATCCGCCAC | |
| | AACATCGAGGACGGCAGCGTGCAGCTCGCCGACCACTACCAGCAGAACACCCCCATCGGCG | |
| | ACGGCCCCGTGCTGCTGCCCGACAACCACTACCTGAGCACCCAGTCCGCCCTGAGCAAAGA | |
| | CCCCAACGAGAAGCGCGATCACATGGTCCTGCTGGAGTTCGTGACCGCCGCCGGGATCACT | |
| | CTCGGCATGGACGAGCTGTACAAGTAATGATAGGTGCCCTGCCCCTGTCTCTGCCCCCCTT | |
| | CCCCAGCCAGCATCACCAACTGCGACTGTGACCTAGAGACTTCACCCGGGGGTGAAGGGGT | |
| | AAACCCGACTGAAACTGGAACCCTTGTCCTCCGCTGGTGCGGGATGGACAGAGGGCCGTGA | |
| | GGGGTCCCCCTGCTTGTCTTCACCCCTGCCAGAGCCTCTGGGCCCCCTCCTCCCTCCTGTA | |
| | GCTCTCCCTAGGCTGCCCACTCTCCATCCTCCCCAGGGGTAGAGGCTGGGGGCTCCACCCC | |
| | AGCCCATGTACGTCCCCACGAACTGGCCTGGCCAGCACCCCACACTGGAGCCATCTCTTCC | |
| | TCATATTTCAGCAGTGCAGCCGGGGGGCAGGGAAGGGCAGGCAGGGTCTGTTGGGGTCTCT | |
| | TTTTATCCTTATTCCTCCCCCGACCTAATTGTCTTTGTTCTGTGATTATTGGGGGACACCC | |
| | GGCTCCCTCCAGACAATGCCAGCATAAATCCATCCATCCAAAGGCAGAGAACCAAAGGGGC | |
| | CATGGAAGGTTCTCTGTGCTCCTCCTACCCTTCCAGTGCCCTAGGCCTGGCGACTGCCCCT | |
| | GCCTTTTAGACCCGCCCTCCCCTTTTATACCTGCTCTTGTTCTACTGAGAAAAGCCTCTCC | |
| | AGCAATAATGTTTTCTAGTCACTTCCTCCGTCTCCGTGACGGCGTGCCTGGACACTGTACC | |
| | GACTTTGATAGATTTCTACACTGAGGTTTGAATTCATATCGCCTGAGTTGCTTTTACTTCT | |
| | CTATACAAAATGATTTTGAAGAGATTTTAAAGACGTTCCCTTTTGTATTCTCTTCCTCATC | |
| | CACCGCCACTGGGCCTGTCACTGATGGTGGCTCTGGTGTGAAGTTTGCTTTGTACTGAGGG | |
| | TTGGGGTGGGGAAGCAATTTGTATTTTATTGTTTCTTAGCACAAGCAGGTGAACTGGGAGC | |
| | AGCTCTGTGACTCCCCCTCTTTCACTTCATAGCTCACCAGGACTGTTTTATAAACT | |
| SEC61B | TTTAAATGGGCCCACACTAAAGTTAGAGAACCACAGGCTCGCTCACAACCCTGACTTCTCC | 36 |
| | ATGTCAGTTCCGATCTTTGCGAACCGCAGACAGGGAAGGTCTTCTCTCAGGGGTCATGCCC | |
| | GCGGCCGCCCTCCACGGCGAGGTCCGCACTCGCGCAGCCGGCCCCGCGGCCGCCTCACCTG | |
| | GTCGCACACTACCACGTCGAACTCCTCGTCGGCGAGGAACAGCACGTAGAGCGCCAGGAAA | |
| | ACCATGCGCACGTAGGCGCAGACGGCGGCGCCGCGGCCGCCCCAGCCCAGGCCTCGCGGCA | |
| | GCCAGTCCCCGGCACAGCGCACCGGTAGCTCGCGGCTCTCGGCGAAACAGTGGCCCGGGTC | |
| | GTAGTGCGCTGTCCAGATCTTCACGCTACACCCGCGCGCCTGCAGCGCCAGCGCCGCGTCC | |
| | AACACCAGCCGCTCAGCGCCGCCCACGCCCAGGTCTGGGTGGAGGAACAGCACCGACGGCT | |
| | TGGGAACCGAGTCCCGTTCCCGGCCCTGCTCCTCCGCCATGGCCCTGGAGCCGCAACTGCA | |
| | CCCCGCACCCTGATGGGGGTCTTCTGCGCAAGCTCCGCGCTCGTAGCTCCCAGCTGGCCAC | |
| | TGCGGGCCGACCCCGCCCTGCCGTACGTGCGTCAGTTAGGCCACATCAGCGCAAATCTGTG | |
| | AGGGTCTAGTAACTGCCTGAGAAAATATCTTGTCTGACCCCGGTTATATTTTTCCTTCGGT | |
| | AGGGATTGGACTTTCTGAAGGACGTTGTGATCCAAAGGAAGGAGGCCGGAGGTCTCTACTT | |
| | CCCATACAGCAGGTAACTAAGTTGTCTGTAGCAGACTGTCTACAGGCATATCGTGAGACGA | |
| | CCCAGGCGTCCCTGGGGTCAGAGAGGACCTTGCCTGCAAGTCCGGGGGCGGGGCCTGAGTC | |
| | AGTCTCGCCAGCTGCCGGTCTTTCGGGGGCTCCGTAACTTTCTATCCGTCCGCGTCAGCGC | |
| | CTTGCCACACTCATCTCCAATATGGTGAGCAAGGGCGAGGAGCTGTTCACCGGGGTGGTGC | |
| | CCATCCTGGTCGAGCTGGACGGCGACGTAAACGGCCACAAGTTCAGCGTGTCCGGCGAGGG | |
| | CGAGGGCGATGCCACCTACGGCAAGCTGACCCTGAAGTTCATCTGCACCACCGGCAAGCTG | |
| | CCCGTGCCCTGGCCCACCCTCGTGACCACCCTGACCTACGGCGTGCAGTGCTTCAGCCGCT | |
| | ACCCCGACCACATGAAGCAGCACGACTTCTTCAAGTCCGCCATGCCCGAAGGCTACGTCCA | |
| | GGAGCGCACCATCTTCTTCAAGGACGACGGCAACTACAAGACCCGCGCCGAGGTGAAGTTC | |
| | GAGGGCGACACCCTGGTGAACCGCATCGAGCTGAAGGGCATCGACTTCAAGGAGGACGGCA | |
| | ACATCCTGGGGCACAAGCTGGAGTACAACTACAACAGCCACAACGTCTATATCATGGCCGA | |
| | CAAGCAGAAGAACGGCATCAAGGTGAACTTCAAGATCCGCCACAACATCGAGGACGGCAGC | |
| | GTGCAGCTCGCCGACCACTACCAGCAGAACACCCCCATCGGCGACGGCCCCGTGCTGCTGC | |
| | CCGACAACCACTACCTGAGCACCCAGTCCAAGCTGAGCAAAGACCCCAACGAGAAGCGCGA | |
| | TCACATGGTCCTGCTGGAGTTCGTGACCGCCGCCGGGATCACTCTCGGCATGGACGAGCTG | |
| | TACAAGAGCGGCCTGAGAAGCATGGTATGGCGGCCCTTCCATGATCCCCGCCTCTCCCAGA | |
| | AGCCCTGACTCCTCCTGCTTTGCGCCGTGCTTTTCCTCTGTAGCTCCCTTGCTTCCCCCAG | |
| | CCTCGGGTGTGGGTGTCTAGGCCGGGGTTCTGGGGCAGGCCTGCCGCGCTCACCCGTCTGT | |
| | CTGCTTGTCTCCCTCTACAGCCTGGTCCGACCCCCAGTGGCACTAACGTGGGATCCTCAGG | |
| | GCGCTCTCCCAGCAAAGCAGTGGCCGCCCGGGCGGCGGGATCCACTGTCCGGCAGAGGTAA | |
| | GGAACCCTGCAGTTCGTTCGCTTCCAGACTCGGAGATAGGACCCAGAACCTCGCTGATTCT | |
| | GGGGTGGAGACCCTAGCATGTGAAGATTGACAAAGGCAAAATGAGCTTCTAGTGACGTGGC | |
| | CGTGGGAGTAGTTAAAGGCCTTTTGGGAGGAAGGCGACATTTTTTTTCTCGTTGCTCAGTT | |
| | TAGGGCACTACTCTTAAAAAAGGAAAGTTAACAAACTGGAATAGAGTCAGAGATAACTTTG | |
| | AGAAAACCGATGTCATTAAACTGGTGTCTCTGGACCTGAGGTTTGCACTCACATTTCCATC | |
| | TGGCGGCCCCATAAGCAATCTGTCCTACAGATAACTCGTCCTACACAAAACTTAGTCTCTT | |
| | TTCAGCTCAGCTCTCTCACTCTCAATTATATCTCCTTACTTCCATATGGCACTGTTGTACA | |
| | CTCATTTACTCAGAGCCAGAAACGTCAGCGTCATCTTGGATTTTTCTTATGCTCTTTCTCT | |
| | CTCTAGTCATATGCCAGACTTTAAACTCTGCTTGAAAGCTTTCTCATAAGCTCTTTCCTTT | |
| | TCCCTTTCTACTGCTTTGCATTTGCTACTTAACCCTTTTCTTCAGGCTGTTTGCTTTCCAG | |
| | TCCATCGTTCGCTCTGCTGTTACTCTTCTGCGTAGTTTCTGTTACTTGTTGCTGAAC | |
| TOMM20 | GATGATAGGAAGTATTTACAGAACTTTATAGTTAGTAACTGACTGGTTAATTTTTCAAACT | 37 |
| | GATTTTTACTCAACTGAATTAGAAAAGGACTGGAAAGAAAGTAAAGATCCCAACGACTTGA | |
| | GGGAACAAGTTGGACAACCAAGGACTTTGTCTAAATTGTTTTTATTTAGACTAATGTGGTT | |
| | CTAGTTCTAGAGGATTCATACTGGAATCATCGGTTTAATATTACGCTATTTGAAAGGCAGC | |
| | ATAGTATAGTACTTTTGGAAAATTGGCCTGAGGGTGATGTCTTTTGGAATATTTGTGAATT | |
| | CACTAGGAAGCCTAATTCCTTAAAAATGACCTCCTTCACTCAATTATCAGTGTTCTTGGTT | |
| | TGCCTGGGAGTGAAAAGAGATCTTAAAATCTTTTTGGTTTTAGTTACATAATTGACTGATG | |
| | TAATATTATGTAATGATGGCTGTACACAGTGTCTCATGCCCTATAATCCTAGCACTCATTT | |
| | GAGCTCAGGAGTTCAAGGCCAGCCTGGGCAACATGGTAAAACCCTGTCTGCACCAAAAATA | |
| | AAAAAAAAATTAGCCGGGCATGGTGGCATGAGTCTGTGGTTCCAGCTACTCAGGAGGCTGA | |
| | GGTGGGGGAGGATCGCTTGAGCCCAGGAGTCAGAGGTTGCAGTGAGCTGAGATTGTGCTAC | |
| | TGCACTCCAGCCTGGGTTACAGAGACCCCATCTGAATTAAAAACATATATAATGTAATGAT | |
| | CTGCCTCCTTTGTTAACTTGACTTTTGAAATGGGATTGTCAGTAGTATGATCATTGTTTTC | |
| | TTGGATGCCGACTGTGTGTAAAGTGTTACATTTTGAATTAAATGTCAGAATGGGTGAACTT | |
| | TACTAAGATTCAATTCTTTGAATACAAAGAGCATTTTATTTTGAAGTTAGAATACTAATTA | |
| | AATGCTTATGACACTTTAAAAAATTATTTTTTTTTTCTTTCAGAGAATTGTAAGTGCACAG | |
| | TCCTTGGCTGAAGATGATGTGGAAGGCGGTAGCGGGGATCCACCGGTCGCCACCGTGAGCA | |
| | AGGGCGAGGAGCTGTTCACCGGGGTGGTGCCCATCCTGGTCGAGCTGGACGGCGACGTAAA | |
| | CGGCCACAAGTTCAGCGTGTCCGGCGAGGGCGAGGGCGATGCCACCTACGGCAAGCTGACC | |
| | CTGAAGTTCATCTGCACCACCGGCAAGCTGCCCGTGCCCTGGCCCACCCTCGTGACCACCC | |
| | TGACCTACGGCGTGCAGTGCTTCAGCCGCTACCCCGACCACATGAAGCAGCACGACTTCTT | |
| | CAAGTCCGCCATGCCCGAAGGCTACGTCCAGGAGCGCACCATCTTCTTCAAGGACGACGGC | |
| | AACTACAAGACCCGCGCCGAGGTGAAGTTCGAGGGCGACACCCTGGTGAACCGCATCGAGC | |
| | TGAAGGGCATCGACTTCAAGGAGGACGGCAACATCCTGGGGCACAAGCTGGAGTACAACTA | |
| | CAACAGCCACAACGTCTATATCATGGCCGACAAGCAGAAGAACGGCATCAAGGTGAACTTC | |
| | AAGATCCGCCACAACATCGAGGACGGCAGCGTGCAGCTCGCCGACCACTACCAGCAGAACA | |
| | CCCCCATCGGCGACGGCCCCGTGCTGCTGCCCGACAACCACTACCTGAGCACCCAGTCCAA | |
| | GCTGAGCAAAGACCCCAACGAGAAGCGCGATCACATGGTCCTGCTGGAGTTCGTGACCGCC | |
| | GCCGGGATCACTCTCGGCATGGACGAGCTGTACAAGTAATGAGAAACAAATGTCAACATAA | |
| | TAAAATCTCAGTTAAAAATATTTTAAAAATTCTTGGTAGTTGAGCAGCTCTGGGGTAATAA | |
| | GGGCAAATATGCTTGTTATGAACTACACTGAAATCTACCAAAGTTAATGTTTACTTTGTGT | |
| | AGATCCATTTGTCTATTTTATTTATTTTTCCCAGTGAAAAGTGTATTTTGATAGAGAACTT | |
| | TTCATTCTATAAATACACTATGAGTTACTAAAATATCATGGATTTTGTTTATTCCTGAAAC | |
| | ATAGTTACATAGTTAAACTGTACATATGACATGGCTTATGTTAAAAATACCCAGTGCTCAG | |
| | TTTTGAAAGATAGGCAAAAAAAAAAAAGTATAGGAGAAACTGAAGAATGTACACTTTTTTA | |
| | GAGGGCACATTTTGCTGTAAATCTGGAAATTTGATAGACTTGACTGTGTTTGTGAAAACTG | |
| | AGCATTAAAGGTTTTGATTGATCCTTTCTTTCCATTTAATCTCTGAGACGTAAATATGTGA | |
| | GGTGTGCTGCTGTGCTGGGTTAACAGCTTCCTTCCCTTTCTGTGTAGCAGTCTTGAAATGT | |
| | TCTGTTTAAATCAGTAGGCTTAATGTGTTCTGGGTATTTATCTCCTTGTATTTTAAATATA | |
| | TGTAGTTGCAAATAGCACCAGGAATTAGATTTCTGTACACCCCTAATCTAGCCTTGTGAGC | |
| | TTCGCTAGTTAATGTGTGCTCACTTTCCCTCCATTTGTTACGTGAGAGAATGCGTCTGCTG | |
| | ATCACTGAAGTGTCCCTTTTAGCTTCTGATTCATTGGGTTCTGTTGGGCATCTTTAAATCC | |
| | ACCTTAACCTGAGGAATGTATGTGGGCAACCAGGCCCTGCATTTTTTTATATTCTGAATTT | |
| | TGCATGCTTGCCTGACTTAGTATTTCTGAATTGATGTTTTTTTTAATGGTATAACTATCTT | |
| | GATTTTCACTGAAATTATATGGTTCTGTCACTACTCTGTAAATTAATCCGAAACTTTTAAG | |
| | GT | |
| TUBA1B | GAGTGTTCTTTTTTTGATGAAAGCAATAAGAGGACTGCGGAAGAGCTCCCTGTCAATGTAC | 34 |
| | CGCTCTACACCAGTGTATTACGACAGTTCGTACACAACAGTCTGTAGAGGCCACCTGTCTC | |
| | TCCCTGCTGCGTTAGGAATTCAGGGGAGCAGGTGGTGGCAGTAAGGGATTTTGAGGGAACG | |
| | GAAATCGGATCTTGACCCAGATCTGGGCCGCCGATAATCTCCTACTGCGCTCAGACTGCTG | |
| | TGGAGGTGTTAGGCTGAGCCCGATGCCGGCAGGCAAGGGAGGATGGGCGGCTTGGGCAGCG | |
| | CCTTTGCAGACGTGGCCATTTCGTGCCTCTGCAGCACCGCCGGGGGGCGCAAGAGCGCGCG | |
| | CCCGGAATTGCTCATTCATCCTGTGCCGCAGAGCCCCGCCCCTTGTCCCTGCGGACAGACA | |
| | TTTCTTCTGCGCTGGTCTGGCCACGTGCTTCCTGTGCTAGGAGCTGCCCGGAAATGTGACC | |
| | ACCTAGTCTAAAGTGGGCTTCTGGGGCCTGAGCGCTGGATGGATGCCCACCTTCCTGTCTT | |
| | GGTCCTCCAAAGGAGGAAGCTGTGACTGAGCTGTCTTGGTCTGGAAGGAGGCCTTCCCGGT | |
| | TTAGGATGGGAAGGTAACATTCATTAAAAGCAACGTAGACTATAGTGTAGCTGTTCTCAAA | |
| | AGTAGTACATCTTAGAAAAGGATCTTTAGAAAAGATCGCTTTAGAAAAGGAAATTCGTTTT | |
| | CAGATTACGTGAGTAGCCTAGGTAACACAGCCAGACCTCATCTCCACAAAAAAAATGAAAA | |
| | AATTAGCCAGCTTGGTGGTCTGTGCCTGTGGTCCCAGCTGCTCCAGAGGCTGAGGTGGGGG | |
| | GATGACTGGAGCCTAGGCTGCAGTGAGCCTAGATGGCATCACTGCACTCAAGACTGGGCGA | |
| | CAGACCTTATCTCTAAAAAAATAAAGATTGCATGAGTATTTTGTTCCACTTGACAGTCATC | |
| | AATAGATTGGTTTAAATTGTGATATCTTTTTTACTTACCGCAGGTGAGCAAGGGCGAGGAG | |
| | CTGTTCACCGGGGTGGTGCCCATCCTGGTCGAGCTGGACGGCGACGTAAACGGCCACAAGT | |
| | TCAGCGTGTCCGGCGAGGGCGAGGGCGATGCCACCTACGGCAAGCTGACCCTGAAGTTCAT | |
| | CTGCACCACCGGCAAGCTGCCCGTGCCCTGGCCCACCCTCGTGACCACCCTGACCTACGGC | |
| | GTGCAGTGCTTCAGCCGCTACCCCGACCACATGAAGCAGCACGACTTCTTCAAGTCCGCCA | |
| | TGCCCGAAGGCTACGTCCAGGAGCGCACCATCTTCTTCAAGGACGACGGCAACTACAAGAC | |
| | CCGCGCCGAGGTGAAGTTCGAGGGCGACACCCTGGTGAACCGCATCGAGCTGAAGGGCATC | |
| | GACTTCAAGGAGGACGGCAACATCCTGGGGCACAAGCTGGAGTACAACTACAACAGCCACA | |
| | ACGTCTATATCATGGCCGACAAGCAGAAGAACGGCATCAAGGTGAACTTCAAGATCCGCCA | |
| | CAACATCGAGGACGGCAGCGTGCAGCTCGCCGACCACTACCAGCAGAACACCCCCATCGGC | |
| | GACGGCCCCGTGCTGCTGCCCGACAACCACTACCTGAGCACCCAGTCCAAGCTGAGCAAAG | |
| | ACCCCAACGAGAAGCGCGATCACATGGTCCTGCTGGAGTTCGTGACCGCCGCCGGGATCAC | |
| | TCTCGGCATGGACGAGCTGTACAAGGGAGGTTCAGGAGGCAGCGAGTGCATCTCCATCCAC | |
| | GTTGGCCAGGCTGGTGTCCAGATTGGCAATGCCTGCTGGGAGCTCTACTGCCTGGAACACG | |
| | GCATCCAGCCCGATGGCCAGATGCCAAGTGACAAGACCATTGGGGGAGGAGATGACTCCTT | |
| | CAACACCTTCTTCAGTGAGACGGGCGCTGGCAAGCACGTGCCCCGGGCTGTGTTTGTAGAC | |
| | TTGGAACCCACAGTCATTGGTGAGTTGACCTCAGTAACCTGAGATCCCAGGATGCTGGGAC | |
| | AGGAGGTCTGTCCAGGGGCTTCTCTTGTCACTCACTCACTCCCTCCGTCCTTCTCTCCCTC | |
| | CTCCAGATGAAGTTCGCACTGGCACCTACCGCCAGCTCTTCCACCCTGAGCAGCTCATCAC | |
| | AGGCAAGGAAGATGCTGCCAATAACTATGCCCGAGGGCACTACACCATTGGCAAGGAGATC | |
| | ATTGACCTTGTGTTGGACCGAATTCGCAAGCTGGTAAGCACCACATATAAATATGCATTTA | |
| | ATGTGGTGTGATAGTTCCAGTGCAAGTTGGGTGGAGTGACTGACATCATTCATTCTTTGGC | |
| | ACCTACCAAAATGTGGAATAGGCTGCTTGCTATATTAATTGGACTTCTAAATCAGATAGTC | |
| | CCTAGGTTATGGACAGTTTGTGGATATGTCTGTTTTGCCAATTCCTTGTGCTTACATCAGT | |
| | GAGATATGGTTCGTAATCTAAAAAGTTGAAATAGAAATTCTAAGATAATGTGTCCTGGCAT | |
| | TAAAATATTACATTTTTTTATTCCCCTACAGGCTGACCAGTGCACCGGTCTTCAGGGCTTC | |
| | TTGGTTTTCCACAGCTTTGGTGGGGGAACTGGTTCTGGGTTCACCTCCCTGCTCATGGAAC | |
| | GTCTCTCAGTTGATTATGGCAAGAAGTCCAAGCTGGAGTTCTCCATTTACCCAGCACCCCA | |
| | GGTTTCCACAGCTGTAGTTGAGCCCTACAACTCCATCCTCACCACCCACACCACCCTGGAG | |
| | CACTC | |
| LMNB1 | TAAAGGCTGGTACTTGGAACCTGCAAGCCGTGCATTTGGAACCTCGGACTCAAGTGCCTAT | 39 |
| | TACGTAATTCCACAGCGTCCCGGCCTCCAGGCCGTTTCCCGAGCCCTCCAGCGGAGCGGGG | |
| | GATAAGGTTACCACGCCCGCGGTGGCCGGGGACACTCTGAGTTTCGCGTGTGGCTTTTAGG | |
| | GACGTTTATATTTGAATTTCCCTGAACCGCCGAGTGTGGGCGGTGGCGCAGATCCGTCCCG | |
| | GAAACCTCCGGGCTCCTTCCCGCCTTTCTCAGGCCCGGCCCCTCCAAGGGGTCCCCGCGGG | |
| | GCGGCGGGAGGGCCCTGGGCCCAGAGCCGCGCGGGTGGGCAGTCCCAGGCGTCCTTCCTTA | |
| | CAGCCCTGAGCCTGGTCCGGGAACCGCCCAGCCGGGAGGGCCGAGCTGACGGTTGCCCAAG | |
| | GGCCAGATTTTAAATTTACAGGCCCGGCCCCCGAACCGCCGAAGCGCGCTGCCTGCTCCCC | |
| | ATTGGCCCATGGTAGTCACGTGGAGGCGCCGGGGCGTGCCGGCCATGTTGGGGAGTGCGGC | |
| | GCCGCGGCCCGCGCCACCTCCGCCCCCCGCGGCTTGCCTCCAGCCCGCCCCTCCCGGCCCT | |
| | CCTCCCCCCGCCCGCCGCTCCGTGCAGCCTGAGAGGAAACAAAGTGCTGCGAGCAGGAGAC | |
| | GGCGGCGGCGCGAACCCTGCTGGGCCTCCAGTCACCCTCGTCTTGCATTTTCCCGCGTGCG | |
| | TGTGTGAGTGGGTGTGTGTGTTTTCTTACAAAGGGTATTTCGCGATCGATCGATTGATTCG | |
| | TAGTTCCCCCCCGCGCGCCTTTGCCCTTTGTGCTGTAATCGAGCTCCCGCCATCCCAGGTG | |
| | CTTCTCCGTTCCTCTAAACGCCAGCGTCTGGACGTGAGCGCAGGTCGCCGGTTTGTGCCTT | |
| | CGGTCCCCGCTTCGCCCCCTGCCGTCCCCTCCTTATCACGGTCCCGCTCGCGGCCTCGCCG | |
| | CCCCGCTGTCTCCGCCGCCCGCCATGGTGAGCAAGGGCGAGGAGCTGTTCACCGGGGTGGT | |
| | GCCCATCCTGGTCGAGCTGGACGGCGACGTAAACGGCCACAAGTTCAGCGTGTCCGGCGAG | |
| | GGCGAGGGCGATGCCACCTACGGCAAGCTGACCCTGAAGTTCATCTGCACCACCGGCAAGC | |
| | TGCCCGTGCCCTGGCCCACCCTCGTGACCACCCTGACCTACGGCGTGCAGTGCTTCAGCCG | |
| | CTACCCCGACCACATGAAGCAGCACGACTTCTTCAAGTCCGCCATGCCCGAAGGCTACGTC | |
| | CAGGAGCGCACCATCTTCTTCAAGGACGACGGCAACTACAAGACCCGCGCCGAGGTGAAGT | |
| | TCGAGGGCGACACCCTGGTGAACCGCATCGAGCTGAAGGGCATCGACTTCAAGGAGGACGG | |
| | CAACATCCTGGGGCACAAGCTGGAGTACAACTACAACAGCCACAACGTCTATATCATGGCC | |
| | GACAAGCAGAAGAACGGCATCAAGGTGAACTTCAAGATCCGCCACAACATCGAGGACGGCA | |
| | GCGTGCAGCTCGCCGACCACTACCAGCAGAACACCCCCATCGGCGACGGCCCCGTGCTGCT | |
| | GCCCGACAACCACTACCTGAGCACCCAGTCCAAGCTGAGCAAAGACCCCAACGAGAAGCGC | |
| | GATCACATGGTCCTGCTGGAGTTCGTGACCGCCGCCGGGATCACTCTCGGCATGGACGAGC | |
| | TGTACAAGAGCGGCCTGAGAAGCAGAGCCCAGGCCAGCGCGACTGCGACCCCCGTGCCGCC | |
| | GCGGATGGGCAGCCGCGCTGGCGGCCCCACCACGCCGCTGAGCCCCACGCGCCTGTCGCGG | |
| | CTCCAGGAGAAGGAGGAGCTGCGCGAGCTCAATGACCGGCTGGCGGTGTACATCGACAAGG | |
| | TGCGCAGCCTGGAGACGGAGAACAGCGCGCTGCAGCTGCAGGTGACGGAGCGCGAGGAGGT | |
| | GCGCGGCCGTGAGCTCACCGGCCTCAAGGCGCTCTACGAGACCGAGCTGGCCGACGCGCGA | |
| | CGCGCGCTCGACGACACGGCCCGCGAGCGCGCCAAGCTGCAGATCGAGCTGGGCAAGTGCA | |
| | AGGCGGAACACGACCAGCTGCTCCTCAAGTGAGTGCTAGCTGGCGGCCGCGTTAGCGCCAA | |
| | GGAGGGGCGGGGGCGCAACCGCGGCGACCAGCTCACCGGGTTCTGCCGTGGGGAGGGAGCA | |
| | GAGGCCAGGATGCACGCGTCCTTCTGAAGGAACAGGGTCTCGGTCTCCGGAAAGGAGAAAG | |
| | AATCTAGAGTTCATAGCGGAGCAGGGGTCGCGGAGGGGGCTCGAGCTGTAGCGCTGGGGGG | |
| | CCGTGATGCCCATTTCTAGATTTTGGATACCCGCTGGGACGTGGTAAGTGCGCGCCTGGGA | |
| | CTGCCGAGAAGGAGCTCCCGCTTTCGCACTCGAATCCGGGGAGCCGGCGCGGAGAGGCGGC | |
| | CCCTCAGGCCCCAGGTGCGGGGAGCTGGAGCGCGAGCGCGCGCTCGCGTGCGCGCCCCAGT | |
| | TTCCGGCCGGCGCGAGACAAAGCGTCTAGCGGATTTGCAGTGCCGGGATGGGCGGCCGGGG | |
| | AGGACTGGCAGCCCGCCTCTAGAATGAATGAGCTTCGCGCGGGCAGAGAGAGGAAGGGGAG | |
| | GGACCTTCCCGCAGCATCCGCGTCTCCTGGGGGTGGGTCCCGCTTTGGCGCGCTCAGTCTT | |
| | GGCCCTGTGACGTTTTGCGAAGATTCTACGCCTGCTTTAGGCGGGAGAGAGAGGCGGAG | |
| FBL | TTTATTTTTATTTTTATTTTTTTGAGATGGAGTCTTACTCTGTCACCAGGTTGGAGTGCAG | 42 |
| | TGGTACGATCTCGGCTCACTGCAACCTCCAGTTCCTGGGTTCAAGCGATTCTCCTGCGTCA | |
| | GCTTCCCGAGTAGGTGGGACTACAGGTGTGCGCCACCACACCCGACTAATTTTTGTATTTT | |
| | TAGTAGAGATAGGGTTTCACCGTGTTGGCCAGGATGGTCTCAATCTCCTGATTTCGTGATT | |
| | GAGCCACCTCGGCCTCCCAAAGTGCTGGGATTACAGGCGTGAGCCACCACGCCCAGCCTTA | |
| | GACTGGGTAATTTATAATGAATGGAAATTTATTTGGCTCCCAGTTCCAAAGGCTGGAAAGT | |
| | CCAAGATTGGAGGTCTGAATCTGGCGAGGGCCTTCTTGCTGTCATCCATTGGCAGAAGGGT | |
| | GAGAGCAAGATAGAAAGGGGGCATAATCATCCTTTTAATCAGCAACCCACTCTTGTGATAA | |
| | TAGCATTACTCTATTCAGGAAGGCAGAGGCCTCATGACCTGAATCATCTCTCGAAGGTCCC | |
| | ACCTCTCAACTCTTGCATTTAAGGGTTACGTTTCCAACACATGAACTTTGGGGGACACACT | |
| | AGAACCATAGCACTGAGTTTTACTTGAATTAATAATGAAAACATCTGGTTTAAAGAGCACA | |
| | CAAGAGAAAAACAGCCCAAAGCCCTGTTGTAGACATTAGTCCTTTCTCCTCTTTAGGCCAA | |
| | CTGCATTGACTCCACAGCCTCAGCCGAGGCCGTGTTTGCCTCCGAAGTGAAAAAGATGCAA | |
| | CAGGAGAACATGAAGCCGCAGGAGCAGTTGACCCTTGAGCCATATGAAAGAGACCATGCCG | |
| | TGGTCGTGGGAGTGTACAGGTGAGCAGGGGCCCAGCAATACACCAAGACAGACATCTCTGT | |
| | CCCTTGCACCCCGAGTGCCATGATCCTGGGGACCCTCCTTCATCACCTATCTTCCTCTCAC | |
| | AGGCCACCTCCTAAAGTGAAGAACAAGCCCAACAGCGCCGTGGACGGCACCGCCGGCCCCG | |
| | GCGTGAGCAAGGGCGAGGAGCTGTTCACCGGGGTGGTGCCCATCCTGGTCGAGCTGGACGG | |
| | CGACGTAAACGGCCACAAGTTCAGCGTGTCCGGCGAGGGCGAGGGCGATGCCACCTACGGC | |
| | AAGCTGACCCTGAAGTTCATCTGCACCACCGGCAAGCTGCCCGTGCCCTGGCCCACCCTCG | |
| | TGACCACCCTGACCTACGGCGTGCAGTGCTTCAGCCGCTACCCCGACCACATGAAGCAGCA | |
| | CGACTTCTTCAAGTCCGCCATGCCCGAAGGCTACGTCCAGGAGCGCACCATCTTCTTCAAG | |
| | GACGACGGCAACTACAAGACCCGCGCCGAGGTGAAGTTCGAGGGCGACACCCTGGTGAACC | |
| | GCATCGAGCTGAAGGGCATCGACTTCAAGGAGGACGGCAACATCCTGGGGCACAAGCTGGA | |
| | GTACAACTACAACAGCCACAACGTCTATATCATGGCCGACAAGCAGAAGAACGGCATCAAG | |
| | GTGAACTTCAAGATCCGCCACAACATCGAGGACGGCAGCGTGCAGCTCGCCGACCACTACC | |
| | AGCAGAACACCCCCATCGGCGACGGCCCCGTGCTGCTGCCCGACAACCACTACCTGAGCAC | |
| | CCAGTCCAAGCTGAGCAAAGACCCCAACGAGAAGCGCGATCACATGGTCCTGCTGGAGTTC | |
| | GTGACCGCCGCCGGGATCACTCTCGGCATGGACGAGCTGTACAAGTAATGAAGTTCAGCCC | |
| | TGAGCGGATTGCGAGAGATGTGTGTTGATACTGTTGCACGTGTGTTTTTCTATTAAAAGAC | |
| | TCATCCGTCTCCCATGTCTGCTGCTCATTCCTCCCCTTGACCTGCTGACACAGGGAGCACG | |
| | CACCCTTGGTCAATTTTGCGGGGTTGGGTAAATTCTCACTCGGTCACAGAGCGCATGCTCC | |
| | GTTTCTAGCTGCCTTTGCGCAGCGGCAGCCTGGATTTCGGTTCTTGGGTGGGATTGGTAGC | |
| | TCGCTGCGCATGCGTGCAGGTAAGCGGCCATCTCGCGCAGGCGGAGTGTCAGTGTGGGTCA | |
| | CGTGAGGGGAGCGGAGAGGGAGGGATGGGGGCGGAGTCCAGGGCGTGGGGGGGCCGGTTTG | |
| | TTGTGGTCGCCATTTTGCTGGTTGCATTACTGGGTAATCGGGGCCCTGGCTTGCCGCGTCC | |
| | GCCGGATACCCTCAGCCAGTGGGCAGGTCTGAGCTCGGGCTCCCCGAGCAGTTTGAGTCCC | |
| | CTTGCCCGCTCCTTCAGGTAACGGCGCGGGGACGGGTGGGGCGGCAAGCGGTCGCAGGGAG | |
| | GTGGGCAGGACGGGATCCGCCCTGCTCCCGTCGCCGTGAGACTTAGCACGAGGCCAAGGGA | |
| | GGAGAGGAGGGGGGTGGCAGGCAGGTGCGGGCCCTGCCTGGCTATTCATAGTTGAATTCCT | |
| | GGAACCGGCCAAGCCCGAGGAAGCAGTTGCAGGAGGGAGGCTGGGAGGGGGTAGCCGGGCC | |
| | CCACTCCCGCCCTTTGTTTGGGCTCAGCTCCGCGGGCCGCTTCTTCGTCGCCTAGCAACAG | |
| | CTGCCCTAGGCTGTGATTGGCTGAGCTCTTGGCACCAGCGACCAATGGTACAGTTGTTGCC | |
| | ATGGCAGGTGCCGATTGCCAAGCTCAGTCGGGCCCCGCCTTCCGGTCTCAGCAGGCCCAGG | |
| | AGGGCCTCCTGGGTGGGGGGCGGGACGCCGGGTCCCTAGGGGCTGGTGGTCACTCAGGGTG | |
| | GGGCGTGTCGC | |
| ACTB | GCTCGAGCGGCCGCGGCGGCGCCCTATAAAACCCAGCGGCGCGACGCGCCACCACCGCCGA | 44 |
| | GACCGCGTCCGCCCCGCGAGCACAGAGCCTCGCCTTTGCCGATCCGCCGCCCGTCCACACC | |
| | CGCCGCCAGGTAAGCCCGGCCAGCCGACCGGGGCAGGCGGCTCACGGCCCGGCCGCAGGCG | |
| | GCCGCGGCCCCTTCGCCCGTGCAGAGCCGCCGTCTGGGCCGCAGCGGGGGGCGCATGGGGG | |
| | GGGAACCGGACCGCCGTGGGGGGCGCGGGAGAAGCCCCTGGGCCTCCGGAGATGGGGGACA | |
| | CCCCACGCCAGTTCGGAGGCGCGAGGCCGCGCTCGGGAGGCGCGCTCCGGGGGTGCCGCTC | |
| | TCGGGGCGGGGGCAACCGGCGGGGTCTTTGTCTGAGCCGGGCTCTTGCCAATGGGGATCGC | |
| | AGGGTGGGCGCGGCGGAGCCCCCGCCAGGCCCGGTGGGGGCTGGGGCGCCATTGCGCGTGC | |
| | GCGCTGGTCCTTGGGGCGCTAACTGCGTGCGCGCTGGGAATTGGCGCTAATTGCGCGTGCG | |
| | CGCTGGGACTCAAGGCGCTAACTGCGCGTGCGTTCTGGGGCCCGGGGTGCCGCGGCCTGGG | |
| | CTGGGGCGAAGGCGGGCTCGGCCGGAAGGGGTGGGGTCGCCGCGGCTCCCGGGCGCTTGCG | |
| | CGCACTTCCTGCCCGAGCCGCTGGCCGCCCGAGGGTGTGGCCGCTGCGTGCGCGCGCGCCG | |
| | ACCCGGCGCTGTTTGAACCGGGCGGAGGCGGGGCTGGCGCCCGGTTGGGAGGGGGTTGGGG | |
| | CCTGGCTTCCTGCCGCGCGCCGCGGGGACGCCTCCGACCAGTGTTTGCCTTTTATGGTAAT | |
| | AACGCGGCCGGCCCGGCTTCCTTTGTCCCCAATCTGGGCGCGCGCCGGCGCCCCCTGGCGG | |
| | CCTAAGGACTCGGCGCGCCGGAAGTGGCCAGGGCGGGGGCGACCTCGGCTCACAGCGCGCC | |
| | CGGCTATTCTCGCAACTGACAATGGTGAGCAAGGGCGAGGAGCTGTTCACCGGGGTGGTGC | |
| | CCATCCTGGTCGAGCTGGACGGCGACGTAAACGGCCACAAGTTCAGCGTGTCCGGCGAGGG | |
| | CGAGGGCGATGCCACCTACGGCAAGCTGACCCTGAAGTTCATCTGCACCACCGGCAAGCTG | |
| | CCCGTGCCCTGGCCCACCCTCGTGACCACCCTGACCTACGGCGTGCAGTGCTTCAGCCGCT | |
| | ACCCCGACCACATGAAGCAGCACGACTTCTTCAAGTCCGCCATGCCCGAAGGCTACGTCCA | |
| | GGAGCGCACCATCTTCTTCAAGGACGACGGCAACTACAAGACCCGCGCCGAGGTGAAGTTC | |
| | GAGGGCGACACCCTGGTGAACCGCATCGAGCTGAAGGGCATCGACTTCAAGGAGGACGGCA | |
| | ACATCCTGGGGCACAAGCTGGAGTACAACTACAACAGCCACAACGTCTATATCATGGCCGA | |
| | CAAGCAGAAGAACGGCATCAAGGTGAACTTCAAGATCCGCCACAACATCGAGGACGGCAGC | |
| | GTGCAGCTCGCCGACCACTACCAGCAGAACACCCCCATCGGCGACGGCCCCGTGCTGCTGC | |
| | CCGACAACCACTACCTGAGCACCCAGTCCAAGCTGAGCAAAGACCCCAACGAGAAGCGCGA | |
| | TCACATGGTCCTGCTGGAGTTCGTGACCGCCGCCGGGATCACTCTCGGCATGGACGAGCTG | |
| | TACAAGGCCGGCTCCGGTACCGATGATGATATCGCAGCGCTCGTCGTCGACAACGGCTCCG | |
| | GCATGTGCAAGGCCGGCTTCGCGGGCGACGATGCCCCCCGGGCCGTCTTCCCCTCCATCGT | |
| | GGGGCGCCCCAGGCACCAGGTAGGGGAGCTGGCTGGGTGGGGCAGCCCCGGGAGCGGGCGG | |
| | GAGGCAAGGGCGCTTTCTCTGCACAGGAGCCTCCCGGTTTCCGGGGTGGGGGCTGCGCCCG | |
| | TGCTCAGGGCTTCTTGTCCTTTCCTTCCCAGGGCGTGATGGTGGGCATGGGTCAGAAGGAT | |
| | TCCTATGTGGGCGACGAGGCCCAGAGCAAGAGAGGCATCCTCACCCTGAAGTACCCCATCG | |
| | AGCACGGCATCGTCACCAACTGGGACGACATGGAGAAAATCTGGCACCACACCTTCTACAA | |
| | TGAGCTGCGTGTGGCTCCCGAGGAGCACCCCGTGCTGCTGACCGAGGCCCCCCTGAACCCC | |
| | AAGGCCAACCGCGAGAAGATGACCCAGGTGAGTGGCCCGCTACCTCTTCTGGTGGCCGCCT | |
| | CCCTCCTTCCTGGCCTCCCGGAGCTGCGCCCTTTCTCACTGGTTCTCTCTTCTGCCGTTTT | |
| | CCGTAGGACTCTCTTCTCTGACCTGAGTCTCCTTTGGAACTCTGCAGGTTCTATTTGCTTT | |
| | TTCCCAGATGAGCTCTTTTTCTGGTGTTTGTCTCTCTGACTAGGTGTCTAAGACAGTGTTG | |
| | TGGGTGTAGGTACTAACACTGGCTCGTGTGACAAGGCCATGAGGCTGGTGTAAAGCGGCCT | |
| | TGGAGTGTGTATTAAGTAGGCGCACAGTAGGTCTGAACAGACTCCCCATCCCAAGACCCCA | |
| | GCACACTTAGCCGTGTTCTTTGCACTTTCTGCATGTCCCCCGTCTGGCCTGGCTGTCCCCA | |
| | GTGGCTTCCCCAGTGTGACATGGTGTATCTCTGCCTTACAGATCATGTTTGAGACCTTCAA | |
| | CACCCCAGCCATGTACGTTGCTATCCAGGCTGTGCTATCCCTGTA | |
| DSP | TGTTGACAGGAAGTTCTTTGATCAGTACCGATCCGGCAGCCTCAGCCTCACTCAATTTGCT | 38 |
| | GACATGATCTCCTTGAAAAATGGTGTCGGCACCAGCAGCAGCATGGGCAGTGGTGTCAGCG | |
| | ATGATGTTTTTAGCAGCTCCCGACATGAATCAGTAAGTAAGATTTCCACCATATCCAGCGT | |
| | CAGGAATTTAACCATAAGGAGCAGCTCTTTTTCAGACACCCTGGAAGAATCGAGCCCCATT | |
| | GCAGCCATCTTTGACACAGAAAACCTGGAGAAAATCTCCATTACAGAAGGTATAGAGCGGG | |
| | GCATCGTTGACAGCATCACGGGTCAGAGGCTTCTGGAGGCTCAGGCCTGCACAGGTGGCAT | |
| | CATCCACCCAACCACGGGCCAGAAGCTGTCACTTCAGGACGCAGTCTCCCAGGGTGTGATT | |
| | GACCAAGACATGGCCACCAGGCTGAAGCCTGCTCAGAAAGCCTTCATAGGCTTCGAGGGTG | |
| | TGAAGGGAAAGAAGAAGATGTCAGCAGCAGAGGCAGTGAAAGAAAAATGGCTCCCGTATGA | |
| | GGCTGGCCAGCGCTTCCTGGAGTTCCAGTACCTCACGGGAGGTCTTGTTGACCCGGAAGTG | |
| | CATGGGAGGATAAGCACCGAAGAAGCCATCCGGAAGGGGTTCATAGATGGCCGCGCCGCAC | |
| | AGAGGCTGCAAGACACCAGCAGCTATGCCAAAATCCTGACCTGCCCCAAAACCAAATTAAA | |
| | AATATCCTATAAGGATGCCATAAATCGCTCCATGGTAGAAGATATCACTGGGCTGCGCCTT | |
| | CTGGAAGCCGCCTCCGTGTCGTCCAAGGGCTTACCCAGCCCTTACAACATGTCTTCGGCTC | |
| | CGGGCTCCCGCTCCGGCTCCCGCTCGGGATCTCGCTCCGGATCTCGCTCCGGGTCCCGCAG | |
| | TGGGTCCCGGAGAGGAAGCTTTGACGCCACAGGGAATTCTTCCTACTCTTATTCATACTCA | |
| | TTTAGCAGTAGTAGCATTGGGCACCACGACCCCCCCGTTGCTACGGTGAGCAAGGGCGAGG | |
| | AGCTGTTCACCGGGGTGGTGCCCATCCTGGTCGAGCTGGACGGCGACGTAAACGGCCACAA | |
| | GTTCAGCGTGTCCGGCGAGGGCGAGGGCGATGCCACCTACGGCAAGCTGACCCTGAAGTTC | |
| | ATCTGCACCACCGGCAAGCTGCCCGTGCCCTGGCCCACCCTCGTGACCACCCTGACCTACG | |
| | GCGTGCAGTGCTTCAGCCGCTACCCCGACCACATGAAGCAGCACGACTTCTTCAAGTCCGC | |
| | CATGCCCGAAGGCTACGTCCAGGAGCGCACCATCTTCTTCAAGGACGACGGCAACTACAAG | |
| | ACCCGCGCCGAGGTGAAGTTCGAGGGCGACACCCTGGTGAACCGCATCGAGCTGAAGGGCA | |
| | TCGACTTCAAGGAGGACGGCAACATCCTGGGGCACAAGCTGGAGTACAACTACAACAGCCA | |
| | CAACGTCTATATCATGGCCGACAAGCAGAAGAACGGCATCAAGGTGAACTTCAAGATCCGC | |
| | CACAACATCGAGGACGGCAGCGTGCAGCTCGCCGACCACTACCAGCAGAACACCCCCATCG | |
| | GCGACGGCCCCGTGCTGCTGCCCGACAACCACTACCTGAGCACCCAGTCCAAGCTGAGCAA | |
| | AGACCCCAACGAGAAGCGCGATCACATGGTCCTGCTGGAGTTCGTGACCGCCGCCGGGATC | |
| | ACTCTCGGCATGGACGAGCTGTACAAGTAATAGTAGTCAGTTGCGAGTGGTTGCTATACCT | |
| | TGACTTCATTTATATGAATTTCCACTTTATTAAATAATAGAAAAGAAAATCCCGGTGCTTG | |
| | CAGTAGAGTGATAGGACATTCTATGCTTACAGAAAATATAGCCATGATTGAAATCAAATAG | |
| | TAAAGGCTGTTCTGGCTTTTTATCTTCTTAGCTCATCTTAAATAAGCAGTACACTTGGATG | |
| | CAGTGCGTCTGAAGTGCTAATCAGTTGTAACAATAGCACAAATCGAACTTAGGATTTGTTT | |
| | CTTCTCTTCTGTGTTTCGATTTTTGATCAATTCTTTAATTTTGGAAGCCTATAATACAGTT | |
| | TTCTATTCTTGGAGATAAAAATTAAATGGATCACTGATATTTTAGTCATTCTGCTTCTCAT | |
| | CTAAATATTTCCATATTCTGTATTAGGAGAAAATTACCCTCCCAGCACCAGCCCCCCTCTC | |
| | AAACCCCCAACCCAAAACCAAGCATTTTGGAATGAGTCTCCTTTAGTTTCAGAGTGTGGAT | |
| | TGTATAACCCATATACTCTTCGATGTACTTGTTTGGTTTGGTATTAATTTGACTGTGCATG | |
| | ACAGCGGCAATCTTTTCTTTGGTCAAAGTTTTCTGTTTATTTTGCTTGTCATATTCGATGT | |
| | ACTTTAAGGTGTCTTTATGAAGTTTGCTATTCTGGCAATAAACTTTTAGACTTTTGAAGTG | |
| | TTTGTGTTTTAATTTAATATGTTTATAAGCATGTATAAACATTTAGCATATTTTTATCATA | |
| | GGTCTAAAAATATTTGTTTACTAAATACCTGTGAAGAAATACCATTAAAAAACTATTTGGT | |
| | TCTGAATTCTTACTAGAAGGTGGTCTTTTGAAGTTAGTCCTTTCGGTACTTCTCAGATGCC | |
| | TGTCATGTACCCGATGGAGTCCTTGGAAAGAAGGCCTGTGTAAAGAGGCCAGCCTGGAGGT | |
| | CAATAACCTGTTCTAGTTTATTCTGGACATTGAGTACCAAGTAGCATTGGCAAA | |
| TJP1 | AGCCTTGGCAGTCGGCGCCGGTGAACGAGAGCAACGCTTCTGACCCTGCCGGAGCTCCTCG | 52 |
| | GAGATGAAAGCCATGACGCGCCTTGCAGAAAATGCATTCCGCCTTCCGTGGGAACAACGCC | |
| | GAGGCACGCGGTGACAGCCGTGACCATGCTGTTTGCCCAGTGAAGGAAACAACTGTCGGGT | |
| | ATCGGCTCTGCCGGCCTTTCCAGCCGCACTCATGCATGGGGCTCACCCCATGATGTGCGTG | |
| | GCTTGTCGAGGAGCAAGTGGACAAGTCTCTTAAGGAAAGCTTTGGTGCACAGGCGCTTTCT | |
| | CCTTGGGGGCGAATTCTGCCAGACCTTGGATAAAAACAAACAGGAAGACTCGCACGGCAGC | |
| | GGAAACTGTCTTCCAAGTTACTTGGGTTACCCGGCTTTTCCTTCCGCGCTTGGGGTCGGGA | |
| | CCCCGGCCGCTCGTCCCGCCCCCTCCCCCGCCGCGGCCCCGCCCCCTCCCCGCCTCGCCTC | |
| | GCCTCGCCTCGTCCAGCCCCGCCCCCGCCGGGCCGGGCATGCTCAGTGGGCCGGGCCGGCA | |
| | GGTTTGCGTGGCCGCTGAGTTGCCGGCGCCGGCTGAGCCAGCGGACGCCGCGTTCCTTGGC | |
| | GGCCGCCGGTTCCCGGGAAGTTACGTGGCGAAGCCGGCTTCCGAGGAGACGCCGGGAGGCC | |
| | ACGGGTGCTGCTGACGGGCGGGCGACCGGGCGAGGCCGACGTGGCCGGGCTGCGAAAGCTG | |
| | CGGGAGGCCGAGTGGGTGGCCGCGCTCGGAGGGAGGTGCCGGTCGGGCGCGCCCCGTGGAG | |
| | AAGACCCGGGCGGGGCGGGCGCTTCCCGGACTTTTGTCCGAGTTGAATTCCCTCCCCCTGG | |
| | GCCGGGCCCTTCCGGCCGCCCCCGCCCGTGCCCCGCTCGCTCTCGGGAGATGTTTATTTGG | |
| | GCTGTGGCGTGAGGAGCGGGCGGGCCAGCGCCGCGGAGTTTCGGGTCCGAGGAGTTGCGCG | |
| | CGGCGCTGGAGAGAGACAAGATGGTGAGCAAGGGCGAGGAGCTGTTCACCGGGGTGGTGCC | |
| | CATCCTGGTCGAGCTGGACGGCGACGTAAACGGCCACAAGTTCAGCGTGTCCGGCGAGGGC | |
| | GAGGGCGATGCCACCTACGGCAAGCTGACCCTGAAGTTCATCTGCACCACCGGCAAGCTGC | |
| | CCGTGCCCTGGCCCACCCTCGTGACCACCCTGACCTACGGCGTGCAGTGCTTCAGCCGCTA | |
| | CCCCGACCACATGAAGCAGCACGACTTCTTCAAGTCCGCCATGCCCGAAGGCTACGTCCAG | |
| | GAGCGCACCATCTTCTTCAAGGACGACGGCAACTACAAGACCCGCGCCGAGGTGAAGTTCG | |
| | AGGGCGACACCCTGGTGAACCGCATCGAGCTGAAGGGCATCGACTTCAAGGAGGACGGCAA | |
| | CATCCTGGGGCACAAGCTGGAGTACAACTACAACAGCCACAACGTCTATATCATGGCCGAC | |
| | AAGCAGAAGAACGGCATCAAGGTGAACTTCAAGATCCGCCACAACATCGAGGACGGCAGCG | |
| | TGCAGCTCGCCGACCACTACCAGCAGAACACCCCCATCGGCGACGGCCCCGTGCTGCTGCC | |
| | CGACAACCACTACCTGAGCACCCAGTCCAAGCTGAGCAAAGACCCCAACGAGAAGCGCGAT | |
| | CACATGGTCCTGCTGGAGTTCGTGACCGCCGCCGGGATCACTCTCGGCATGGACGAGCTGT | |
| | ACAAGAGCGGCCTGAGAAGCAGAGCCCTGGAGAGAGACAAGAGTGCCAGAGCTGCGGCCGC | |
| | AAAGGTGAGAACCTCCGCGGCCGCCAGGGCCAGACCGGGCCGACCGTCGCCGCCCGCCCAC | |
| | CGGCATCTGGCCCGCGTCCCGCCCTCCCTCGCTGGCGGCTGTCTGGGCCCCGGGGCGGCGG | |
| | GGTGGGCAGGGCTGGCGCGGGGCCGCGGGCCGCGGGCCGCGGGCCGCGGGGAGCCCCTCGG | |
| | GCGGGGGCGGCGCGGGCCGCACTGGGGGCGGCCGGGGAGGGGGCTGCGGGCGCCCGGCCGC | |
| | CGTACTGGGCAGGTGCATAGCTGCCGGCGCCTGTGCCTGGCTGCGGCTCGCTGAGGGCGGG | |
| | GACACGCAACAGGTCCCTCGCGGAGAAACTCGGCTCCAGTGAGGGTTCGGGGGCTGGAAGC | |
| | CGGCTCTCAGCGGGTCGGGGCTTGGGGTGCCACCTCCTGCTGGCCGGGAGCTGCTGTCTTT | |
| | GGAGGAGTGGTTGGTCCCCGGCGAAACCCTGTAGTTTCGATCTGATGTCACTCCCTGCGGT | |
| | ATGCGCACGCCAGCGATAAGGCTTTGAGACTGCAAAACACTCCACTCAGCCTGTGAGGCGT | |
| | AGTAGGTCGGGTTTTCTTTCATGCTGTATTACTTATTTAAGGTAACTTTGAAAATAACCTC | |
| | TTTAACATTTAATAATTTAACTTGAATTAAACTTTCACAAGTAATACAAAGTATTCCTACG | |
| | AATGGACAATAAGATGAGCACTTAAAAATTAGTAAAGGCCGGTGAGTTCAGCCGAAAAAAG | |
| | TAACGTTTTTCCTGTTACTTTTCCTATGTGCTCTGAAATATTATTGCATTTTCCCATTGCT | |
| | TTGAAACTAACTTGTGTATTACATTAAAAAGCCAAAGTTCCTGAAAAACAGCTAGGATGCT | |
| | CCTCCCATTTTGTATATTAATTTTTTCATCATAAAATAGTACTTGTTATTTCAAACAAAGG | |
| | AATACAGAAATGTGAGGAGTAAAAAATCTCCCCTTTAAAGAATATCAATTCATTACTTCAA | |
| | ATAGT | |
| MYH10 | AAGTGATGGAGCTTCCTCTGCTAGCCCTTTGTGAGCCAATGGTAAATGGGTGCTAAATAAA | 53 |
| | ACAACTAGGTCTTGAGATACATTAATTGTAAATGTCACAGAACCAGTACTTTCCTCAATGT | |
| | GGCTAAGATAGTTGATGGTTCCTTTTTCTTCTGCACTGGTCAGACCATATCTGGGCTATGA | |
| | TGTTTGCTTCTGGGCCACACACTTTAGAGGGAAGACAAGCAGCATGTTGGAGTCTGTTTAG | |
| | GCGAGAGATCCGGCAGGGGAAGGAGTCTTGGTGAATGAGGGGTGGAGGAGCTGCAGGATGG | |
| | GAATAGAGGCCTGAACTGCTACCATGACATATTCAAAAGGCTGCCGTGTGAAGCCAAGTTA | |
| | TGCTTGTCTTTTGTGGTCCCAGTTGATCACATTAAGACCTCATGGGGCCATAGAAAAGCTA | |
| | GAGGGAGACTGATTTGGGTTATTCATAAGAAGAACTTTAAGTCTGTTATCTGAGGGTAGAA | |
| | TGAGAGGCATGTTTTAGATTCTTTAGATTCTTTACTCTTCTGACAATCATGTGTTTTGGTA | |
| | GCTGTTTCCTTGTGGTCATATTAATTCTGGTACCACTTCATGAACCTTTTATTACCCATCT | |
| | TTGTTTTCTTTTTTTTTTTCCCTTCTTAACTCCCTGTTTAATTTGTGGTGAGGGTGAAAGA | |
| | GGAGATAAAGAAAAAAAAGGGTCAACTTGTAACTTTGCCTTTTCTTTTCTTTTCTTTTCTT | |
| | TTTTTTGCCCTCAGTAACTGAGGGCAAACCCATCAGACAACCAGAGCCATAATTTGTGGTC | |
| | ACCGCTGAAATTTACCTTGGAAACTCGGTTAGTATGGCTGTGAAGAGGTATACCCCAGCTC | |
| | CTTAACACAGAGTTAATGCTTAATCTAAGGTTTTAAGTTTCTTAGAAAAGAAAAACGTGTA | |
| | CATTCTTTTGTTTCTTAAACATCTAATTTTGCCCTCCTCCTCTTCTCTTAGAGGCAATTGC | |
| | TTTTGGATCGTTCCATTTACAATGGTGAGCAAGGGCGAGGAGCTGTTCACCGGGGTGGTGC | |
| | CCATCCTGGTCGAGCTGGACGGCGACGTAAACGGCCACAAGTTCAGCGTGTCCGGCGAGGG | |
| | CGAGGGCGATGCCACCTACGGCAAGCTGACCCTGAAGTTCATCTGCACCACCGGCAAGCTG | |
| | CCCGTGCCCTGGCCCACCCTCGTGACCACCCTGACCTACGGCGTGCAGTGCTTCAGCCGCT | |
| | ACCCCGACCACATGAAGCAGCACGACTTCTTCAAGTCCGCCATGCCCGAAGGCTACGTCCA | |
| | GGAGCGCACCATCTTCTTCAAGGACGACGGCAACTACAAGACCCGCGCCGAGGTGAAGTTC | |
| | GAGGGCGACACCCTGGTGAACCGCATCGAGCTGAAGGGCATCGACTTCAAGGAGGACGGCA | |
| | ACATCCTGGGGCACAAGCTGGAGTACAACTACAACAGCCACAACGTCTATATCATGGCCGA | |
| | CAAGCAGAAGAACGGCATCAAGGTGAACTTCAAGATCCGCCACAACATCGAGGACGGCAGC | |
| | GTGCAGCTCGCCGACCACTACCAGCAGAACACCCCCATCGGCGACGGCCCCGTGCTGCTGC | |
| | CCGACAACCACTACCTGAGCACCCAGTCCAAGCTGAGCAAAGACCCCAACGAGAAGCGCGA | |
| | TCACATGGTCCTGCTGGAGTTCGTGACCGCCGCCGGGATCACTCTCGGCATGGACGAGCTG | |
| | TACAAGTACAGCGATCTGGAGCTGAAGCTGCGGATCCCTGCACAAAGGACAGGGCTGGAGG | |
| | ATCCAGAGAGGTACCTGTTCGTCGATCGTGCTGTCATCTACAACCCTGCCACTCAAGCTGA | |
| | TTGGACAGCTAAAAAGCTAGTGTGGATTCCATCAGAACGCCATGGTTTTGAGGCAGCTAGT | |
| | ATCAAAGAAGAACGGGGAGATGAAGTTATGGTGGAGTTGGCAGAGAATGGAAAGAAAGCAA | |
| | TGGTCAACAAAGATGATATTCAGAAGATGAACCCACCTAAGTTTTCCAAGGTGGAGGATAT | |
| | GGCAGAATTGACATGCTTGAATGAAGCTTCCGTTTTACATAATCTGAAGGATCGCTACTAT | |
| | TCAGGACTAATCTATGTAAGTATTTCTTCCAAATAATCATGTGAAGTGGTAGCTAGGAATT | |
| | AATGTAAATTATACATCTTGTCATAATCAAATGAGAATGTGGAATACCCAAACTCTCTGTT | |
| | TAACATTTCTATTTCTCTTTAAGATAGAAAGATTTGTTGCTTGCTTACCCATGTCTTGCTT | |
| | TTCTTTGAATCTTAACACATTAAGTTTAAATAATACAGGCTGCAATTACATATAATAAAAT | |
| | GGCATTTGAAGACTTTTGTAGTGGTCTTCTGGAGCATAATAAGGTGGGAGAGAGCATGTAA | |
| | CAGGAAGACCAGAAGGTTTAATAAGGTAAAGAGAGTTGCATTAATTGGACGCAGACAGCAA | |
| | AACGGTCAAAAATCAAGTGCATACCCAAGAGTAAAGTGGAGGGGCTGTAAGCTGAGAAATT | |
| | TCTGTGGACAGCATGAACAGCTTCACTGGATGTAGTAGGGAAGTAGGAAAGATGAATGCTG | |
| | AGGTTTTTAAGAGGAAACAATTAGGGTAAGATTGAGGCTGGCTGGGGTCGTCCTGTGGTTA | |
| | GCAGCTGACATGAATGTTGGAGTCACCGACTTTGTCACTGACCATGTAGAAGAAGTTATTG | |
| | AAAATCATAAGGGATAATGTAGAGAGGGATAATGTAGAGAGGAAAAATGTAAGCCAGATAC | |
| | TA | |

### CRISPR/Cas9 RNP System

Wild type (WT) S. pyogenes Cas9 (spCas9) protein was purchased from UC Berkeley QB3 Macrolab and was pre-complexed in vitro with synthetic CRISPR RNA (crRNA) and trans-activating crRNA (tracrRNA) duplexes to generate a CRISPR/Cas9 ribonucleoprotein (crRNP). Briefly, the crRNA and tracrRNA oligonucleotides were reconstituted to 100 μM in TE at pH 7.5 (catalog #11-01-02-02, IDT). The crRNA and tracrRNA oligonucleotides were then combined in a sterile PCR at a final concentration of 40 μM in Duplex Buffer (100 mM potassium acetate; 30 mM HEPES, pH 7.5). Using a thermocycler or heat block, the crRNA and tracrRNA mixture was heated to 95° C. for 5 min to generate a crRNA:tracrRNA duplex. After heating, the crRNA:tracrRNA duplex was allowed to cool at room temperature for a minimum of two hours, after which the crRNA:tracrRNA duplex was kept on ice. crRNA:tracrRNA duplexes were then diluted to a working concentration of 10 μM in TE. All dilutions and stocks were kept on ice throughout the protocol. Alternatively, the crRNA:tracrRNA duplexes were stored at −20° C. for later use.

spCas9 was stored at −80C and was thawed on ice or at 4° C. until no ice pellet was visible, approximately 2-5 min. spCas9 was then diluted to a working concentration of 10 μM in TE in preparation for use. Alternatively, working concentrations of Cas9 protein were stored at −20° C. for up to 2 weeks and multiple freeze-thaw cycles were avoided (<3 freeze-thaw cycles recommended).

crRNPs were generated by combining the solution of crRNA:tracrRNA duplexes and Cas9 protein in a 1.5 mL eppendorf tube and gently pipetting up and down three times. A separate crRNP was generated for each reaction to be performed. crRNPs were incubated a room temperature for a minimum of 10 minutes and no longer than 1 hour prior to the addition of the complexes to cells.

### Cell Culture and Transfection

WTC iPSCs were cultured according to described methods. Briefly, WTC11 iPSCs were cultured in a feeder free system on tissue culture plates or dishes coated with pheno red-free GFR Matrigel (Corning) diluted 1:30 in DMEM/F12 (Gibco) in mTeSR1 media (StemCell Technologies) supplemented with 1% (v/v) Penicillin-streptomycin (P/S) (Gibco). Cells were not allowed to reach confluency greater than 85% and were passaged every 3-4 days by dissociation into single-cell suspension using StemPro® Accutase® (Gibco). When in single cell suspension, cells were counted using a Vi-CELL® Series Cell Viability Analyzer (Beckman Coulter). After splitting, cells were re-plated in mTeSR1 supplemented with 1% P/S and 10 μM ROCK inhibitor (RI) (Stemolecule Y-27632, Stemgent) for 24 h. Expired media was replenished with fresh mTeSR1 media supplemented with 1% P/S daily at 37° C. and 5% CO₂.

Prior to transfection, mTeSR1 media (400 mL basal media with provided 100 mL 5× supplement (catalog #05850, Stem Cell Technologies) with added 5 mL (1% v/v) Penicillin/Streptomycin (catalog #15140-122, Gibco) was prepared and sterile filtered with a 0.22 μm filter prior to use. mTeSR1 media was brought to room temperature on the bench top, and was not warmed in a 37° C. water bath. mTeSR1+ROCK inhibitor (Ri) media was prepared by adding 10 mM Ri to mTeSR1 media at a 1:1000 dilution. Accutase was warmed in a 37° C. water bath. Previously prepared Matrigel-coated vessels (stored at 4′C) were brought to room temperature. 6-well plates were prepared by aspirating and discarding any excess Matrigel liquid, and adding 4 mL of RT mTeSR1+Ri media to each well. Plates with media were kept in an incubator at 37° C. and 5% CO₂ until ready to plate cells after the transfection procedure.

Cells were aliquoted in mTeSR+Ri into separate 1.5 mL eppendorf tubes. Cells were pelleted by centrifuging in a micro-centrifuge at 211×g for 3 min at room temperature. Various delivery methods including CrisprMax, GeneJuice, Amaxa and Neon were evaluated before concluding that Neon electroporation resulted in favorable co-introduction of protein, RNA, and plasmid into hiPSCs as measured by transfection of a control reporter plasmid and T7 assays as a read out for Cas9 activity (data not shown). Supernatant was aspirated and discarded and cells were resuspended in Buffer R from the Neon Transfection Kit. 8×10⁵ cells were resuspended in 100 μL Neon Buffer R with 2 μg donor plasmid, 2 μg Cas9 protein duplexed with a crRNA:tracrRNA at a 1:1 molar ratio to Cas9, then electroporated with one pulse at 1300 V for 30 ms, and plated onto Matrigel-coated 6-well dishes with mTeSR1 media supplemented with 1% P/S and 10 μM RI. Transfected cells were cultured as previously described for 3-4 days until the transfected culture had recovered to ˜70% confluent. Transfected cells were incubated for at least 24 hours before changing the media to mTeSR1 without Ri. Successfully transfected cells were identified and harvested by FACS sorting for use in downstream applications after reaching a healthy confluency and maturity (approximately 3-4 days) (FIG. 1C).

### Example 2—Generating Clonal Lines of GFP-Tagged hiPSCs

### Enrichment of Gene-Edited Cells

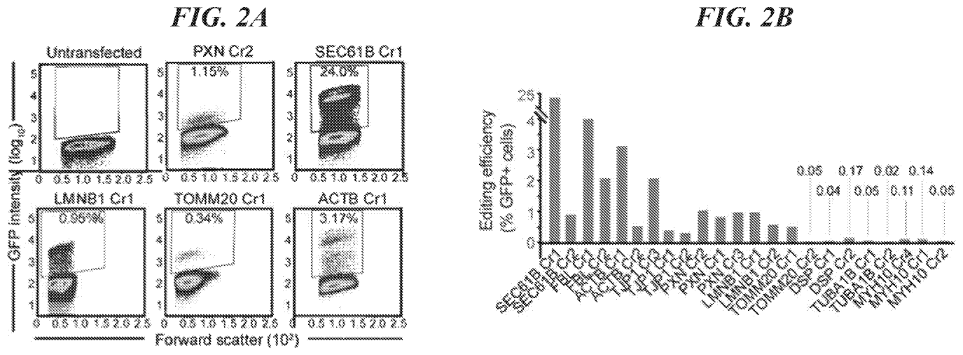

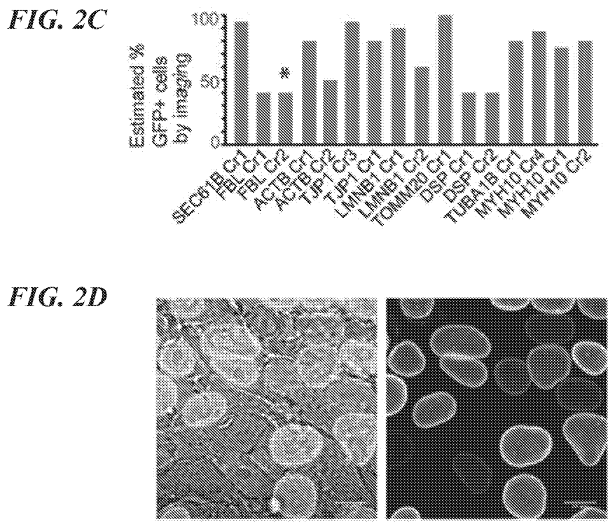

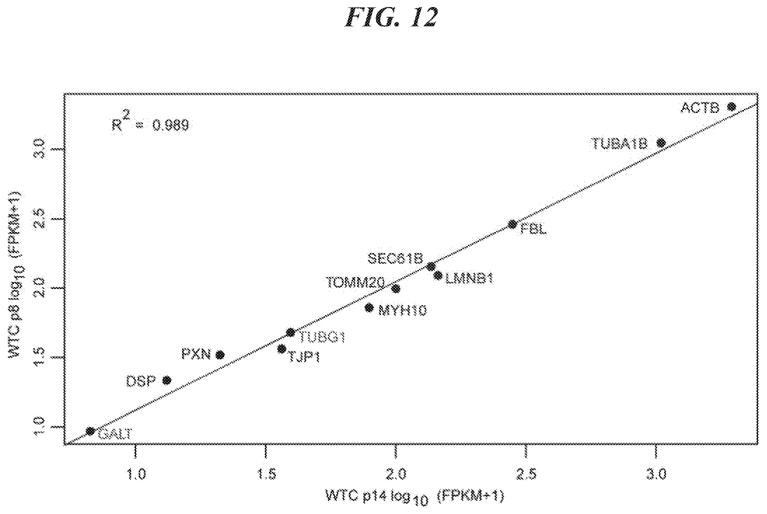

Fluorescence-activated cell sorting (FACS) was used to enrich the population of gene edited cells after transfection and to evaluate rates of HDR (FIG. 2A). The cell suspension (0.5-1.0×10⁶ cells/mL in mTeSR1+RI) was filtered through a 40 μM mesh filter into polypropylene round bottom tube. As expected for tagging experiments targeting diverse cellular proteins, a range of GFP fluorescent intensity was observed in edited populations (FIG. 2A and FIG. 2B). The GFP intensity determined by FACS correlated with transcription levels of the target protein observed by RNAseq analysis from the WTC parental cell line (RNA-seq analysis shown in FIG. 12). The percentage of GFP+ cells above the background defined by untransfected, unedited cells was used as a measure of HDR-mediated knock-in efficiency (FIG. 2B). Successful GFP-tagging was observed with at least one crRNA in 10 of the 12 target loci even when HDR was inefficient (<1%). Of the successful edits, editing efficiency was variable across the genomic loci with the majority of the experiments yielding <0.1%-4% GFP+ cells. Sec61b was a notable exception, wherein 20% of the treated cells were GFP+(FIG. 1D). The observed efficiency at each locus was consistent between experiments. These data indicate that HDR efficiency at a given locus depends significantly on the crRNA used, as in several experiments only one crRNA gave rise to a GFP+ population of cells (FIG. 1D).

In all gene targeting experiments, flow-based selection resulted in the recovery and enrichment of GFP-tagged clones, even when HDR was inefficient (<1%). For example, weak GFP signal was observed in some experiments where the target gene transcript was relatively scarce (such as PXN) or where the protein is known to localize to small foci in cells corresponding to cell junctions (DSP) or substrate adhesion sites (PXN). However, enriched populations of cells edited at these loci were able to be obtained, despite the low percentages of GFP+ cells after transfection (FIG. 2A). Experiments were also performed to assess HDR efficiency as a function of variable homology arm lengths in the donor plasmid. Among the three loci tested, there was a range of efficiencies with the standard 1 kb homology arms. However, the 1 kb arms flanking the intended protein tag sequence resulted in the best and most reliable efficiency compared to the shorter (200 bp or 50 bp arms) (data not shown).

After FACS enrichment, approximately >70% of the cells were GFP+ even after a period of recovery and scale up post sorting, indicating that flow cytometry is an efficient method for isolation of GFP+ cells. To ensure the knock-in of GFP to the targeted genomic locus resulted in appropriate localization of the resulting fusion proteins, the cells were analyzed by live fluorescence imaging prior to generating clonal lines. Each population displayed localization of the GFP signal to the anticipated cellular structure (FIG. 2C). FIG. 2D shows a representative image of the LMNB1 Cr1 FACS-enriched population showing an enrichment of GFP+ cells.

Clonal cell lines generated from these edited, enriched cell populations were then generated to identify and isolate precisely edited cells. Briefly, cells from the FACS-enriched population were seeded at a density of 10⁴ cells in a 10 cm Matrigel-coated tissue culture plate. After 5-7 days clones were manually picked with a pipette and transferred into individual wells of 96-well Matrigel-coated tissue culture plates and expanded clonally. Greater than 90% of these clones survived colony picking. After 3-4 days, colonies were dispersed with Accutase and transferred into a fresh 96-well plate. After recovery, the plate was divided into plates for ongoing culture or freezing and gDNA isolation. When cells were 60-85% confluent they were dissociated and pelleted in 96-well V-bottom plates for cryopreservation. Cells were then resuspended in 60 μL mTeSR1 supplemented with 1% P/S and 10 μM RI. Two sister plates were frozen using 30 μL cell suspension per plate, added to 170 μL CryoStor® CS10 (StemCell Technologies) in non-Matrigel coated 96-well tissue culture plates. Plates were sealed with Parafilm and introduced to the −80C freezer in a room temperature Styrofoam box. Plates were stored long term at −80° C. for up to 8 weeks before thawing. Few clones (<5% across experiments) spontaneously differentiated after isolation, splitting, and freezing and a majority of clones were able to be scaled up for genetic and quality control experiments. A schematic of the overall selection and quality control process is shown in FIG. 1D.

### Example 3—Genetic Screening of Edited Clones

Genetic screening analyses were performed in order to identify clones in which GFP tagging was performed precisely, without damage to endogenous untagged alleles (if present) and without permanent incorporation of the plasmid donor backbone into the genome. A genetic screening strategy was used to rapidly discriminate between precisely and imprecisely edited clones. Criteria for precise editing were as follows:

(a) Incorporation of the GFP tag in-frame with the targeted exon;

(b) The absence of random or on-target donor plasmid backbone integration; and

(c) No unintended mutations in either allele.

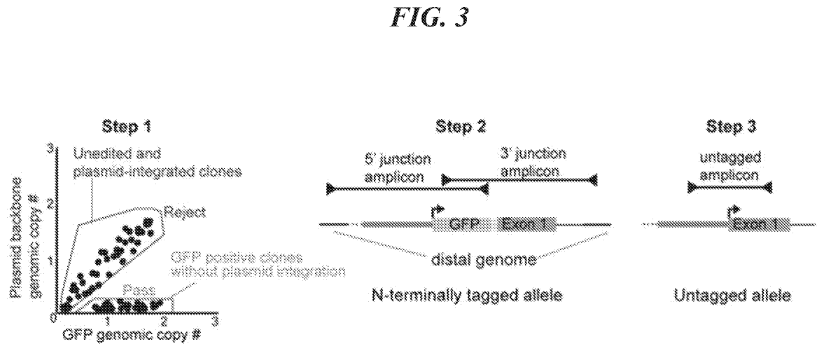

An overview of the genetic screening process is shown in FIG. 3A-FIG. 3C, including digital droplet PCR (ddPCR. FIG. 3A), tiled junctional PCR assays (FIG. 3B), and sequencing analysis of inserted amplicons (FIG. 3C).

Digital Droplet PCR (ddPCR)

Because primers and probes for GFP, the donor plasmid backbone, and the RPP30 reference gene could be used to analyze all gene edits, a droplet digital PCR (ddPCR) assay was used to rapidly interrogate large sets of clones in parallel without having to optimize parameters specifically for each target gene, a significant advantage for our high throughput platform. During clonal expansion, a sample of cells was pelleted and total gDNA was extracted using the PureLink Pro 96 Genomic DNA Purification Kit (Life Technologies). ddPCR was performed using the Bio-Rad QX200 Droplet Reader, Droplet Generator, and QuantaSoft software.

Assays were designed to measure three DNA sequences common to each experiment: (1) the GFP tag sequence to measure tag incorporation; (2) the ampicillin or kanamycin resistance gene to assess stable integration of the plasmid backbone; and (3) a two-copy genomic reference locus (RPP30) to calculate genomic copy number. These sequences were used to identify clones with a GFP:RPP30 signature of ˜0.5 or ˜1.0, suggesting monoallelic or biallelic stable integration of the GFP sequence into the host cell genome. Clones with an elevated AmpR/KanR:RPP30 ddPCR signature (>0.1) suggested stable integration of the donor plasmid backbone and were rejected.

First, GFP-tagged clones lacking plasmid backbone integration were identified using ddPCR, with equivalently amplifying primer sets and probes corresponding both to the GFP tag and the donor plasmid backbone. The abundance of the GFP tag sequence was quantified (x-axis in FIG. 3A) and normalized to a known 2-copy genomic reference gene (RPP30) in order to calculate genomic GFP copy number in the sample. The reference assay for the 2-copy, autosomal gene RPP30 was purchased from Bio-Rad. The assay for mEGFP detection was as follows:

a Primers:
(i)
(SEQ ID NO: 187)
5′-GCCGACAAGCAGAAGAACG-3′
(ii)
(SEQ ID NO: 188)
5′-GGGTGTTCTGCTGGTAGTGG-3′
(iii)
(b) Probe:
(SEQ ID NO: 189)
/56-FAM/AGATCCGCC/ZEN/ACAACATCGAGG/3IABkFQ/.

The copy number of a marker sequence in the donor plasmid (AMP or KAN resistance genes) in each clone (y-axis in FIG. 3A) was also calculated. The assay for AMP was as follows:

(a) Primers:
(i)
(SEQ ID NO: 190)
5′-TTTCCGTGTCGCCCTTATTCC-3′
(ii)
(SEQ ID NO: 191)
5′-ATGTTAACCCACTCGTGCACCC-3′
(b) Probe:
(SEQ ID NO: 192)
/5HEX/TGGGTGAGC/ZEN/AAAAACAGGAAGGC/3IABkFQ/

The reported final copy number of mEGFP per genome was calculated as the ratio of [(copies/μL mEGFP)—(copies/μL nonintegrated AMP)]/(copies/μL RPP30), where a ratio of 0.5 indicated monoallelic insertion (˜1 copy per genome) and a ratio of 1 indicated biallelic insertion (˜2 copies/genome). The AMP sequence was used to normalize mEGFP signal only when integration into the genome was ruled out during primary screening. For primary screening [(copies/μLmEGFP)/(copies/μLRPP30) was plotted against [(copies/μLAMP)/(copies/μLRPP30) in order to identify cohorts of clones for ongoing analysis.

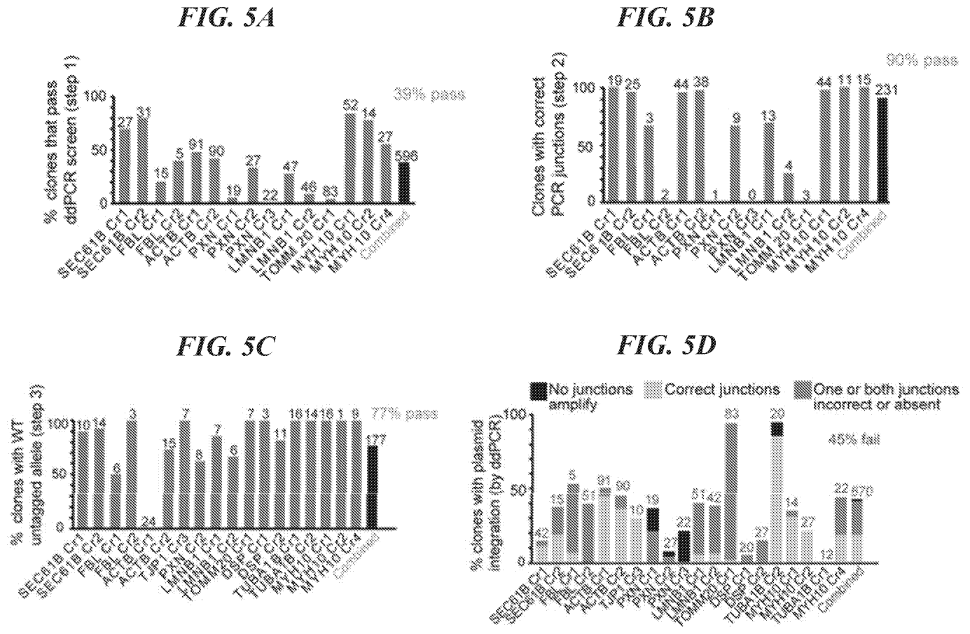

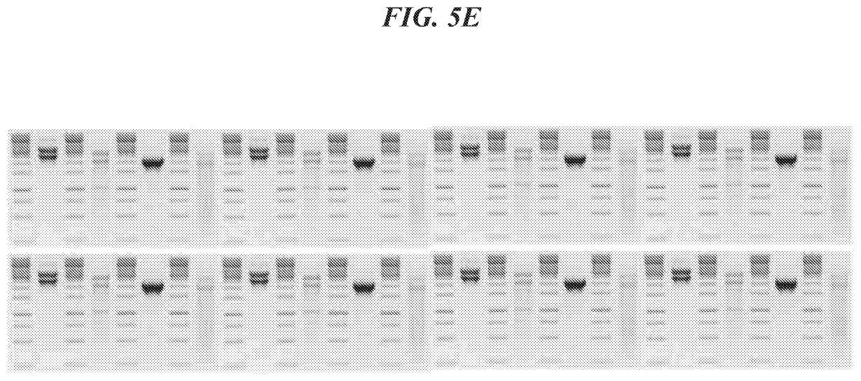

Clones with a GFP copy number of ˜1.0 (monoallelic) or ˜2.0 (biallelic) and AMP/KAN<0.2 were putatively identified as correctly edited clones. Combining data across all successful editing experiments, 39% of clones were retained as candidates using this assay (FIG. 5A). Clones with a GFP copy number 0.2-1 were considered possible mosaics of edited and unedited cells and were rejected. Clones with a GFP copy number between ˜1 and ˜2 were further screened to identify potential biallelic clones from mixed cultures.

The screening strategy also identified several faulty outcomes in the editing and selection process including unedited clones co-purified during flow cytometry selection, and clones harboring plasmid backbone in the targeted locus and enabled selection of successfully edited clones. These results demonstrate that the addition of the ddPCR assay to the genetic screening process enabled selection of successfully edited clones and eliminated unsuccessful or off-target edits from downstream analyses.

### Tiled-Functional PCR

Clones whose ddPCR signature indicated the stable presence of GFP in the genome (GFP:RPP30 values ˜0.5 or 1) and the absence plasmid backbone integration (AmpR/KanR:RPP30<0.1) were further analyzed by tiled-junctional PCR to determine the presence of the predicted tagged alleles and sequences.

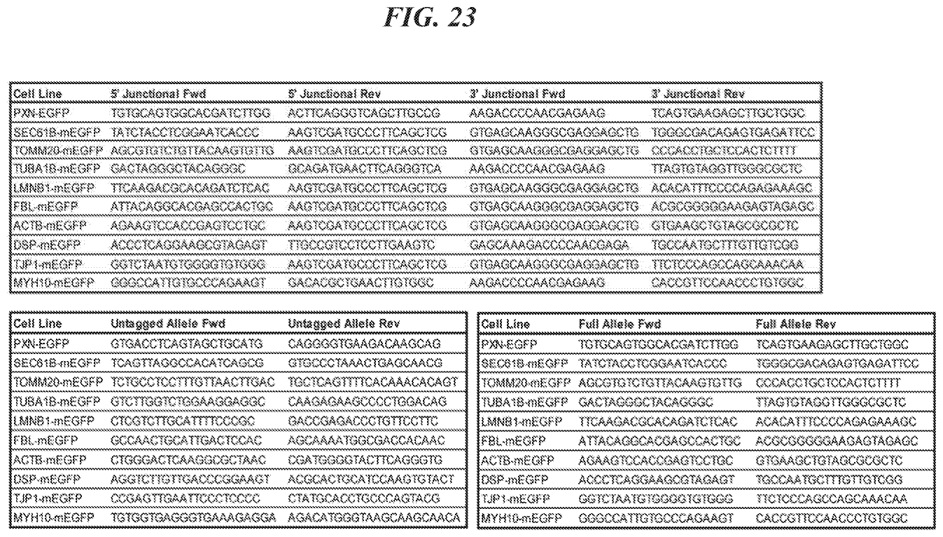

Primer sequences used in each PCR reaction are shown in FIG. 23. All primers are listed in 5′ to 3′ orientation. PCR was used to amplify the tagged allele in two tiled reactions spanning the left and right homology arms, the mEGFP and linker sequence, and portions of the distal genomic region 5′ of the left homology arm and 3′ of the right homology arm using PrimeStar® (Clontech) PCR reagents and gene-specific primers. Both tiled junctional PCR products were Sanger sequenced bidrectionally with PCR primers when their size was validated as correct by gel electrophoresis and/or Fragment Analyzer (FIG. 5E). This enabled confirmation of GFP tag incorporation without large insertions or deletions the tagged allele. 90% (n=231) of the overall clones tested in this assay contained expected junctional PCR products after initial confirmation by ddPCR (FIG. 5B). Furthermore, the majority of the clones rejected based on ddPCR signature (e.g., clones with >0.1 AmpR/KanR RPP30 ratios) also contained inappropriate junctions. Sanger sequencing of the junctional amplicons from a subset of these clones (n=107) confirmed correct sequences in all cases (data not shown).

### Sequencing Analysis of Inserted Amplicons

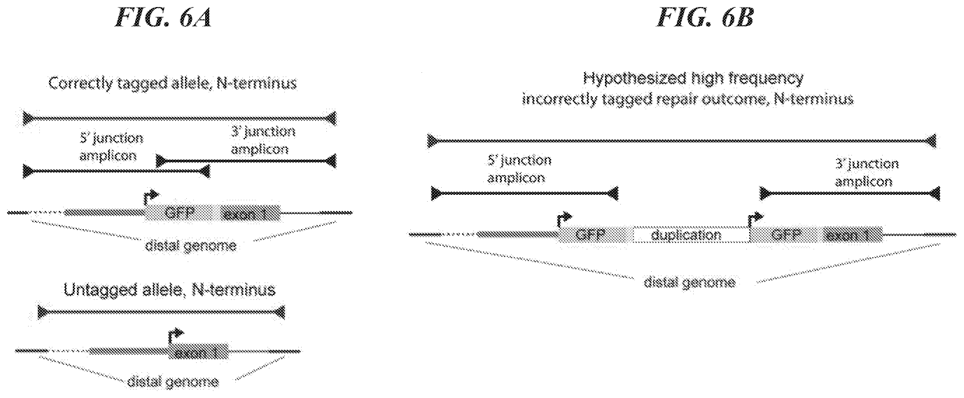

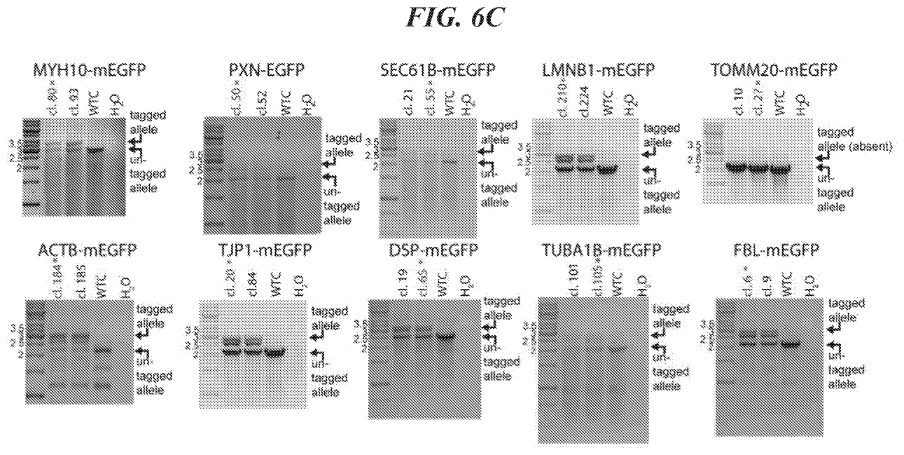

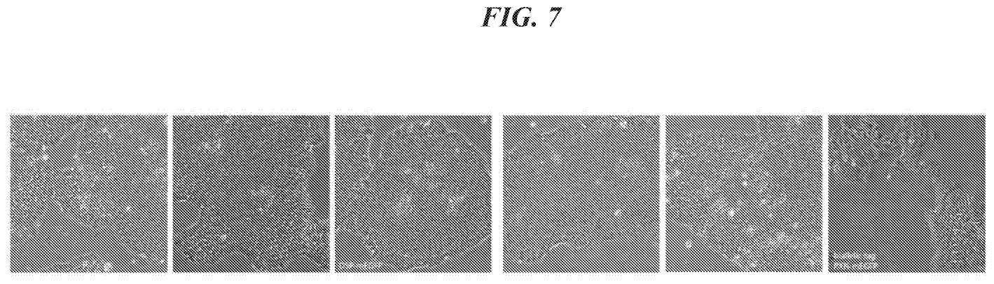

The untagged allele (for monoallelic GFP-tagged clones) was amplified and sequenced to ensure that no mutations had been introduced via the NHEJ repair pathway at the binding site of the crRNA used for editing). 77% (n=177) of the clones analyzed from all experiments contained a wild type untagged allele (FIG. 5C) and a subset of these clones was chosen for further analysis in additional quality control assays. A subset of clones confirmed by ddPCR and junctional PCR from each gene edit were selected and analyzed by Sanger sequencing of the amplicon corresponding to the untagged allele in order to rule out unanticipated mutations at the tagged locus (FIG. 3C). Clones with mutations caused by NHEJ in the untagged allele were rejected. Among clones with correct junctional product sizes, the correct sequence was confirmed in the overwhelming majority of clones (>95%). To rule out the possibility of misleading junctional PCR outcomes in the final clones, such as rearrangements and duplications, a single PCR reaction designed to amplify both the tagged and untagged allele across both homology arm junctions was used (FIG. 6A-FIG. 6B). In 9 out of 10 cases, the presence of the expected products for both the tagged and untagged alleles was confirmed (FIG. 6C).

### CONCLUSIONS

Clones were frequently rejected due to stable integration of plasmid backbone sequence and these rejected clones were further analyzed. In many cases, clones were derived from FACS-enriched populations in which most cells displayed the correct anticipated subcellular GFP tag localization, but nevertheless harbored the GFP tag and donor plasmid backbone at equivalent copy number. It is possible that non-random HDR-mediated incorporation of both the tag and the donor plasmid backbone at the targeted locus result in this pattern. Such an outcome would result in a tagged protein, but also unintended insertions of exogenous sequence into the locus (Rouet et al., 1994; Hockemeyer et al., 2009). This possibility was evaluated by performing the tiled junctional PCR assay (FIG. 3B) on clones rejected by ddPCR due to integrated plasmid backbone, in the same manner as clones putatively confirmed by ddPCR

FIG. 5D shows the percentage of clones in each experiment with KAN/AMP copy number ≥0.2 (y-axis). Stacked bars represent 3 observed subcategories of rejected clones: (i) clones with one correct and one incorrect or missing junctions (interpreted as plasmid backbone integration at the targeted locus); (ii) clones in which no junctions are amplified (interpreted to contain random integration of the donor plasmid); and (iii) clones in which both junctions are correct (interpreted to contain duplications of the GFP tag sequence at the targeted locus). A large majority of clones gave rise to at least one junctional PCR amplicon, suggesting that plasmid integration occurs at the target locus. Clones with no amplified junctions, as expected in the case of donor plasmid integration at random genomic locations, were uncommon (4% of failed clones). Much more frequently (51% of failed clones), junctions from rejected clones failed to amplify or were aberrantly large on one side of the tag but intact on the other side (FIG. 5D). 45% of the plasmid-integrated clones rejected by ddPCR (which were 45% of all clones) had correct junctions on both sides of the tag (FIG. 5D “combined”).

It is possible that these categories of clones harbor insertions and/or duplications derived from the donor cassette sequence delivered by HDR to non-coding regions flanking the GFP tag at the target locus. The prevalence of clones with this flawed editing outcome may underlie heterogeneity in the GFP signal intensity observed in some experiments. However, the ddPCR results largely correlated with the presence or absence of appropriate junctions (FIG. 5B) and validates the use of ddPCR as an efficient screening assay. Although clones deemed acceptable based on ddPCR signature largely overlapped with those with correct tiled PCR junction products (e.g. ZO-1, PXN), suggesting that it may be possible to use this approach as the primary screening method instead of ddPCR, this was not the case. Confirmation of clones with amplification of both junctions does not, on its own, exclude the possibility of incorrect repair at the targeted locus (FIG. 5D).

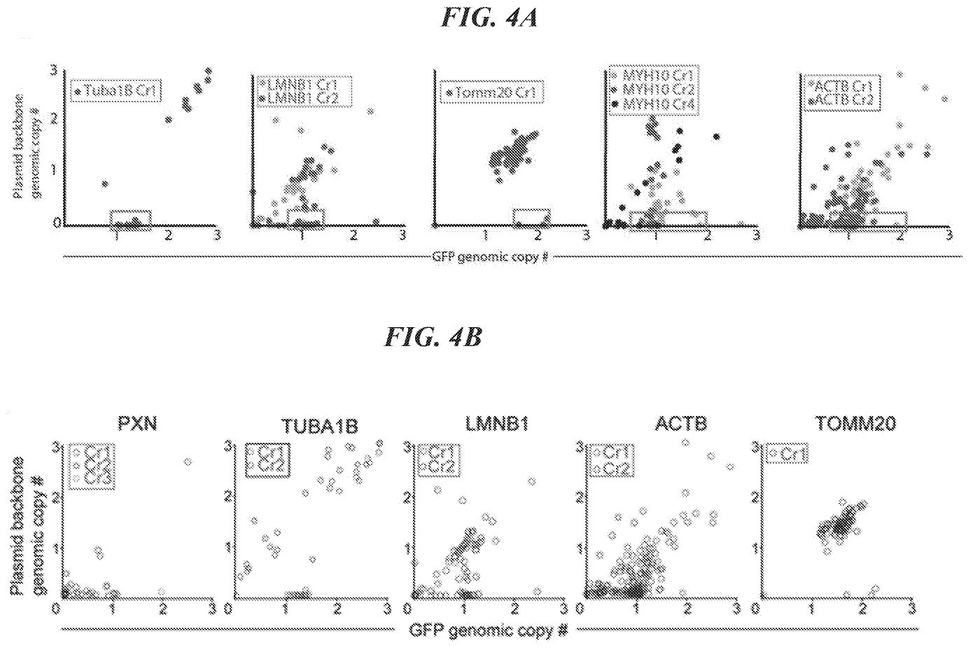

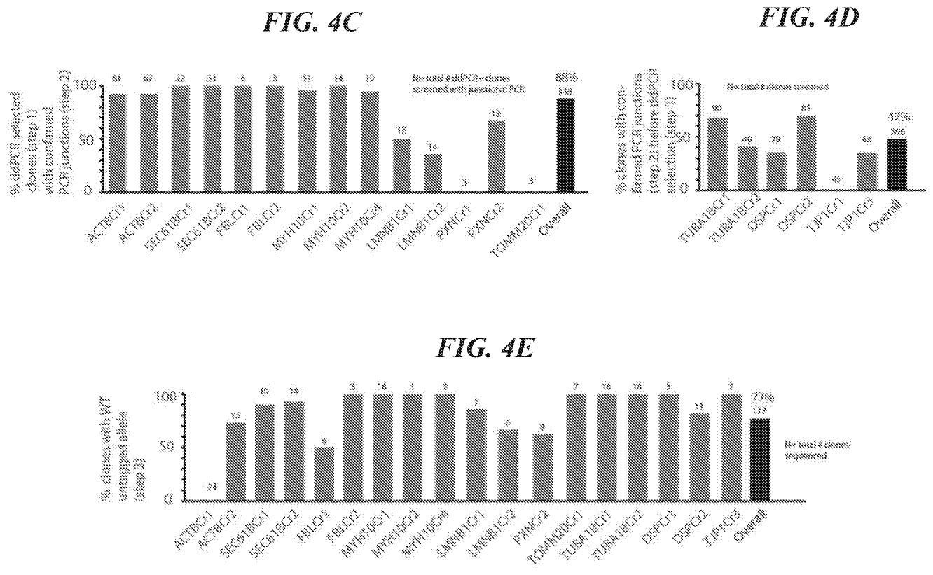

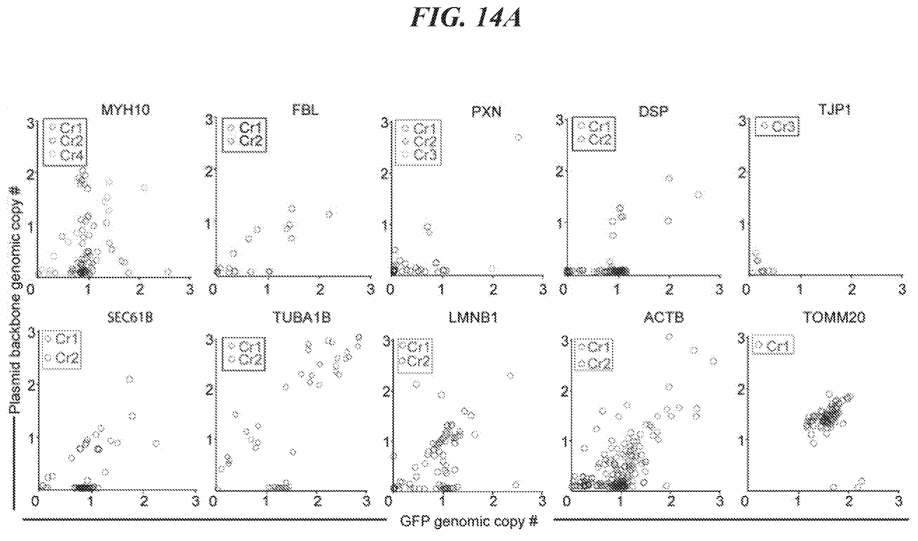

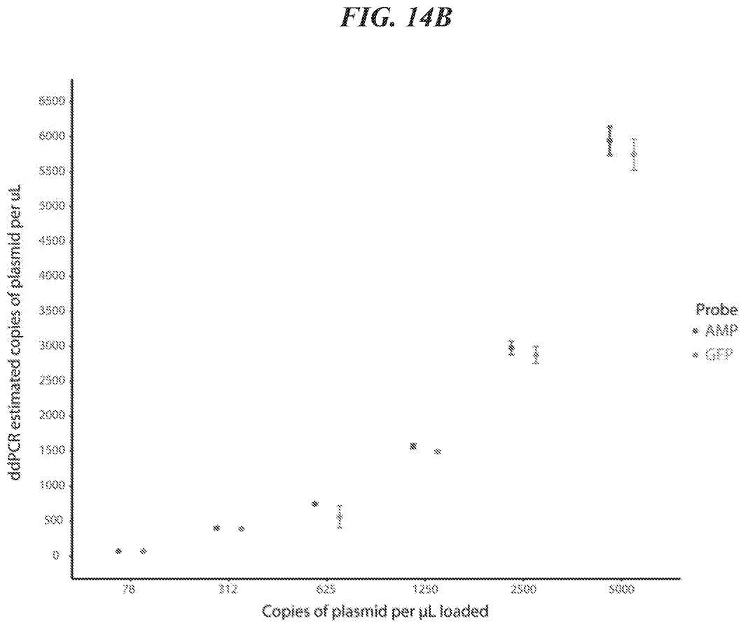

The relative rates of putative clonal confirmation and rejection in this assay varied widely based both on the locus and the crRNA used (FIG. 5A). For example, TOMM20 editing yielded GFP+ cells from only one crRNA (Crl), all of which contained integrated plasmid (80/83) and/or faulty junctions (3/83) (FIG. 4B and FIG. 5A-5B, FIG. 14A, FIG. 6C). In the absence of precise editing at this locus, several TOMM20 clones with evidence of plasmid backbone insertion in the non-coding sequences at the TOMM20 locus were selected for expansion and downstream quality control analysis. The large majority of TUBA1B clones edited with Cr2 contained integrated plasmid, while most clones from Crl were unaffected (FIG. 4B). Similarly, the frequency and type of mutations found in the unedited allele were also target and locus specific, with ACTB Crl a notable outlier case in which NHEJ-mediated mutations in the untagged allele occurred in all analyzed clones (n=24) unlike ACTB Cr2 (FIG. 5C).

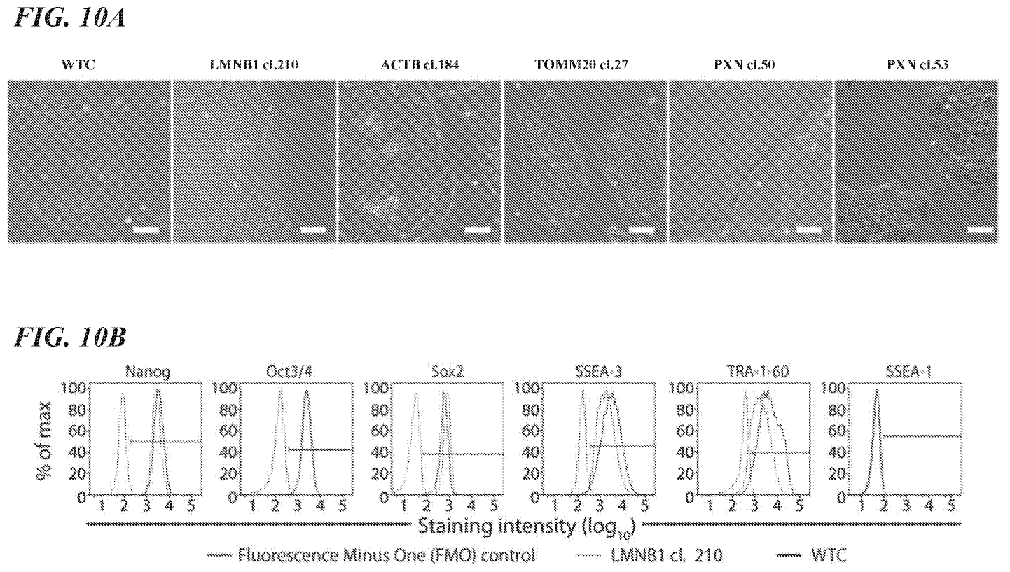

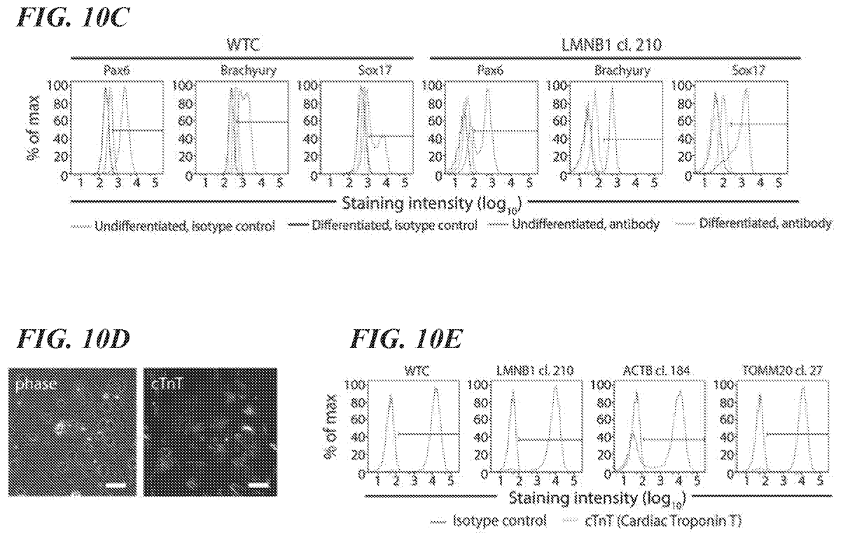

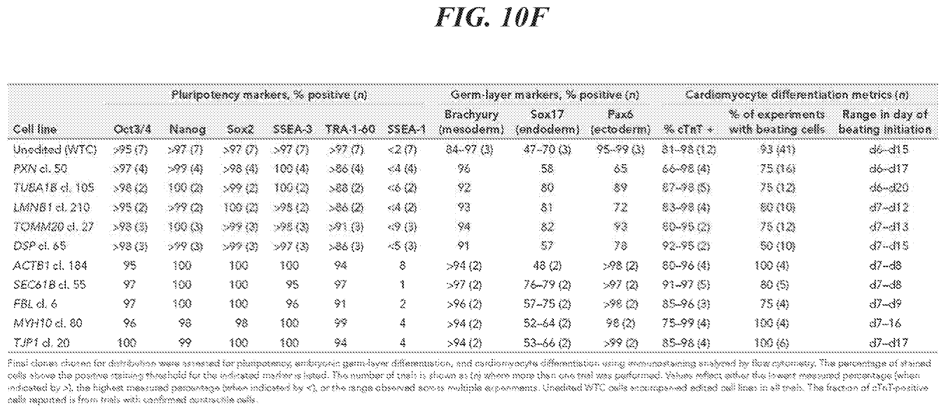

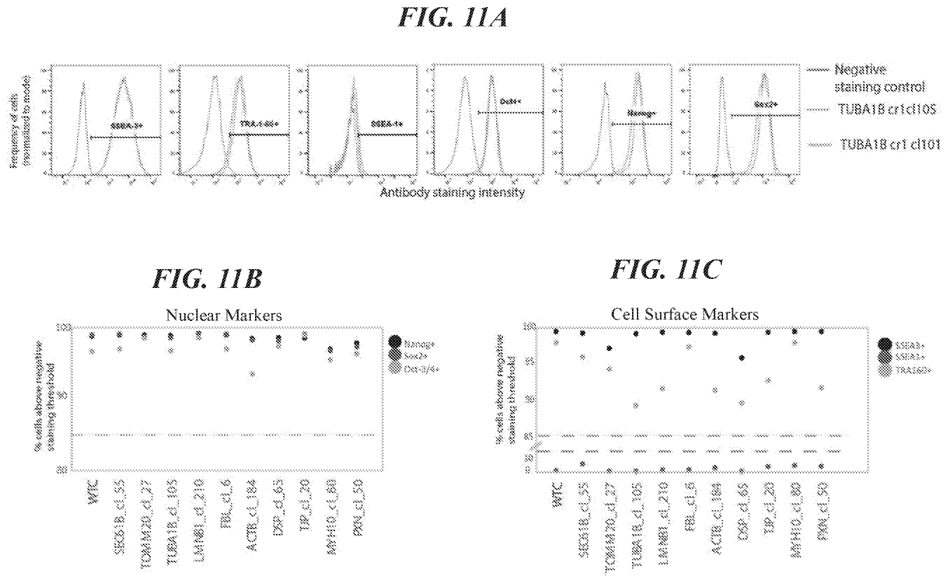

Putatively confirmed clones were almost exclusively tagged at one allele, while clones with putative biallelic edits with no plasmid incorporation were rare (FIG. 4A and FIG. 4B). Clones with ddPCR signatures consistent with biallelic editing (GFP copy number ˜2) were observed at low frequency across all experiments (total n=8) (FIG. 4A, FIG. 14A). Only one clone (PXN Cr2 cl. 53) was confirmed as a biallelic edit with predicted junctional products (data not shown), but was later rejected due to poor morphology (FIG. 10A). Other suspected biallelic clones were rejected due to incorrect junctional products and/or presence of the untagged allele (data not shown) indicating that these clones did not precisely incorporate the GFP tag in both alleles. The frequency of faulty HDR demonstrated by these data underscores the importance of multi-step genomic screening to identify precisely edited clones and confirm monoallelic editing.

Taken together, confirmation rates of 39% (GFP incorporation with no plasmid), 90% (correct junctions), and 77% (wild type untagged allele) were observed in each of the three screening steps across all gene targeting experiments (FIG. 5A-5C). Thus ˜25% of the clones screened in this manner met all three of these precise editing criteria. Donor plasmid integration was the most common category of imprecise editing, affecting 45% of all clones (FIG. 5D). These data suggest that this frequently occurs at the edited locus as a faulty byproduct of the editing process and that screening by junctional PCR alone, without a method to directly detect the plasmid backbone, leads to misidentification of clones with imprecise editing, despite appropriate localization of the tagged protein resulting from the edit (Jasin and Rothstein, 2013; Oceguera-Yanez et al., 2016).

### Example 4—Further Genomic and Proteomic Validation of Candidate Clones

The analyses described above resulted in the identification of a refined set of candidate clones, wherein both tagged and untagged alleles were validated for the correct sequence identity. These candidate clones were further validated in a number of lower throughput downstream assays.

To assess whether the clones that met the above gene editing criteria contained off-target mutations due to non-specific CRISPR/Cas9 activity, several final candidate clones from each experiment were analyzed for mutations at off-target sites predicted by Cas-OFFinder (FIG. 13A) (Bae et al., 2014). Potential off-target sites for each crRNA were prioritized for screening based both on their similarity to the on-target site and their proximity to genic regions. Five sites with the greatest similarity in sequence to the on-target site within the seed region and the protospacer-adjacent motif (PAM) and five sites that were the most similar within genic regions (within 2 kb of an annotated exon) were chosen for analysis. Approximate 200 bp of sequence flanking the predicted off-target site was amplified by PCR and the product was Sanger sequenced. PCR amplification of these regions followed by Sanger sequencing was performed to identify potential mutations in 3-5 final candidate clones for all 10 genome editing experiments (6-12 sequenced sites per clone) across 142 unique sites. Among a total of 406 sequenced loci, no off-target editing events were identified (FIG. 13). Follow-up exome sequencing of the final clones confirmed the absence of any mutations at predicted genic sites captured at adequate depth (data not shown). However, during this exercise, SNPs were identified that were subsequently confirmed to be present in the WTC parental cell line, indicating the ability of this method to uncover alternative alleles.

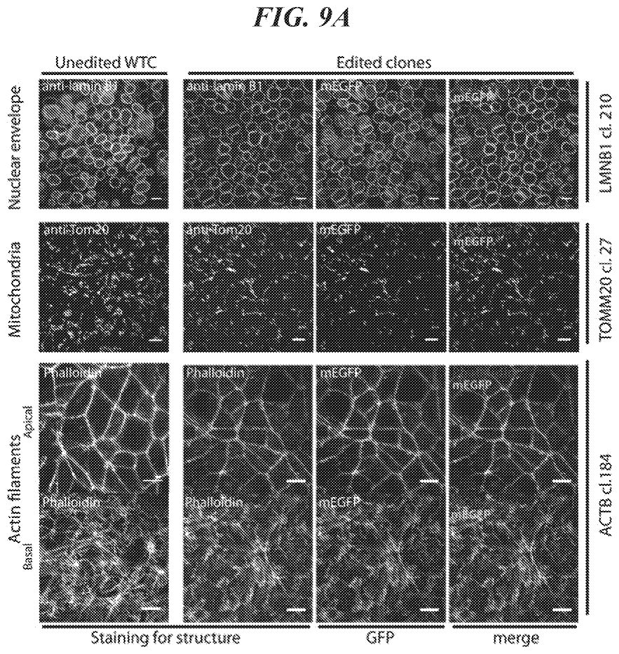

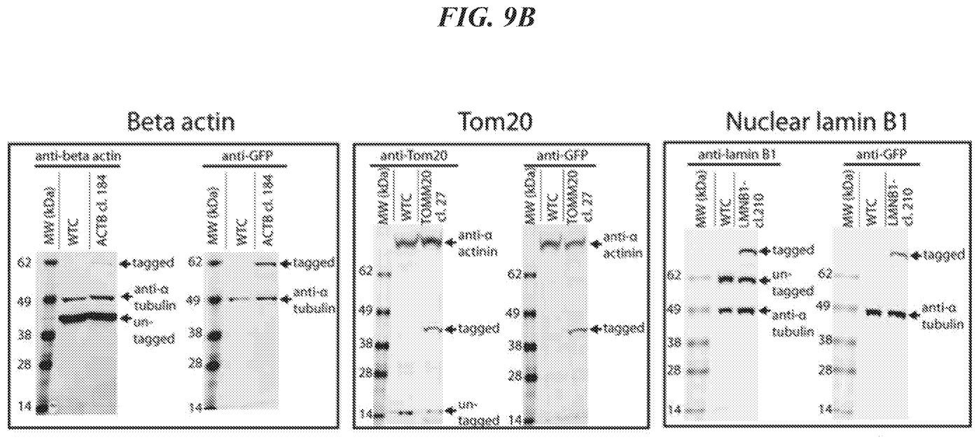

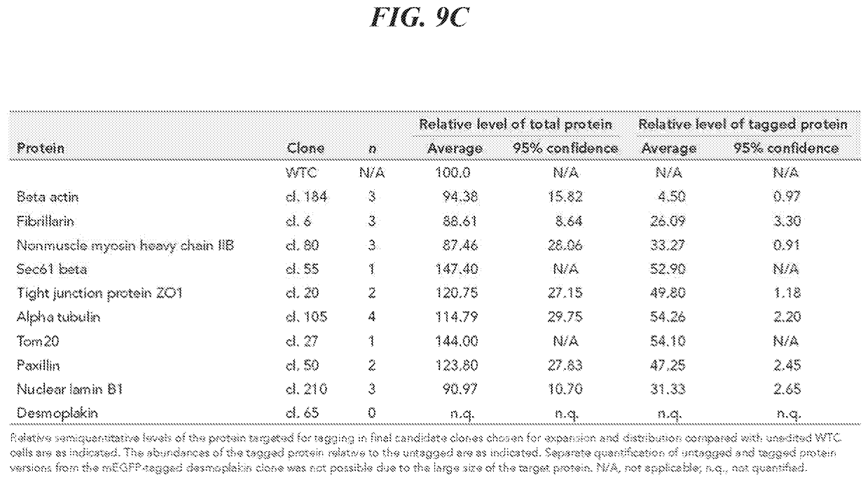

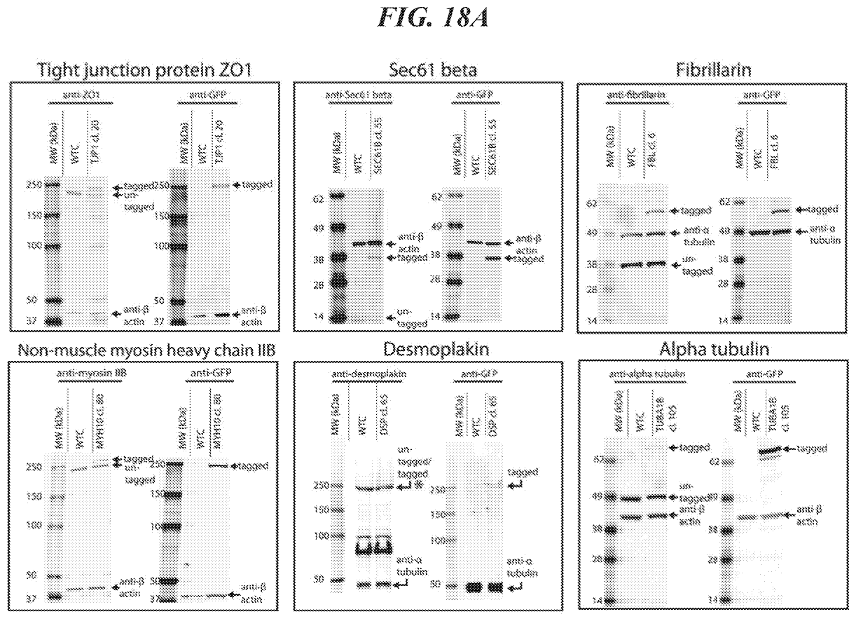

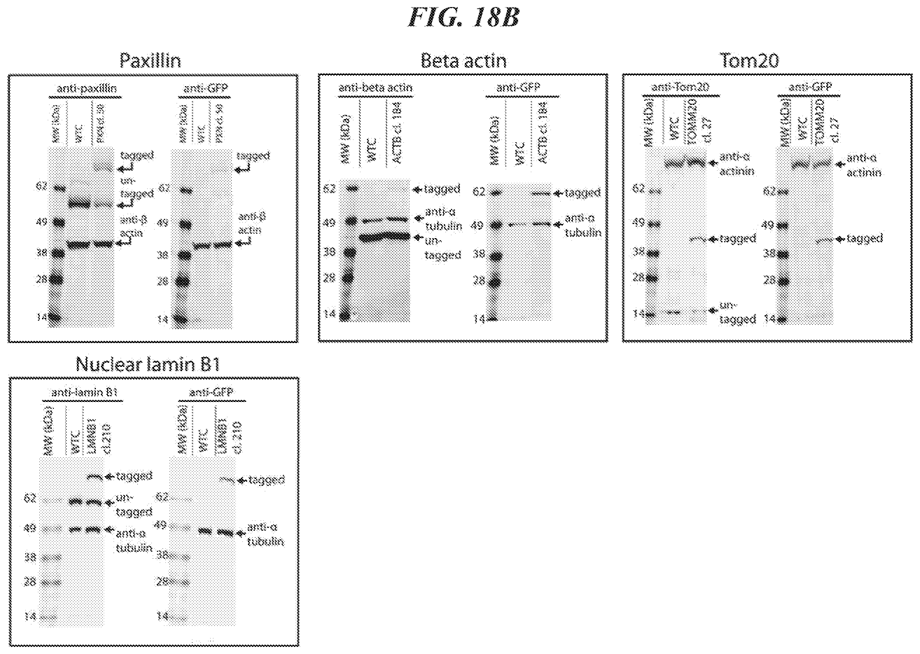

Western blot analysis was performed on lysates from each candidate clone in order to confirm that the observed shift in molecular weight of the tagged vs. untagged peptide was consistent with the known molecular weight of the linker and GFP tag (FIG. 9B and FIG. 18A). Immunoblotting with antibodies against the endogenous protein yielded products consistent with both the anticipated molecular weight of the tagged and untagged proteins and was further confirmed in all cases using an anti-GFP antibody (FIG. 9B and FIG. 18A). In FIG. 9B, lysates from ACTB cl. 184 (left), TOMM20 cl. 27 (middle), and LMNB1 cl. 210 (right) were compared to unedited WTC cell lysate by western blot. In all cases, blots with antibodies against the respective proteins (beta actin, Tom20, and nuclear lamin B1) are shown in the left blot, and blots with anti-GFP antibodies are shown in the right blot, as indicated. Loading controls were either alpha tubulin or alpha actinin, as indicated.

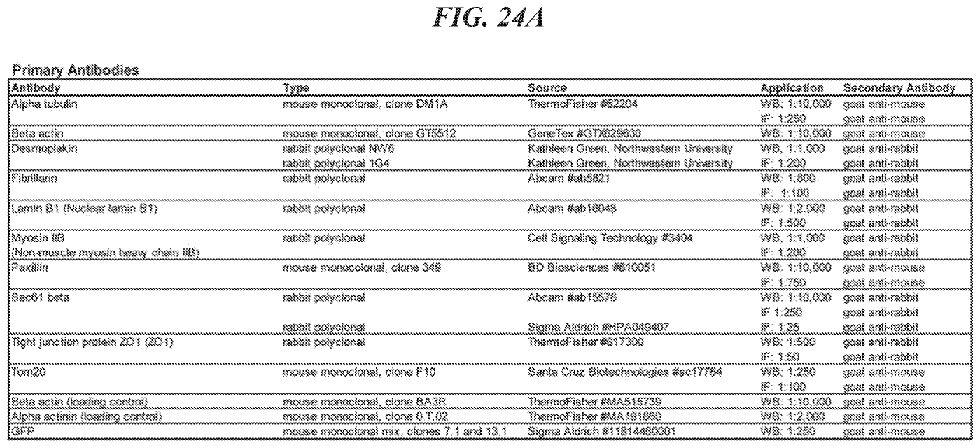

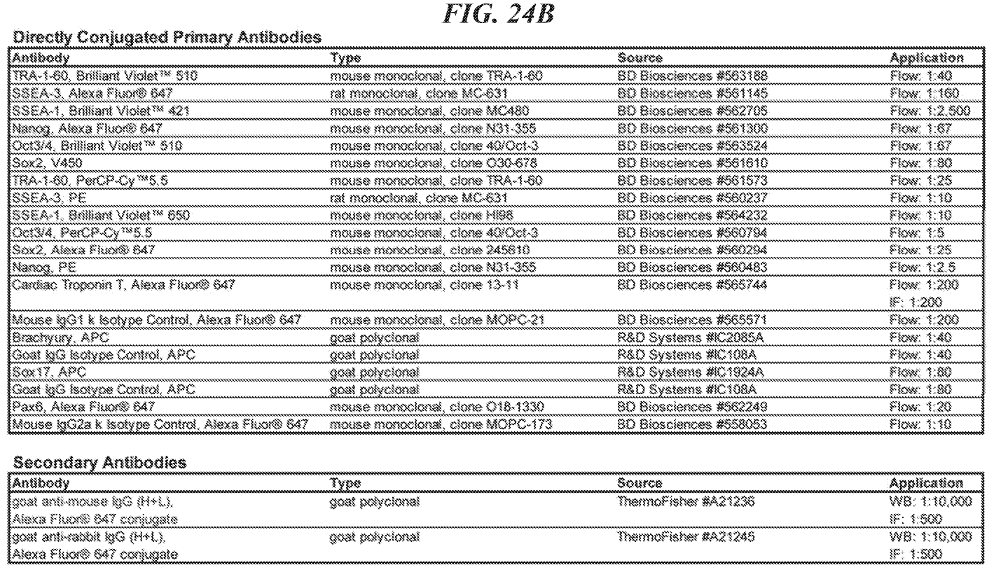

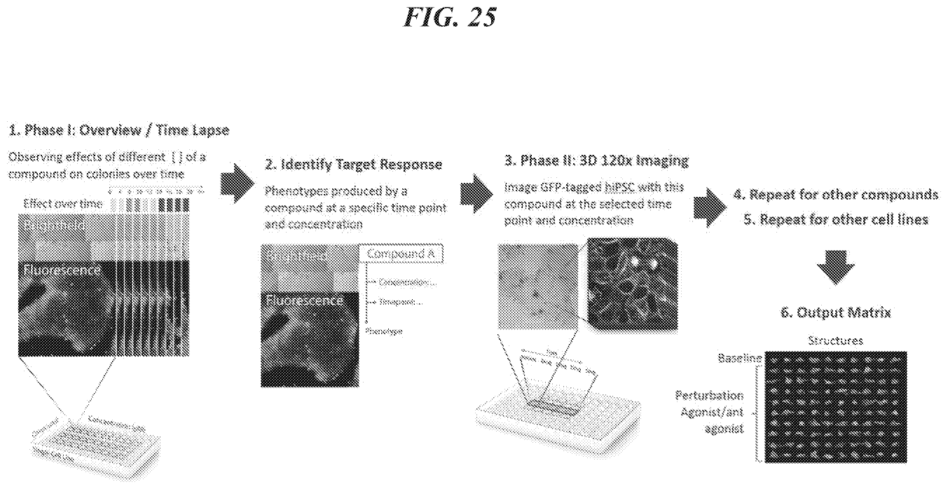

Semi-quantitative imaging of the blot was also used to determine the relative abundance of protein products derived from each allele. In all cases, immunoblotting with antibodies against the endogenous protein yielded products consistent with both the anticipated molecular weight of the tagged and untagged peptides. Notably, the appropriate Tom20-GFP fusion protein product was obtained despite our inability to identify a precisely edited clone, suggesting that the additional plasmid backbone sequence did not disrupt the coding sequence of the TOMM20 gene. Antibodies used in these experiments are described in FIG. 24A and FIG. 24B.

The western blot data was used to quantify the abundance of the GFP-tagged protein copy relative to the total abundance of the targeted protein (FIG. 9C). Relative levels of the tagged/untagged protein varied by experiment, but was highly reproducible. While many clones expressed the tagged protein at ˜50% of the total protein in the cell, as expected for monoallelic tagging, others did not (FIG. 9C). In the most extreme example, although the final tagged beta actin clone expressed total levels of beta actin similar to the levels found in unedited cells, only 5% of the detected protein was tagged. This suggested that these cells adapted to any compromised function of the tagged allele while retaining normal viability and behavior.

### Biallelic Edits

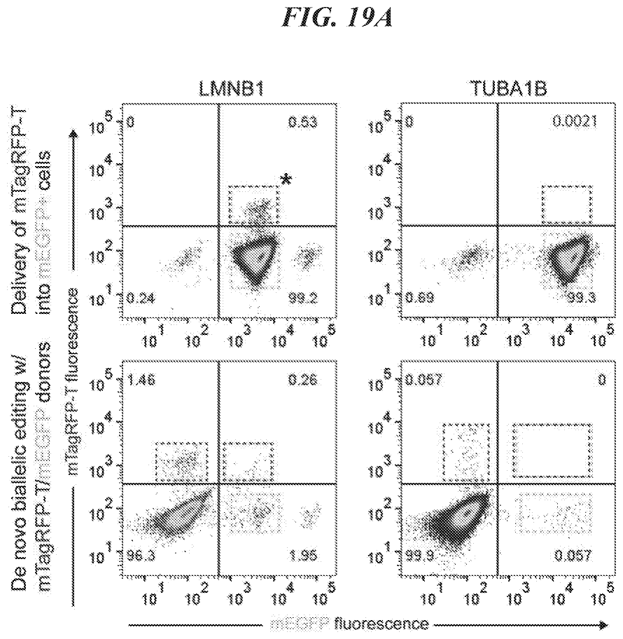

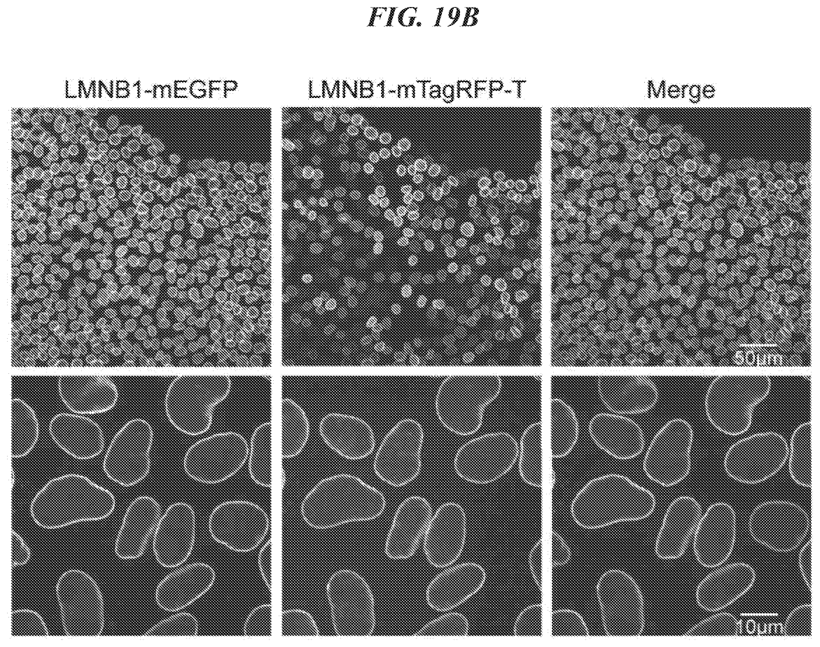

The observation that the tagged allele had reduced expression in some experiments coupled with the rarity of biallelic edits in these experiments raised the possibility that the tagged protein copy has reduced function. The tolerance of biallelic tagging (and thus whether the tagged protein has sufficient function) was tested by introducing a spectrally distinct red fluorescent protein tag (mTagRFP-T) into the unedited allele of two different tagged clonal cell lines, LMNB1-mEGFP and TUBA1B-mEGFP (FIG. 19A).

Putative biallelically edited cells were FACS-isolated, expanded, and imaged to confirm localization of both tags to the nuclear envelope in the enriched population (FIG. 19B). Additional experiments were performed to test whether transfection of two unique donor plasmids (one to deliver mEGFP and another for mTagRFP-T) simultaneously could produce biallelically edited cells in a single step in unedited cells using the RNP methods described above. Both methods produced populations of mTagRFP-T+/GFP+ cells, indicating tolerance of biallelic tagging at this locus despite previously observed reduced expression of the tagged protein (FIG. 19A).

In contrast to LMNB1, mTagRFP-T+/GFP+ cells were not able to be recovered after attempted editing of the TUBA1B-mEGFP clonal cell line with the TUBA1B-mTagRFP-T donor plasmid, nor were mTagRFP-T+/GFP+ cells able to be isolated when both donors were co-delivered to unedited cells, despite the prevalence of both mTagRFP-T+ and GFP+ cells as separate edited populations (FIG. 19A, right panels). These data suggest that genomic loci vary widely in their tolerance for biallelic tagging and that cells may compensate for monoallelic tags by reducing expression of the tagged protein, as observed (FIG. 9C). However, although the ratio of the expression of tagged protein to untagged protein varied by the edited line, the total amount of a protein (tagged plus untagged) in an edited line remained similar to the (untagged) amount in unedited cells (FIG. 9C, FIG. 18A-18B).

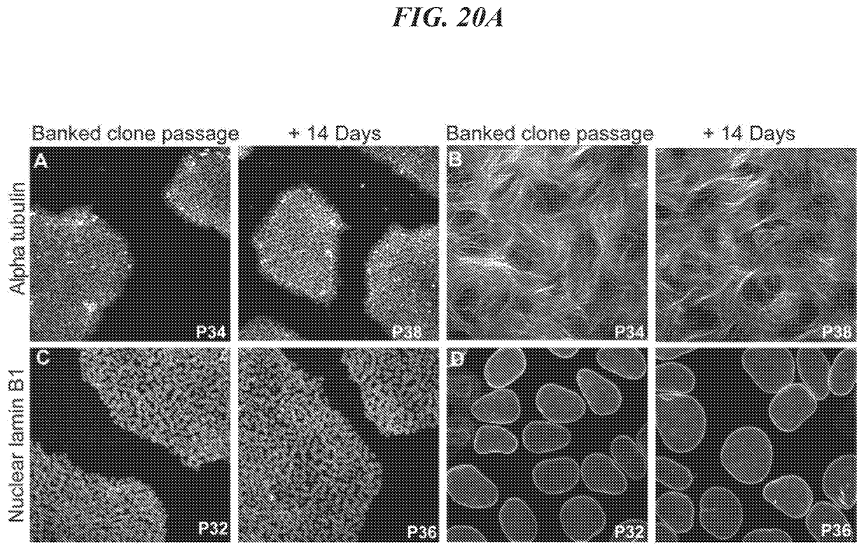

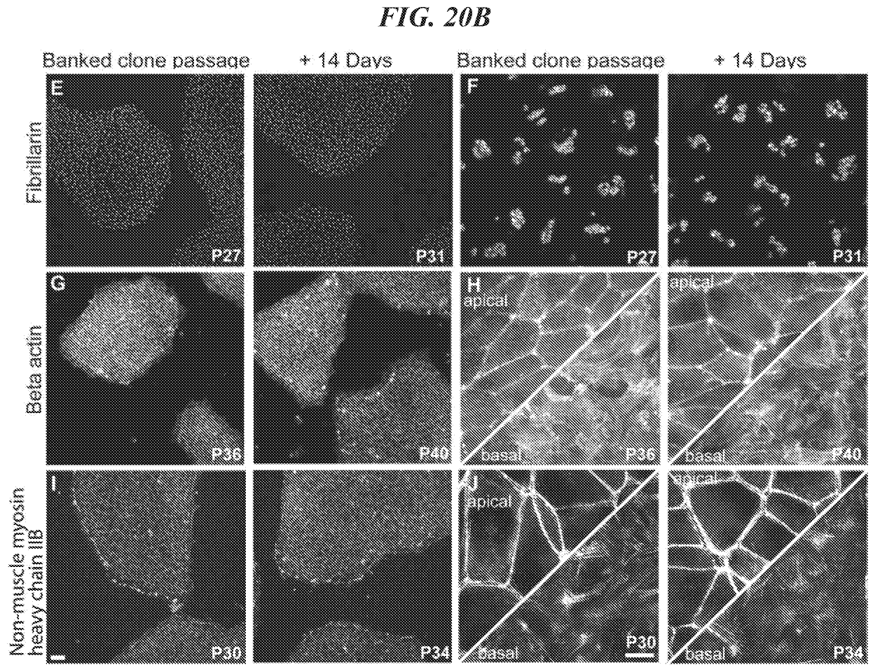

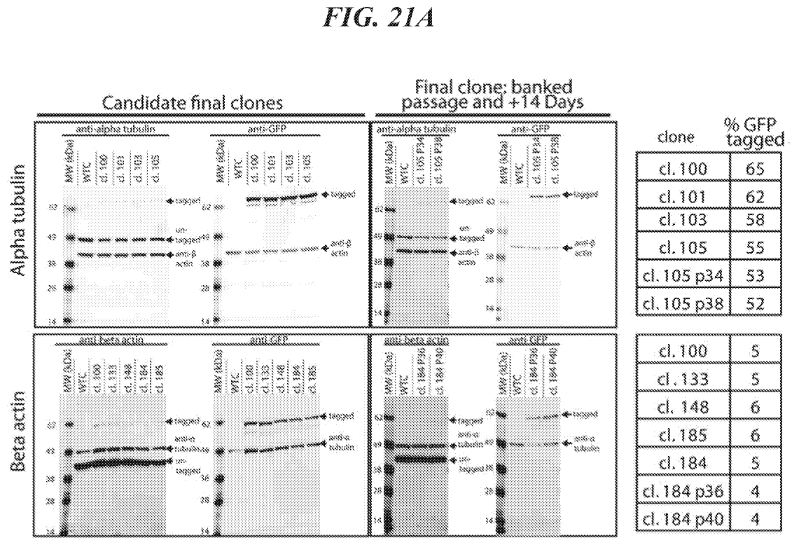

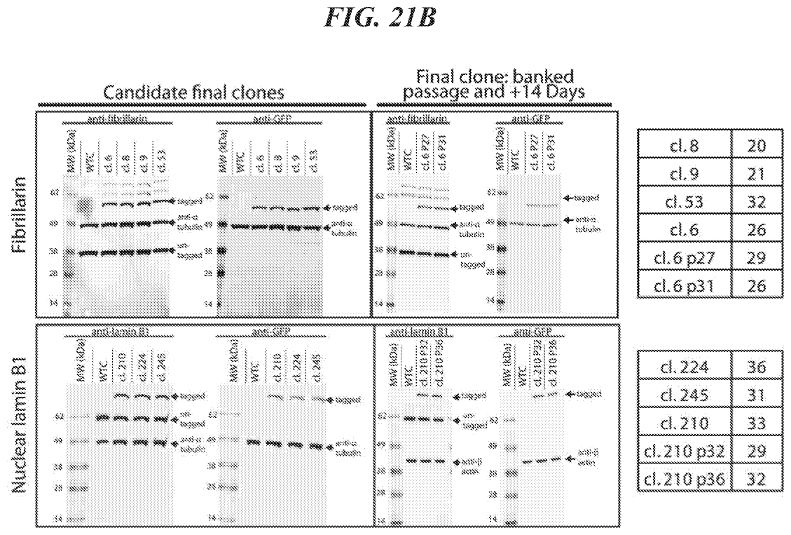

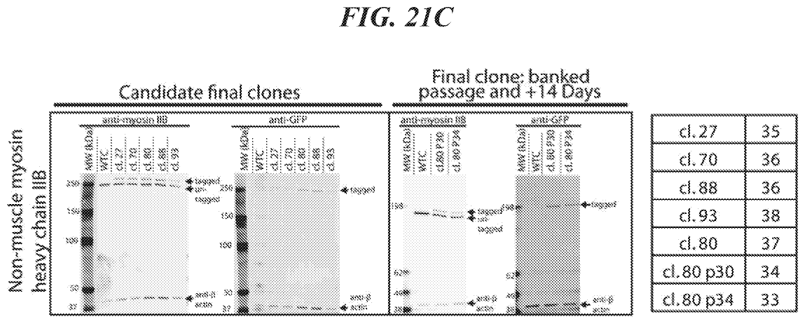

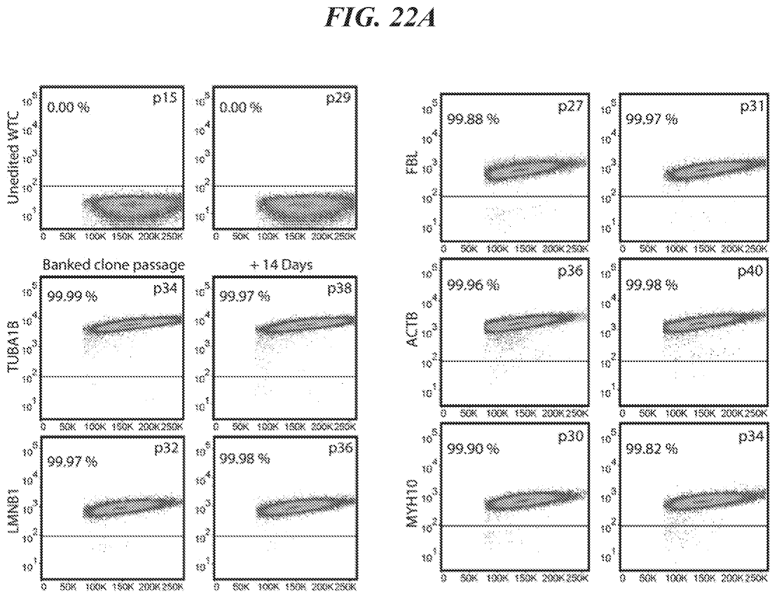

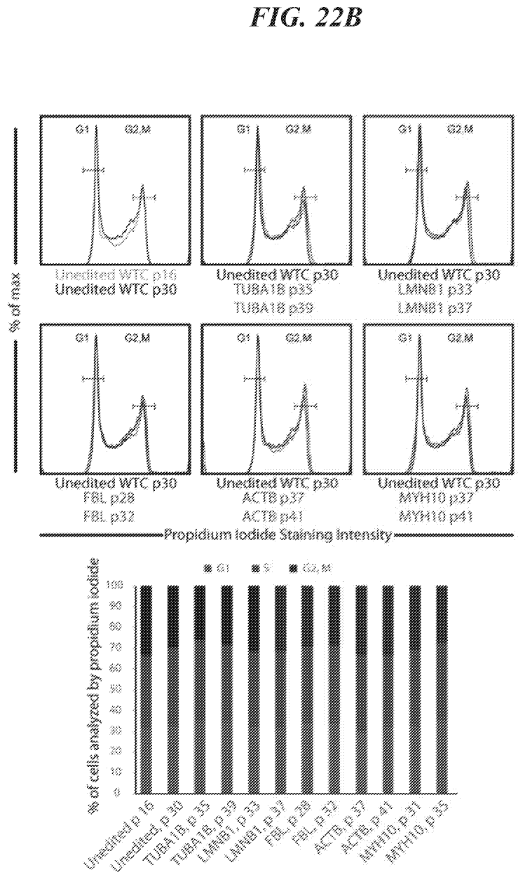

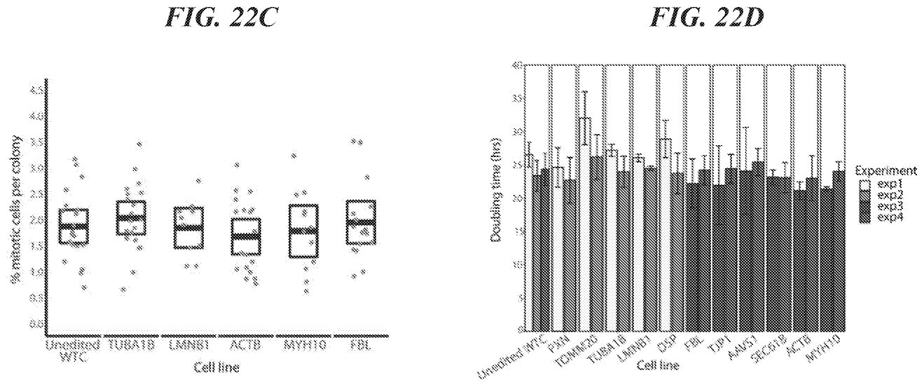

To assess the possibility of allele-specific loss of expression in clonally derived cultures due to perturbed function of the tagged protein copy this, two cultures of the four cell lines displaying unequal tagged/untagged protein copy abundance (and TUBA1B-mEGFP as a control) were maintained for different amounts of time. These two sets of cultures were then imaged. As shown in FIG. 20, no difference in the signal intensity or tag localization in cultures separated by four passages (14 days culture time). Similarly, no significant difference in the relative abundance of the tagged and untagged protein were observed in immunoblotting experiments performed on cultures that differed with respect to length of passage time (FIG. 21). Additionally, the ratio of tagged to untagged protein abundance in 4-5 independently edited clonal lines was consistent between the final clone chosen for expansion and alternative, independently generated clones (FIG. 21). Flow cytometry confirmed that GFP-negative cells were indistinguishably scarce in cultures at both passage numbers in each of five experiments and that the overall fluorescence intensity of the GFP-tagged protein was unaltered (FIG. 22A). The consistency in expression across clones and passaging time provided further confidence in the stability of expression.

### Example 5—Phenotypic and Functional Validation of Candidate Clones

Upon validating the expression and localization of the GFP-tagged protein in each of the genome-edited lines, experiments were performed to ensure that each expanded candidate clonal line retained stem cell properties comparable to the unedited WTC cells. Assays included morphology, growth rate, expression of pluripotency markers, and differentiation potential (FIG. 10, FIG. 22D). Undifferentiated stem cell morphology was defined as colonies retaining a smooth, defined edge and growing in an even, homogeneous monolayer (FIG. 10A). Clones with morphology consistent with spontaneous differentiation were rejected (Thomson et al., 1998; Smith, 2001; Brons et al., 2007; Tesar et al., 2007). Such cultures typically displayed colonies that were loosely packed with irregular edges and larger, more elongated cells compared to undifferentiated cells, as observed with one PXN clone (a confirmed biallelic edit) (FIG. 10A right-most image). Expression of established pluripotency stem cell markers was also determined, including the transcription factors Oct3/4, Sox2 and Nanog, and cell surface markers SSEA-3 and TRA-1-60 (FIG. 10B, FIG. 10F). High levels of penetrance in the expression of each marker (>86% of cells) were observed in all final clonal lines from the 10 different genome edits, similar to that of the unedited cells (FIG. 10B, FIG. 10F). Consistent with these results, low penetrance (<9% of cells) of the early differentiation marker SSEA-1 was observed by flow cytometry in both the edited and control WTC cells (FIG. 10B, FIG. 10F). All 39 clones satisfied commonly used guidelines of >85% pluripotency marker expression and <15% cells expressing the differentiation marker SSEA-1 used by various stem cell banks (Baghbaderani et al., 2015).

### Candidate Clones Retain Expression of Pluripotency Markers

Assays were performed to ensure that the clones identified to have precise edits retained stem cell properties during the process of gene editing and expansion. As such, the expression of established stem cell markers, including the transcription factors Oct3/4, Sox2 and Nanog, cell surface pluripotency markers Tra-160 and Tra 181, and the pro-differentiation marker SSEA3 were measured by flow cytometry (FIG. 5A). Briefly, cells were dissociated Accutase as previously described, fixed with CytoFix Fixation Buffer™ (BD Bioscience), and frozen in KnockOut™ Serum Replacement (Gibco) with 10% DMSO. Cells were washed with 2% BSA in DPBS and half of the cells were stained with anti-TRA-1-60 Brilliant Violet™ 510, anti-SSEA-3 AlexaFluor® 647, and anti-SSEA-1 Brilliant Violet™ 421 (all BD Bioscience). The other half of the cells were permeabilized with 0.5% Triton-X100 and 2% BSA in DPBS and stained with anti-Nanog AlexaFluor® 647, anti-Sox2 V450, and anti-Oct-3/4 Brilliant Violet™ 510 (all BD Bioscience). Cells were acquired on a FACSAria Fusion (BD Bioscience) equipped with 405, 488, 561, and 637 nm lasers and analyzed using FlowJo software V.10.2 (Treestar, Inc.). Doublets were excluded using forward scatter and side scatter (height versus width), then marker-specific gates were set according to corresponding fluorescence-minus-one (FMO) controls to obtain the percent positive for each marker.

In all candidate clones tested, each nuclear marker was expressed well above the commonly used thresholds of >85%+ for stem cell markers and <15%+ for differentiation markers and comparable to the parental WTC line (FIGS. 5A and 5B). When compared to the WTC reference line, all clones displayed negligible changes in the mean expression intensity of each nuclear marker. Cell surface pluripotency markers displayed similarly robust expression when analyzed in this manner, albeit with greater variability (FIG. 5A and FIG. 5C). This analysis was conducted for a total of approximately 50 clones and only 10% were rejected due to changes in the expression profile of these markers. Although comparable, there was sufficient variability within each set of candidate clones candidate clones could be ranked relative to each other to determine those that were most similar to the WTC parent line.

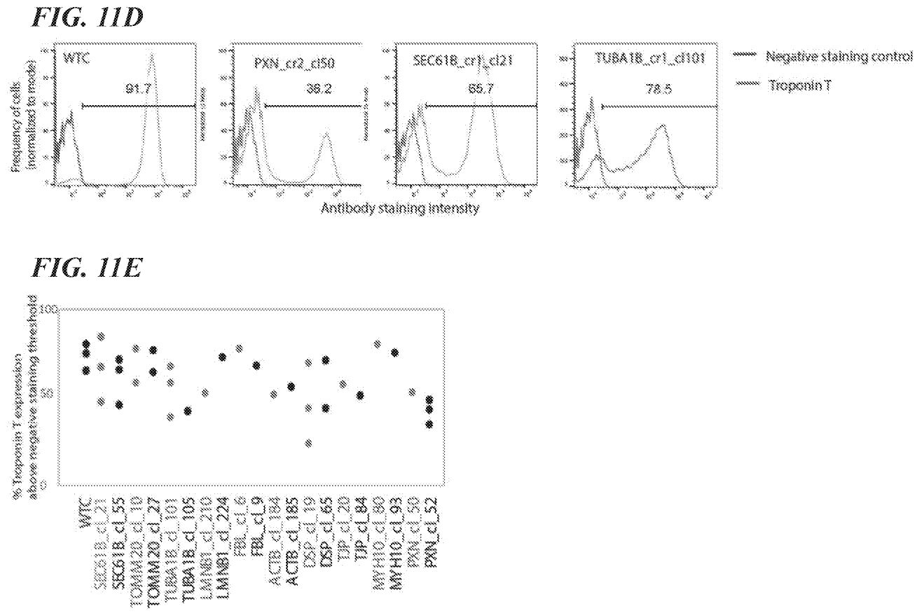

In vitro differentiation assays to confirm the pluripotency of the cell lines were performed. Directed germ layer differentiation was compared between unedited cells and the final selected edited clonal line representing each of the 10 targeted structures. Each cell line was differentiated for 5-7 days under defined conditions to mesoderm, endoderm, and ectoderm using differentiation media specific to each lineage. The cells were stained for early markers of germ layer differentiation (Brachyury, Sox 17, and Pax6) and analyzed by flow cytometry (FIG. 10C, FIG. 11A-11C, FIG. 10F) (Showell et al., 2004; Murry and Keller, 2008. Zhang et al., 2010; Viotti et al., 2014). While the differentiation into each germ layer was variable, all three germ layer markers in the edited clones showed increased expression relative to undifferentiated cells (FIG. 10C). In all edited clones tested, ≥91% of cells expressed Brachyury after mesodermal differentiation, ≥47% expressed Sox17 after differentiation to endoderm, and ≥65% expressed Pax6 upon ectoderm differentiation (FIG. 10C, FIG. 10F). Directed differentiation of edited clones into each germ layer lineage was generally comparable to unedited cells.

### Gene Edited Candidate Clones are Capable of Cardiomyocyte Differentiation

Additional experiments were performed to assess whether each clone could robustly differentiate into cardiomyocytes. Each edited clone's differentiation potential was assessed by directing it to a cardiomyocyte fate using established protocols using a combination of growth factors and small molecules (Lian et al., 2015; Palpant et al., 2015) and evaluated cultures for spontaneous beating (days 6-20) and cardiac Troponin T (cTnT) expression (days 20-25), in order to evaluate the robustness of cardiomyocyte differentiation. Briefly, cells were seeded onto Matrigel-coated 6-well tissue culture plates at a density ranging from 0.5-2×10⁶ cells per well in mTeSR1 supplemented with 1% P/S, 10 μM RI, and 1 μM CHIR99021 (Cayman Checmical). The following day (designated day 0), directed cardiac differentiation was initiated by treating the cultures with 100 ng/mL ActivinA (R&D) in RPMI media (Invitrogen) containing 1:60 diluted GFR Matrigel (Corning), and insulin-free B27 supplement (Invitrogen). After 17 hours (day 1), cultures were treated with 10 ng/mL BMP4 (R&D systems) in RPMI media containing 1 μM CHIR99021 and insulin-free B27 supplement. At day 3, cultures were treated with 1 μM XAV 939 (ToCris) in RPMI media supplemented with insulin-free B27 supplement. On day 5, the media was replaced with RPMI media supplemented with insulin-free B27. From day 7 onto about day 20, media was replaced with RPMI media supplemented with B27 with insulin (Invitrogen). Cells were harvested using 0.5% Trypsin-EDTA (Gibco), filtered with a 40 μm cell strainer, fixed with CytoFix Fixation Buffer™, permeabilized with BD PermiWash™ buffer, stained with anti-Cardiac Troponin T AlexaFluor® 647 (BD Bioscience) or isotype control, acquired on a FACSAria Fusion and analyzed using FlowJo software V.10.2.

Clonal lines generally displayed successful cardiomyocyte differentiation, with cTnT expression and qualitative spontaneous contractility comparable to the parental WTC line (FIG. 10D, 10E, FIG. 10F). Variability was observed both between clones and between differentiation experiments within a given clone. In order to address this variation, the initial density of the cells was varied. Initial beating, homogeneity of beating in the culture, and perceived strength of contraction were used as qualitative markers to rank clones relative to each other. Additionally, Troponin T expression after 20 days in culture was used as a quantitative measurement of the cells' commitment to cardiomyocyte identity (FIGS. 11D and 11E). The total fraction of cells in each culture that was positive for Troponin T varied significantly between experiments, but in all cases >30% Troponin T+ cells were obtained. Data for cell lines with GFP-tagged PXN, TOM20, TUBA1B, LMNB1, and DSP can be found at the Allen Institute for Cell Science's website under the cell-line catalog section, which is incorporated by reference in its entirety.

This cardiomyocyte differentiation data combined with pluripotency marker expression and germ layer differentiation data, support the conclusion that fusing GFP with these endogenously expressed proteins via monoallelic tagging does not appear to disrupt pluripotency or differentiation potential of these edited hiPSC cells.

Additional experiments can be performed according to protocols known in the art (e.g., Methods Mol Biol. 2014; 1210:131-41; Biomed Rep. 2017 April; 6(4): 367-373; Methods Mol Biol. 2017; 1597:195-206; Nat Commun. 2015 Oct 23; 6:8715; Mol Psychiatry. 2017 Apr. 18. doi: 10.1038/mp.2017.56; Scientific Reports volume 7, Article number: 42367 (2017)) and illustrated in FIG. 32 to determine the ability of the clonal cell lines to differentiate into hepatocytes, renal cells, neuronal cells, or other cells.

### Edited Clones are Karyotypically Stable

Establishing clonal hiPSC lines and culturing them long term is known to carry the risk of fixing somatic mutations and/or chromosomal aneuploidies (Weissbein et al., 2014). The possibility exists that the additional stressors inherent to gene editing heighten this risk. To address this concern, karyotype analysis was performed on each candidate clone. Karyotype analysis was performed by Diagnostic Cytogenetics Inc. (DCI, Seattle Wash.). At minimum of 20 metaphase cells were analyzed per clone. Of the ˜50 candidate clones tested, only two instances where karyotypic abnormalities became fixed in the culture were detected (data not shown). In one instance, a single candidate clone from an experiment was rejected. Further, all clones identified as candidates from the experiment targeting ACTN1 displayed the same aneuploidy event, suggesting that it had become fixed early in the editing process. These data indicate that that aneuploidy occurs at a rate that is non-negligible, and that chromosomal abnormalities must be ruled out in each experiment. However, these data suggest that the rate of aneuploidy is permissively low for high-throughput editing using these methodologies.

### Transcriptome-Wide Analysis of Edited Candidate Clones

Transcriptome-wide analysis of two final candidate clones from a number of experiments was performed. This analysis was performed to determine whether hiPSC clones maintained over 10-15 passages and harboring potentially disruptive tags on key cellular proteins demonstrated similar global gene expression patterns to the unedited reference line, or if they had alternatively evolved into globally distinct cell lines in a manner not distinguishable by the above described quality control assays (data not shown).

In order to further characterize global gene expression changes between each edited clone and the reference line, genes whose expression differs by greater than 2-fold are annotated and compared between experiments and expression from control cell lines. Cluster analysis on these data sets is also performed to determine the most statistically significant GO term categories among edited clones. RNA-seq analysis is also performed to confirm the absence of detectable mutations in expressed sequences due to potential off-target Cas9 activity in the final clones. These findings are further confirmed by next generation exome sequencing. Analysis for additional clones is also be performed.

Phenotypic Characterization of GFP-Tagged iPSC Lines

These results indicate that the stem cells and stably tagged stem cell clones and differentiated cells therefrom of the invention can be used for three-dimensional live cell imaging of intracellular proteins. In further embodiments, the methods allow for use of the cells for screening, observing cellular dysplasia, disease staging, monitoring disease progression or improvement or cellular stress in response to a test agent.

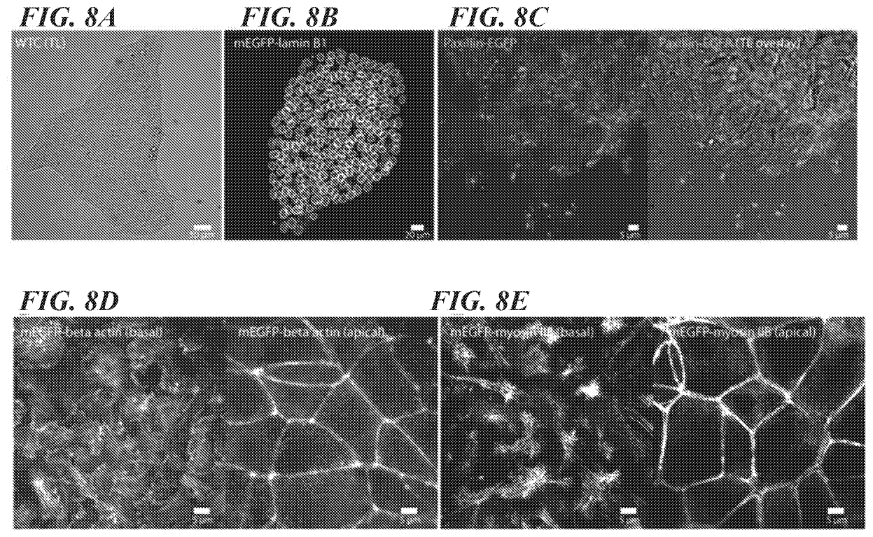

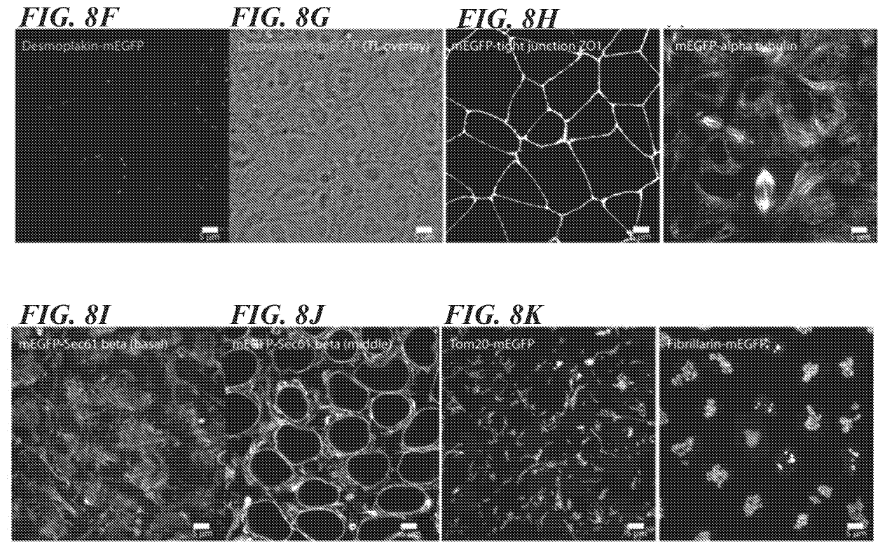

As a final characterization step, live imaging on preferred candidate clones was performed. Cells were maintained with phenol red free mTeSR1 media (STEMCELL Technologies) one day prior to live-cell imaging. Stably tagged stem cell clones can be imaged using spinning disk confocal microscopy. Cells were imaged using spinning disk confocal microscopy at low (10× or 20×) and high (100×) magnification. Microscopes were outfitted with a humidified environmental chamber to maintain cells at 37° C. with 5% CO₂ during imaging. Healthy, undifferentiated WTC hiPSCs ranged from 5-20 μm in diameter and 10-20 μm in height and grew in tightly packed colonies (FIG. 8A, 8B). The resulting endogenously tagged lines allowed for the observation of tagged proteins and corresponding organelles with exceptional clarity due to their endogenous regulation and absence of fixation and staining artifacts. Without exception, distinct localization patterns of the tagged protein were observed when compared to cells transiently transfected with constructs expressing GFP fusion proteins.

For example, paxillin was observed in the matrix adhesions formed between substrate contact points and the basal surface of cells, as well as at the dynamic edges of colonies (FIG. 8C). Beta actin localized to the basal surface of colonies both in prominent filaments (stress fibers) and at the periphery of cell protrusions (lamellipodia), as well as in an apical actin band at cell-cell contacts, a feature common in epithelial cells (FIG. 8D). Non-muscle myosin heavy chain IIB had similar localization in actomyosin bundles, including at basal stress fibers and in an apical band (FIG. 8D, 8E). Desmoplakin localized to distinct puncta at apical cell-cell boundaries as expected of desmosomes, which form junctional complexes in epithelial cells (FIG. 8F). Tight junction protein ZO1 also localized apically to cell-cell contacts where tight junctions are formed (FIG. 8G). These observations suggest the presence of multiple distinct epithelial junction complexes and an overall apical junction zone in edited hiPSC colonies. In addition, alpha tubulin was both diffuse, as unpolymerized tubulin, and localized to microtubules, which exhibited apicobasal polarity in non-dividing cells with many microtubules extending parallel to the z-direction as reported for some epithelial cell types (FIG. 8H) (Musch, 2004; Toya and Takeichi, 2016).

Sec61 beta localized to endoplasmic reticulum (FIG. 8I), and Tom20 localized to mitochondria (FIG. 8J) and were distributed throughout the cytoplasm, often with greatest density in a cytoplasmic ‘pocket’ near the top of the cell and at lowest density in the central periphery of the cell. The center region of the cell was almost entirely occupied by the nucleus, which was observed outlined by nuclear lamin B1 (FIG. 8B). Fibrillarin was localized to nucleoli within the center of the nuclei (FIG. 8K).

These observations are consistent with the epithelial nature of tightly packed undifferentiated WTC hiPSCs grown on 2D surfaces. All final candidate clones, spanning 10 editing experiments, exhibited predicted subcellular localization of their tagged proteins (FIG. 8). Taken together, these data demonstrate the ability to identify clonal lines in which genome editing did not interfere with the expected localization of the tagged proteins to their respective structures. Furthermore, live-cell time-lapse imaging demonstrated that proper localization occurred throughout the cell cycle and the presence of the tagged protein did not noticeably interfere with cell behavior.

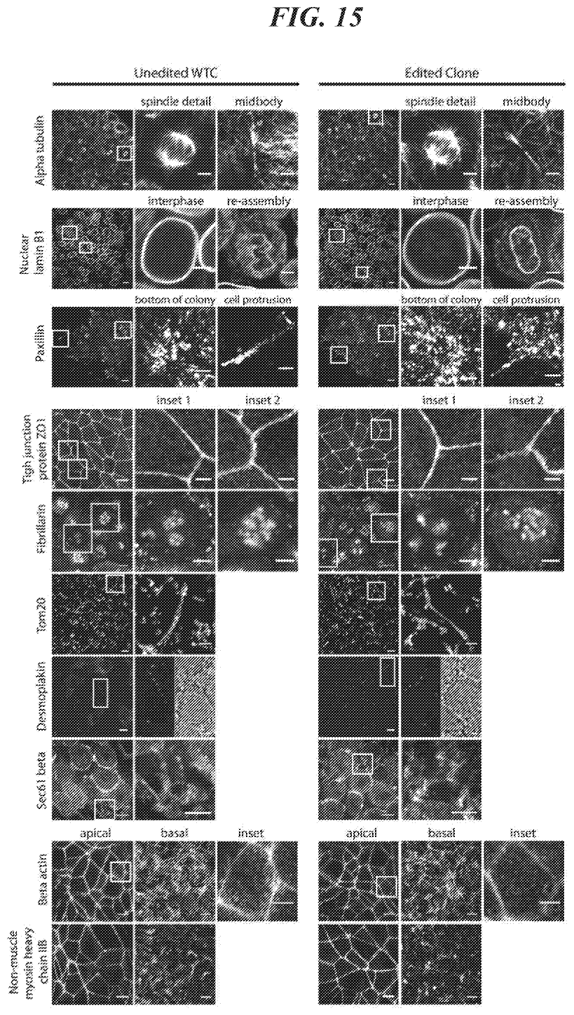

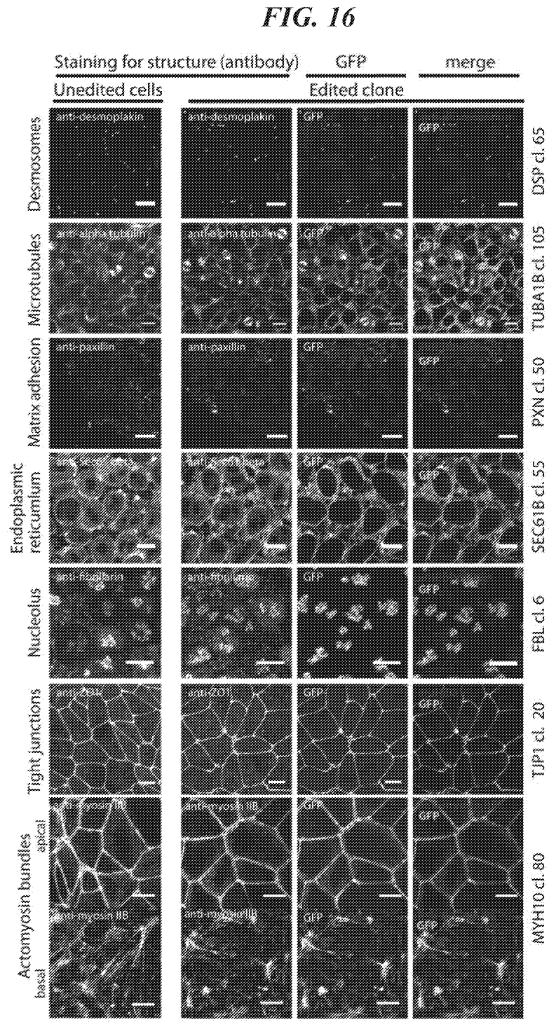

The impact of the tag on correct localization of the targeted protein compared to the localization of the native, unedited protein was also assessed. Edited clones were fixed alongside unedited cells and immunocytochemistry or phalloidin staining was performed. In all 10 experiments, no detectable differences in the pattern of antibody labeling between the unedited cells and the edited cell line were observed (FIG. 9A, FIG. 15, and FIG. 16). Within all edited cell lines, localization of the GFP-tagged protein was also compared to the pattern of antibody labeling, which was predicted to label both the GFP-tagged and untagged protein fractions within the same cell. In all cases, this revealed extensive co-localization (FIG. 15, and FIG. 16).

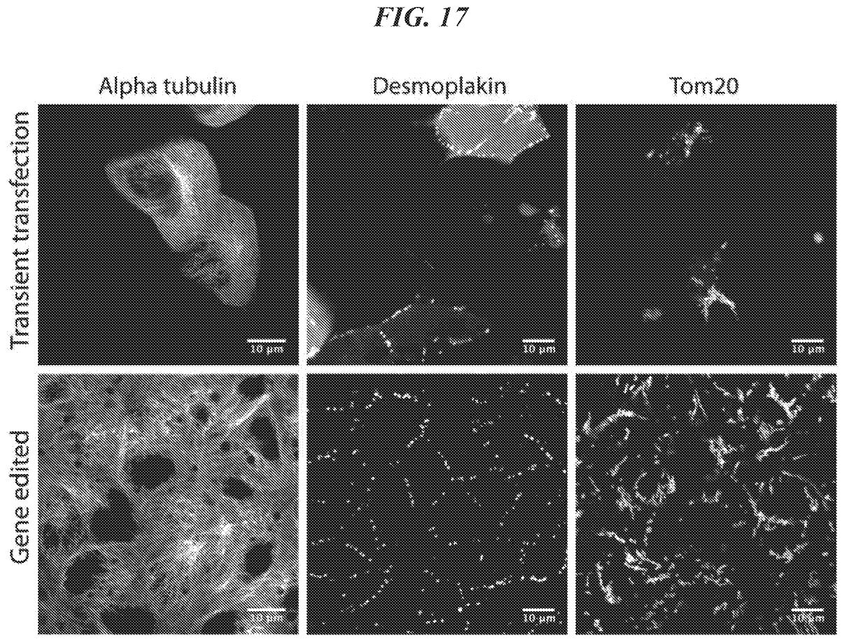

As endogenously GFP-tagged proteins in live imaging experiments generate more interpretable localization data than that produced in fixed and immunostained cells (Allen Institute for Cell Science, 2017), endogenous localization in edited lines was directly compared to cells transiently transfected with constructs expressing FP-fusion proteins (EGFP or mCherry) (FIG. 17). Although transient transfection, like fixation and immunostaining, is vulnerable to artifacts, cells with low transient transgene expression exhibited similar tag localization to that observed in the gene edited cell lines. In other cases, high transient transgene expression led to artifacts, including high diffuse cytosolic background and aggregation of the tagged protein. Intensity level was used as a proxy to distinguish between low- and high-level transgene overexpression, though low-level expressing cells were often rare. As examples, transfected cells with low EGFP-tubulin transgene expression were comparable to the gene edited alpha tubulin cells (TUBA1B-mEGFP), although the transfected cells contained higher cytosolic signal. Transfected cells with low desmoplakin-EGFP transgene expression revealed a similar pattern to that observed in the DSP-mEGFP gene-edited line, but the transfected cell population also contained other cells, likely expressing the transgene to a greater extent, with high cytosolic signal and increased number and size of desmosome-like puncta. Transfection and overexpression of Tom20 led to cell death and perturbed mitochondrial morphology, while the endogenously tagged cells displayed intact mitochondrial networks with both normal morphology and cell viability. These results highlight the importance of using multiple techniques to validate the localization of tagged proteins in gene edited cell lines. They also demonstrate the advantages to using genome editing to observe cellular structures rather than conventional methods that rely on overexpression, fixation, and antibody staining.

### Example 6—Development of Image-Based Drug-Induced Protein Signatures

The collection of the gene-edited hiPS cells described herein was used to develop image-based drug-induced protein signatures. Experiments were conducted with 12 known reference compounds that disrupt various key cellular structures and processes including cell division, microtubule organization, actin dynamics, vesicle trafficking, cell signaling, DNA replication, calcium regulation, ion channel regulators, and statins. Agents used in these experiments are shown in FIG. 26A.

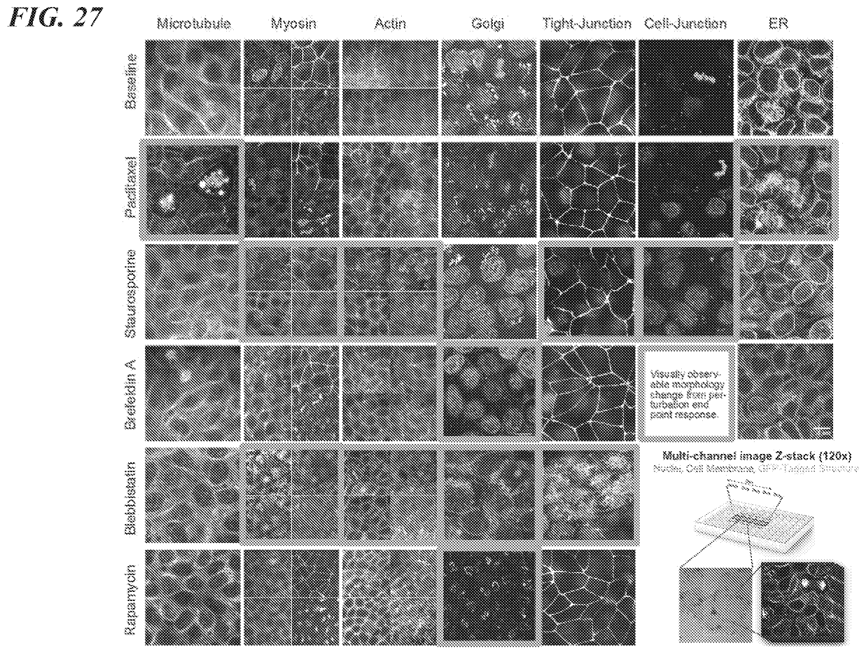

The pipeline was prototyped using a small suite of well-characterized compounds that include brefeldin A, paclitaxel, rapamycin, wortmannin and staurosporine (FIG. 26A). Low-resolution imaging (24× magnification) was used to test a matrix of concentrations and time points for each compound of interest to establish an initial set of conditions for each perturbation. hiPSC colonies were monitored for morphologic changes using transmitted light (FIG. 26B) and an endogenously GFP-tagged structure, such as microtubules (FIG. 26C). After establishing an end point response for several compounds, high-resolution (120× magnification) imaging of multiple cell lines was performed under standardized perturbation parameters, in the presence of dyes to label the nucleus and cell membrane for reference purposes (FIG. 27). FIG. 27 shows representative image planes from z-stacks collected at 120× of the GFP-tagged cell lines with nucleus and cell membrane markers. Cells were treated with the indicated perturbation agent at a pre-selected concentration and time point established in phase I.

These perturbations showed alterations roughly analogous to those seen in other cell types. For example, the microtubule stabilizing agent paclitaxel increased microtubule bundle thickness and altered the shape and position of the mitotic spindle during hiPS cell division. In addition, paclitaxel, also induced aberrant reorganization of the ER in cells undergoing mitosis, while showing minimal effects on the bulk organization of the actin bundles and cell junctions. Other drugs, such as staurosporine, a broad kinase inhibitor, had major effects on colony and cell morphology, inducing rearrangements in cell packing and shape. It also induced re-localization of desmosomes, indicating that the cell-cell junctions undergo substantial rearrangement.

Fluorescence quantification of the 3D images were used to analyze drug-induced Golgi reorganization, cytoskeleton reorganization, and cell junction reorganization. To quantify the relative abundance of each structure of interest (e.g. Golgi as presented above), the pixel intensities of the GFP channel (488 nm) were summed across the entire z-stack. For each experiment, the same threshold was used to exclude background intensity noise across the control (DMSO) and experimental (perturbation agent) groups. The data were plotted by averaging z-stack data from a time interval (30-minute) and compared to the control DMSO data. Dunnett's multiple comparison test was used to perform one-way ANOVA between the different time intervals against the control group.

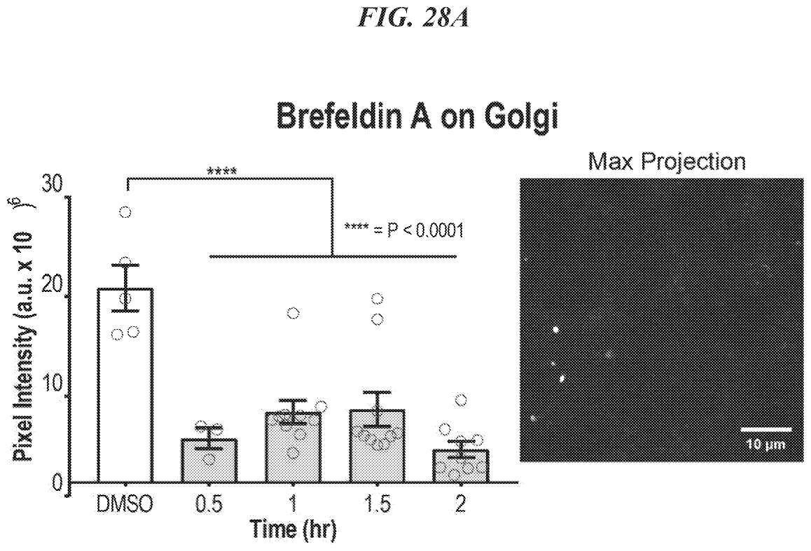

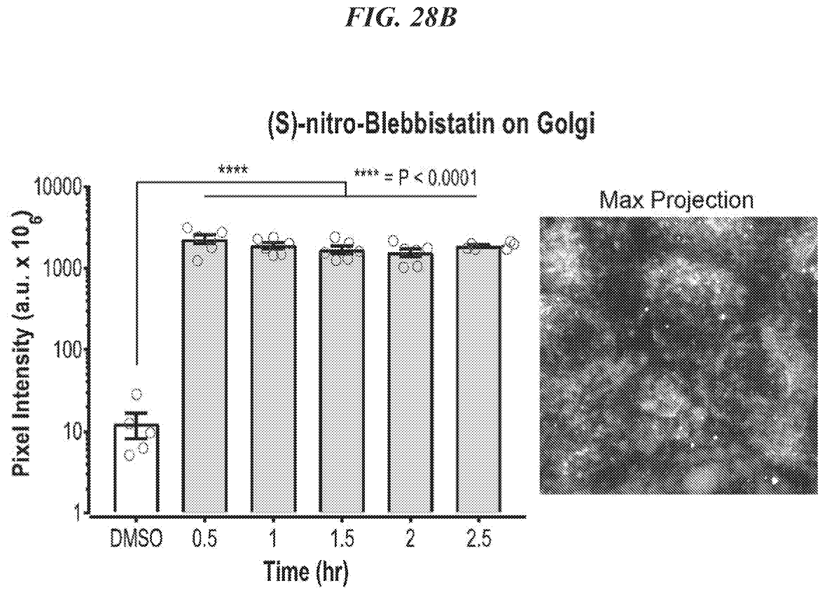

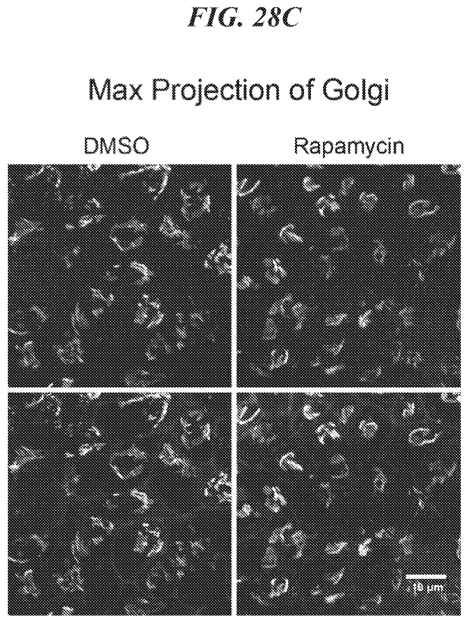

As shown in FIG. 28, Brefeldin A induced dissociation of the golgi within 30 minutes (FIG. 28A), while (S)-nitro blebbistatin induced fragmentation of the organelle (FIG. 28B). Additionally, rapamycin induced morphological reorganization of the golgi (FIG. 28C).

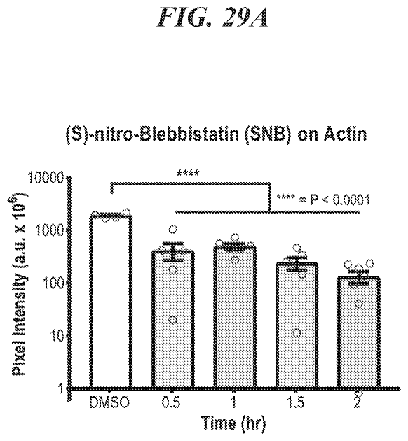

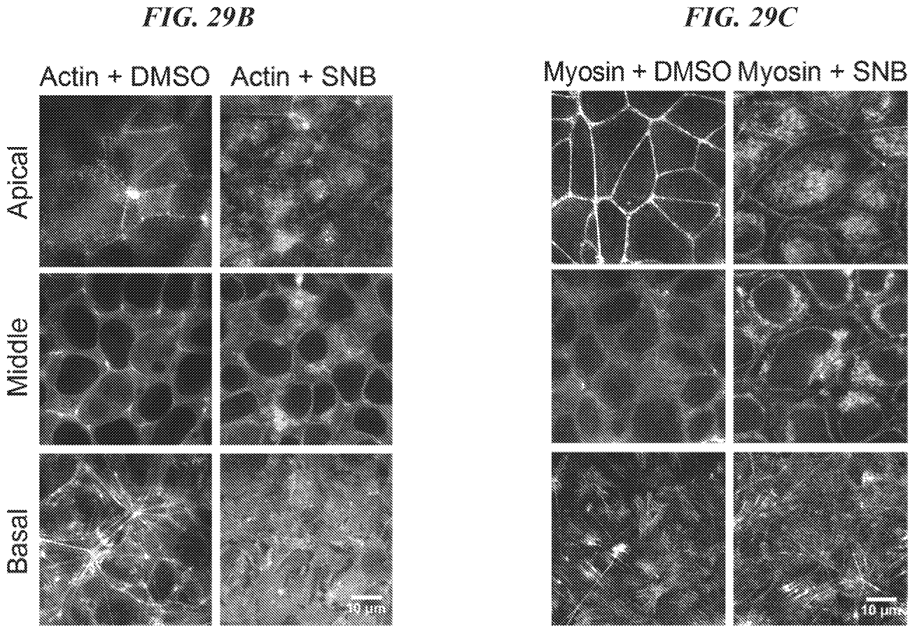

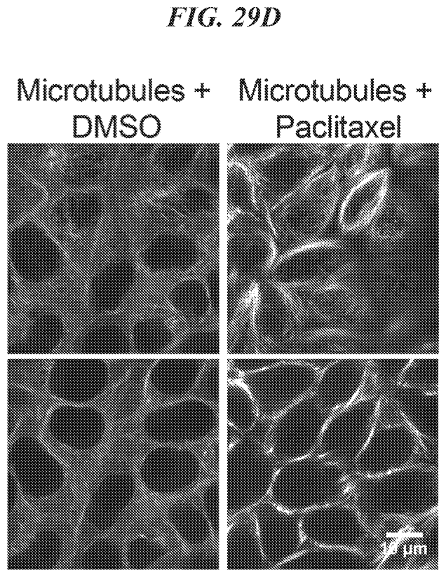

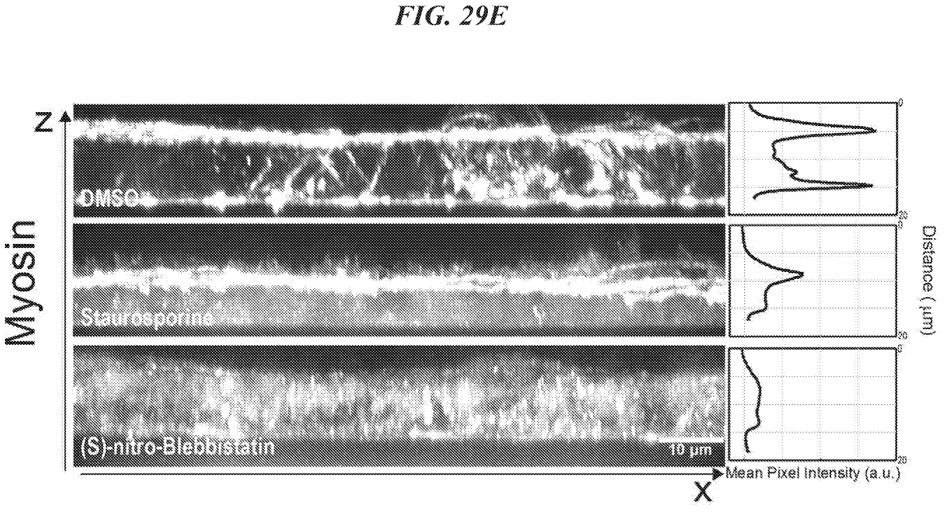

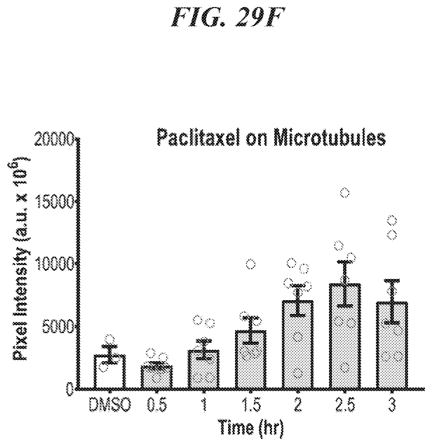

Relative protein abundance of actin and myosin were also quantified. As shown in FIG. 29, a reorganization and relative decrease in actin (FIGS. 29A and 29B) and myosin protein abundance was observed in the presence of (S)-nitro blebbistatin (FIG. 29C). In addition, paclitaxel stabilized the microtubules by enhancing polymerization oftubulin, which was reflected in a trend of increased relative localized fluorescence intensity over time (FIGS. 29D and 29F). Further, both staurosporine and (S)-nitro blebbistatin induced reorganization of the myosin through the thickness of the cell (FIG. 29E).

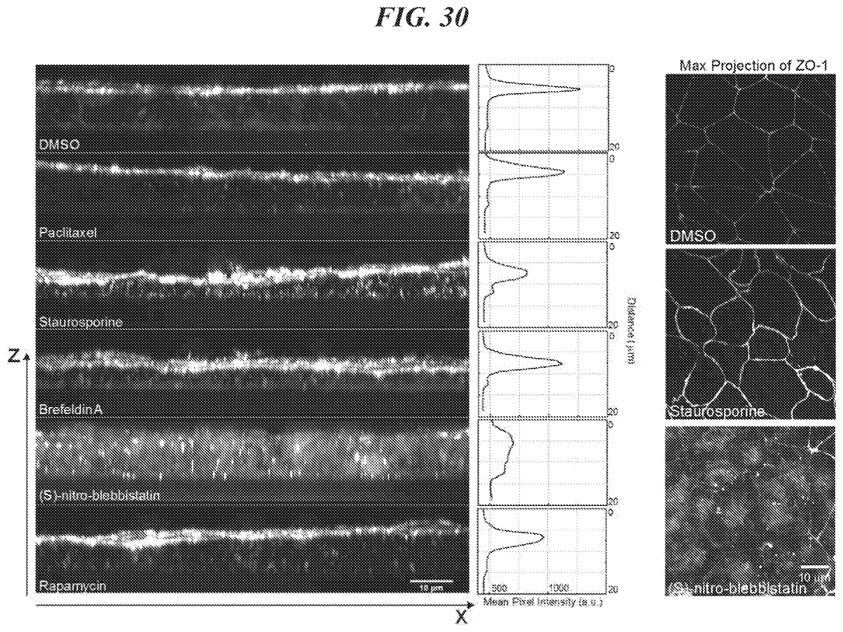

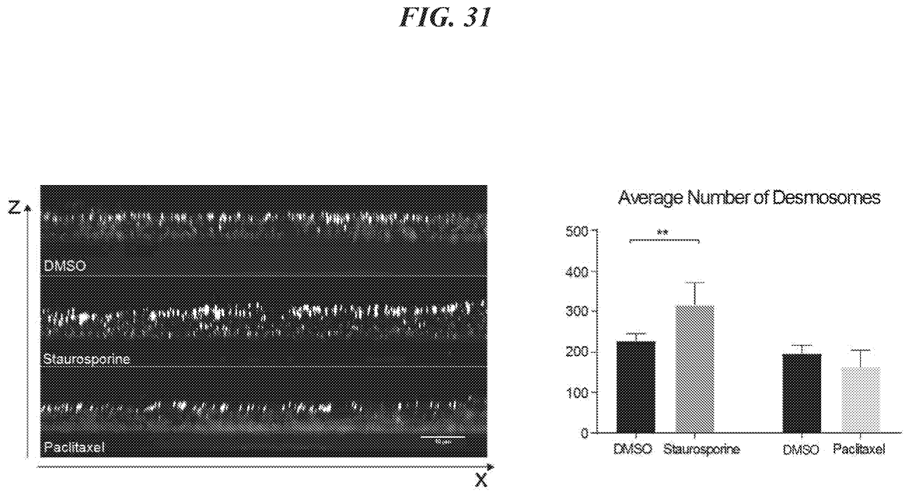

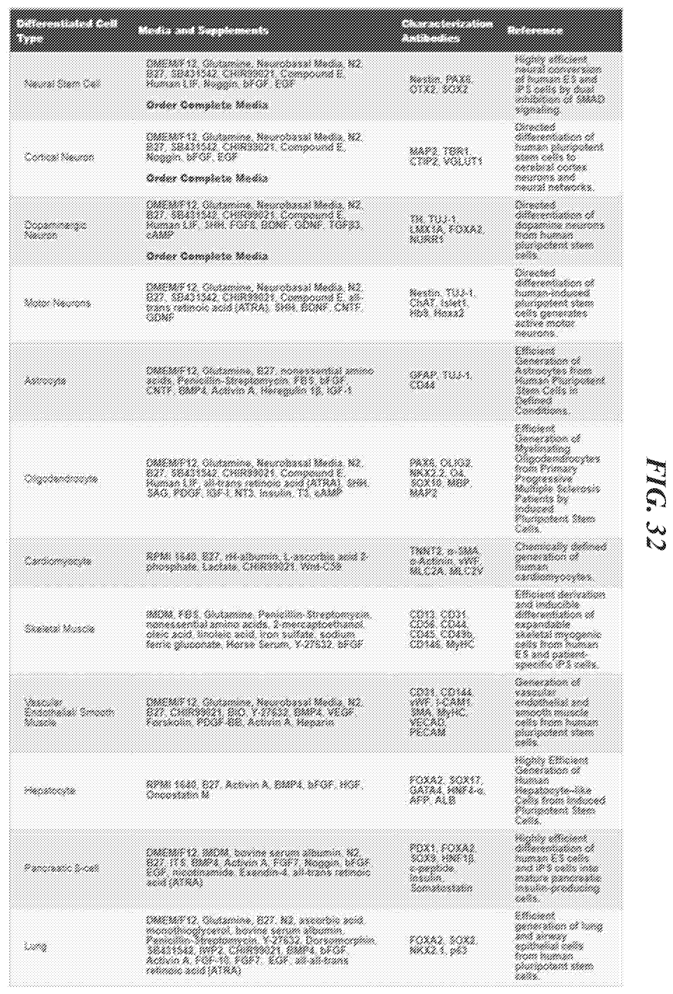

For drug-induced effects on cell junction reorganization, representative maximum intensity projections of a z-stack along the x-z axis are shown in FIG. 30. From these projections, the mean pixel intensity for the GFP channel along the x-axis, from the top of the image to the bottom, was measured to generate an intensity profile plot. These plots show the redistribution of ZO-1 along the z-axis in the presence of both staurosporine and (S)-nitro-blebbistatin. In presence of staurosporine, desmosomes relocalized throughout the cell, and the number of DSP-positive plaques increased in number (FIG. 31). To analyze the change in desmosome number, the number of 3D objects in a z-stack were counted using the 3D Object Counter tool in Fiji. The images were thresholded by size and minimum pixel intensity such that ˜95% of the objects were captured. Data were analyzed by Student's t-test (** p<0.01).

These data demonstrate that image-based 3D data sets of fluorescently tagged structures in human induced pluripotent stem cells (hiPSC), generated by a scalable and reproducible imaging pipeline, identifies signature profiles for a range of well-characterized small molecules and can be used to generate a predictive model of the dynamic organization and behavior of cells. These unique data can be used to train predictive models to identify the effects of perturbing target pathways, ascertain “off-target” effects and the mode of action of unknown compounds, and identify likely pathways influenced by mutations. By building complete combinations of image-based observations of many structures/lines in the presence of a large number of standardized biochemical perturbations, a comprehensive database of drug signatures on hiPS cells in their normal, pathological and regenerative (developmental) states can be generated.

To generate the predictive model, the resulting imaging data from each compound per stably tagged stem cell clone or differentiated cell derived therefrom, can be compared to the negative controls (untreated and vehicle controls) to determine effect on various criteria including cell and subcellular morphology, localization of tagged structure, and dynamics. By testing each compound in multiple gene edited iPSC lines (where each line has one structure tagged with GFP), the effect of that compound on multiple structures can be assessed within the cell. First, the intended effect of each compound with the relevant gene edited cell line can be confirmed as described in the assays above. The effect of that compound on all other structures can be assessed using the suite of gene edited iPSC lines to create a unique “fingerprint” or signature for that compound in relation to multiple structures. The data generated with these established set of compounds can be used as an initial training set for assays with compounds with unknown function. These profiles can serve as a reference database that can be used for screening novel and previously uncharacterized compound libraries to identify targets, help guide mechanistic studies, and determine specificity. Additionally, the combination of using human, diploid, non-transformed cells with live imaging using these gene edited iPSCs can provide a much better platform for performing toxicology screening. Further, these predictive models based on the stem cells and stably tagged stem cell clones and differentiated cells therefrom of the present invention can be used for screening, observing cellular dysplasia, disease staging, monitoring disease progression or improvement or cellular stress in response to a test agent.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A method for producing a stem cell comprising at least one tagged endogenous protein comprising:
(a) providing a ribonucleoprotein (RNP) complex comprising a Cas protein, a CRISPR RNA (crRNA) and a trans-activating RNA (tracrRNA), wherein the crRNA is specific for a target genomic locus and wherein the crRNA and the tracrRNA are separate RNA molecules;
(b) providing a donor plasmid comprising a first polynucleotide sequence encoding a detectable tag, a second polynucleotide sequence encoding a 5′ homology arm, and a third polynucleotide sequence encoding a 3′ homology arm, wherein the 5′ homology arm and 3′ homology arms are at least about 1 kb in length; and
(c) transfecting the complex of (a) and the donor plasmid of (b) into a stem cell such that the polynucleotide sequence encoding the detectable tag is inserted into a target genomic locus to generate a tagged endogenous protein, thereby producing a stem cell comprising at least one tagged endogenous protein.

2. The method of claim 1, wherein the polynucleotide sequence encoding the detectable tag further comprises a polynucleotide sequence encoding a flexible linker.

3-6. (canceled)

7. The method of claim 1, wherein the detectable tag is a fluorescent protein, a luminescent protein, a photoactivatable protein, a FLAG tag, a SNAP tag, or a Halo tag.

8. The method of claim 7, wherein the fluorescent protein is selected from the group comprising green fluorescent protein (GFP), blue fluorescent protein, cyan fluorescent protein, yellow fluorescent protein, or red fluorescent protein.

9. The method of claim 1, wherein the RNP comprises a crRNA, tracrRNA, and Cas9 protein complexed at a ratio of 1:1:1.

10. The method of claim 1, wherein the Cas protein is a wild-type Cas9 protein or a Cas9-nickase protein.

11. The method of claim 1, wherein the crRNA sequence is selected to minimize off-target cleavage of genomic DNA sequences and/or insertion of the detectable tag.

12. The method of claim 11, wherein the off-target cleavage of genomic DNA sequences and/or insertion of the detectable tag is less than 1.0%.

13. The method of claim 1, wherein transfecting the CRISPR/Cas9 RNP and the donor plasmid into a stem cell results in a double stranded break at the target genomic locus.

14. The method of claim 13, wherein the double stranded break is repaired by homology directed repair (HDR).

15. The method of claim 14, wherein the polynucleotides encoding 5′ homology arm, the detectable tag, and the 3′ homology arm act as a repair template during HDR.

16. The method of claim 1, wherein protospacer adjacent motif (PAM) sequences are removed from the polynucleotide backbone of the donor plasmid.

17. The method of claim 1, wherein the donor plasmid further comprises an antibiotic-resistance gene.

18. The method of claim 17, wherein the antibiotic-resistance gene confers resistance to ampicillin and/or kanamycin.

19. The method of claim 1, wherein the stem cell is an induced pluripotent stem cell (iPSC) derived from a healthy donor.

20. The method of claim 19, wherein the iPSC is a WTC cell or a WTB cell.

21. The method of claim 1, wherein transfecting the CRISPR/Cas9 RNP and the donor plasmid occurs by electroporating the stem cells.

22-26. (canceled)

27. The method of claim 1, wherein the target genomic locus is a locus within a gene encoding a structural protein.

28. The method of claim 27, wherein the structural protein is selected from paxillin, alpha tubulin, lamin B1, Tom20, desmoplakin, beta actin, Sec61B, fibrillarin, myosin, centrin2, ZO-1, Safe-harbor-GFP, ST6Gal1, vimentin, LAMP1, LC3, Safe harbor-CAAX, and PMP34.

29. The method of claim 1, wherein a plurality of detectable tags are inserted into a plurality of target loci.

30. The method of claim 29, wherein a plurality polynucleotides encoding a plurality of detectable tags are inserted into one donor plasmid.

31. The method of claim 30, wherein two or more polynucleotides encoding two or more detectable tags are inserted into one donor plasmid.

32. The method of claim 30, wherein a first plurality of polynucleotides encoding two or more detectable tags are inserted into a first donor plasmid and a second plurality of polynucleotides encoding two or more detectable tags are inserted into a second donor plasmid.

33. The method of claim 29, wherein a first polynucleotide encoding a first detectable tag is inserted into a first donor plasmid and a second polynucleotide encoding a second detectable tag is inserted into a second donor plasmid.

34-35. (canceled)

36. The method of claim 32, further comprising about 10 polynucleotides each encoding a unique detectable tag and each inserted into one of about 10 different donor plasmids.

37. The method of claim 36, wherein the one of about 10 different donor plasmids are introduced to the cell at the same time.

38. The method of claim 36, wherein one of about 10 different donor plasmids are introduced to the cell sequentially.

39-43. (canceled)

44. A method for producing a stable stem cell comprising at least one tagged endogenous protein comprising:
(a) providing a ribonucleoprotein (RNP) complex comprising a Cas protein, a CRISPR RNA (crRNA) and a trans-activating RNA (tracrRNA), wherein the crRNA is specific for a target genomic locus and wherein the crRNA and the tracrRNA are separate RNA molecules;
(b) providing a donor plasmid comprising a first polynucleotide sequence encoding a detectable tag, a second polynucleotide sequence encoding a 5′ homology arm, and a third polynucleotide sequence encoding a 3′ homology arm, wherein the 5′ homology arm and 3′ homology arms are at least about 1 kb in length; and
(c) transfecting the complex of (a) and the donor plasmid of (b) into a stem cell such that the polynucleotide sequence encoding the detectable tag is inserted into a target genomic locus to generate a tagged endogenous protein,
thereby producing a stem cell comprising at least one tagged endogenous protein, wherein the stably tagged stem cell:
includes mono- or bi-allelic insertion of the first polynucleotide sequence encoding a detectable tag into the target genomic locus,
is able to differentiate into all three germ layers; and
lacks additional mutations or alterations in the stem cell's endogenous genome.

45-48. (canceled)

49. The method of claim 44, wherein the stem cell comprising at least one tagged protein expresses at least one protein associated with pluripotency.

50. The method of claim 49, wherein the protein associated with pluripotency is selected from the group comprising Oct3/4, Sox2, Nanog, Tra-160, and Tra-181, SSEA3/4.

51. The method of claim 49, wherein expression level of the at least one protein associated with pluripotency is comparable to the expression level of the same protein in an unmodified stem cell.

52. The method of claim 44, wherein the stem cell comprising at least one tagged protein maintains a differentiation potential that is comparable to an unmodified stem cell.

53. The method of claim 52, wherein the stem cell comprising at least one tagged protein is capable of differentiating into mesoderm, endoderm, or ectoderm.

54. The method of claim 53, wherein the expression of the at least one tagged protein is maintained in a differentiated cell derived from the stem cell comprising at least one tagged protein.

55. The method of claim 44, wherein the morphology, viability, potency, and endogenous cellular functions of the stem cells comprising at least one tagged protein and/or differentiated cells derived from stem cells comprising at least one tagged protein are not substantially changed compared to unmodified stem cells and differentiated cells thereof.

56. A method for screening the effects of one or more test agents on one or more cellular structures in one or more cell types comprising:
providing one or more cultures of one or more stem cells and/or differentiated cells derived therefrom produced by the method of claim 1, wherein the stem cells or differentiated cells derived therefrom comprise a tagged endogenous protein;
adding one or more test agent to one or more of the cultures;
assaying the culture at one or more time points before and/or after the addition of the one or more test agent; and
determining the effects of the one or more test agent on one or more cellular structures in the one or more cell types.

57-76. (canceled)

77. A method for visualizing a stem cell produced by the method of claim 1, comprising: (a) plating the stem cells on plates; and
(b) imaging the cells by microscope.

78-80. (canceled)

81. A donor polynucleotide comprises a first polynucleotide sequence encoding a detectable tag, a second polynucleotide sequence encoding a 5′ homology arm, and a third polynucleotide sequence encoding a 3′ homology arm, wherein the 5′ homology arm and 3′ homology arm are each about 1 kb in length.

82-97. (canceled)

98. A stably tagged stem cell clone comprising at least one tagged endogenous protein,
wherein the stably tagged stem cell clone:
includes mono- or bi-allelic insertion of the first polynucleotide sequence encoding a detectable tag into the target genomic locus,
is able to differentiate into all three germ layers; and
lacks additional mutations or alterations in the endogenous stem cell genome.

99. (canceled)

100. A method of generating a signature for a test agent comprising:
(a) admixing the test agent with one or more stably tagged stem cell clones of claim 98;
(b) detecting a response in the one or more stem cell clone;
(c) detecting a response in a control stem cell;
(d) detecting a difference in the response in the one or more stem cell clones from the control stem cell; and
(e) generating a data set of the difference in the response.

101-110. (canceled)
